SEQUENCE LISTING (118 sequences)

SEQ ID NO: 1 - DNA, 261 bp, Homo sapiens
atgattccagcagtggtcttgctcttactccttttggttgaacaagcagcggccctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacagtag

SEQ ID NO: 2 - DNA, 54 bp, Homo sapiens
atgattccagcagtggtcttgctcttactccttttggttgaacaagcagcggcc

SEQ ID NO: 3 - DNA, 15 bp, Homo sapiens
ctgggagagcctcag

SEQ ID NO: 4 - DNA, 63 bp, Homo sapiens
ctctgctatatcctggatgccatcctgtttctgtatggaattgtcctcaccctcctctactgt

SEQ ID NO: 5 - DNA, 126 bp, Homo sapiens
cgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 6 - DNA, 690 bp, Homo sapiens
atgcaggatgaagatggatacatcaccttaaatattaaaactcggaaaccagctctcatctccgttggctctgcatcctcctcctggtggcgtgtgatggctttgattctgctgatcctgtgcgtggggatggttgtcgggctggtggctctggggatttggtctgtcatgcagcgcaattacctacaaggtgagaatgaaaatcgcacaggaactctgcaacaattagcaaagcgcttctgtcaatatgtggtaaaacaatcagaactaaagggcactttcaaaggtcataaatgcagcccctgtgacacaaactggagatattatggagatagctgctatgggttcttcaggcacaacttaacatgggaagagagtaagcagtactgcactgacatgaatgctactctcctgaagattgacaaccggaacattgtggagtacatcaaagccaggactcatttaattcgttgggtcggattatctcgccagaagtcgaatgaggtctggaagtgggaggatggctcggttatctcagaaaatatgtttgagtttttggaagatggaaaaggaaatatgaattgtgcttattttcataatgggaaaatgcaccctaccttctgtgagaacaaacattatttaatgtgtgagaggaaggctggcatgaccaaggtggaccaactaccttaa

SEQ ID NO: 7 - DNA, 99 bp, Homo sapiens
atgcaggatgaagatggatacatcaccttaaatattaaaactcggaaaccagctctcatctccgttggctctgcatcctcctcctggtggcgtgtgatg

SEQ ID NO: 8 - DNA, 63 bp, Homo sapiens
gctttgattctgctgatcctgtgcgtggggatggttgtcgggctggtggctctggggatttgg

SEQ ID NO: 9 - DNA, 525 bp, Homo sapiens
tctgtcatgcagcgcaattacctacaaggtgagaatgaaaatcgcacaggaactctgcaacaattagcaaagcgcttctgtcaatatgtggtaaaacaatcagaactaaagggcactttcaaaggtcataaatgcagcccctgtgacacaaactggagatattatggagatagctgctatgggttcttcaggcacaacttaacatgggaagagagtaagcagtactgcactgacatgaatgctactctcctgaagattgacaaccggaacattgtggagtacatcaaagccaggactcatttaattcgttgggtcggattatctcgccagaagtcgaatgaggtctggaagtgggaggatggctcggttatctcagaaaatatgtttgagtttttggaagatggaaaaggaaatatgaattgtgcttattttcataatgggaaaatgcaccctaccttctgtgagaacaaacattatttaatgtgtgagaggaaggctggcatgaccaaggtggaccaactacct

SEQ ID NO: 10 - DNA, 954 bp, Homo sapiens
atgactatggagacccaaatgtctcagaatgtatgtcccagaaacctgtggctgcttcaaccattgacagttttgctgctgctggcttctgcagacagtcaagctgcagctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 11 - DNA, 99 bp, Homo sapiens
atgactatggagacccaaatgtctcagaatgtatgtcccagaaacctgtggctgcttcaaccattgacagttttgctgctgctggcttctgcagacagt

SEQ ID NO: 12 - DNA, 552 bp, Homo sapiens
caagctgcagctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatgggg

SEQ ID NO: 13 - DNA, 69 bp, Homo sapiens
atcattgtggctgtggtcattgcgactgctgtagcagccattgttgctgctgtagtggccttgatctac

SEQ ID NO: 14 - DNA, 231 bp, Homo sapiens
tgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataac

SEQ ID NO: 15 - DNA, 1020 bp, Homo sapiens
atgtctccatccccgaccgccctcttctgtcttgggctgtgtctggggcgtgtgccagcgcagagtggaccgctccccaagccctccctccaggctctgcccagctccctggtgcccctggagaagccagtgaccctccggtgccagggacctccgggcgtggacctgtaccgcctggagaagctgagttccagcaggtaccaggatcaggcagtcctcttcatcccggccatgaagagaagtctggctggacgctaccgctgctcctaccagaacggaagcctctggtccctgcccagcgaccagctggagctcgttgccacgggagtttttgccaaaccctcgctctcagcccagcccggcccggcggtgtcgtcaggaggggacgtaaccctacagtgtcagactcggtatggctttgaccaatttgctctgtacaaggaaggggaccctgcgccctacaagaatcccgagagatggtacagggctagttttcccatcatcacggtgaccgccgcccacagcggaacctaccgatgctacagcttctccagcagggacccatacctgtggtcagcccccagcgaccccctggagcttgtggtcacaggaacctctgtgacccccagccggttaccaacagaaccaccttccccggtagcagaattctcagaagccaccgctgaactgaccgtctcattcacaaacgaagtcttcacaactgagacttctaggagtatcaccgccagtccaaaggagtcagactctccagctggtcctgcccgccagtactacaccaagggcaacctggtccggatatgcctcggggctgtgatcctaataatcctggcggggtttctggcagaggactggcacagccggaggaagcgcctgcggcacaggggcagggctgtgcagaggccgcttccgcccctcccgcccctcccgctgacccggaaatcaaacgggggtcaggatggaggccgacaggatgttcacagccgcgggttatgttcatga

SEQ ID NO: 16 - DNA, 60 bp, Homo sapiens
atgtctccatccccgaccgccctcttctgtcttgggctgtgtctggggcgtgtgccagcg

SEQ ID NO: 17 - DNA, 741 bp, Homo sapiens
cagagtggaccgctccccaagccctccctccaggctctgcccagctccctggtgcccctggagaagccagtgaccctccggtgccagggacctccgggcgtggacctgtaccgcctggagaagctgagttccagcaggtaccaggatcaggcagtcctcttcatcccggccatgaagagaagtctggctggacgctaccgctgctcctaccagaacggaagcctctggtccctgcccagcgaccagctggagctcgttgccacgggagtttttgccaaaccctcgctctcagcccagcccggcccggcggtgtcgtcaggaggggacgtaaccctacagtgtcagactcggtatggctttgaccaatttgctctgtacaaggaaggggaccctgcgccctacaagaatcccgagagatggtacagggctagttttcccatcatcacggtgaccgccgcccacagcggaacctaccgatgctacagcttctccagcagggacccatacctgtggtcagcccccagcgaccccctggagcttgtggtcacaggaacctctgtgacccccagccggttaccaacagaaccaccttccccggtagcagaattctcagaagccaccgctgaactgaccgtctcattcacaaacgaagtcttcacaactgagacttctaggagtatcaccgccagtccaaaggagtcagactctccagctggtcctgcccgccagtactacaccaag

SEQ ID NO: 18 - DNA, 63 bp, Homo sapiens
ggcaacctggtccggatatgcctcggggctgtgatcctaataatcctggcggggtttctggca

SEQ ID NO: 19 - DNA, 153 bp, Homo sapiens
gaggactggcacagccggaggaagcgcctgcggcacaggggcagggctgtgcagaggccgcttccgcccctcccgcccctcccgctgacccggaaatcaaacgggggtcaggatggaggccgacaggatgttcacagccgcgggttatgttca

SEQ ID NO: 20 - DNA, 1032 bp, Artificial Sequence
  Description of Artificial Sequence Chimeric ITAM receptor
atgattccagcagtggtcttgctcttactccttttggttgaacaagcagcggccgacatccagatgacccagaccaccagcagcctgagcgccagcctgggcgacagagtgaccatcagctgcagagccagccaggacatcagcaagtacctgaactggtaccagcagaagcccgacggcaccgtgaagctgctgatctaccacaccagcagactgcacagcggcgtgcccagcagattcagcggcagcggcagcggcaccgactacagcctgaccatcagcaacctggagcaggaggacatcgccacctacttctgccagcagggcaacaccctgccctacaccttcggcggcggcaccaagctggagatcaccggcagcaccagcggcagcggcaagcccggcagcggcgagggcagcaccaagggcgaggtgaagctgcaggagagcggccccggcctggtggcccccagccagagcctgagcgtgacctgcaccgtgagcggcgtgagcctgcccgactacggcgtgagctggatcagacagccccccagaaagggcctggagtggctgggcgtgatctggggcagcgagaccacctactacaacagcgccctgaagagcagactgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacagtag

SEQ ID NO: 21 - DNA, 951 bp, Artificial Sequence
  Description of Artificial Sequence Chimeric ITAM receptor
atgcaggatgaagatggatacatcaccttaaatattaaaactcggaaaccagctctcatctccgttggctctgcatcctcctcctggtggcgtgtgatggctttgattctgctgatcctgtgcgtggggatggttgtcgggctggtggctctggggatttggtctgtcatgcagcgcgaatctaagtacggaccgccctgccccccttgccctgacatccagatgacccagaccaccagcagcctgagcgccagcctgggcgacagagtgaccatcagctgcagagccagccaggacatcagcaagtacctgaactggtaccagcagaagcccgacggcaccgtgaagctgctgatctaccacaccagcagactgcacagcggcgtgcccagcagattcagcggcagcggcagcggcaccgactacagcctgaccatcagcaacctggagcaggaggacatcgccacctacttctgccagcagggcaacaccctgccctacaccttcggcggcggcaccaagctggagatcaccggcagcaccagcggcagcggcaagcccggcagcggcgagggcagcaccaagggcgaggtgaagctgcaggagagcggccccggcctggtggcccccagccagagcctgagcgtgacctgcaccgtgagcggcgtgagcctgcccgactacggcgtgagctggatcagacagccccccagaaagggcctggagtggctgggcgtgatctggggcagcgagaccacctactacaacagcgccctgaagagcagactgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagctaa

SEQ ID NO: 22 - DNA, 1188 bp, Artificial Sequence
  Description of Artificial Sequence Chimeric ITAM receptor
atgactatggagacccaaatgtctcagaatgtatgtcccagaaacctgtggctgcttcaaccattgacagttttgctgctgctggcttctgcagacagtgacatccagatgacccagaccaccagcagcctgagcgccagcctgggcgacagagtgaccatcagctgcagagccagccaggacatcagcaagtacctgaactggtaccagcagaagcccgacggcaccgtgaagctgctgatctaccacaccagcagactgcacagcggcgtgcccagcagattcagcggcagcggcagcggcaccgactacagcctgaccatcagcaacctggagcaggaggacatcgccacctacttctgccagcagggcaacaccctgccctacaccttcggcggcggcaccaagctggagatcaccggcagcaccagcggcagcggcaagcccggcagcggcgagggcagcaccaagggcgaggtgaagctgcaggagagcggccccggcctggtggcccccagccagagcctgagcgtgacctgcaccgtgagcggcgtgagcctgcccgactacggcgtgagctggatcagacagccccccagaaagggcctggagtggctgggcgtgatctggggcagcgagaccacctactacaacagcgccctgaagagcagactgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgcccttcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 23 - DNA, 1065 bp, Artificial Sequence
  Description of Artificial Sequence Chimeric ITAM receptor
atgtctccatccccgaccgccctcttctgtcttgggctgtgtctggggcgtgtgccagcggacatccagatgacccagaccaccagcagcctgagcgccagcctgggcgacagagtgaccatcagctgcagagccagccaggacatcagcaagtacctgaactggtaccagcagaagcccgacggcaccgtgaagctgctgatctaccacaccagcagactgcacagcggcgtgcccagcagattcagcggcagcggcagcggcaccgactacagcctgaccatcagcaacctggagcaggaggacatcgccacctacttctgccagcagggcaacaccctgccctacaccttcggcggcggcaccaagctggagatcaccggcagcaccagcggcagcggcaagcccggcagcggcgagggcagcaccaagggcgaggtgaagctgcaggagagcggccccggcctggtggcccccagccagagcctgagcgtgacctgcaccgtgagcggcgtgagcctgcccgactacggcgtgagctggatcagacagccccccagaaagggcctggagtggctgggcgtgatctggggcagcgagaccacctactacaacagcgccctgaagagcagactgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctcagtactacaccaagggcaacctggtccggatatgcctcggggctgtgatcctaataatcctggcggggtttctggcagaggactggcacagccggaggaagcgcctgcggcacaggggcagggctgtgcagaggccgcttccgcccctcccgcccctcccgctgacccggaaatcaaacgggggtcaggatggaggccgacaggatgttcacagccgcgggttatgttcatga

SEQ ID NO: 24 - DNA, 1581 bp, Homo sapiens
atggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaa

SEQ ID NO: 25 - DNA, 102 bp, Homo sapiens
atggggcacctctcagccccacttcacagagtgcgtgtaccctggcaggggcttctgctcacagcctcacttctaaccttctggaacccgcccaccactgcc

SEQ ID NO: 26 - DNA, 1182 bp, Homo sapiens
cagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggg

SEQ ID NO: 27 - DNA, 72 bp, Homo sapiens
gccattgctggcattgtgattggagtagtggccctggttgctctgatagcagtagccctggcatgttttctg

SEQ ID NO: 28 - DNA, 222 bp, Homo sapiens
catttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttattcagaagtaaaaaagcag

SEQ ID NO: 29 - DNA, 726 bp, Homo sapiens
atggctgtgtttctgcagctgctaccgctgctgctctcgagggcccaagggaaccctggggcttctctggacggccgccctggggaccgggtgaatctctcctgcggaggagtctctcatcccatccgctgggtctgggcacccagcttcccggcctgcaagggcctgtccaaaggacgccgaccgatcctgtgggcctcttcgagcgggacccccaccgtgcctcccctccagcctttcgtcggccgcctacgctccctggactctggtatccggcggctggagctcctcttgagcgcgggggactcgggcacttttttctgcaagggccgccacgaggacgagagccgtacagtgcttcacgtgctgggggacaggacctattgcaaggcccccgggcctacccatgggtccgtgtatccccagctcctgatcccgctgctgggcgctgggttggtgctcggactgggagctttgggcctggtctggtggctgcacaggcgcctgcccccgcaaccgattcgaccactccctagatttgctccacttgtgaaaaccgagccccagaggccagtaaaggaggaagagcccaagattccaggggacctggaccaggaaccgagcctgctctatgcggatctggaccatctagccctcagcaggccccgccggctgtccacagcggaccctgctgatgcctccaccatctatgcagttgtagtttga

SEQ ID NO: 30 - DNA, 51 bp, Homo sapiens
atggctgtgtttctgcagctgctaccgctgctgctctcgagggcccaaggg

SEQ ID NO: 31 - DNA, 375 bp, Homo sapiens
aaccctggggcttctctggacggccgccctggggaccgggtgaatctctcctgcggaggagtctctcatcccatccgctgggtctgggcacccagcttcccggcctgcaagggcctgtccaaaggacgccgaccgatcctgtgggcctcttcgagcgggacccccaccgtgcctcccctccagcctttcgtcggccgcctacgctccctggactctggtatccggcggctggagctcctcttgagcgcgggggactcgggcacttttttctgcaagggccgccacgaggacgagagccgtacagtgcttcacgtgctgggggacaggacctattgcaaggcccccgggcctacccatgggtccgtgtatccccag

SEQ ID NO: 32 - DNA, 63 bp, Homo sapiens
ctcctgatcccgctgctgggcgctgggttggtgctcggactgggagctttgggcctggtctgg

SEQ ID NO: 33 - DNA, 234 bp, Homo sapiens
tggctgcacaggcgcctgcccccgcaaccgattcgaccactccctagatttgctccacttgtgaaaaccgagccccagaggccagtaaaggaggaagagcccaagattccaggggacctggaccaggaaccgagcctgctctatgcggatctggaccatctagccctcagcaggccccgccggctgtccacagcggaccctgctgatgcctccaccatctatgcagttgtagtt

SEQ ID NO: 34 - DNA, 1797 bp, Homo sapiens
atgacccccatcgtcacagtcctgatctgtctcgggctgagtctgggccccaggacccacgtgcagacagggaccattcccaagcccaccctgtgggctgagccagactctgtgatcacccaggggagtcccgtcaccctcagttgtcaggggagccttgaagcccaggagtaccgtctatatagggagaaaaaatcagcatcttggattacacggatacgaccagagcttgtgaagaacggccagttccacatcccatccatcacctgggaacacacagggcgatatggctgtcagtattacagccgcgctcggtggtctgagctcagtgaccccctggtgctggtgatgacaggagcctacccaaaacccaccctctcagcccagcccagccctgtggtgacctcaggaggaagggtgaccctccagtgtgagtcacaggtggcatttggcggcttcattctgtgtaaggaaggagaagaagaacacccacaatgcctgaactcccagccccatgcccgtgggtcgtcccgcgccatcttctccgtgggccccgtgagcccgaatcgcaggtggtcgcacaggtgctatggttatgacttgaactctccctatgtgtggtcttcacccagtgatctcctggagctcctggtcccaggtgtttctaagaagccatcactctcagtgcagccgggtcctgtcgtggcccctggggaaagcctgaccctccagtgtgtctctgatgtcggctatgacagatttgttctgtacaaggagggggaacgtgaccttcgccagctccctggccggcagccccaggctgggctctcccaggccaacttcaccctgggccctgtgagccgctcctacgggggccagtacagatgctacggtgcacacaacctctcctctgagtgctcggcccccagcgaccccctggacatcctgatcacaggacagatccgtggcacacccttcatctcagtgcagccaggccccacagtggcctcaggagagaacgtgaccctgctgtgtcagtcatggcggcagttccacactttccttctgaccaaggcgggagcagctgatgccccactccgtctaagatcaatacacgaatatcctaagtaccaggctgaattccccatgagtcctgtgacctcagcccacgcggggacctacaggtgctacggctcactcaactccgacccctacctgctgtctcaccccagtgagcccctggagctcgtggtctcaggaccctccatgggttccagccccccacccaccggtcccatctccacacctgcaggccctgaggaccagcccctcacccccactgggtcggatccccaaagtggtctgggaaggcacctgggggttgtgatcggcatcttggtggccgtcgtcctactgctcctcctcctcctcctcctcttcctcatcctccgacatcgacgtcagggcaaacactggacatcgacccagagaaaggctgatttccaacatcctgcaggggctgtggggccagagcccacagacagaggcctgcagtggaggtccagcccagctgccgacgcccaggaagaaaacctctatgctgccgtgaaggacacacagcctgaagatggggtggagatggacactcgggctgctgcatctgaagccccccaggatgtgacctacgcccagctgcacagcttgaccctcagacggaaggcaactgagcctcctccatcccaggaaagggaacctccagctgagcccagcatctacgccaccctggccatccactag

SEQ ID NO: 35 - DNA, 63 bp, Homo sapiens
atgacccccatcgtcacagtcctgatctgtctcgggctgagtctgggccccaggacccacgtg

SEQ ID NO: 36 - DNA, 1320 bp, Homo sapiens
cagacagggaccattcccaagcccaccctgtgggctgagccagactctgtgatcacccaggggagtcccgtcaccctcagttgtcaggggagccttgaagcccaggagtaccgtctatatagggagaaaaaatcagcatcttggattacacggatacgaccagagcttgtgaagaacggccagttccacatcccatccatcacctgggaacacacagggcgatatggctgtcagtattacagccgcgctcggtggtctgagctcagtgaccccctggtgctggtgatgacaggagcctacccaaaacccaccctctcagcccagcccagccctgtggtgacctcaggaggaagggtgaccctccagtgtgagtcacaggtggcatttggcggcttcattctgtgtaaggaaggagaagaagaacacccacaatgcctgaactcccagccccatgcccgtgggtcgtcccgcgccatcttctccgtgggccccgtgagcccgaatcgcaggtggtcgcacaggtgctatggttatgacttgaactctccctatgtgtggtcttcacccagtgatctcctggagctcctggtcccaggtgtttctaagaagccatcactctcagtgcagccgggtcctgtcgtggcccctggggaaagcctgaccctccagtgtgtctctgatgtcggctatgacagatttgttctgtacaaggagggggaacgtgaccttcgccagctccctggccggcagccccaggctgggctctcccaggccaacttcaccctgggccctgtgagccgctcctacgggggccagtacagatgctacggtgcacacaacctctcctctgagtgctcggcccccagcgaccccctggacatcctgatcacaggacagatccgtggcacacccttcatctcagtgcagccaggccccacagtggcctcaggagagaacgtgaccctgctgtgtcagtcatggcggcagttccacactttccttctgaccaaggcgggagcagctgatgccccactccgtctaagatcaatacacgaatatcctaagtaccaggctgaattccccatgagtcctgtgacctcagcccacgcggggacctacaggtgctacggctcactcaactccgacccctacctgctgtctcaccccagtgagcccctggagctcgtggtctcaggaccctccatgggttccagccccccacccaccggtcccatctccacacctgcaggccctgaggaccagcccctcacccccactgggtcggatccccaaagtggtctgggaaggcacctgggggtt

SEQ ID NO: 37 - DNA, 63 bp, Homo sapiens
gtgatcggcatcttggtggccgtcgtcctactgctcctcctcctcctcctcctcttcctcatc

SEQ ID NO: 38 - DNA, 2217 bp, Homo sapiens
atgcagccgaggtgggcccaaggggccacgatgtggcttggagtcctgctgacccttctgctctgttcaagccttgagggtcaagaaaactctttcacaatcaacagtgttgacatgaagagcctgccggactggacggtgcaaaatgggaagaacctgaccctgcagtgcttcgcggatgtcagcaccacctctcacgtcaagcctcagcaccagatgctgttctataaggatgacgtgctgttttacaacatctcctccatgaagagcacagagagttattttattcctgaagtccggatctatgactcagggacatataaatgtactgtgattgtgaacaacaaagagaaaaccactgcagagtaccaggtgttggtggaaggagtgcccagtcccagggtgacactggacaagaaagaggccatccaaggtgggatcgtgagggtcaactgttctgtcccagaggaaaaggccccaatacacttcacaattgaaaaacttgaactaaatgaaaaaatggtcaagctgaaaagagagaagaattctcgagaccagaattttgtgatactggaattccccgttgaggaacaggaccgcgttttatccttccgatgtcaagctaggatcatttctgggatccatatgcagacctcagaatctaccaagagtgaactggtcaccgtgacggaatccttctctacacccaagttccacatcagccccaccggaatgatcatggaaggagctcagctccacattaagtgcaccattcaagtgactcacctggcccaggagtttccagaaatcataattcagaaggacaaggcgattgtggcccacaacagacatggcaacaaggctgtgtactcagtcatggccatggtggagcacagtggcaactacacgtgcaaagtggagtccagccgcatatccaaggtcagcagcatcgtggtcaacataacagaactattttccaagcccgaactggaatcttccttcacacatctggaccaaggtgaaagactgaacctgtcctgctccatcccaggagcacctccagccaacttcaccatccagaaggaagatacgattgtgtcacagactcaagatttcaccaagatagcctcaaagtcggacagtgggacgtatatctgcactgcaggtattgacaaagtggtcaagaaaagcaacacagtccagatagtcgtatgtgaaatgctctcccagcccaggatttcttatgatgcccagtttgaggtcataaaaggacagaccatcgaagtccgttgcgaatcgatcagtggaactttgcctatttcttaccaacttttaaaaacaagtaaagttttggagaatagtaccaagaactcaaatgatcctgcggtattcaaagacaaccccactgaagacgtcgaataccagtgtgttgcagataattgccattcccatgccaaaatgttaagtgaggttctgagggtgaaggtgatagccccggtggatgaggtccagatttctatcctgtcaagtaaggtggtggagtctggagaggacattgtgctgcaatgtgctgtgaatgaaggatctggtcccatcacctataagttttacagagaaaaagagggcaaacccttctatcaaatgacctcaaatgccacccaggcattttggaccaagcagaaggctagcaaggaacaggagggagagtattactgcacagccttcaacagagccaaccacgcctccagtgtccccagaagcaaaatactgacagtcagagtcattcttgccccatggaagaaaggacttattgcagtggttatcatcggagtgatcattgctctcttgatcattgcggccaaatgttattttctgaggaaagccaaggccaagcagatgccagtggaaatgtccaggccagcagtaccacttctgaactccaacaacgagaaaatgtcagatcccaatatggaagctaacagtcattacggtcacaatgacgatgtcagaaaccatgcaatgaaaccaataaatgataataaagagcctctgaactcagacgtgcagtacacggaagttcaagtgtcctcagctgagtctcacaaagatctaggaaagaaggacacagagacagtgtacagtgaagtccggaaagctgtccctgatgccgtggaaagcagatactctagaacggaaggctcccttgatggaacttag

SEQ ID NO: 39 - DNA, 81 bp, Homo sapiens
atgcagccgaggtgggcccaaggggccacgatgtggcttggagtcctgctgacccttctgctctgttcaagccttgagggt

SEQ ID NO: 40 - DNA, 1722 bp, Homo sapiens
caagaaaactctttcacaatcaacagtgttgacatgaagagcctgccggactggacggtgcaaaatgggaagaacctgaccctgcagtgcttcgcggatgtcagcaccacctctcacgtcaagcctcagcaccagatgctgttctataaggatgacgtgctgttttacaacatctcctccatgaagagcacagagagttattttattcctgaagtccggatctatgactcagggacatataaatgtactgtgattgtgaacaacaaagagaaaaccactgcagagtaccaggtgttggtggaaggagtgcccagtcccagggtgacactggacaagaaagaggccatccaaggtgggatcgtgagggtcaactgttctgtcccagaggaaaaggccccaatacacttcacaattgaaaaacttgaactaaatgaaaaaatggtcaagctgaaaagagagaagaattctcgagaccagaattttgtgatactggaattccccgttgaggaacaggaccgcgttttatccttccgatgtcaagctaggatcatttctgggatccatatgcagacctcagaatctaccaagagtgaactggtcaccgtgacggaatccttctctacacccaagttccacatcagccccaccggaatgatcatggaaggagctcagctccacattaagtgcaccattcaagtgactcacctggcccaggagtttccagaaatcataattcagaaggacaaggcgattgtggcccacaacagacatggcaacaaggctgtgtactcagtcatggccatggtggagcacagtggcaactacacgtgcaaagtggagtccagccgcatatccaaggtcagcagcatcgtggtcaacataacagaactattttccaagcccgaactggaatcttccttcacacatctggaccaaggtgaaagactgaacctgtcctgctccatcccaggagcacctccagccaacttcaccatccagaaggaagatacgattgtgtcacagactcaagatttcaccaagatagcctcaaagtcggacagtgggacgtatatctgcactgcaggtattgacaaagtggtcaagaaaagcaacacagtccagatagtcgtatgtgaaatgctctcccagcccaggatttcttatgatgcccagtttgaggtcataaaaggacagaccatcgaagtccgttgcgaatcgatcagtggaactttgcctatttcttaccaacttttaaaaacaagtaaagttttggagaatagtaccaagaactcaaatgatcctgcggtattcaaagacaaccccactgaagacgtcgaataccagtgtgttgcagataattgccattcccatgccaaaatgttaagtgaggttctgagggtgaaggtgatagccccggtggatgaggtccagatttctatcctgtcaagtaaggtggtggagtctggagaggacattgtgctgcaatgtgctgtgaatgaaggatctggtcccatcacctataagttttacagagaaaaagagggcaaacccttctatcaaatgacctcaaatgccacccaggcattttggaccaagcagaaggctagcaaggaacaggagggagagtattactgcacagccttcaacagagccaaccacgcctccagtgtccccagaagcaaaatactgacagtcagagtcattcttgccccatggaagaaa

SEQ ID NO: 41 - DNA, 57 bp, Homo sapiens
ggacttattgcagtggttatcatcggagtgatcattgctctcttgatcattgcggcc

SEQ ID NO: 42 - DNA, 354 bp, Homo sapiens
aaatgttattttctgaggaaagccaaggccaagcagatgccagtggaaatgtccaggccagcagtaccacttctgaactccaacaacgagaaaatgtcagatcccaatatggaagctaacagtcattacggtcacaatgacgatgtcagaaaccatgcaatgaaaccaataaatgataataaagagcctctgaactcagacgtgcagtacacggaagttcaagtgtcctcagctgagtctcacaaagatctaggaaagaaggacacagagacagtgtacagtgaagtccggaaagctgtccctgatgccgtggaaagcagatactctagaacggaaggctcccttgatggaact

SEQ ID NO: 43 - DNA, 936 bp, Homo sapiens
atgggcctcaccctgctcttgctgctgctcctgggactagaaggtcagggcatagttggcagcctccctgaggtgctgcaggcacccgtgggaagctccattctggtgcagtgccactacaggctccaggatgtcaaagctcagaaggtgtggtgccggttcttgccggaggggtgccagcccctggtgtcctcagctgtggatcgcagagctccagcgggcaggcgtacgtttctcacagacctgggtgggggcctgctgcaggtggaaatggttaccctgcaggaagaggatgctggcgagtatggctgcatggtggatggggccagggggccccagattttgcacagagtctctctgaacatactgcccccagaggaagaagaagagacccataagattggcagtctggctgagaacgcattctcagaccctgcaggcagtgccaaccctttggaacccagccaggatgagaagagcatccccttgatctggggtgctgtgctcctggtaggtctgctggtggcagcggtggtgctgtttgctgtgatggccaagaggaaacaagggaacaggcttggtgtctgtggccgattcctgagcagcagagtttcaggcatgaatccctcctcagtggtccaccacgtcagtgactctggaccggctgctgaattgcctttggatgtaccacacattaggcttgactcaccaccttcatttgacaataccacctacaccagcctacctcttgattccccatcaggaaaaccttcactcccagctccatcctcattgccccctctacctcctaaggtcctggtctgctccaagcctgtgacatatgccacagtaatcttcccgggagggaacaagggtggagggacctcgtgtgggccagcccagaatccacctaacaatcagactccatccagctaa

SEQ ID NO: 44 - DNA, 45 bp, Homo sapiens
atgggcctcaccctgctcttgctgctgctcctgggactagaaggt

SEQ ID NO: 45 - DNA, 441 bp, Homo sapiens
cagggcatagttggcagcctccctgaggtgctgcaggcacccgtgggaagctccattctggtgcagtgccactacaggctccaggatgtcaaagctcagaaggtgtggtgccggttcttgccggaggggtgccagcccctggtgtcctcagctgtggatcgcagagctccagcgggcaggcgtacgtttctcacagacctgggtgggggcctgctgcaggtggaaatggttaccctgcaggaagaggatgctggcgagtatggctgcatggtggatggggccagggggccccagattttgcacagagtctctctgaacatactgcccccagaggaagaagaagagacccataagattggcagtctggctgagaacgcattctcagaccctgcaggcagtgccaaccctttggaacccagccaggatgagaagagcatcccc

SEQ ID NO: 46 - DNA, 63 bp, Homo sapiens
ttgatctggggtgctgtgctcctggtaggtctgctggtggcagcggtggtgctgtttgctgtg

SEQ ID NO: 47 - DNA, 384 bp, Homo sapiens
atggccaagaggaaacaagggaacaggcttggtgtctgtggccgattcctgagcagcagagtttcaggcatgaatccctcctcagtggtccaccacgtcagtgactctggaccggctgctgaattgcctttggatgtaccacacattaggcttgactcaccaccttcatttgacaataccacctacaccagcctacctcttgattccccatcaggaaaaccttcactcccagctccatcctcattgccccctctacctcctaaggtcctggtctgctccaagcctgtgacatatgccacagtaatcttcccgggagggaacaagggtggagggacctcgtgtgggccagcccagaatccacctaacaatcagactccatccagc

SEQ ID NO: 48 - DNA, 360 bp, Homo sapiens
gaggtgaagctgcaggagagcggccccggcctggtggcccccagccagagcctgagcgtgacctgcaccgtgagcggcgtgagcctgcccgactacggcgtgagctggatcagacagccccccagaaagggcctggagtggctgggcgtgatctggggcagcgagaccacctactacaacagcgccctgaagagcagactgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 49 - DNA, 54 bp, Homo sapiens
ggcagcaccagcggcagcggcaagcccggcagcggcgagggcagcaccaagggc

SEQ ID NO: 50 - DNA, 321 bp, Homo sapiens
gacatccagatgacccagaccaccagcagcctgagcgccagcctgggcgacagagtgaccatcagctgcagagccagccaggacatcagcaagtacctgaactggtaccagcagaagcccgacggcaccgtgaagctgctgatctaccacaccagcagactgcacagcggcgtgcccagcagattcagcggcagcggcagcggcaccgactacagcctgaccatcagcaacctggagcaggaggacatcgccacctacttctgccagcagggcaacaccctgccctacaccttcggcggcggcaccaagctggagatcacc

SEQ ID NO: 51 - DNA, 36 bp, Homo sapiens
gaatctaagtacggaccgccctgccccccttgccct

SEQ ID NO: 52 - DNA, 348 bp, Homo sapiens
ctccgacatcgacgtcagggcaaacactggacatcgacccagagaaaggctgatttccaacatcctgcaggggctgtggggccagagcccacagacagaggcctgcagtggaggtccagcccagctgccgacgcccaggaagaaaacctctatgctgccgtgaaggacacacagcctgaagatggggtggagatggacactcgggctgctgcatctgaagccccccaggatgtgacctacgcccagctgcacagcttgaccctcagacggaaggcaactgagcctcctccatcccaggaaagggaacctccagctgagcccagcatctacgccaccctggccatccac

SEQ ID NO: 53 - DNA, 15 bp, Homo sapiens
tctgtcatgcagcgc

SEQ ID NO: 54 - DNA, 15 bp, Homo sapiens
tcttcaccaatgggg

SEQ ID NO: 55 - DNA, 15 bp, Homo sapiens
cagtactacaccaag

SEQ ID NO: 56 - DNA, 20 bp, Artificial Sequence
  grna1_HPS1_1r
ggggtgaatcagtcgctcca

SEQ ID NO: 57 - DNA, 20 bp, Artificial Sequence
  grna2_HPS1_2
gtcaacaccagccccgagcg

SEQ ID NO: 58 - DNA, 20 bp, Artificial Sequence
  grna3_HPS1_3
gctggagcggcacgtcatcc

SEQ ID NO: 59 - DNA, 20 bp, Artificial Sequence
  grna4_HPS1_4r
cttggagtgcacgagcagga

SEQ ID NO: 60 - DNA, 20 bp, Artificial Sequence
  grna5_ITGA2B_1r
cagtagccgtcgaagtactc

SEQ ID NO: 61 - DNA, 20 bp, Artificial Sequence
  grna6_ITGA2B_2
attttctcgagttaccgccc

SEQ ID NO: 62 - DNA, 20 bp, Artificial Sequence
  grna7_ITGA2B_3r
ctcgagaaaatatccgcaac

SEQ ID NO: 63 - DNA, 20 bp, Artificial Sequence
  grna8_ITGA2B_4r
gggaggacacgtgccacaaa

SEQ ID NO: 64 - DNA, 20 bp, Artificial Sequence
  grna9_GP6_1
gggcgtggacctgtaccgcc

SEQ ID NO: 65 - DNA, 20 bp, Artificial Sequence
  grna10_GP6_2r
acgagctccagctggtcgct

SEQ ID NO: 66 - DNA, 20 bp, Artificial Sequence
  grna11_GP6_3r
cggaggtccctggcaccgga

SEQ ID NO: 67 - DNA, 20 bp, Artificial Sequence
  grna12_GP6_4
ccagtgaccctccggtgcca

SEQ ID NO: 68 - DNA, 20 bp, Artificial Sequence
  grna13_Par1_1r
ggagctggtcaaatatccgg

SEQ ID NO: 69 - DNA, 20 bp, Artificial Sequence
  grna14_Par1_2r
ttcctgagaagaaatgaccg

SEQ ID NO: 70 - DNA, 20 bp, Artificial Sequence
  grna15_Par1_3r
acactccggtgtacacagat

SEQ ID NO: 71 - DNA, 20 bp, Artificial Sequence
  grna16_Par1_4r
acgatggccatgatgtttag

SEQ ID NO: 72 - DNA, 20 bp, Artificial Sequence
  grna17_Par4_1r
acttggcctgggtagccgcg

SEQ ID NO: 73 - DNA, 20 bp, Artificial Sequence
  grna18_Par4_2
ggtgcccgccctctatgggc

SEQ ID NO: 74 - DNA, 20 bp, Artificial Sequence
  grna19_Par4_3
tggtggggctgccggccaat

SEQ ID NO: 75 - DNA, 20 bp, Artificial Sequence
  grna20_Par4_4r
agcagtgcccgtgagctgtc

SEQ ID NO: 76 - DNA, 20 bp, Artificial Sequence
  grna21_Cox1_1
acttctggcaagatgggtcc

SEQ ID NO: 77 - DNA, 20 bp, Artificial Sequence
  grna22_Cox1_2
tcaccaaggccttgggccat

SEQ ID NO: 78 - DNA, 20 bp, Artificial Sequence
  grna23_Cox1_3r
tgtctccataaatgtggccg

SEQ ID NO: 79 - DNA, 20 bp, Artificial Sequence
  grna24_Cox1_4
aactgcggctctttaaggat

SEQ ID NO: 80 - DNA, 20 bp, Artificial Sequence
  grna29_P2Y12_1r
gtagtctctggtgcacagac

SEQ ID NO: 81 - DNA, 20 bp, Artificial Sequence
  grna30_P2Y12_2r
gaaagaaaatcctcatcgcc

SEQ ID NO: 82 - DNA, 20 bp, Artificial Sequence
  grna31_P2Y12_3
attcttagtgatgccaaact

SEQ ID NO: 83 - DNA, 20 bp, Artificial Sequence
  grna32_P2Y12_4r
gatcgatagttatcagtccc

SEQ ID NO: 84 - DNA, 20 bp, Artificial Sequence
  grna40_B2M_1r
aagtcaacttcaatgtcgga

SEQ ID NO: 85 - DNA, 20 bp, Artificial Sequence
  grna41_B2M_2r
agtcacatggttcacacggc

SEQ ID NO: 86 - DNA, 20 bp, Artificial Sequence
  grna42_B2M_3
acttgtctttcagcaaggac

SEQ ID NO: 87 - DNA, 20 bp, Artificial Sequence
  grna43_B2M_4
tcacgtcatccagcagagaa

SEQ ID NO: 88 - DNA, 20 bp, Artificial Sequence
  RocO1_sHPS1_F1
atctggtgcagagtccaagc

SEQ ID NO: 89 - DNA, 20 bp, Artificial Sequence
  RocO2_sHPS1_R1
tggaggaggtgattcttggc

SEQ ID NO: 90 - DNA, 20 bp, Artificial Sequence
  RocO3_ITGA2B_F1
ggctcctggcggctattatt

SEQ ID NO: 91 - DNA, 18 bp, Artificial Sequence
  RocO4_ITGA2B_R1
cttaggcggtgggttggc

SEQ ID NO: 92 - DNA, 16 bp, Artificial Sequence
  RocO5_GP6_F1
agcagcggggtccagg

SEQ ID NO: 93 - DNA, 16 bp, Artificial Sequence
  RocO6_GP6_R1
cgtggcaccaccaccc

SEQ ID NO: 94 - DNA, 25 bp, Artificial Sequence
  RocO7_Par1_F1
acccactctcctagtaagaaaacat

SEQ ID NO: 95 - DNA, 20 bp, Artificial Sequence
  RocO8_Par1_R1
caaactgccaatcactgccg

SEQ ID NO: 96 - DNA, 20 bp, Artificial Sequence
  RocO9_Par4_F1
atgtccagctgtttcccacc

SEQ ID NO: 97 - DNA, 18 bp, Artificial Sequence
  RocO10_Par4_R1
gcaggtggtaggcgatcc

SEQ ID NO: 98 - DNA, 20 bp, Artificial Sequence
  RocO11_Cox1_F1
ccaaccagggaagaagcagt

SEQ ID NO: 99 - DNA, 19 bp, Artificial Sequence
  RocO12_Cox1_R1
tggcacaagcttcccactc

SEQ ID NO: 100 - DNA, 20 bp, Artificial Sequence
  RocO15_P2Y12_F1
gaggaggctgtgtccaaaaa

SEQ ID NO: 101 - DNA, 20 bp, Artificial Sequence
  RocO16_P2Y12_R1
ggctgcctgttggtcagaat

SEQ ID NO: 102 - DNA, 20 bp, Artificial Sequence
  RocO58_B2M_F1
tgacaccaagttagccccaa

SEQ ID NO: 103 - DNA, 20 bp, Artificial Sequence
  RocO59_B2M_R1
gggatgggactcattcaggg

SEQ ID NO: 104 - protein, 514 aa, Artificial Sequence
  MART1_B2M_ HLA-A*0201_FCERG-TM_FCERG-CYTO
MIPAVVLLLLLLVEQAAAELAGIGILTVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPILGEPQLCYILDAILFLYGIVLTLLYCRLKIQVRKAAITSYEKSDGVYTGLSTRNQETYETLKHEKPPQ

SEQ ID NO: 105 - protein, 551 aa, Artificial Sequence
  MART1_B2M_ HLA-A*0201_FCGR2A-TM_FCGR2A-CYTO
MIPAVVLLLLLLVEQAAAELAGIGILTVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPISSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 106 - protein, 551 aa, Artificial Sequence
  MART1_B2M_ HLA-A*0201_HLA-TM_FCERG-CYTO
MIPAVVLLLLLLVEQAAAELAGIGILTVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSGGEGVKDRKGGSYTQAASSDSAQGSDVSLTACKVRLKIQVRKAAITSYEKSDGVYTGLSTRNQETYETLKHEKPPQ

SEQ ID NO: 107 - protein, 586 aa, Artificial Sequence
  MART1_B2M_ HLA-A*0201_HLA-TM_FCGR2A-CYTO
MIPAVVLLLLLLVEQAAAELAGIGILTVGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSGGEGVKDRKGGSYTQAASSDSAQGSDVSLTACKVCRKKRISANSTDPVKAAQFEPPGRQMIAIRKRQLEETNNDYETADGGYMTLNPRAPTDDDKNIYLTLPPNDHVNSNN

SEQ ID NO: 108 - protein, 513 aa, Artificial Sequence
  NYESO1_B2M_ HLA-A*0201_FCERG-TM_FCERG-CYTO
MIPAVVLLLLLLVEQAAASLLMWITQCGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPILGEPQLCYILDAILFLYGIVLTLLYCRLKIQVRKAAITSYEKSDGVYTGLSTRNQETYETLKHEKPPQ

SEQ ID NO: 109 - protein, 550 aa, Artificial Sequence
  NYESO1_B2M_ HLA-A*0201_FCGR2A-TM_FCGR2A-CYTO
MIPAVVLLLLLLVEQAAASLLMWITQCGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPISSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 110 - protein, 550 aa, Artificial Sequence
  NYESO1_B2M_ HLA-A*0201_HLA-TM_FCERG-CYTO
MIPAVVLLLLLLVEQAAASLLMWITQCGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSGGEGVKDRKGGSYTQAASSDSAQGSDVSLTACKVRLKIQVRKAAITSYEKSDGVYTGLSTRNQETYETLKHEKPPQ

SEQ ID NO: 111 - protein, 585 aa, Artificial Sequence
  NYESO1_B2M_ HLA-A*0201_HLA-TM_FCGR2A-CYTO
MIPAVVLLLLLLVEQAAASLLMWITQCGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSGGEGVKDRKGGSYTQAASSDSAQGSDVSLTACKVCRKKRISANSTDPVKAAQFEPPGRQMIAIRKRQLEETNNDYETADGGYMTLNPRAPTDDDKNIYLTLPPNDHVNSNN

SEQ ID NO: 112 - RNA, 20 nt, Artificial Sequence
  B2M_1
aagucaacuucaaugucgga

SEQ ID NO: 113 - RNA, 20 nt, Artificial Sequence
  B2M_2
agucacaugguucacacggc

SEQ ID NO: 114 - RNA, 20 nt, Artificial Sequence
  B2M_3
acuugucuuucagcaaggac

SEQ ID NO: 115 - RNA, 20 nt, Artificial Sequence
  B2M_4
ucacgucauccagcagagaa

SEQ ID NO: 116 - RNA, 20 nt, Artificial Sequence
  gRNA99
ccugagcugcaacagcaucg

SEQ ID NO: 117 - RNA, 20 nt, Artificial Sequence
  gRNA100
gccaccagcacucagcgcag

SEQ ID NO: 118 - protein, 383 aa, Artificial Sequence
  Minimal BASP1_mCherry_L7Ae
MGGKLSKKKGGSGGGSGGGSGVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKGGSGGGSGGGSGMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

(19) United States
(12) Patent Application Publication
(10) Pub. No.: US 2024/0279301 A1
(43) Pub. Date: Aug. 22, 2024

(54) METHODS AND COMPOSITIONS

(71) Applicant: XAP THERAPEUTICS LIMITED, Cambridge (GB)

(72) Inventors: James PATTERSON, Cambridge (GB); Laurent JESPERS, Cambridge (GB)

(21) Appl. No.: 18/570,795

(22) PCT Filed: Jun. 15, 2022
(86) PCT No.: PCT/GB2022/051512

§ 371 Date: Dec. 15, 2023

(30) Foreign Application Priority Data
Jun. 16, 2021 (GB) 2108585.7

(51) Int. Cl. C07K 14/705; A61K 47/69; C12N 15/11; C12N 15/64

(52) U.S. Cl. CPC C07K 14/705; A61K 47/6901; C12N 15/11; C12N 15/64; C07K 2319/03

(57) ABSTRACT

The present invention provides engineered platelets with chimeric platelet receptors (CPR) with a desired target specificity. Additionally, the engineered platelets may comprise cargo which may be released upon activation of the platelet. Additionally, the platelets may be generated in vitro from megakaryocytes engineered to generate non-thrombogenic platelets.

### FIELD OF THE INVENTION

The invention relates to targeted delivery systems.

### BACKGROUND OF THE INVENTION

Platelets are small and enucleated and cannot divide or reproduce. In the human body, they perform the important function of recognising injured tissue and releasing their contents to reduce or prevent bleeding. Thrombopoietin from the kidneys and liver contact a myeloid stem cell causing differentiation into a megakaryoblasorphant, and additional signals result in differentiation of the megakaryoblast into a progenitor megakaryocyte. Progenitor megakaryocytes are large cells with platelet precursor extensions that bud off fragments as they divide and proliferate to create platelets.

Mitochondria, microtubules, and vesicles are contained within the platelets, and the platelets have a life span of about 10 days before clearance by macrophages. Platelets have a volume of about 7 μm³ and a diameter of 300 nm. They are metabolically active and can alter gene expression through post-transcriptional control of preloaded mRNA expression (e.g. by miRNAs). Platelets comprise intracellular vesicles termed granules. On activation, degranulation is stimulated to alter the shape and release the contents of the granules.

Platelets contain three primary subtypes of vesicles: α-granules (50 to 80 per platelet), dense granules (3 to 8 per platelet), and large dense core vesicles (LDCV) (about 10,000 per platelet). Different mutations can selectively disrupt the biogenesis of each vesicle subtype. The contents of granules (including exosomes, a sub-set of platelet extracellular vesicles (PEVs) which are predominantly stored in alpha-granules) are released by exocytosis. A huge variety of products are released on platelet degranulation.

PEVs are membrane-bound entities that are produced by and released from platelets in response to an activating signal. These PEVs represent the majority of extracellular vesicles in the circulatory system. Platelets primarily release two vesicle families, —a) microvesicles; and b) exosomes (Heijnen, H. F. G., Schiel, A. E., Fijnheer, R., Geuze, H. J., & Sixma, J. J. (1999). Activated platelets release two types of membrane vesicles: Microvesicles by surface shedding and exosomes derived from exocytosis of multivesicular bodies and α-granules. Blood, 94(11), 3791-3799. https://doi.org/10.1182/blood.v94.11.3791).

Microvesicles are produced by membrane shedding and capture a sample of the platelet's cytoplasmic content. Exosomes, in contrast, are stored within platelet α-granules, and are released upon platelet stimulation mediated degranulation. Because of their distinct biogenesis pathways, exosomes and microvesicles deliver distinct subsets of cargo and feature distinct surface protein compositions and physical sizes.

TABLE 1

| | Size | Biogenesis | Surface markers |
|---|---|---|---|
| Microvesicles | 100-1000 nm | Activation | Equivalent to |
| | | dependent plasma | platelet surface |
| | | membrane budding | markers (as it is an |
| | | and release | outbudding of the |
| | | | plasma membrane) |
| Exosomes | 50-100 nm | Production within | CD9, CD63, CD81, |
| | | multivesicular | PTGFRN, Syntaxin |
| | | bodies which further | and many more |
| | | mature into alpha | (Jankovic̆ová, J., et |
| | | granules within the | al (2020) . . . |
| | | megakaryocyte | International |
| | | and platelet | Journal of |
| | | | Molecular |
| | | | Sciences, |
| | | | 21(20), 1-30.) |

Exosomes naturally transport a diverse range of cargoes between cells, including protein, RNA, RNPs and chemical messengers. Exosome cargo represents both a stochastic sampling of the cytoplasmic contents of the cell, in addition to featuring specific, enriched cargoes. The specific mechanisms of exosome biogenesis allow for targeting of exogenous cargo to them, and thus the production of designer therapeutic exosomes. These have been produced in a range of cell types, but importantly have been subsequently purified from these cells before delivery is attempted. Systemic delivery of exosomes has potential issues. Due to their small size, they can passively escape the circulatory system, thus limiting their uptake in target cells or tissues. Targeting exosomes to specific cells or tissues also relies upon either engineered surface markers (Duan, L., Xu, L., Xu, X., Qin, Z., Zhou, X., Xiao, Y., Liang, Y., & Xia, J. (2021). Exosome-mediated delivery of gene vectors for gene therapy. Nanoscale, 13(3), 1387-1397. https://doi.org/10.1039/d0nr07622h) or the natural target cell tropism of an exosome from a particular producer cell. Thus, there is still a need for high-efficiency exosome targeting technologies to permit systemic delivery.

Platelets respond to a variety of extra cellular signals through a diverse set of signaling pathway receptors. Receptors act both to trigger intracellular signaling cascades resulting in platelet degranulation, and effector release and to cause platelet aggregation and adhesion. Glycoprotein VI platelet (GPVI) signaling functions analogously to many immune cell receptors—such as the TCR. Interestingly, platelets also express toll-like receptors (TLRs) and can mediated targeted killing of bacteria via peptide secretion and immune system activation.

α-granules have a diameter of about 200 to 500 nm and make up about 10% of the platelet's volume. Exosomes are stored in the α-granules. Most effector proteins are found in α-granules. For example, effector proteins released from α-granules include: integral membrane proteins, such as P-selectin, αIIbβ, and GPIbα; coagulants/anticoagulants and fibrinolytic proteins, such as factor V, factor IX, and plasminogen; adhesion proteins, such as fibrinogen and von Willebrand Factor (vWF); chemokines, such as CXCL4 (cytokine (C-X-C motif) ligand 4), also known as platelet factor 4 or PF4, and CXCL12 (cytokine (C-X-C motif) ligand 12), also known as stromal cell-derived factor 1 alpha or SDF-1α; growth factors, such as elongation growth factor (EGF) and insulin-like growth factor 1 (IGF); angiogenic factors/inhibitors, such as vascular endothelial growth factor (VEGF), platelet-derived growth factor (PDGF), and angiostatins; and immune mediators, such as immunoglobulin G (IgG) and complement precursors.

Dense granules have a diameter of about 150 nm and make up about 1% of the platelet's volume. Effector proteins released from dense granules include cations, such as Ca²⁺ and Mg²⁺; polyphosphates; bioactive amines, such as serotonin and histamine; and nucleotides, such as adenosine diphosphate (ADP) and adenosine triphosphate (ATP).

LDCVs have a diameter in the range of about 150 nm to about 300 nm and make up about 13.5% of the platelet's volume. Effector proteins released from LDCVs include structural proteins (e.g., granins and glycoproteins); vascoregulators (e.g., cateholamines, vasostatins, renin-angiotensin); paracrine signaling factors (e.g., guanylin, neurotensin, chromogranin B); immune mediators (e.g., enkelytin and ubiquitin); opiods (e.g., enkephalins and endorphins); ions (e.g., Ca²⁺, Na+, Cl—), and nucleotides and polyphosphates (e.g., adenosine monophosphate (AMP), guanosine diphosphate (GDP), uridine-5′-triphosphate (UTP)).

Current cell therapies based on engineered chimeric antigen receptor T cells (CAR-T cells) have shown promise in treating cancer; however, concerns regarding their safety, specifically oncogenic transformation in the patient, and the limited ability to generate a generic or universal therapeutic product have restricted their use to a small number of patients. There is a long felt need in the art for a new type of therapy with the potential to treat cancer, autoimmune conditions, and infections, free from the safety, cost, and patient matching issues which plague current cell therapeutic products.

### SUMMARY OF THE INVENTION

The invention provides various components, compositions and methods that can be used in the safe delivery of a cargo to a subject—in preferred embodiments the safe delivery is a targeted safe delivery. The various components, compositions and methods described herein can also be used to stimulate T cells in addition to, or instead of, delivering a cargo to a subject. The cell or cell-like entities that are used to generate the delivery entities, and the delivery entities themselves are collectively termed “chassis” herein. For example the chassis may be an “effector-chassis” which is the chassis that is actually administered to a subject in need thereof, with the aim of either delivering a particular cargo, for example delivering a particular cargo in a targeted manner; or with the aim of engaging specific receptors of the invention that are present in the membrane of the effector-chassis with the corresponding target in the subject—i.e. the effector-chassis does not have to comprise a cargo for it to be useful.

The chassis described herein may also be a “producer-chassis”. A producer chassis is a chassis that is directly able to produce platelets, or platelet-like membrane-bound cell fragments, or anucleate cell fragments. For example in some embodiments a producer-chassis produces platelets, or platelet-like membrane-bound cell fragments, or anucleate cell fragments via extension of the plasma membrane to form protoplatelets which are then fragmented in to platelets, or platelet-like membrane-bound cell fragments, or anucleate cell fragments.

In some embodiments, by “anucleate cell fragments” we do not include the meaning of red blood cells (erythrocytes), or fragments of red blood cells. It is also clear that in some preferred embodiments “anucleate cell fragment” is not intended to include within the meaning extracellular vesicles or other lipid-bound vesicles. The skilled person is readily able to understand what is intended by “anucleate cell fragments”.

Accordingly, in some embodiments the anucleate cell fragments are not red blood cells. In some embodiments the anucleate cell fragments are not fragments of red blood cells. In some embodiments the anucleate cell fragments are not extracellular vesicles. In some embodiments the anucleate cell fragments are not exosomes.

In preferred embodiments the anucleate cell fragments are produced from producer-chassis as described herein.

It is clear to the skilled person from the disclosure herein that many of the modifications that result in the production of “effector-chassis” described herein can be made far upstream in the maturation of the producer-chassis that subsequently produces the effector-chassis. It is clear therefore that it is appropriate that in some instances the chassis described herein may be a “progenitor-chassis”. A progenitor-chassis is in preferred embodiments an immortal cell that can be reliably used to generate producer-chassis. As is apparent, in some preferred embodiments the progenitor-chassis is an immortal cell such as an iPSC that has been engineered to differentiate into a particular producer-chassis, such as a megakaryocyte. A progenitor-chassis also includes immortalised cells such as adipocytes and cells that are the result of transdifferentiation of otherwise mature cells such as dipose-derived mesenchymal stromal/stem cell line (ASCL) (see Tozawa et al 2019 Blood 133:633-643).

The in vivo differentiation pathway from myeloid stem cell to a megakaryoblast to a megakaryocyte, or the in vitro differentiation of iPSC to a megakaryocyte is well defined, and the skilled person knows how to produce platelets or platelet-like membrane-bound cell fragments, and knows which cells are progenitors of the platelets or platelet-like membrane-bound cell fragments. For example, there are various ways to drive a progenitor cell as described herein, such as an iPSC, to differentiate in to a producer-chassis as described herein. One such method is known as “forward programming” and drives the differentiation of iPSC directly to megakaryocytes (see for example Forward Programming Megakaryocytes from Human Pluripotent Stem Cells, Thomas Moreau, BBTS Annual Conference, Glasgow 2017 1045 thu lomond moreau (1).pdf) and typically involves the expression of one or more transcription factors (Gata1, Tal1 and Fli1) that drive differentiation to megakaryocytes.

Exemplary chassis described herein include a myeloid stem cell, an iPSC, a megakaryoblast, a megakaryocyte, a megakaryocyte-like cell, adipocyte, adipose-derived mesenchymal stromal/stem cell line (ASCL), a platelet, a platelet-like membrane-bound cell fragment or an anucleate cell fragment.

Exemplary progenitor-chassis include a myeloid stem cell, an iPSC, adipocyte, adipose-derived mesenchymal stromal/stem cell line (ASCL), and a cancer cell-line or other immortal cell that is capable of producing a producer-chassis as described herein.

Exemplary producer-chassis include a megakaryoblast, a megakaryocyte, a megakaryocyte-like cell, or a cancer cell line or other immortal cell that is capable of forming a platelet, a platelet-like membrane-bound cell fragment or an anucleate cell fragment, such as a MEG01 or DAMI cancer cell line.

Exemplary effector-chassis include a platelet, a platelet-like membrane-bound cell fragment or an anucleate cell fragment.

The chassis described herein also include any immortal versions of these cells/cell-like entities, that have been driven to differentiate into any one or more producer cells as described herein, for example in some embodiments the chassis has been “forward programmed”, i.e. engineered so as to knockin or knockout particular genes (or otherwise modify gene expression such as through the use of RNAi) to direct differentiate into megakaryocytes.

Any of the chassis described herein may be modified to express one or more receptors of the invention, for example any progenitor-chassis, producer-chassis or effector-chassis may be modified so as to express any one or more chimeric platelet receptors (CPRs), universal chimeric platelet receptors (universal CPRs), complexes of universal CPRs and tagged targeting peptides, synthetic antigen presenting receptors (SAPRs), or engineered protease activated receptors (ePARS) described herein. By “express” we include the meaning of transcription and translation, or translation alone. For example, the receptors described herein must be displayed on the surface of the chassis (i.e. in the plasma membrane). In some embodiments the chassis has been modified at the nucleic acid level so as to introduce a nucleic acid that encodes for the receptor. In this instance the chassis must transcribe and translate the nucleic acid to produce the functional protein. In other instances, the chassis may be modified to introduce an mRNA that is translated into a functional receptor of the invention. Accordingly in the context of expressing a receptor of the invention, the intention is to ensure that functional receptor protein is produced.

In some embodiments the chassis has only been engineered to express one or more receptors of the invention, for example any progenitor-chassis, producer-chassis or effector-chassis may be modified so as to express any one or more chimeric platelet receptors (CPRs), universal chimeric platelet receptors (universal CPRs), complexes of universal CPRs and tagged targeting peptides, synthetic antigen presenting receptors (SAPRs), or engineered protease activated receptors (ePARS) described herein—i.e. in some embodiments no further engineering steps have been performed on the chassis.

Any of the chassis described herein may be engineered so as to modulate any one or more different pathways, for example any progenitor-chassis, producer-chassis or effector-chassis may be engineered to as to:
i) to disrupt a platelet inflammatory signaling pathway;
ii) to make the engineered chassis less immunogenic;
iii) to enhance or disrupt one or more base functions of the chassis, wherein the one or more or base functions are involved in the innate and/or adaptive immune response, inflammation, angiogenesis, atherosclerosis, lymphatic development and tumour growth; and/or
iv) engineered to disrupt a platelet thrombogenic pathway.

In some embodiments, the:
i) platelet inflammatory signaling pathway
ii) pathway that when modulated makes the engineered chassis less immunogenic;
iii) base function involved in the innate and/or adaptive immune response, inflammation, angiogenesis, atherosclerosis, lymphatic development and tumour growth; and/or
iv) platelet thrombogenic pathway

is a pathway that is found in any one or more of:
a) an engineered progenitor-chassis for example a myeloid stem cell; an iPSC; a cancer cell-line that is capable of producing a producer-chassis; adipocyte; adipose-derived mesenchymal stromal/stem cell line (ASCL); or other immortal cell that is capable of producing a producer-chassis;
b) an engineered producer-chassis for example a megakaryoblast; a megakaryocyte; a megakaryocyte-like cell; a cancer cell line that is capable of forming a platelet for example a MEG01 or DAMI cancer cell line, a platelet-like membrane-bound cell fragment or an anucleate cell fragment; or other immortal cell that is capable of forming a platelet, a platelet-like membrane-bound cell fragment or an anucleate cell fragment; or
c) an engineered effector-chassis for example a platelet, a platelet-like membrane-bound cell fragment or anucleate cell fragment.

In some embodiments the chassis has only been engineered so as to modulate any one or more different pathways, for example any progenitor-chassis, producer-chassis or effector-chassis may be engineered to as to:
i) to disrupt a platelet inflammatory signaling pathway;
ii) to make the engineered chassis less immunogenic;
iii) to enhance or disrupt one or more base functions of the chassis, wherein the one or more or base functions are involved in the innate and/or adaptive immune response, inflammation, angiogenesis, atherosclerosis, lymphatic development and tumour growth; and/or
iv) engineered to disrupt a platelet thrombogenic pathway. i.e. in some embodiments no further engineering steps have been performed on the chassis.

Any of the chassis described herein may be engineered so as to express one or more receptors of the invention and has been engineered so as to modulate any one or more different pathways i.e. in some embodiments the invention provides any of the chassis as described herein that has been engineered to:
A) modulate any one or more different pathways, for example any progenitor-chassis, producer-chassis or effector-chassis may be engineered to as to:
   i) to disrupt a platelet inflammatory signaling pathway;
   ii) to make the engineered chassis less immunogenic;
   iii) to enhance or disrupt one or more base functions of the chassis, wherein the one or more or base functions are involved in the innate and/or adaptive immune response, inflammation, angiogenesis, atherosclerosis, lymphatic development and tumour growth; and/or
   iv) engineered to disrupt a platelet thrombogenic pathway;
and
B) express any one or more chimeric platelet receptors (CPRs), universal chimeric platelet receptors (universal CPRs), complexes of universal CPRs and tagged targeting peptides, synthetic antigen presenting receptors (SAPRs), or engineered protease activated receptors (ePARS) described herein.

Effector-chassis as described herein include platelets, platelet-like membrane-bound cell fragments, and anucleate cell fragments, that in some instances have been produced from a producer-chassis.

The actual delivery agent (or T cell stimulating agent) that is administered to a subject and that is intended to produce an effect in a subject is herein termed an effector-chassis, and includes within its meaning a platelet, or platelet-like membrane bound cell fragment, other cell fragment or Synlet (as described herein), that is derived from any of the producer-chassis as described herein. A producer-chassis as described herein includes within its meaning any cell (including an engineered cell) that is upstream in the typical differentiation process that directly produces an effector-chassis, i.e. a platelet or platelet-like membrane-bound cell fragment, or Synlet.

The general term “chassis” is intended to encompass all of the progenitor-chassis, the producer-chassis and the effector-chassis that can be derived from the producer-chassis.

In some embodiments, the effector-chassis is not used to deliver cargo, for example in some embodiments a effector-chassis that comprises a SAPR of the invention but that does not comprise a cargo is considered to be useful, as is apparent to the skilled person from the discussion herein.

In a preferred embodiment, the effector-chassis and/or the producer-chassis and/or the progenitor-chassis comprises a chimeric platelet receptor (CPR), a universal CPR, a complex comprising a CPR and a tagged targeting peptide, a SAPR or a ePAR as described herein.

The receptors as described herein essentially re-direct the normal intracellular functioning of a platelet-surface receptor, so that rather than intracellular signaling occurring in response to recognition of the native endogenous cognate target for a particular receptor, the intracellular signaling occurs in response to a different target i.e. the “target binding domain” of the receptor is modified so as to bind to a target of interest, for example a cancer neo-antigen.

In a first aspect the invention provides:

A chimeric platelet receptor wherein the receptor comprises:
a) an intracellular domain that is a platelet modulation domain; and
b) a heterologous target binding domain that recognizes and binds a target.

To be clear, preferences for the target binding domain described here in relation to the CPR are also preferences for the platelet modulation domain of other aspects of the invention, e.g. the Universal CPR, Complex of Universal CPR and tagged targeting peptide, and the SAPR described herein.

Platelets naturally comprise receptors that transduce external signals to effect various functions of the platelet. For example platelet receptors that comprise ITAM domains, once activated by binding to an appropriate target, are considered to activate the platelets' thrombogenic pathways and degranulation pathways. Platelet receptors that comprise ITIM domains, once activated by binding to an appropriate target, are considered to inhibit the activation of the platelet thrombogenic pathways, and thereby inhibit the activation of degranulation. The receptors and chassis as described herein that comprise the receptors are considered to redirect this natural process to particular targets, so that the platelet in some examples degranulates in response to a different target to which it would usual degranulate. It is considered that in order to achieve platelet activation (e.g. degranulation) or inhibition of activation (for example inhibition of activation of degranulation) all that is required is a receptor as described herein that comprises an external domain that binds to the target, and an internal domain that can modulate the behaviour of the platelet (or effector-chassis as described herein). In order for the effector-chassis to respond appropriately to target binding to the receptor, the relevant internal pathways must be functional. For example in some embodiments as described herein the thrombogenic pathway is disrupted, as in some embodiments it is preferred if the effector-chassis does not trigger the usual thrombogenic pathway in response to binding to a target; but for degranulation to occur the effector-chassis must have a functional degranulation pathway.

Degranulation occurs through the generation of IP3. Receptors that comprise ITAMs are phosphorylated upon target binding, which results in the recruitment of Src family kinase (such as Syk). Recruitment of the Src family kinases results in PLC-gamma-2 activation and IP3 generation. Activation of the ePAR as described herein triggers PLC-Beta activation and IP3 generation.

IP3 binds to and activates IP3-Receptors, triggering Ca2+ influx to the platelet cytoplasm from intracellular stores and the extracellular milieu. Ca2+ triggers the exocytosis of alpha-granules (degranulation) and a range of other events, culminating in platelet activation, including degranulation.

Accordingly it is clear that in some preferred embodiments, any of the chassis as described herein, for example the progenitor, producer and effector-chassis comprises the necessary cellular components to effect platelet activation and/or degranulation. For example in some embodiments the chassis as described herein comprises Src family kinases and IP3. In some embodiments the chassis as described herein comprises PLC-beta and IP3. In some embodiments the chassis comprises IP3-receptors.

The heterologous target-binding domain is the external part of a transmembrane protein that also comprises an intracellular signaling domain. The intracellular signaling domain transduces the binding of the target to the target binding domain so as to result in modulation of the platelet, for example activation of the platelet. The skilled person appreciates that some receptors, such as ITAM and ITIM receptors, require some degree of receptor clustering on the membrane surface to effect intracellular signaling and platelet activation. Accordingly, by binding of the target to the target binding domain so as to result in modulation of the platelet, for example activation of the platelet we include the meaning that binding of the target to the target binding domain results in receptor clustering. The degree of receptor clustering required for activation of a platelet is receptor and target dependent. The skilled person is able to determine whether a given CPR of the invention is able to effect platelet modulation using assays known in the art, for example the assay involving P-selectin as described herein.

Since in some embodiments receptor clustering is considered to be necessary, when the CPR is present in the membrane of an effector-chassis, in some embodiments the target to which the receptor binds is a target that when bound by the CPR present in the membrane of the effector-chassis results in CPR receptor clustering and activation of the platelet modulation domain.

For example, in some embodiments, the target is present on a cell surface or a tissue surface. By a heterologous target binding domain we mean that the target binding domain is heterologous to the intracellular platelet modulation domain i.e. the target binding domain is not the usual extracellular domain associated with the intracellular domain. The heterologous target binding domain or heterologous tag binding domain may bind to an endogenous target, for example may bind to a tumour antigen that is endogenous to a subject but, by virtue of the CPR being chimeric, the target binding domain is heterologous to the internal platelet modulation domain.

In some embodiments the target binding domain may be endogenous to the progenitor, producer and/or effector-chassis, but is heterologous to the platelet modulation domain. e.g. the CPR is not found naturally in any cell or progenitor, producer, and/or effector-chassis and has been produced as the result of biological engineering. Accordingly in some preferred embodiments the CPR is not a naturally occurring protein or complex.

In some embodiments the platelet modulation domain is a domain that is found in a base platelet, i.e. if a platelet modulation domain that is naturally found in a platelet.

For instance, in embodiments where the intracellular modulation domain comprises the intracellular domain of Glycoprotein VI (GPVI), the targeting domain is not the extracellular domain of Glycoprotein VI (GPVI), i.e. the domains are heterologous to one another. In some embodiments, C-type lectinlike receptor 2 (CLEC-2) or Fc Fragment of IgG Receptor Ha (FCgR2A) may be altered in a similar way. In other embodiments, where the intracellular domain comprises the intracellular domain of C-type lectinlike receptor 2 (CLEC-2), the extracellular targeting domain is not the extracellular domain of CLEC-2; and in some embodiments where the intracellular domain comprises Fc Fragment of IgG Receptor Ha (FCgR2A), the extracellular targeting domain does not comprise the extracellular domain of FCgR2A.

It is clear that the target binding domain may be a domain that is native to the subject, but is not native to the intracellular domain.

The heterologous target binding domain may be any target binding domain that is able to bind with some specificity to a particular target. Preferably the target binding domain binds specifically to the target or tag. By “binds specifically” we include the meaning that the target binding domain binds to its target in a manner that can be distinguished from binding to non-targets (i.e. off-targets). For example, a target binding domain that is specific for the target may refer to a target binding domain that binds with higher specificity for the intended target compared with that of a non-intended target. Specificity can be determined based on dissociation constant through routine experiments. A target binding domain being “specific for” a target is intended to be synonymous with a target binding domain being “directed against” said target.

Preferably the target binding domain binds only to its respective target, e.g. the immune cell target o, and does not bind to any other molecule in the environment, for example in the human body. However, it is be appreciated that some degree of off-target binding may be tolerated, and the skilled person understands how to determine whether a particular binding activity is of the required specificity or not. Accordingly, the binding domain may bind specifically to the intended target, whilst also binding to some lower level to non-target or non-tag molecules.

In one embodiment the target binding domain does not bind to collagen.

The invention described herein also provides a CPR that in some embodiments comprises a heterologous target binding domain that comprises or consists of any of the sequences or proteins or portions of proteins described as the “second region” in paragraph [0012] on page 3 of PCT/GB2020/053247 which is hereby incorporated by reference.

The CPR of the present invention are proteins and are expressed as a single transcript.

The target can be any target which is able to specifically bind to a proteinaceous sequence or fragment or domain.

In some embodiments, the target binding domain binds to an endogenous target that is found on a tissue in the body of a subject or on a cell or in a particular location of a subject, for example the endogenous target may be a target that is present on tissue, or on a particular subset of tissue, or in plasma or blood of a subject, for example a human subject for example in the blood. In some embodiments the target is a target that is only presented during one or more disease states, for example in some embodiments the target is a neoantigen that arises in a tumour cell. In some embodiment the target is a target that is only present in significant amounts for example abnormal levels on a tissue or cell that does not normally express the target and/or is only present in a localised manner during or more disease states. An effector-chassis of the invention (as described herein) that comprises one or more CPRs of the invention can be used to “survey” for abnormalities that may occur upon the commencement of a state of disease, or progression of disease, and the cargo can be released. The target binding domain can be any domain that can bind to a marker of disease.

The target binding domain preferably binds to an endogenous target as described herein. In some embodiments the target binding domain binds to an artificial or exogenous target—i.e. in some embodiments the target binding domain, in order to achieve activation of the platelet and degranulation, has to bind to an exogenous agent that is provided to the subject. The target binding domain in some embodiments binds to a “designer drug”, and/or the target binding domain has been designed using Designer Receptor Exclusively Activated by Designer Drugs (DREADD) as described in WO 2020072471.

In some embodiments binding of the target binding domain to an endogenous target is sufficient to modulate the platelet via the platelet modulation domain.

In some embodiments the target is present on a cell surface or a tissue surface.

In some embodiments the target is a target such that when the CPR or universal CPR is present in a platelet membrane, after binding of the target to the target binding domain the CPRs or universal CPRs cluster on the plasma membrane. By a platelet in this instance we mean a standard base platelet that has not be engineered to disrupt any signalling pathways for instance, and has only been engineered to express the CPR or universal CPR.

In some embodiments when the CPR or universal CPR is present in a platelet membrane, after binding of the target to the target binding domain the platelet modulation domain is activated. By a platelet in this instance we mean a standard base platelet that has not be engineered to disrupt any signalling pathways for instance, and has only been engineered to express the CPR or universal CPR. This is a test that the skilled person can readily perform to determine whether a given CPR or universal CPR is a CPR of the invention, since in some embodiments when a base platelet has been engineered to express one or CPRs or universal CPRs, binding of the target binding domain to the target:
a) results in degranulation of the platelet;
b) results in the release of contents from the platelet;
c) results in the presence of intraplatelet contents on the plasma membrane of the platelet;
d) results in the release of extracellular vesicles via blebbing from the plasma membrane; and/or a small molecule drug, imaging agent, radionucleotide drugs, radionucleotide tagged antibodies, or conjugate any thereof;
e) results in a change of shape of the platelet from a biconcave disk to fully spread cell fragments; and/or
f) results in an influx of calcium into the platelet.

In some embodiments when the CPR or universal CPR is present in a platelet plasma membrane, after binding of the target binding domain to the target the CPRs or universal CPRs cluster on the surface of the platelet plasma membrane, wherein said clustering is sufficient to activate the platelet modulation domain. By a platelet in this instance we mean a standard base platelet that has not be engineered to disrupt any signalling pathways for instance, and has only been engineered to express the CPR or universal CPR.

In addition to target binding to a target binding domain of a CPR or universal CPR present in a base platelet causing receptor clustering, platelet activation, or activation of the platelet modulation domain, it is preferred if target binding to a target binding domain CPR or a universal CPR in an effector-chassis as described herein (e.g. a platelet, platelet-like membrane-bound cell fragment or anucleated cell fragment), for example that may or may not have been engineered to modulate one or more pathways such as:
i) to disrupt a platelet inflammatory signaling pathway;
ii) to make the engineered chassis less immunogenic; and/or
iii) to enhance or disrupt one or more base functions of the chassis, wherein the one or more or base functions are involved in the innate and/or adaptive immune response, inflammation, angiogenesis, atherosclerosis, lymphatic development and/or tumour growth; and/or
iv) engineered to disrupt a platelet thrombogenic pathway;
   also causes activation of the effector-chassis, activation of the CPR or universal CPR platelet modulation domain; and/or CPR clustering on the surface of the effector-chassis.

Accordingly, in some embodiments the target is a target such that when the CPR or universal CPR is present in an effector chassis as described herein, after binding of the target to the target binding domain of the CPR or universal CPR, the CPRs or universal CPRs cluster on the plasma membrane. The effector-chassis may or may not also have been engineered to disrupt one or more pathways as described herein.

In some embodiments when the CPR or universal CPR is present in an effector-chassis membrane, after binding of the target to the target binding domain the platelet modulation domain of the CPR or universal CPR is activated. The effector-chassis may or may not also have been engineered to disrupt one or more pathways as described herein. In some embodiments binding of the target to the target binding domain of the CPR or universal CPR:
a) results in degranulation of the effector-chassis;
b) results in the release of contents from the effector-chassis;
c) results in the presence of intraplatelet contents on the plasma membrane of the effector-chassis;
d) results in the release of extracellular vesicles via blebbing from the plasma membrane; and/or a small molecule drug, imaging agent, radionucleotide drugs, radionucleotide tagged antibodies, or conjugate any thereof;
e) results in a change of shape of the effector-chassis from a biconcave disk to fully spread cell fragments; and/or
f) results in an influx of calcium into the effector-chassis.

In some embodiments when the CPR or universal CPR is present in an effector-chassis plasma membrane, after binding of the target binding domain to the target the CPRs or universal CPRs cluster on the surface of the effector-chassis plasma membrane, wherein said clustering is sufficient to activate the platelet modulation domain of the CPR or universal CPR. The effector-chassis may or may not also have been engineered to disrupt one or more pathways as described herein.

In some embodiments the target binding domain comprises a human target binding domain sequence or a sequence that has at least 75%, 80%, 85%, 90%, 92%, 94%, 96%, 98% or 100% sequence identity to a human target binding domain sequence.

In some embodiments the target binding domain comprises a non-human target binding domain sequence, optionally:
a humanised sequence; or
a sequence from a mouse.

In some embodiments the target binding domain comprises a target-binding ligand or fragment thereof that binds specifically to said target.

In some embodiments, the target is an antigen and the targeting domain is able to bind to the antigen, for example a neoantigen on tumour cells or a tumour specific antigen (TSA).

In some embodiments the target binding domain may recognize CD19 to deliver the cargo, for example a cargo that is a chemotherapeutic, locally. CD19 is a well-known B cell surface molecule, which upon B cell receptor activation enhances B-cell antigen receptor induced signaling and expansion of B cell populations. CD19 is broadly expressed in both normal and neoplastic B cells.

Malignancies derived from B cells such as chronic lymphocytic leukemia, acute lymphocytic leukemia and many non-Hodgkin lymphomas frequently retain CD19 expression. This near universal expression and specificity for a single cell lineage has made CD19 an attractive target for immunotherapies.

In some embodiments the target binding domain comprises a linked cytokine that binds to the cytokine receptor present on target cells.

In some embodiments, the target binding domain is a natural ligand (or fragment thereof) of a target.

In some embodiments, the target binding domain does not bind to collagen.

In some embodiments, the target binding domain is an antibody or an antigen binding fragment thereof that is able to bind to the target of interest. For example, the target binding domain may include a variable heavy chain domain of an antibody and/or may include a variable light chain domain of an antibody and/or may include a kappa light chain or a fragment thereof, for example to target CD19.

In some embodiments, the target binding domain is an antibody or the antibody fragment thereof is chosen from Table 11 presented on pages 64-77 of PCT/GB2020/053247 which is hereby incorporated by reference. The antibodies are listed with their DrugBank identifier (DB ID). The target of each of these antibodies, along with exemplary diseases which can be treated with each of the antibodies is described on pages 77-92 of PCT/GB2020/053247 which is hereby incorporated by reference.

In some embodiments, the target binding domain is a human target binding domain sequence or a sequence that has at least 75%, 80%, 85%, 90%, 92%, 94%, 96%, 98% or 100% sequence identity to a human target binding domain sequence, e.g. derived from a human protein, for example from a human antibody or antibody fragment thereof. Alternatively, the target binding domain may be derived from a non-human animal, or may be an entirely synthetic domain, for example may be an antibody or the antibody fragment thereof may be from a non-human animal, such as a mouse. In some embodiments, the target binding domain may be a humanized sequence, for example the target binding domain may be an antibody or antigen binding fragment thereof that is humanized.

The target binding domain can be an antibody, variant or fragment thereof. An antibody, variant, or fragment thereof can be generated using routine recombinant DNA technology techniques known in the art.

In some embodiments, the target binding domain is an antibody or antibody fragment thereof that is able to bind a protein selected from Table 2 on pages 23-31 of PCT/GB2020/053247 which is hereby incorporated by reference. In some embodiments, the antibody or the antibody fragment thereof may bind a protein encoded by IL2 (interleukin 2; ENSG00000109471). In some embodiments, the antibody or antibody fragment thereof may bind a histone complex. In some embodiments, the antibody or antibody fragment thereof may bind a protein encoded by kallikrein (KLK; ENSG00000167759). In some embodiments, the antibody or antibody fragment thereof may bind amyloid. In some embodiments, the antibody or antibody fragment thereof may bind a Notch receptor. In some embodiments, the antibody or antibody fragment thereof may bind a protein encoded by oxidized low density receptor 1(OLR1; ENSG00000173391).

Exemplary target binding domains are described as extracellular domains in Table 7 on page 46 of PCT/GB2020/053247 which is hereby incorporated by reference.

In some embodiments, the target binding domain may bind to CD276, for example the target binding domain may be an antibody or antigen binding fragment thereof that binds to CD276.

As used herein, the terms “antibody” or “antibodies” refer to molecules that contain an antigen binding site, e.g. immunoglobulin molecules and immunologically active fragments of immunoglobulin molecules that contain an antigen binding site. Immunoglobulin molecules can be of any type (e.g. IgG, IgE, IgM, IgD, IgA and IgY), class (e.g. IgG1, IgG2, IgG3, IgG4, IgA1 and IgA2) or a subclass of immunoglobulin molecule. Antibodies include, but are not limited to, synthetic antibodies, monoclonal antibodies, single domain antibodies, single chain antibodies, recombinantly produced antibodies, multi-specific antibodies (including bispecific antibodies), human antibodies, humanized antibodies, chimeric antibodies, intrabodies, scFvs (e.g. including mono-specific and bi-specific, etc.), Fab fragments, F(ab′) fragments, disulfide-linked Fvs (sdFv), anti-idiotypic (anti-Id) antibodies, and epitope-binding fragments of any of the above.

As used herein, the term “antibody fragment” is a portion of an antibody such as F(ab′)₂, F(ab)₂, Fab′, Fab, Fv, scFv and the like. Regardless of structure, an antibody fragment binds with the same antigen that is recognized by the intact antibody. For example, an anti-OX40 antibody fragment binds to OX40. The term “antibody fragment” also includes isolated fragments consisting of the variable regions, such as the “Fv” fragments consisting of the variable regions of the heavy and light chains and recombinant single chain polypeptide molecules in which light and heavy variable regions are connected by a peptide linker (“scFv proteins”). As used herein, the term “antibody fragment” does not include portions of antibodies without antigen binding activity, such as Fc fragments or single amino acid residues.

By “Fab fragment”, we include Fab fragments (comprising a complete light chain and the variable region and CH1 region of a heavy chain) which are capable of binding the same antigen that is recognized by the intact antibody. Fab fragment is a term known in the art, and Fab fragments comprise one constant and one variable domain of each of the heavy and the light chain.

In one embodiment the target binding domain comprises at least one of:
a) FCERG EC domain, CLEC1 EC domain, FCGR2 EC domain, GPVIA EC domain, CEACAM1 EC domain, G6b-B EC domain, LILRB2 EC domain, PECAM1 EC domain TLT1 EC domain and/or a sequence that has at least 75%, 80%, 85%, 90%, 92%, 94%, 96%, 98% or 100% sequence identity to a FCERG EC domain, CLEC1 EC domain, FCGR2 EC domain, GPVIA EC domain, CEACAM1 EC domain, G6b-B EC domain, LILRB2 EC domain, PECAM1 EC domain and/or TLT1 EC domain; and/or
b) the target binding domain comprises any one or more of the domains or portions thereof set out on page 46 to 49 of PCT/GB2020/053247 which is hereby incorporated by reference, or a sequence that has at least 75%, 80%, 85%, 90%, 92%, 94%, 96%, 98% or 100% sequence identity to any one or more of the domains or portions thereof set out on page 46 to 49 of PCT/GB2020/053247 which is hereby incorporated by reference.

In some embodiments, the target binding domain comprises a peptide associated with autoimmunity, optionally:
a peptide or portion of any one or more of the following proteins: MOG, GAD65, MAG, PMP22, TPO, VGKC, PLP, AChR, TRIB2, NMDA, GluR, GAD2, ARMC9, CYP21A2, CASR, NASP, insulin, TSHR, thyroperoxidase, asioglycoprotein receptor, CYP2D6, LF, TTG, H/K ATP-ase, Factor XIII, Beta2-GPI, ITGB2, G-CSF, GP IIb/IIa, COLII, FBG beta alpha, MPO, CYO, PRTN3, TGM, COLVII, COIL, DSG1, DSG3, SOX10, 70SNRNP70, SAG and a3(IV)NC1 collagen; or
a peptide or portion that has at least 75%, 80%, 85%, 90%, 92%, 94%, 96%, 98% or 100% sequence identity to any one or more of the following proteins: MOG, GAD65, MAG, PMP22, TPO, VGKC, PLP, AChR, TRIB2, NMDA, GluR, GAD2, ARMC9, CYP21A2, CASR, NASP, insulin, TSHR, thyroperoxidase, asioglycoprotein receptor, CYP2D6, LF, TTG, H/K ATP-ase, Factor XIII, Beta2-GPI, ITGB2, G-CSF, GP IIb/IIa, COLII, FBG beta alpha, MPO, CYO, PRTN3, TGM, COLVII, COIL, DSG1, DSG3, SOX10, 70SNRNP70, SAG and a3(IV)NC1 collagen.

In this embodiment the effector-chassis comprising the receptor is targeted to antigen-specific B cells (see Cabaletta et al 2016 Science DOI: 10.1126/science.aaf6756).

For example, a target binding domain that comprises a desmoglein3-ITAM may be used to target pemphigus vulgans B cells. Alternatively, SAPRs of the invention that express an MHC class 1-ITAM chimeric platelet receptor or MHC class 2-ITAM chimeric platelet receptor, such that the MHC class 1 or the MHC class 2 may be loaded with a peptide from the list above on the surface of the platelet to target autoimmune mediating T cells for destruction or for suppression through the release of anti-inflammatory cytokines, such as TGF-β. Additionally, RNA encoding transcription factors may be released, such as FOXP3 to transdifferentiate bound T cells into Tregs.

In some embodiments, as described above, the target binding domain may target the receptor to a specific tissue associated with an autoantigen. For example, the target binding domain may bind to an antigen present on: adipose tissue, adrenal gland, ascites, bladder, blood, bone, bone marrow, brain, cervix, connective tissue, ear, embryonic tissue, esophagus, eye, heart, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, stomach, testis, thymus, thyroid, tonsil, trachea, umbilical cord, uterus, vascular, and spleen.

The CPR described herein comprises a platelet modulation domain. To be clear, preferences for the platelet modulation domain described here in relation to the CPR are also preferences for the platelet modulation domain of other aspects of the invention, e.g. the Universal CPR, Complex of Universal CPR and tagged targeting peptide, and the SAPR described herein.

In some embodiments the platelet modulation domain is endogenous to the progenitor, producer and/or effector-chassis in which the receptor is to be used, for example endogenous to the iPSC, megakaryocyte, adipocyte, adipose-derived mesenchymal stromal/stem cell line (ASCL) or platelet.

By “modulation domain” we include the meaning of domains that trigger platelet activation, and we include the meaning of domains that inhibit or prevent the triggering of platelet activation.

The platelet activities that are activated or, that are not activated where activation is inhibited by activation of an inhibitory platelet activation domain that prevents activation of a platelet include:
a) degranulation of the platelet, for example with the release of alpha-granules;
b) the release of contents from the platelet;
c) presenting intracellular contents on the plasma membrane of the platelet;
d) releasing of extracellular vesicles via blebbing from the plasma membrane; and/or
e) changing the shape of the platelet from a biconcave disk to fully spread cell fragments.

By “activation of the platelet modulation domain” we mean that platelet modulation domain is able to modulate a platelet that comprises the platelet modulation domain.

The skilled person can readily determine whether a receptor (e.g. CPR, universal CPR, complex of universal CPR and tagged targeting peptide, SAPR and/or ePAR according to the invention) is able to result in activation of degranulation, or inhibition or degranulation or inhibition of activation of degranulation. For example, the skilled person may contact a platelet expressing the receptor with a cell that expresses the corresponding target and measure a shape change or exposure of P-Selectin. A change in shape of the platelet or exposure of P-Selectin indicates that the particular receptor is able to activate the platelet upon binding to the target. To determine the ability of a receptor of the invention to inhibit the activation of a platelet, a similar assay may be performed. The skilled person may contact a platelet that expresses the receptor with a cell that expresses the corresponding target and which also expresses an endogenous target that would typically result in activation of a platelet (e.g. collagen). A change in shape or exposure of P-Selectin indicates that the receptor under investigation is unable to inhibit activation of the platelet via the usual pathway. A failure to change shape or expose P-Selectin indicates that the receptor is able to successfully prevent activation of the platelet. The dose response should be measured, and where, for instance, the EC50 of the dose response of natural platelet agonist vs. e.g. P-Selectin exposure is higher in the presence of an engineered inhibitory receptor target, this indicates the construction of a functional inhibitory receptor. Conversely, an IC50 could be calculated of the inhibitory target on the receptor could be calculated in a similar fashion, by holding cognate agonist concentrations constant and varying the amount of potentially inhibitory stimulus.

In preferred embodiments, by platelet activation we mean causing platelet degranulation. Accordingly, in some embodiments the platelet modulation domain is a degranulation triggering domain, and in some embodiments the modulating domain is a domain that prevents triggering of platelet degranulation. In some embodiments the platelet modulation domain is an activation domain that triggers the release of alpha-granules. In some embodiments the platelet modulation domain is a domain that prevents the triggering of the release of alpha-granules.

In the same or different embodiments, by platelet activation we mean causing the release of contents from the platelet. Accordingly, in some embodiments the platelet modulation domain is a platelet content release domain, and in some embodiments the modulation domain is a domain that prevents the release of the platelet contents.

In the same or different embodiments, by “activation” we mean that the platelet releases extracellular vesicles via blebbing from the plasma membrane.

In the same or different embodiments, by platelet activation we mean causing the presentation of intraplatelet contents on the plasma membrane. Accordingly, in some embodiments the platelet modulation domain is a domain that causes the presentation of intraplatelet contents on the plasma membrane, and in some embodiments the modulation domain is a domain that prevents the presentation of intraplatelet contents on the plasma membrane.

In the same or different embodiments, by platelet activation we mean causing the release of extracellular vesicles via blebbing from the plasma membrane. In some embodiments calcium influx is another measurable parameter to indicate platelet activation. Accordingly, in some embodiments the platelet modulation domain is a domain that causes the release of extracellular vesicles via blebbing from the plasma membrane, and in some embodiments the modulation domain is a domain that prevents the release of extracellular vesicles via blebbing from the plasma membrane. Accordingly, in some embodiments the platelet modulation domain is a domain that causes in influx of calcium into the platelet, and in some embodiments the modulation domain is a domain that prevents an influx of calcium into the platelet.

In the same or different embodiments, by platelet activation we mean causing a change in the shape of the platelet from a biconcave disk to fully spread cell fragments. Accordingly, in some embodiments the platelet modulation domain is a domain that causes a change in the shape of the platelet from a biconcave disk to fully spread cell fragments, and in some embodiments the modulation domain is a domain that prevents a change in the shape of the platelet from a biconcave disk to fully spread cell fragments.

In the same or different embodiments, by “activation” we include the meaning that the platelet changes shape from a biconcave disk to fully spread cell fragments. During this process, platelets extend filopodia and generate lamellipodia, resulting in a dramatic increase in the platelet surface area. The skilled person is able to identify the shape changes typical of platelet activation, for example see Asian et al 2012 Methods Mol Biol 788: 91-100.

In preferred embodiments by “activation” we include the meaning that the platelet releases, or exposes on the platelet cell surface, a cargo that has been introduced into the platelet (either introduced endogenously via genetic manipulation of the platelet pre-cursor, e.g. megakaryocyte or iPSC or introduced exogenously). Preferences for the cargo and methods of introducing the cargo into the progenitor, producer and/or effector-chassis for example the platelet are as described herein.

Examples of platelet activation domains include domains that comprise ITAM motifs or that include an domains that have at least 75%, 80%, 85%, 90%, 92%, 94%, 96%, 98% or 100% sequence identity to an ITAM comprising domain, for example a platelet ITAM comprising domain.

Examples of degranulation inhibitory domains include domains that comprise ITIM motifs or that comprise a domain that has at least 75%, 80%, 85%, 90%, 92%, 94%, 96%, 98% or 100% sequence identity to an ITIM comprising domain.

By “triggering degranulation” or “preventing the triggering of degranulation” we include the meaning that degranulation is triggered, or is prevented from the being triggered, when the target binding domain binds to its corresponding target. As described above, in some embodiments some degree of receptor clustering is necessary for activation of the platelet modulation domain.

Whether the effector-chassis, for example a platelet as described herein, degranulates in response to the target binding domain of the CPR (or universal CPR, complex of universal CPR and tagged targeting peptide, SAPR as described herein) binding to the target depends on whether the platelet modulation domain is a platelet activation domain for example a degranulation triggering domain or an inhibition of platelet activation domain, or the combination of different types of domains.

As described further herein, by using a combination of different receptors with different platelet modulation domains (for example different CPRs, universal CPRs, complexes of universal CPRs and tagged targeting peptides, or SAPRs or ePARs described herein) that comprise platelet activation domains (e.g. degranulation triggering domains) and inhibition of platelet activation domains (e.g. degranulation inhibitory domains) in a single effector-chassis for example a platelet as described herein, it is possible to build up complex logic circuits, such as AND/OR/NOR etc and so has the ability to integrate and compute a variety of stimuli before making the decision to activate (degranulate).

In some embodiments of the intracellular domain that is a platelet modulation domain is a platelet inhibition domain. In some embodiments, the platelet inhibition domain comprises an immunoreceptor tyrosine-based inhibition motif (ITIM)-containing receptor domain. Non-limiting examples of mM receptors include platelet and endothelial cell adhesion molecule 1 (PECAM1), triggering receptor expressed on myeloid cells like 1 (TLT1), leukocyte immunoglobulin like receptor B2 (LILRB2), carcinoembryonic antigen related cell adhesion molecule 1 (CEACAM1), megakaryocyte and platelet inhibitory receptor G6b (G6b-B).

Inhibition of platelet activation can be useful in instances such as preventing the activation of off-target cells that the on-target antigen; or the inhibit platelet activation in response to normal agents found in clotting, e.g. the ITAM domain in GPVI could be swapped for an mM domain and switch off platelet activation at clotting sites.

G6b-B clustering by antibody inhibits platelet activation through GPVI and CLEC-2 as shown in Mori et al. “G6b-B inhibits constitutive and agonist-induced signaling by glycoprotein VI and CLEC-2”. JBC, 2008, which is hereby incorporated by reference in its entirety. Adding a chimeric “off” receptor may be used to improve specificity of the targeted effector-chassis described herein, for example synthetic platelets described herein. A CPR, universal CPR, complex of universal CPR and tagged targeting peptide, SAPR and/or ePAR that comprises an immunoreceptor tyrosine-based inhibition motif (ITIM) receptor would allow logic gate construction when used in combination with other CPRs.

In one embodiment, domains of ITIM receptors LILRB2 (SEQ ID NO: 34), PECAM1 (SEQ ID NO: 38), TLT1 (SEQ ID NO: 43), and CEACAM1 (SEQ ID NO: 24) shown in Table 5 on page 44 of PCT/GB2020/053247 which is hereby incorporated by reference and the corresponding explanatory paragraph [0063] which is also hereby incorporated by reference; or domains that have at least 75%, 80%, 85%, 90%, 92%, 94%, 96%, 98% or 100% sequence identity to an ITIM domain of receptors LILRB2 (SEQ ID NO: 34), PECAM1 (SEQ ID NO: 38), TLT1 (SEQ ID NO: 43), and CEACAM1 (SEQ ID NO: 24) shown in Table 5 on page 44 of PCT/GB2020/053247 which is hereby incorporated by reference and the corresponding explanatory paragraph [0063] which is also hereby incorporated by reference.

In one embodiment, domains of ITIM receptors may be combined with T cell receptor domains to form chimeric ITIM receptors which are also referred to as chimeric platelet receptors.

It is clear that any platelet inhibition domain, i.e. any domain that transduces target binding to inhibit the activation of platelet degranulation is suitable for use in the receptors described herein, e.g. the CPR, universal CPR, complex of universal CPR and tagged targeting peptide and SAPR described herein.

In some embodiments the platelet modulation domain is a platelet activation domain. In preferred embodiments the platelet activation domain is a degranulation triggering domain. In some embodiments, the platelet activation domain is an immunoreceptor tyrosine-based activation motif (ITAM)-containing receptor domain. ITAM receptors mediate platelet activation and stimulate an immune response. Glycoprotein VI (GPVI) binds to collagen and is a central mediator of platelet activation. It features extracellular IgG like domains, and the internal tyrosine kinase signaling pathway is triggered by receptor clustering through the Fc receptor (FcR) gamma chain. Non-limiting examples of ITAM receptors include glycoprotein VI platelet (GPVIA), high affinity immunoglobulin epsilon receptor subunit gamma (FCERG), C-Type lectin domain family 1 (CLEC1), and Fc fragment of IgG receptor II (FCGR2)

In some embodiments then the platelet modulation domain is a platelet degranulation triggering domain and comprises:
one or more domains from an immunoreceptor tyrosine based activation motif (ITAM) receptor, optionally comprises one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2); or
a domain that has at least 75%, 80%, 85%, 90%, 92%, 94%, 96%, 98% or 100% sequence identity to an ITAM comprising domain, for example a platelet ITAM comprising domain, optionally has at least 75%, 80%, 85%, 90%, 92%, 94%, 96%, 98% or 100% sequence identity to one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2).

In one embodiment, the intracellular domain does not comprise domains from an immunoreceptor tyrosine based activation motif (ITAM) receptor. In one embodiment the intracellular domain does not comprise one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2). In one embodiment the intracellular domain does not comprise an ITAM domain that comprises or consists of the SEQ ID NO: 5, 7, 14 and/or 19 of PCT/GB2020/053247 which is hereby incorporated by reference.

In one embodiment, the intracellular domain does comprise domains from an immunoreceptor tyrosine based activation motif (ITAM) receptor. In one embodiment the intracellular domain does comprise one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2); or
a domain that has at least 75%, 80%, 85%, 90%, 92%, 94%, 96%, 98% or 100% sequence identity to one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2).

In one embodiment the intracellular domain does comprise an ITAM domain that comprises or consists of the SEQ ID NO: 5, 7, 14 and/or 19 of PCT/GB2020/053247 which is hereby incorporated by reference or that comprises or consists of a sequence that has at least 75%, 80%, 85%, 90%, 92%, 94%, 96%, 98% or 100% sequence identity to SEQ ID NO: 5, 7, 14 and/or 19 of PCT/GB2020/053247 which is hereby incorporated by reference.

It is clear to the skilled person that domains from an ITAM receptor that is not typically expressed in platelets is still expected to function in the invention, since the ITAM domains are still capable of activating the same downstream signaling components as ITAM receptors are endogenously found in platelets. For example it is known that T-Cell CARs can be used in macrophages and NK cells.

It is clear that any platelet modulating domain, i.e. any domain that transduces target binding to platelet modulation, for example to platelet degranulation, is suitable for use as a platelet modulation domain.

In some embodiments, the platelet modulation domain is not a naturally occurring domain. For example, in some embodiments the modulation domain, for example the ITAM, ITIM domains comprises one or more mutations, insertions or deletions that boost or dampen the response to target binding. In some instances, the platelet modulation domain comprises at least 75%, 80%, 85%, 90%, 92%, 94%, 96%, 98% or 100% sequence identity to a naturally occurring modulation domain, for example comprises at least 75%, 80%, 85%, 90%, 92%, 94%, 96%, 98% or 100% sequence identity to any of the modulation domain sequences described herein.

For example, in some embodiments the platelet modulation domain is a modified platelet modulation domain that has been modified so as to have increased sensitivity as compared to the unmodified platelet modulation domain. For example a receptor of the invention comprising such a modified platelet modulation domain (e.g. a CPR, universal CPR, complex of universal CPR and tagged targeting peptide or SAPR as described herein) is expected to react to a lower amount of target, for example if the platelet modulation domain is a modified platelet activation domain, an effector-chassis comprising the receptor would be expected to degranulate in response to a lower amount of target than is required to make an effector-chassis comprising a receptor comprising an unmodified modulation domain (e.g. the CPR, universal CPR, complex of universal CPR and tagged targeting peptide or SAPR of the invention comprising an unmodified platelet modulation domain) degranulate. In some embodiments, the modified platelet modulation domain has been modified so as to have decreased sensitivity as compared to the unmodified platelet modulation domain. For example a receptor of the invention comprising such a modified platelet modulation domain (e.g. a CPR, universal CPR, complex of universal CPR and tagged targeting peptide or SAPR as described herein) is expected to not react (e.g. degranulate) in response to an amount of target that would make an effector-chassis comprising a receptor with an unmodified platelet modulation domain react (e.g. degranulate). In some embodiments then the platelet modulation domain is a modified platelet modulation domain. By modified we include the meaning of any alteration to the sequence that encodes the domain, for example, insertions, deletions and/or substitutions. In some embodiments the platelet modulation domain comprises one or more ITAM domains, wherein the ITAM domains comprises one or more modifications, for example one or more insertions, deletions or substitutions. In some embodiments the platelet modulation domain comprises one or more ITIM domains, wherein the ITAM domains comprises one or more modifications, for example one or more insertions, deletions or substitutions.

It is clear to the skilled person that the platelet modulation domain does not have to be a human platelet modulation domain. For example ITAM or mM containing domains from species that are not human are considered to be useful in the present invention. For example, ITAM containing domains from humans have been shown to be active in mice species have been shown to function in CAR-T situations (Robles-Carrillo et al 2010 J Immunol 185:1577-1583).

In some embodiments, the target binding domain is the native target binding domain of a receptor that has a platelet modulation domain, but the platelet modulation has been altered so as to have the opposite function. For example, in some embodiments the receptor described herein (e.g. the CPR, universal CPR, or complex of universal CPR and tagged targeting peptide) comprises the target binding domain of an ITAM platelet modulation domain comprising receptor, but wherein the ITAM domain has been swapped to an ITIM domain. For example in some embodiments the CPR or universal CPR or complex of universal CPR and tagged targeting peptide comprises the external target binding domain of any of Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2) but wherein the ITAM comprising domain of the protein has been changed to an ITIM comprising domain. This applies to any embodiment described herein.

In some embodiments the CPR can be considered to be a universal CPR. By a universal CPR we include the meaning of a CPR that, by virtue of the targeting binding domain being a tag binding domain, it is possible to use a single CPR to direct a progenitor, producer and/or effector-chassis of the invention to any target.

Accordingly, in a further aspect, the invention also provides:

A universal chimeric platelet receptor wherein the receptor comprises:
a) an intracellular domain that is a platelet modulation domain; and
b) a heterologous tag binding domain.

Preferably binding of the tag on the targeting peptide to the universal CPR is not sufficient to activate the platelet modulation domain. The platelet modulation domain should only be activated upon subsequent binding of the CPR/targeting peptide complex to the target.

Preferences for features of the universal CPR are as described elsewhere herein. For example preferences for the platelet modulation domain are as described in relation to the first aspect, i.e. the CPR.

The universal CPR has a tag binding domain that is able to specifically bind to a proteinaceous sequence or fragment or domain.

A tag binding domain being “specific for” a tag is intended to be synonymous with a tag binding domain being “directed against” said tag.

Preferably the tag binding domain binds only to its respective target, e.g. the tag on the tagged targeting peptide, and does not bind to any other molecule in the environment, for example in the human body. However, it is to be appreciated that some degree of off-target binding may be tolerated, and the skilled person understands how to determine whether a particular binding activity is of the required specificity or not. Accordingly, the binding domain may bind specifically to the intended target, whilst also binding to some lower level to non-tag molecules.

In preferred embodiments, the tag is a peptide tag and is expressed as part of the larger targeting peptide and is an integral part of the larger targeting peptide i.e. in such embodiments the tagged targeting peptide is a single peptide that comprises both the tag and the target binding domain.

The concept of “tags” is well known in the molecular biology field, where it is routine to express a peptide or polypeptide sequence of interest wherein the sequence has been extended to include a relatively short additional sequence, encoding the tag. Examples of suitable peptide tags include the FLAG-tag, V5-tag, Myc-tag, HA-tag, Spot-tag, T7-tag, NE-tag and a leucine-zipper (Hwan et al 2018 Cell 173: 1426-1438).

In some embodiments the tagged targeting peptide may be tagged with a non-peptide tag, for example any moiety that can acts as a binding partner for the tag binding domain of the universal CPR. Thus, a non-peptide tag can be any chemical entity to which the tag binding domain has affinity. The tag can be selected from, for example, any organic molecule, a small molecule, or a hapten. Tags can for example take the form of nucleic acids, for example aptamers.

Peptide tags as described herein are typically short peptide sequences (i.e. sequences of amino acids). In preferred embodiments the tags described herein are peptide or protein tags, for example short sequences of amino acids. The tag can be of any sequence provided it is able to be bound, preferably specifically bound by the tag binding domain of the universal CPR of the invention.

It is preferred that the universal CPR is not activated upon binding to the tagged targeting peptide, in the absence of concomitant binding of the target binding domain to the target. As described above, the skilled person appreciates that some receptors, such as ITAM and ITIM receptors, require some degree of receptor clustering on the membrane surface to effect intracellular signaling and platelet activation. When a universal CPR of the invention binds to a soluble tagged targeting peptide, there is no clustering of the receptors, and so binding of the tagged targeting peptide to the universal CPR does not trigger activation of the platelet modulation domain. It is only when the complex of the universal CPR and tagged targeting peptide binds the target that receptor clustering occurs, and so activation of the platelet modulation domain.

It is preferred then that the tagged targeting peptide is a soluble peptide, since it is considered that the binding of a soluble peptide, in the absence of simultaneous binding to the target, does not trigger activation of the platelet modulation domain.

As described above, in some embodiments some degree of receptor clustering is required to active the platelet modulation domains. In some embodiments the universal CPR comprises a tag binding domain that binds to a tag present on a tagged targeting peptide, and wherein when the Universal CPR is located in a platelet plasma membrane binding of the tagged targeting peptide to the universal CPR in the absence of simultaneous binding of the tagged target binding domain to the target does not cause sufficient receptor clustering to lead to activation of the platelet modulation domain.

In other embodiments the tag, for example a peptide tag may be conjugated to the larger targeting peptide, following expression of the larger targeting peptide, to produce the tagged targeting peptide.

In addition to the universal CPR, the invention also provides a corresponding tagged targeting peptide. The tagged targeting peptides comprises a tag and a target binding domain, optionally wherein the tagged targeting peptide is a soluble peptide. Preferences for the target binding domain are as described elsewhere herein, for example in relation not the first aspect (i.e. the CPR).

The invention also provides a complex comprising:
a) a universal CPR that comprises:
   i) a heterologous tag binding domain; and
   ii) a platelet modulation domain; and
b) a tagged targeting peptide that comprises a tag capable of biding to the tag binding domain of the CPR, and a targeting domain.

The invention also provides:

A synthetic antigen presenting receptor (SAPR) comprising a heterologous target binding domain wherein the target binding domain comprises:
a) an extracellular domain comprising:
   i) the MHC-1 protein or fragment thereof, or a protein or fragment thereof that has at least 75%, 80%, 85%, 90%, 92%, 94%, 96%, 98% or 100% sequence identity to a human MHC-1 protein or fragment thereof; or
   ii) the MHC-2 protein or fragment thereof or a protein or fragment thereof that has at least 75%, 80%, 85%, 90%, 92%, 94%, 96%, 98% or 100% sequence identity to a human MHC-2 protein or fragment thereof; and
b) an intracellular platelet modulation domain,

wherein said:
MHC-1 protein or fragment thereof or a protein or fragment thereof that has at least 75%, 80%, 85%, 90%, 92%, 94%, 96%, 98% or 100% sequence identity to a human MHC-1 protein or fragment thereof; or
MHC-2 protein or fragment thereof or a protein or fragment thereof that has at least 75%, 80%, 85%, 90%, 92%, 94%, 96%, 98% or 100% sequence identity to a human MHC-2 protein or fragment thereof;

is able to bind to a T Cell Receptor (TCR).

Preferences for features of this aspect are as described elsewhere, for example the platelet modulation domain may be a platelet activation domain, optionally an ITAM comprising domain, optionally a platelet ITAM comprising domain, optionally is domain that has at least 75%, 80%, 85%, 90%, 92%, 94%, 96%, 98% or 100% sequence identity to an ITAM comprising domain optionally a platelet ITAM comprising domain; or a platelet activation domain, optionally wherein the platelet activation domain is a degranulation triggering domain; or is an inhibition of platelet activation domain that prevents activation of a platelet, optionally wherein the inhibition of platelet activation domain is an ITIM comprising domain, optionally is a domain that has at least 75%, 80%, 85%, 90%, 92%, 94%, 96%, 98% or 100% sequence identity to an ITIM comprising domain. In some embodiments the platelet modulation domain is endogenous to an iPSC, a megakaryocyte or a platelet.

In some embodiments the SAPR of the present invention can be used to trigger activation of T cells and the induction of a response to a particular antigen. In these instances, the extracellular domain of the SAPR comprises or consists of an amino acid sequence that is an antigen towards which it is desirous to trigger the T cell response, for example an antigen from a pathogen, and an amino acid sequence that encodes the MHC-1 protein or the MHC-II protein or fragment thereof. MHC-1 would activate CD8+ T Cell,s MHC-2 would activate CD4+ T cells T cells comprise a T Cell Receptor (TCR) that binds to a complex of MHC-1/antigen that is usually expressed on the surface of antigen-presenting cells. In this way, the SAPR of the invention as described herein can be considered to be a synthetic antigen-presenting receptor. In addition, a progenitor, producer and/or effector-chassis expressing a SAPR of the invention that is loaded with an antigenic peptide, such as those described herein and presented on the surface of the effector-chassis can be used to target autoimmune mediating T cells for destruction or for suppression through the release of anti-inflammatory cytokines, such as TGF-β. Additionally, RNA encoding transcription factors may be released, such as FOXP3 to transdifferentiate bound T cells into Tregs.

It is clear to the skilled person that the MHCs are single chain variants that have been engineered.

Accordingly, the invention provides:

A SAPR as described above wherein said extracellular domain comprises:
a) the MHC-1 protein or fragment thereof, or a protein or fragment thereof that has at least 75%, 80%, 85%, 90%, 92%, 94%, 96%, 98% or 100% sequence identity to a human MHC-1 protein or fragment thereof. and an antigenic peptide, wherein said MHC-1 protein or fragment thereof, or a protein or fragment thereof that has at least 75%, 80%, 85%, 90%, 92%, 94%, 96%, 98% or 100% sequence identity to a human MHC-1 protein or fragment thereof and antigenic peptide is able to bind to a TCR; and/or
b) the MHC-2 protein or fragment thereof, or a protein or fragment thereof that has at least 75%, 80%, 85%, 90%, 92%, 94%, 96%, 98% or 100% sequence identity to a human MHC-1 protein or fragment thereof. and an antigenic peptide, wherein said MHC-2 protein or fragment thereof, or a protein or fragment thereof that has at least 75%, 80%, 85%, 90%, 92%, 94%, 96%, 98% or 100% sequence identity to a human MHC-1 protein or fragment thereof and antigenic peptide is able to bind to a TCR.

In these embodiments, a useful effect is obtained when the SAPR that comprises an extracellular domain that comprises or consists of an MHC-1/antigen complex or an MHC-2/antigen complex binds to the TCR (where the TCR is the target in this instance)—the result is the activation of the T cell response directed towards the particular antigen.

In some embodiments the antigenic peptide comprises a peptide or antigenic portion thereof:
a) associated with cancer, an autoimmune condition, genetic disease, cardiovascular disease and/or an infection; and/or
b) selected from:
   i) the antigenic peptides listed in Table F on page 206-207; Table G on page 208; Table H on page 208-209; Table I on page 209-211; Table J page 212; Table 4 page 219-221; Table 5 page 221-230; Table 6 page 231-234; Table 7 page 235-242 and Table 89 page 243 of WO 2015153102 which is hereby incorporated by reference these sections of which are hereby incorporated by reference; or
   ii) a sequence that has at least 75%, 80%, 85%, 90%, 92%, 94%, 96%, 98% or 100% sequence identity to the antigenic peptides listed in Table F on page 206-207; Table G on page 208; Table H on page 208-209; Table I on page 209-211; Table J page 212; Table 4 page 219-221; Table 5 page 221-230; Table 6 page 231-234; Table 7 page 235-242 and Table 89 page 243 of WO 2015153102 these sections of which are hereby incorporated by reference; and/or
c) selected from:
   i) the antigenic peptides listed in Table 1 page 47-86; Table 14 page 321; Table 15 page 321; Table 16 page 327; Table 17 page 328; Table 18 page 328-329; Table 19 page 221-223; Table 20 page 333-334; Table 21 page 340-342; Table 22 page 344-347; Table 23 page 347-348; and Table 24 page 349-352 of WO 2019/126818, these sections of which are hereby incorporated by reference; or
   ii) a sequence that has at least 75%, 80%, 85%, 90%, 92%, 94%, 96%, 98% or 100% sequence identity to the antigenic peptides listed the antigenic peptides listed in Table 1 page 47-86; Table 14 page 321; Table 15 page 321; Table 16 page 327; Table 17 page 328; Table 18 page 328-329; Table 19 page 221-223; Table 20 page 333-334; Table 21 page 340-342; Table 22 page 344-347; Table 23 page 347-348; and Table 24 page 349-352 of WO 2019/126818, these sections of which are hereby incorporated by reference;
d) selected from:
   i) any one or more of the following proteins: MOG, GAD65, MAG, PMP22, TPO, VGKC, PLP, AChR, TRIB2, NMDA, GluR, GAD2, ARMC9, CYP21A2, CASR, NASP, insulin, TSHR, thyroperoxidase, asioglycoprotein receptor, CYP2D6, LF, TTG, H/K ATP-ase, Factor XIII, Beta2-GPI, ITGB2, G-CSF, GP IIb/IIa, COLII, FBG beta alpha, MPO, CYO, PRTN3, TGM, COLVII, COIL, DSG1, DSG3, SOX10, 70SNRNP70, SAG and a3(IV)NC1 collagen; or
   ii) a sequence that has at least 75%, 80%, 85%, 90%, 92%, 94%, 96%, 98% or 100% sequence identity to any one or more of the following proteins: MOG, GAD65, MAG, PMP22, TPO, VGKC, PLP, AChR, TRIB2, NMDA, GluR, GAD2, ARMC9, CYP21A2, CASR, NASP, insulin, TSHR, thyroperoxidase, asioglycoprotein receptor, CYP2D6, LF, TTG, H/K ATP-ase, Factor XIII, Beta2-GPI, ITGB2, G-CSF, GP IIb/IIa, COLII, FBG beta alpha, MPO, CYO, PRTN3, TGM, COLVII, COIL, DSG1, DSG3, SOX10, 70SNRNP70, SAG and a3(IV)NC1 collagen.

The extracellular domain may comprise a human target binding domain sequence; a non-human target binding domain sequence, optionally a humanised sequence or a sequence from a mouse. It is considered useful if upon binding to a TCR the SAPR triggers activation of a platelet modulation domain. For example where the platelet modulation domain is a platelet activation domain, the platelet releases cargo, or where the modulation domain is an inhibition of activation domain, activation of the platelet is inhibited preventing release of cargo. Triggering degranulation when the SAPR is bound to a T cell can be advantageous when the particular targeted T cell is involved in the autoimmune response—for example in these situations it can be beneficial for the platelet to comprise toxic agents that are released locally upon T-cell engagement, to ultimately destroy the T cell.

In other embodiments, the cargo that is released on degranulation can stimulate the T cell to differentiate in a particular way.

In preferred embodiments, where the platelet modulation domain is a platelet activation domain, and when the SAPR is present in the membrane of a platelet, and when activated, the platelet activation domain:
a) results in degranulation of the platelet;
b) results in the release of contents from the platelet;
c) results in the presence of intracellular contents on the plasma membrane of the platelet; d) results in the release of extracellular vesicles via blebbing from the plasma membrane; and/or
e) results in a change of shape of the platelet from a biconcave disk to fully spread cell fragments.

In some embodiments, where the platelet modulation domain is a inhibition of platelet activation domain, and when the SAPR is present in the membrane of a platelet, and when activated, the inhibition of platelet activation domain:
a) prevents degranulation of the platelet;
b) prevents the release of contents from the platelet;
c) prevents the presence of intracellular contents on the plasma membrane of the platelet; d) prevents the release of extracellular vesicles via blebbing from the plasma membrane; and/or
e) prevents a change of shape of the platelet from a biconcave disk to fully spread cell

As described above, the invention provides a SAPR that comprises a heterologous target binding domain wherein the target binding domain comprises the MHC-1 or MHC-2 protein or fragment thereof, wherein said MHC-1 or MHC-2 protein or fragment thereof is able to bind to a T Cell Receptor (TCR).

In some instances the CPR, universal CPR, or complex of universal CPR and tagged targeting peptide or SAPR may also comprise a signal peptide, and/or a linker. Non-limiting examples of signal peptides include:

TABLE 1

| Sequence Identifier | Description |
|---|---|
| 2 | FCERG Signal Peptide |
| 11 | FCGR2 Signal Peptide |
| 16 | GPVIA Signal Peptide |
| 25 | CEACAM1 Signal Peptide |
| 30 | G6b-B Signal Peptide |
| 35 | LILRB2 Signal Peptide |
| 39 | PECAM1 Signal Peptide |
| 44 | TLT1 Signal Peptide |

The CPR, universal CPR, complex of universal CPR and tagged targeting peptide, SAPR and/or ePAR may include a portion of the signal peptide in Table 6 or a signal peptide known in the art. The portion may be 10-30, 10-15, 10-20, 10-25, 15-20, 15-25, 15-30, 20-25, or 20-30, nucleotides of any of the sequences in Table 6 such as, but not limited to, SEQ ID NO: 2, 11, 16, 25, 30, 35, 39, and 44. The portion may be 10, 11, 12, 13, 14, 15, 16, 17, 18, 19, 20, 21, 22, 23, 24, 25, 26, 27, 28, 29, or 30 nucleotides of any of the sequences in Table 7 on page 46 of PCT/GB2020/053247 which is hereby incorporated by reference, such as, but not limited to, SEQ ID NO: 2, 11, 16, 25, 30, 35, 39, and 44 as described in Table 7 on page 46 of PCT/GB2020/053247 which is hereby incorporated by reference.

In some embodiments the CPR, universal CPR, complex of universal CPR and tagged targeting peptide and/or SAPR comprises a transmembrane domain. In some embodiments the transmembrane domain comprises or consists of any one or more of the transmembrane domains or portions thereof as set out on page 49-50 of PCT/GB2020/053247 which is hereby incorporated by reference.

In some embodiments the CPR, universal CPR, complex of universal CPR and tagged targeting peptide and/or SAPR comprises an intracellular domain that comprises or consists of the intracellular domains or a portion thereof as set out on page 50 and 51 of PCT/GB2020/053247 which is hereby incorporated by reference.

In some embodiments the CPR, universal CPR, complex of universal CPR and tagged targeting peptide and/or SAPR comprises a linker. In some embodiments the linker comprises or consists of the linkers or portions thereof as set out on page 51 of PCT/GB2020/053247 which is hereby incorporated by reference.

In some embodiments, the CPR, universal CPR, complex of universal CPR and tagged targeting peptide and/or SAPR of the invention comprises or consists of a combination of domains as set out on pages 41-63 of PCT/GB2020/053247 which is hereby incorporated by reference.

The invention provides further receptors to direct the activation of platelets towards particular sites/targets. These receptors are based on protease activated receptors (PARs).

The invention provides an engineered protease activated receptor (ePAR) wherein the protease recognition site is engineered to be cleaved by a protease that is not the protease that cleaves the native recognition site. For example where the ePAR is an engineered PAR1, the ePAR is not cleaved by thrombin.

In some embodiments, when the ePAR is present in the plasma membrane of a platelet, cleavage of the protease recognition site results in:
a) degranulation of the platelet;
b) the release of contents from the platelet;
c) the presence of intracellular contents on the plasma membrane of the platelet;
d) the release of extracellular vesicles via blebbing from the plasma membrane; and/or
e) a change of shape of the platelet from a biconcave disk to fully spread cell fragments.

By platelet in this instance we mean a base platelet that has not been engineered other than to express the ePAR—i.e. in a normally functioning platelet, the cleavage of the ePAR results in any one or more of (a) to (e) above. This is one straightforward means by which the skilled person can determine whether an ePAR is an ePAR of the invention and has the appropriate functional properties. Of course it is apparent to the skilled person that in preferred embodiments the ePAR of the invention results in the any one or of (a) to (e) above when present in a chassis of the invention, for example an effector-chassis as described herein that has for example been engineered to modulate one or more pathways such as:
i) to disrupt a platelet inflammatory signaling pathway;
ii) to make the engineered chassis less immunogenic; and/or
iii) to enhance or disrupt one or more base functions of the chassis, wherein the one or more or base functions are involved in the innate and/or adaptive immune response, inflammation, angiogenesis, atherosclerosis, lymphatic development and/or tumour growth; and/or
iv) engineered to disrupt a platelet thrombogenic pathway.

Accordingly in some embodiments when the ePAR of the invention is present in the membrane of an effector-chassis of the invention, cleavage of the protease site results in:
a) degranulation of the platelet;
b) the release of contents from the platelet;
c) the presence of intracellular contents on the plasma membrane of the platelet;
d) the release of extracellular vesicles via blebbing from the plasma membrane; and/or
e) a change of shape of the platelet from a biconcave disk to fully spread cell fragments.

In some embodiments, cleavage of the protease results in release of a fragment of the ePAR and wherein the fragment of the ePAR is a signalling molecule and effects intracellular signalling.

In some embodiments the ePAR is engineered to be cleaved by a protease that is typically found in the tumour microenvironment. For example in some instances the ePAR is engineered to be cleaved by matrix metalloproteases, metallopeptidases, Cathepsin B, Urokinases or Capsases.

In some embodiments the ePAR is engineered to be cleaved by an orthogonal protease. Proteases that are considered to be non-orthogonal to a human subject include viral proteases such as Tobacco Etch Virus nuclear-inclusion-a endopeptidase (TEV protease), NS2-3 protease of hepatitis C virus (HCV protease), or tobacco vein mottling virus (TVMV protease). Protease recognition sites to be introduced into engineered protease receptors include viral protease recognition sites of TEV protease, HCV protease, and/or TVMV protease.

For example, where the ePAR is to be used in the context of the human body, the ePAR is engineered to be cleaved by a protease that is not a human protease, i.e. in these embodiments, the ePAR cannot be cleaved in the subject unless the subject is also administered or otherwise exposed to a corresponding “exogenous” protease. As is clear from the disclosure herein, such an exogenous protease can be a cargo present in a second chassis (e.g. a second progenitor-chassis, producer or effector-chassis as described herein), In this case, a first chassis (e.g. progenitor-chassis, a progenitor, producer or effector-chassis as described herein) comprises a first cargo and an ePAR of the invention, and a second chassis (e.g. progenitor-chassis, a progenitor, producer or effector-chassis as described herein) comprises a CPR according to the invention and a cargo that is a protease that can cleave the ePAR. Upon CPR binding and platelet activation, the protease is released, cleaving ePAR that are in the vicinity, leading to release of the first cargo, which may be for example a toxic cargo. In this way the first cargo is release only an the intended target site. This double fail-safe approach can be considered to be an important safety feature, reducing off-target effects. The cargo is only released if the second effector-chassis is in the vicinity of the protease which itself is only present in the vicinity of the target.

This is just one of many examples of networks and systems that can be put together using the progenitor-chassis, producer or effector-chassis as described herein and receptors of the invention.

This mechanism can be exploited to build signaling networks between progenitor-chassis, producer or effector-chassis as described herein expressing different combinations of ePAR, CPRs, complexes of universal CPRs and tagged targeting peptides, SAPRs and/or ePARs. In some embodiments, the cleaved ePAR propagates a signal through the transmembrane and/or intracellular domains. Activation of an ePAR in this manner thus induces a downstream signaling event including, in the case of engineered platelets or engineered platelet-like membrane bound effector-chassis, platelet recruitment, platelet activation, or release of a cargo such as a therapeutic molecule into the tumor microenvironment.

In some embodiments, the ePAR is an engineered GPCR. In some embodiments the ePAR is PAR1, PAR2, PAR3, or PAR4 wherein the protease site has been engineered to be cleaved by a protease other than thrombin, optionally cleaved by MMPs, Cathepsin B, Urokinases or Capsases. Upon cleavage by the protease, in some embodiments a portion of the ePAR is released. In some embodiments the portion of the ePAR that is released upon cleavage is a signaling molecule.

In some embodiments the ePAR is a GPCR, optionally is an engineered PAR1, PAR2, PAR3 or PAR4, optionally has at least 75%, 80%, 85%, 90%, 92%, 94%, 96%, 98% or 100% sequence identity to the sequence of PAR1, PAR2, PAR3 or PAR4.

It is clear from the disclosure herein that the CPR, universal CPR, complex of universal CPR and tagged targeting peptide, SAPR or ePAR of the invention has a role in the treatment or prevention or one or more diseases. For example, the combination of one or more CPRs, universal CPRs, complexes of universal CPRs and tagged targeting peptides, SAPRs or ePARs of the invention and a progenitor-chassis, producer or effector-chassis as described herein can be used to deliver cargo to a specific site in the body, or to a specific tissue or cell type, for example to a cancer cell, by virtue of the target binding domain on the CPR, universal CPR, complex of universal CPR and tagged targeting peptide, SAPR or ePAR. In preferred embodiments, the effector-chassis is a platelet or a platelet-like membrane-bound cell fragment that comprises one or more cargo and expresses one or more CPRs, universal CPRs, complexes of universal CPRs and tagged targeting peptides, SAPRs or ePARs of the invention, and the CPR, universal CPR, complex of universal CPR and tagged targeting peptide, SAPR or ePAR is such that upon binding of the target binding domain to the target the platelet degranulates, and so delivers the cargo to the target site. The cargo may be a therapeutic cargo as described herein.

In some embodiments, the CPR, universal CPR, complex of universal CPR and tagged targeting peptide, SAPR or ePAR of the invention comprises a region recognized by the autoreactive T cells that mediate a disease. For example, the CPR, universal CPR, complex of universal CPR and tagged targeting peptide, SAPR or ePAR comprises an epitope from the molecular target in Tables 12-20 as presented on pages 129-134 of PCT/GB2020/053247 which is hereby incorporated by reference loaded on to an MHC-ITAM fusion to directly target the autoreactive T cells. The engineered platelets may be loaded with cytotoxic or immunosuppressive protein or antibodies, which are released on activation of the platelet.

For instance, some cases of diabetes mellitus type 1 (T1DM) features T cells specific to a particular insulin peptide. Therefore, using the MHC1-ITAM receptor fusion protein with an autoimmune driving peptide, in a platelet designed to release immunosuppressive factors would result in T cell specific immunosuppression. Exposure of an IL-2 receptor (IL-2R) to compete for IL-2, release of TGF-β1 or IL-10, and many other potential options on MHC1-ITAM activation mediates immunosuppression similar to regulatory T (T_{reg}) cells.

In some embodiments, the progenitor, producer or effector-chassis as described herein or engineered progenitor, producer or effector-chassis as described herein comprises a CPR, universal CPR, complex of universal CPR and tagged targeting peptide, SAPR or ePAR with a major histocompatibility complex (MHC) class I or class II and can be used in the treatment of an autoimmune disease. T cells expressing chimeric antigen receptors (CAR) comprising the MHC ligand of a pathogenic T cell receptor as an antigen binding domain of the CAR have been previously shown to be effective in the treatment of type 1 diabetes (T1D) (See, Perez et al., Immunology, 143, 609-617, which is hereby incorporated by reference in its entirety). In T1D, autoreactive CD8 and CD4 T cells selectively destroy insulin-producing B cells in the pancreas (Ibid.). Some of the MHC-II-restricted epitopes recognized by the autoreactive cells have been observed to be derived from insulin/pre-proinsulin, islet-specific glucose-6-phosphatase catalytic subunit-related protein, glutamic acid decarboxylases 65 and 67, heat-shock proteins 60 and 70, insulinoma-associated protein 2, zinc transporter ZnT8, islet amyloid polypeptide, chromogranin A, and other self antigens (Ibid.). Therefore, in some embodiments, the engineered platelets described herein include a CPR with a ligand or fragment thereof that interacts with the autoreactive cells to destroy the cells.

The invention also provides a nucleic acid encoding the CPR, universal CPR, complex of universal CPR and tagged targeting peptide, tagged targeting peptide, SAPR and/or ePAR of the invention. The invention provides a nucleic acid that encodes both the universal CPR and tagged target binding peptide in a single nucleic acid molecule. In preferred instances, the CPR, universal CPR, complex of universal CPR and tagged targeting peptide, SAPR and/or ePAR are not naturally occurring receptors, and so the nucleic acids encoding said receptors are also not a naturally occurring nucleic acid. In some embodiments the nucleic acid encodes the CPR, universal CPR, complex of universal CPR and tagged targeting peptide, SAPR and/or ePAR of the invention and also comprises a heterologous nucleic acid sequence. In some instances the nucleic acid is operatively linked to an expression control sequence. Expression control sequences are considered to include components such as enhancers and promoters. In one embodiment the nucleic acid of the invention comprises a heterologous promoter. In the same or different embodiment the nucleic acid of the invention comprises a heterologous enhancer sequence.

In some embodiments the nucleic acid is DNA. In some embodiments the nucleic acid is RNA for example is an mRNA. In some embodiments the nucleic acid is arranged to be operably controlled by a promoter, i.e. a promoter that drives expression from the nucleic acid. In some embodiments the promoter is not a megakaryocyte-specific or platelet-specific promoter. In other embodiments the promoter comprises a megakaryocyte-specific promoter or a platelet-specific promoter. The terms megakaryocyte-specific promoter and platelet-specific promoter are used synonymously. The skilled person understands what is meant by the terms megakaryocyte-specific promoter and platelet-specific promoter. In some embodiments the nucleic acid is operatively linked to a heterologous expression sequence, optionally a heterologous promoter.

In some embodiments the promoter is an inducible promoter, for example a promoter that is inducible in an intended subject. For example, where the CPR, universal CPR, complex of universal CPR and tagged targeting peptide, tagged targeting peptide, SAPR and/or ePAR is for use in a human subject, the promoter that drives expression of the CPR, universal CPR, complex of universal CPR and tagged targeting peptide, tagged targeting peptide, SAPR and/or ePAR is in some embodiments an inducible promoter.

In some embodiments the promoter is a constitutive promoter, for example a promoter that is constitutive in an intended subject. For example, where the CPR, universal CPR, complex of universal CPR and tagged targeting peptide, tagged targeting peptide, SAPR, or ePAR is for use in a human subject, the promoter that drives expression of the CPR, universal CPR, complex of universal CPR and tagged targeting peptide, tagged targeting peptide, SAPR and/or ePAR is in some embodiments a constitutive promoter.

The invention also provides a vector that comprises a nucleic acid that encodes the CPR, universal CPR, complex of universal CPR and tagged targeting peptide, tagged targeting peptide, SAPR and/or ePAR of the invention. By vector we include the meaning of plasmid. In some embodiments the vector also comprises a heterologous nucleic acid. In some embodiments the vector comprises a promoter, for example a megakaryocyte-specific promoter. In some embodiments the vector comprises a platelet-specific promoter.

The invention also provides a viral particle, or viral vector, comprising any one or more of the nucleic acids of the invention.

By “engineer” we include the meaning of any manipulation that can affect the gene sequence and/or protein sequence—for example we include manipulations made at the nucleic acid level, for example using CRISPR based nucleic acid editing and homologous recombination; and we also include manipulations made at the translational level, for example the repression of translation via RNAi.

As described above, the invention provides a CPR, universal CPR, complex of universal CPR and tagged targeting peptide, SAPR and ePAR. These are all receptors that sit within a surface membrane of a cell or a platelet and bind to a particular target (for example a cancer neo-antigen, or a TCR) and/or are cleaved by specific proteases, which triggers subsequent platelet modulation events, that can result in cargo unloading, activation of T cells or other intracellular signaling events. Accordingly, when used in practice, for example to direct delivery of a cargo to a particular cell or tissue within the body, or to activate T cells, the CPR, universal CPR, complex of universal CPR and tagged targeting peptide, SAPR or ePAR is deployed in the context of an effector-chassis wherein the CPR, universal CPR, complex of universal CPR and tagged targeting peptide, SAPR or ePAR is localised to the plasma membrane of the effector-chassis.

As described above, platelets have unique properties that make them advantageous for use as targeted delivery vehicles.

Modifications intended to drive the differentiation of a progenitor chassis to a producer chassis (for example Forward programming) may be carried out by any method, and can involve the knockin of genes, for example transcription factors. Each particular gene knockin can be introduced in a number of ways, for example a first gene can be introduced to a first allele of a first locus, and/or a first gene can be introduced to a second allele of a first locus. Additionally or alternatively, a first gene can be introduced into a first allele of a first locus and a second gene can be introduced in to a first allele of a second locus. Additionally or alternatively, a first gene can be introduced into a first allele of a first locus and a second gene can be introduced into a second allele of the first locus. These various combinations apply to the introduction of protein coding genes, and also to the introduction of functional RNA coding sequences, such as those that encode RNA sequences involved in RNAi. Each gene can be introduced under a combination of constitutive and inducible promoters.

Engineering steps referred to herein can be performed in any of the progenitor, producer and/or effector-chassis—however the skilled person appreciates that platelets and platelet-like membrane-bound cell fragments do not comprise a nucleus and so it is not possible to engineer the nucleic acid of the platelet or platelet-like membrane-bound cell fragments, and instead, the engineering is typically performed on one or more of the upstream chassis that differentiate into the producer-chassis (for example a megakaryocyte) that produces the platelets or platelet-like membrane-bound cell fragments—i.e. the engineering steps are typically performed in the progenitor-chassis or the producer-chassis. Some means of disrupting pathways, such as the thrombogenic pathway, are compatible with platelets, for example siRNA can be used in platelets to prevent expression from particular mRNAs and so it is appropriate in some instances to engineer the effector-chassis directly.

References herein to “engineering” in terms of engineering a progenitor, producer or effector-chassis as described herein is to be taken to refer to any appropriate means of modulating the function of one or more genes or proteins in the chassis in a desirable way. For example in some embodiments, the engineering is to reduce or inhibit expression of a protein; and in some embodiments the engineering is to increase expression of a particular protein. A progenitor, producer or effector-chassis may be engineered to have any number of modifications, for example be engineered to disrupt or inhibit expression of any number of proteins and/or to increase the expression of any number of proteins. Exemplary means of disrupting gene expression include those that at act at the DNA level, the transcriptional level, the translational level and the post-translational level, and for example include CRISPR/Cas systems, zinc finger nucleases, transcription activator-like effector nucleases (TALENs), a RNA interference construct (RNAi) (e.g., small interfering RNA (siRNA) or microRNA (miRNA)), or a short hairpin RNA (shRNA). Any means of preventing expression of the ultimate gene product (for example a protein where the gene is a protein encoding gene; or an RNA where the gene or nucleic acid encodes an active RNA) is considered to be appropriate (provided the method does not kill the chassis). Intrabody technology can also be used to regulate gene expression.

We include the meaning of any manipulation that can affect the gene sequence and/or protein sequence—for example we include manipulations made at the genomic level, for example making modifications, deletions, substitutions etc in the genomic nucleic acid of a progenitor, producer or effector-chassis, for example using CRISPR based nucleic acid editing and homologous recombination; and we also include manipulations made at the translational level, for example the repression of translation via RNAi or siRNA. For example an engineered progenitor, producer or effector-chassis as described herein may have one or more gene deletions, single mutations or insertions, gene knockins, promoter substitutions etc; or may express one or more regulatory molecules such as an RNAi. We also include making modification at the protein level, for example by making modifications such as the phosphorylation of a particular protein. All methods of modification are encompassed here. Preferably the modifications are made at the genomic level. In view of at least this disclosure, the skilled person understands what is meant by “engineering” in the context of an engineered a progenitor, producer or effector-chassis as described herein.

The ultimate entity that is administered to a subject, is the effector-chassis e.g. a platelet, or a platelet-like membrane-bound cell fragment (e.g. the platelet used to deliver a cargo that has, in preferred embodiments, been engineered so as to disrupt one or more of the native signaling pathways of a platelet, for example to disrupt the thrombogenic pathway) is derived from a series of increasingly differentiated cells, including a myeloid stem cell, a megakaryoblast, a megakaryocyte or iPSC or and don't comprise a nucleus themselves. It is clear then to the skilled person that references to modification of the nucleic acid of a platelet is intended to encompass modification to the progenitor and producer-chassis e.g. the myeloid stem cell, a megakaryoblast, a megakaryocyte or iPSC, since it is modifications in the myeloid stem cell, a megakaryoblast, a megakaryocyte or iPSC that ultimately determines what proteins are expressed in the platelet or platelet-like membrane bound cell fragment.

It is clear that in some instances it may not be necessary to knock out or delete the entire gene in a particular pathway. For example GP1b knockout results in abnormal platelets, however one can delete only the extracellular domain of the receptor (removing its ability to function) while retaining the intracellular domain, resulting in typical platelets that lack the ability to bind to von Willebrand factor the GP1b target). Accordingly in some embodiments, the disruptions, deletions or knockouts described herein are full disruptions, deletions or knockouts of the entire gene. In other embodiments, the disruptions, deletions and knockouts are disruptions deletions and functional knockouts i.e. disruption of the function of the protein, and in some embodiments the deletion is a deletion of the extracellular domain of the proteins.

The term “platelet-like membrane-bound cell fragment” is intended to encompass the fact that in some instances, many of the biological markers and functions that are routinely used to classify a structure as a platelet have been intentionally disrupted and so it may not be possible to classify the engineered platelet as a “platelet” according to standard definitions. For example, platelet aggregation is used to determine platelet function in clinical samples. In some instances as described herein, the thrombogenic system of the platelets has been disrupted, and so the engineered platelets are unable to aggregate. In such instances it may be contrary to standard definition to term such entities “platelets”. Herein, a platelet or a platelet-like membrane-bound cell fragment is defined herein as an entity that is produced from a megakaryocyte or megakaryocyte-like cell by fragmentation in the typical way that platelets are made. Accordingly in one embodiment, a platelet-like membrane-bound cell fragment is defined as the cell fragments produced from a megakaryocyte that has been engineered to disrupt one or more signaling pathways, for example to disrupt the thrombogenic pathway. Similarly, due to the pathways that may be disrupted, an engineered megakaryocyte may not fit the standard definition of a megakaryocyte since in some embodiments one or more of the defining markers or functions may be disrupted in the engineered megakaryocyte. Accordingly, in some instance the term megakaryocyte-like cell may be preferable. The skilled person is be able to determine whether the megakaryocyte, or megakaryocyte-like cell retains the required functions, namely being the ability to produce platelets or platelet-like membrane cell fragments. The engineered megakaryocyte or megakaryocyte-like cell should retain the ability to produce pseudopodal extensions.

Producer-chassis such as megakaryocytes and effector-chassis such as platelets also express (or otherwise comprise) a specific isoform of Tubulin—TUBB1 (beta1-tubulin). TUBB1 is a component of the microtubules that form the platelet cytoskeleton. For example, although platelets do not comprise a nucleus, the platelets still comprise TUBB1 protein, for example via translation of TUBB1 mRNA or by virtue of the platelet being a fragment of a producer-chassis such as a megakaryocyte that does express TUBB1. TUBB1 is necessary for the function of the platelet, and so is considered to be a useful marker for the skilled person to use to determine whether a progenitor, producer or effector-chassis for example an engineered progenitor, producer or effector-chassis that has been engineered to remove some of the markers that are typically used to identify a megakaryocyte or platelet is still actually a progenitor, producer or effector-chassis as described herein. Accordingly, in some embodiments the progenitor, producer or effector-chassis, for example the platelet or platelet-like membrane-bound cell fragment, or the megakaryocyte-like cell expresses TUBB1. See for example Schwer et al 2001 Curr Biol 11: 579-586 and Kunishima et al 2009 Blood 113: 458-461.

The skilled person appreciates that by “expresses TUBB1” we include the meaning of TUBB1 variants that retain the necessary functions of TUBB1 that are required for platelet production. i.e. TUBB1 may comprise a number of mutations or substitutions relative to the naturally occurring TUBB1 sequences but which retain TUBB1 function. Accordingly, in some embodiments it is more appropriate to state that the chassis comprises TUBB1.

The progenitor, producer or effector-chassis described herein may be engineered (in addition to any engineering necessary to direct differentiation to a megakaryocyte, for example engineering to drive the differentiation of the chassis, for example to forward program the cell) to disrupt one or more signaling pathways, or may not be engineered to disrupt one or more signaling pathways. A progenitor, producer or effector-chassis that is not engineered to disrupt one or more signaling pathways may still be engineered to express one or more proteins or to comprise one or more mutations or other modifications. For example a progenitor, producer or effector-chassis that is not engineered to disrupt one or more signaling pathways may still be engineered to express one or more of the CPRs, universal CPRs, complexes of universal CPRs and tagged targeting peptides, SAPRs or ePARs described herein. A progenitor, producer or effector-chassis that is not engineered to disrupt one or more signaling pathways may additionally or alternatively have been engineered to knock in the relevant genes for differentiation, for example genes needed for forward programming.

Accordingly, in one embodiment the invention provides a progenitor, producer or effector-chassis as described herein, for example a myeloid stem cell, a megakaryoblast, a megakaryocyte, megakaryocyte-like cell, an iPSC, adipocyte, adipose-derived mesenchymal stromal/stem cell line (ASCL) a platelet or a platelet-like membrane-bound cell fragment, that comprises:
one or more CPRs, universal CPRs, complexes of universal CPRs and tagged targeting peptides, SAPRs, or ePARS according to the invention;
one or more nucleic acids according to any of the preceding claims that encodes the CPR, universal CPR, SAPR, or ePAR according to the invention;
one or more vectors according to the previous claims that comprises one or more nucleic acids according to any of the preceding claims that encodes the CPR, universal CPR, SAPR, or ePAR according to the invention; and/or
one or more viral vectors according to the invention that comprises one or more nucleic acids according to the invention that encodes the CPR, universal CPR, SAPR, or ePAR according to the invention.

In these embodiments, the progenitor, producer or effector-chassis, for example a myeloid stem cell, a megakaryoblast, a megakaryocyte, megakaryocyte-like cell, an iPSC, adipocyte, adipose-derived mesenchymal stromal/stem cell line (ASCL) a platelet or a platelet-like membrane-bound cell fragment, is very similar to the “base” chassis, for example very similar to a platelet that would be found naturally in the human body, with the difference being that the progenitor, producer or effector-chassishas been engineered to express one or more CPRs, universal CPRs, complexes of universal CPRs and tagged targeting peptides, SAPRs or ePARs described herein. The progenitor, producer or effector-chassis may also have been engineered to drive differentiation, for example may have been forward programmed.

In some embodiments the progenitor, producer or effector-chassis for example myeloid stem cell, a megakaryoblast, a megakaryocyte, megakaryocyte-like cell, an iPSC, adipocyte, adipose-derived mesenchymal stromal/stem cell line (ASCL) a platelet or a platelet-like membrane-bound cell fragment,
has not been engineered:
   to modulate one or more signaling pathways, optionally engineered to disrupt the thrombogenic pathway and/or engineered to disrupt a platelet inflammatory signaling pathway and/or engineered to make the engineered progenitor, producer or effector-chassis less immunogenic; and/or
   to enhance or disrupt one or more base functions of the progenitor, producer or effector-chassis, optionally wherein the one or more or base functions are involved in the base and/or adaptive immune response, inflammation,
   angiogenesis, atherosclerosis, lymphatic development and tumour growth.

A progenitor, producer or effector-chassis of these embodiments, where the progenitor, producer or effector-chassis has not been engineered to modulate one or more signaling pathways or to enhance or disrupt one or more base functions of the progenitor, producer or effector-chassis but which expresses one or more CPRs, universal CPRs, complexes of universal CPRs and tagged targeting peptides, SAPRs or ePARs can be used to target delivery of a cargo to a particular site in the body, tissue or cell. However, since these progenitor, producer or effector-chassis retain properties that are inherent in a platelet, namely thrombogenic potential, upon binding of the CPR, universal CPR, complexes of universal CPR and tagged targeting peptide, SAPR or ePAR to the target, the platelet triggers thrombogenesis. This may be useful in for example the treatment of cancer since the formation of a blood clot around the tumour can starve it of oxygen, or in situations where restricting oxygen to a vessel or organ that has suffered trauma would be useful.

It is important, in all instances when engineering the progenitor, producer or effector-chassis, that the ability of the megakaryocyte or megakaryocyte-like cell to produce platelets, or platelet-like membrane-bound cell fragments is maintained. The skilled person is able to determine whether such an engineered megakaryocyte is able to produce platelets or platelet-like membrane-bound cell fragments.

However, a platelet that has thrombogenic potential has limited use in the body due to safety concerns.

Accordingly, in some preferred embodiments, the progenitor, producer or effector-chassis has been engineered to modulate, for example to disrupt one or more signaling pathways, for example to disrupt the thrombogenic signaling pathway, a platelet inflammatory signaling pathway, and/or to make the engineered progenitor, producer or effector-chassis less immunogenic. Any signaling pathway of the platelet may be modulated. For example the modulation of some signaling pathways can enhance some desirable features of a progenitor, producer or effector-chassis for example of a platelet or a platelet-like membrane-bound cell fragment that are base signaling pathways the progenitor, producer or effector-chassis—for example to modulate pathways involved in the innate and/or adaptive immune response, for example in inflammation, angiogenesis, atherosclerosis, lymphatic development and tumour growth. Accordingly in one embodiment the invention provides an engineered progenitor, producer or effector-chassis that has been engineered to modulate one or more signaling pathways, for example engineered to disrupt the thrombogenic pathway and/or engineered to disrupt a platelet inflammatory signaling pathway and/or engineered to make the engineered progenitor, producer or effector-chassis less immunogenic and/or engineered to enhance or disrupt one or more base functions of the progenitor, producer or effector-chassis.

The engineered progenitor, producer or effector-chassis described herein that have been engineered to modulate one or more signaling pathways, for example that have been engineered to disrupt the thrombogenic pathway and/or engineered to disrupt a platelet inflammatory signaling pathway and/or engineered to make the engineered progenitor, producer or effector-chassis less immunogenic and/or engineered to enhance or disrupt one or more base functions of the progenitor, producer or effector-chassis, have use beyond any effect related to the CPR, universal CPR, complex of universal CPR and tagged targeting peptide, SAPR or ePAR of the invention, and so it is clear that the invention provides an engineered progenitor, producer or effector-chassis which is any of the engineered progenitor, producer or effector-chassis described herein for example a myeloid stem cell, a megakaryoblast, a megakaryocyte, a megakaryocyte-like cell, an iPSC, a platelet, or a platelet-like membrane-bound cell fragment, wherein the progenitor, producer or effector-chassis does not comprise:
one or more CPRs, universal CPRs, complexes of universal CPRs and tagged targeting peptides, SAPRs or ePARs of the invention;
one or more nucleic acids that encode one or more CPRs, universal CPRs, complexes of universal CPRs and tagged targeting peptides, SAPRs or ePARs of the invention;
one or more vectors that comprises one or more nucleic acids that encode one or more CPRs, universal CPRs, complexes of universal CPRs and tagged targeting peptides, SAPRs or ePARs of the invention; and/or
one or more viral vectors that comprises one or more nucleic acids that encode one or more CPRs, universal CPRs, complexes of universal CPRs and tagged targeting peptides, SAPRs or ePARs of the invention.

For example, the invention provides an engineered progenitor, producer or effector-chassis (for example an engineered myeloid stem cell, a megakaryoblast, a megakaryocyte, a megakaryocyte-like cell, an iPSC, a platelet, or a platelet-like membrane-bound cell fragment), wherein the progenitor, producer or effector-chassis has been engineered to modulate one or more signaling pathways (for example that have been engineered to disrupt the thrombogenic pathway and/or engineered to disrupt a platelet inflammatory signaling pathway and/or engineered to make the engineered progenitor, producer or effector-chassis less immunogenic) and/or engineered to enhance or disrupt one or more base functions of the progenitor, producer or effector-chassis, and wherein the progenitor, producer or effector-chassis does not comprise:
a CPR, universal CPR, complex of universal CPR and tagged targeting peptide, SAPR or ePAR of the invention;
one or more nucleic acids that encode one or more CPRs, universal CPRs, complexes of universal CPRs and tagged targeting peptides, SAPRs or ePARs of the invention;
one or more vectors that comprises one or more nucleic acids that encode one or more CPRs, universal CPRs, complexes of universal CPRs and tagged targeting peptides, SAPRs or ePARs of the invention; and/or
one or more viral vectors that comprises one or more nucleic acids that encode one or more CPRs, universal CPRs, complexes of universal CPRs and tagged targeting peptides, SAPRs or ePARs of the invention.

For example some of the engineered progenitor, producer or effector-chassis described herein have reduced thrombogenic potential and/or immunogenicity relative to a progenitor, producer or effector-chassis that has not been engineered to have reduced thrombogenic potential and/or immunogenicity which can be useful in situations that do not involved the CPR, universal CPR, complex of universal CPR and tagged targeting peptide, SAPR, or ePAR of the invention. For example an engineered progenitor, producer or effector-chassis that does not comprise a receptor of the invention is considered to be useful in situations where clotting is not desired, for example in stroke or MI—for example platelets that lack thrombogenic capabilities but comprise the external receptors are recruited to the site of thrombosis, but will interfere with the thrombogenic process. Preferences for features of the CPRs, universal CPRs, complexes of universal CPRs and tagged targeting peptides, SAPRs or ePARs are as described herein.

However, in some advantageous embodiments, the engineered progenitor, producer or effector-chassis as described herein do comprise one or more CPRs, universal CPRs, complexes of universal CPRs and tagged targeting peptides, SAPRs or ePARs. Accordingly, the invention provides any of the engineered progenitor, producer or effector-chassis (for example an engineered myeloid stem cell, a megakaryoblast, a megakaryocyte, a megakaryocyte-like cell, an iPSC, a platelet, or a platelet-like membrane-bound cell fragment), wherein the progenitor, producer or effector-chassis has been engineered to modulate one or more signaling pathways (for example that have been engineered to disrupt the thrombogenic pathway and/or engineered to disrupt a platelet inflammatory signaling pathway and/or engineered to make the engineered progenitor, producer or effector-chassis less immunogenic) and/or engineered to enhance or disrupt one or more base functions of the progenitor, producer or effector-chassis, and wherein the engineered progenitor, producer or effector-chassis comprises any one or more of:
a CPR, universal CPR, complex of universal CPR and tagged targeting peptide, SAPR or ePAR of the invention;
one or more nucleic acids that encode one or more CPRs, universal CPRs, complexes of universal CPRs and tagged targeting peptides, SAPRs or ePARs of the invention;
one or more vectors that comprises one or more nucleic acids that encode one or more CPRs, universal CPRs, complexes of universal CPRs and tagged targeting peptides, SAPRs or ePARs of the invention; and/or
one or more viral vectors that comprises one or more nucleic acids that encode one or more CPRs, universal CPRs, complexes of universal CPRs and tagged targeting peptides, SAPRs or ePARs of the invention.

Preferences for features of the CPRs, universal CPRs, complexes of universal CPRs and tagged targeting peptides, SAPRs or ePARs are as described herein.

It is clear that the nucleic acid that encodes any of the CPR, universal CPR, complex of universal CPR and tagged targeting peptide, SAPR or ePAR of the invention can be introduced in to a progenitor, producer or effector-chassis in a variety of ways. For example, in some embodiments the nucleic acid that encodes any of a CPR, universal CPR, complex of universal CPR and tagged targeting peptide, SAPR or ePAR of the invention is introduced in to the genomic nucleic acid. For example, a nucleic acid encoding a CPR, universal CPR, complex of universal CPR and tagged targeting peptide, SAPR, or ePAR can be introduced to a first allele of a first locus, and/or a the nucleic acid can be introduced to a second allele of a first locus. Additionally or alternatively, the nucleic acid can be introduced into a first allele of a first locus and a second nucleic acid (for example encoding a second CPR, universal CPR, complex of universal CPR and tagged targeting peptide, SAPR, or ePAR) can be introduced in to a first allele of a second locus. Additionally or alternatively, a first nucleic acid can be introduced into a first allele of a first locus and a second nucleic acid can be introduced into a second allele of the first locus.

In some embodiments the nucleic acid that encodes any of a CPR, universal CPR, complex of universal CPR and tagged targeting peptide, SAPR or ePAR of the invention is introduced in to the progenitor, producer or effector-chassis and maintained in the progenitor, producer or effector-chassis episomally.

In some embodiments the nucleic acid that encodes any of a CPR, universal CPR, complex of universal CPR and tagged targeting peptide, SAPR or ePAR of the invention is introduced in to progenitor, producer or effector-chassis via nucleofection.

These various combinations apply to the introduction of protein coding genes, and also to the introduction of functional RNA coding sequences, such as those that encode RNA sequences involved in RNAi.

As described above, the use of platelets and engineered platelets as delivery or targeted delivery vehicles has several advantages over current therapies.

The platelets or engineered platelets described herein, for example platelets that express one or more CPRs, universal CPRs, complexes of universal CPRs and tagged targeting peptides, SAPRs, or ePARS of the invention, may be generated outside the body from megakaryocytes. As the megakaryocyte is maintained in culture outside of the body, it can be extensively edited at the genome level (e.g. by CRISPR/Cas9) without fear of oncogenic transformation in the patient, which is not possible with other competing cell therapy products.

The platelets described herein, for example platelets that express one or more CPRs, universal CPRs, complexes of universal CPRs and tagged targeting peptides, SAPRs, or ePARS of the invention, would have a lifespan in the body of 7-10 days, with little to no potential for continued reproduction, thus little to no chance of forming a tumour itself.

Platelets can be frozen and stored for extended period of time resulting in an extended shelf life, and with currently available technology, engineered platelets could be produced, stored, transported and administered to patients without issue due to their lack of immunogenicity.

In specific embodiments, where the platelet-like membrane-bound cell fragment has been engineered so as to disrupt the thrombogenic potential of the delivery tool, it can be called a Synlet. Synlets can comprise one or more further modifications, and/or disruptions or knockins of signaling pathways.

The progenitor, producer or effector-chassis—for example the myeloid stem cell, a megakaryoblast, a megakaryocyte, adipocyte, adipose-derived mesenchymal stromal/stem cell line (ASCL) or iPSC the platelets, platelet-like membrane-bound delivery tool, or Synlets are preferably produced ex vivo or in vitro. In some instances the progenitor, producer or effector-chassis may be produced in vivo, for example through HSC transplant.

In some embodiments where the progenitor, producer or effector-chassis or engineered progenitor, producer or effector-chassis expresses one or more CPRs, universal CPRs, complexes of universal CPRs and tagged targeting peptides, SAPRs or ePARs of the invention and wherein the one or more CPRs, universal CPRs, complexes of universal CPRs and tagged targeting peptides, SAPRs or ePARs comprises a platelet activation domain e.g. a platelet degranulation triggering domain, in some embodiments it is considered to be necessary that the progenitor, producer or effector-chassis, i.e. the platelet or platelet-like membrane-bound cell fragment, or Synlet degranulates upon binding of the target-binding domain of the one or more CPRs, universal CPRs, complexes of universal CPRs and tagged targeting peptides, SAPRs or ePARs to the target.

In some embodiments where the progenitor, producer or effector-chassis expresses one or more CPRs, universal CPRs, complexes of universal CPRs and tagged targeting peptides, SAPRs or ePARs of the invention and wherein the one or more CPRs, universal CPRs, complexes of universal CPRs and tagged targeting peptides, SAPRs or ePARs comprises a platelet inhibition domain e.g. a domain that prevents the triggering of platelet degranulation, in some embodiments it is considered to be necessary that the progenitor, producer or effector-chassis or the engineered progenitor, producer or effector-chassis, i.e. the platelet or platelet-like membrane-bound cell fragment, or Synlet is able inhibit the activation of degranulation upon binding of the target-binding domain of the one or more CPRs, universal CPRs, complexes of universal CPRs and tagged targeting peptides, SAPRs or ePARs to the target.

Whether a particular effector-chassis needs to retain the ability to activate, i.e. trigger degranulation, or to inhibit the activation of degranulation depends on the nature of the one or more CPRs, universal CPRs, complexes of universal CPRs and tagged targeting peptides, SAPRs or ePARs of the invention that are present in the effector-chassis. Preferences for the CPRs, universal CPRs, complexes of universal CPRs and tagged targeting peptides, SAPRs or ePARs of the invention are as described herein.

In some particular embodiments, it is not considered necessary that the platelet degranulates or does not degranulate, and simply binding of the SAPR to the target is sufficient to produce a useful effect. For example, in some embodiments the target binding domain comprises an MHC/antigen complex. In these embodiments, the SAPR mimics the presentation of an antigen as part of an antigen/MHC complex by antigen presenting cells. T cells can bind, through the T cell receptor (TCR) to the antigen when presented as part of an MHC/antigen complex which results in activation and differentiation of the T cell. This in itself is considered to be an advantageous use of the progenitor, producer or effector-chassis of the invention.

In some embodiments, the invention provides the CPRs as defined by any of SEQ ID NO: 104-111.

In contrast to chimeric antigen receptor T (CAR-T) cells, in some embodiments the present invention provides a progenitor, producer or effector-chassis, for example an engineered myeloid stem cell, a megakaryoblast, a megakaryocyte, a megakaryocyte-like cell, an iPSC, adipocyte, adipose-derived mesenchymal stromal/stem cell line (ASCL), a platelet, or a platelet-like membrane-bound cell fragment that is a universal product which does not require a match to a patient before administration. For example in these embodiments the progenitor, producer or effector-chassis has been engineered so as to have inhibited expression from the beta 2 microglobulin gene, for example through a knockout of the beta 2 microglobulin gene.

Further, platelet or platelet-like membrane-bound cell fragment production in vitro from the progenitors described herein, removes the need to continuously produce virus and edit cells. Due to the short life span of the engineered platelets or engineered platelet-like membrane-bound cell fragments described herein, safety concerns are limited as compared to current gene editing therapeutics. For example, gene editing and genome stability is less of a concern in the present invention than with CAR-T cells because platelets are enucleate and therefore the complexity of the platelet therapy is not limited by the efficiency of editing or culture time limits. Additionally, due to their smaller size, the engineered platelets may provide better access to solid tumors than CAR-T cells.

Enucleated red blood cells, such as those commercially available from Rubius Therapeutics, Inc., have also been contemplated in the art for delivering therapeutic agents. In contrast to red blood cells, the engineered progenitor, producer or effector-chassis, for example engineered platelets or engineered platelet-like membrane-bound cell fragments described herein are highly metabolically active and include signaling systems that can be re-engineered. In fact, more targeted uses are possible with the engineered platelets or engineered platelet-like membrane-bound cell fragments described herein compared to red blood cells.

Vesicle degranulation of the engineered platelets or engineered platelet-like membrane-bound cell fragments described herein also allows for “hiding” of the cargo, for example a cargo protein, until the desired target is engaged, which is not possible with enucleated red blood cells because the biotherapeutic proteins are generally expressed on the surface of the cell.

The engineered platelets or engineered platelet-like membrane-bound cell fragments described herein are also smaller than red blood cells likely resulting in better biodistribution.

Accordingly, in one embodiment binding of the target binding domain of the CPR, universal CPR, complex of universal CPR and tagged targeting peptide, SAPR or ePAR present in a platelet or platelet-like membrane-bound cell fragment to the target or antigen results in degranulation.

To arrive at any of the progenitor, producer or effector-chassis or engineered progenitor, producer or effector-chassis described herein, some modulation of gene expression is used—either to modulate genes that are natively found in a progenitor, producer or effector-chassis, for example an engineered myeloid stem cell, a megakaryoblast, a megakaryocyte, a megakaryocyte-like cell, an iPSC, adipocyte, adipose-derived mesenchymal stromal/stem cell line (ASCL), a platelet, or a platelet-like membrane-bound cell fragment, and/or to introduce non-native genes or other coding sequences to the progenitor, producer or effector-chassis, for example one or more genes encoding one or more CPRs, universal CPRs, complexes of universal CPRs and tagged targeting peptides, SAPRs or ePARs of the invention.

By “modulate” expression we include the meaning of reducing expression levels, completely preventing expression (for example in the case of a gene-knockout), or increasing expression levels.

As described above, a progenitor, producer or effector-chassis as described herein that has thrombogenic potential has limited use in scenarios that involve administering the progenitor, producer or effector-chassis to a human body. Other native signaling pathways may preferably be modulated, for example disrupted or inhibited or enhanced.

Modulation, disruption, inhibition or enhancement of various pathways that are base pathways of the progenitor, producer or effector-chassis is desirable in situations where the progenitor, producer or effector-chassis does not comprise one or more CPRs, universal CPRs, complexes of universal CPRs and tagged targeting peptides, SAPRs or ePARs; and is also desirable in situations where the progenitor, producer or effector-chassis does comprise one or more CPRs, universal CPRs, complexes of universal CPRs and tagged targeting peptides, SAPRs or ePARs. Accordingly, discussion herein regarding the modulation of signaling pathways is to be taken to relate to both embodiments, i.e. the progenitor, producer or effector-chassis of the invention that does and does not comprise the one or more CPRs, universal CPRs, complexes of universal CPRs and tagged targeting peptides of the invention.

Any signaling pathway that is a base signaling pathway of the progenitor, producer or effector-chassis of the invention may be modulated, disrupted, inhibited or enhanced. Exemplary pathways that may be beneficially disrupted include the thrombogenic pathway and/the inflammatory signaling pathway and/or pathways related to platelet immunogenicity. Other exemplary pathways that may be modulated are pathways that are involved in the innate and/or adaptive immune response, inflammation, angiogenesis, atherosclerosis, lymphatic development, tumour growth, platelet adhesion, platelet migration and extravasation.

A particularly advantageous pathway to disrupt is the thrombogenic pathway. Although described herein are some uses of a progenitor, producer or effector-chassis of the invention that retains thrombogenic potential, in preferred embodiments the thrombogenic pathway is disrupted. In more preferred embodiments the entire thrombogenic pathway is disrupted. For example, when targeting a cargo to a particular site in the body it is in most cases preferable that, upon degranulation and release of the cargo, the native thrombogenesis pathway is not triggered—preventing the undesirable formation of clots at the target site. Accordingly, in some embodiments the invention provides an engineered progenitor, producer or effector-chassis, for example an engineered myeloid stem cell, a megakaryoblast, a megakaryocyte, a megakaryocyte-like cell, an iPSC, adipocyte, adipose-derived mesenchymal stromal/stem cell line (ASCL), a platelet, or a platelet-like membrane-bound cell fragment wherein the engineered progenitor, producer or effector-chassis has reduced thrombogenic potential relative to a progenitor, producer or effector-chassis that has not been engineered so as to have reduced thrombogenic potential. In some embodiments the progenitor, producer or effector-chassis has no thrombogenic potential—i.e. are not thrombogenic at all.

In some embodiments, the engineered progenitor, producer or effector-chassis of the invention, for example the engineered myeloid stem cell, a megakaryoblast, a megakaryocyte, a megakaryocyte-like cell, an iPSC, adipocyte, adipose-derived mesenchymal stromal/stem cell line (ASCL), a platelet, or a platelet-like membrane-bound cell fragment is, or produces platelets that are, less thrombogenic than platelets produced from a “natural” engineered myeloid stem cell, a megakaryoblast, a megakaryocyte, a megakaryocyte-like cell, an iPSC, adipocyte, adipose-derived mesenchymal stromal/stem cell line (ASCL), a platelet, or a platelet-like membrane-bound cell fragment—i.e. are less thrombogenic than platelets or platelet-like membrane-bound cell fragments produced from a progenitor, producer or effector-chassis for example from a myeloid stem cell, a megakaryoblast, a megakaryocyte, a megakaryocyte-like cell, or an iPSC that has not been intentionally engineered to have reduced thrombogenicity—for example the engineered progenitor, producer or effector-chassis of this embodiment is less thrombogenic than the corresponding iPSC, megakaryocyte or platelet that is found in vivo, (e.g., platelets from a human donor).

In addition, or alternatively, the engineered progenitor, producer or effector-chassis, for example an engineered iPSC progenitor, adipocyte, adipose-derived mesenchymal stromal/stem cell line (ASCL), megakaryocyte or platelet may contain genetic modifications within the gene components of pathways for platelet adhesion, migration, and extravasation.

In one embodiment, the engineered progenitor, producer or effector-chassis, which as described herein is a progenitor, producer or effector-chassis that has been engineered to modulate one or more signaling pathways, for example an engineered a myeloid stem cell, a megakaryoblast, a megakaryocyte, megakaryocyte-like cell, an iPSC, adipocyte, adipose-derived mesenchymal stromal/stem cell line (ASCL) a platelet or a platelet-like membrane-bound cell fragment has been engineered so as to disrupt one or more functions of the thrombogenic pathway.

An engineered platelet, or engineered platelet-like membrane-bound cell fragment that has been stripped of thrombogenic potential is in some instances also called a SYNLET™ and can act as a blank template in terms of thrombogenicity, effectively functioning as a scaffold, having the capacity to store cargo internally in vesicles, internally in the cytoplasm, or on the outer surface of the plasma membrane. As described herein, expression or one or more CPR, universal CPR, complex of universal CPR and tagged targeting peptide, SAPR or ePAR in the SYNLET (engineered platelet or engineered platelet-like membrane-bound cell fragment that lacks thrombogenic potential) allows the SYNLET to respond to specific antigens or signals. These advantageous engineered platelets and engineered platelet-like membrane-bound cell fragments preferably do not respond to endogenous stimuli that usually result in clot formation, preferably are not recruited by other activated platelets, and on activation, are preferably not be able to recruit and activate endogenous platelets in the patient. It is clear to the skilled person that to produce such advantageous engineered platelets or engineered platelet-like membrane-bound cell fragments, the platelet precursor, i.e. the myeloid stem cell, a megakaryoblast, a megakaryocyte, a megakaryocyte-like cell, adipocyte, adipose-derived mesenchymal stromal/stem cell line (ASCL) or an iPSC, has to be appropriately engineered. Examples of such methods of engineering are described herein.

In some embodiments is may be possible to produce an advantageous platelet or platelet-like membrane-bound cell fragment, for example a platelet that lacks the ability to recruit and activate endogenous platelets in the patient, that is not able to respond to endogenous stimuli that usually result in clot formation, and that are not recruited by other activated platelets, by exogenously treating the platelet or platelet-like membrane-bound cell fragment, for example by exposing the platelet or platelet-like membrane-bound cell fragment to agents that inhibit transcription or translation of the required genes, for example by exposing to siRNA fragments, or CRISPR components targeted to particular transcripts—rather than requiring the progenitor to have been engineered (other than to express the CPR in embodiments of the progenitor, producer or effector-chassis that comprise the CPR).

The thrombogenic pathway comprises a number of pathways that act together to provide the robust thrombogenic response in response to injury, for example. The primary stimuli of thrombosis formation has to be recognised; a secondary stimulus of thrombus formation is recognised; and secondary mediators of thrombus formation are released.

Recognition of primary stimuli of thrombus involves the platelets recognizing factors associated with exposed tissue that becomes exposed upon wounding, for example, recognizing the subendothelium. In typical circumstances, platelets are not exposed to subendothelium. Exposure of the subendothelium allows platelets to recognize ligands such as collagen, von Willebrand factor, fibronectin, thrombospondin via receptors on the platelet surface, such as GPIb/V/IX and GPVI (GP6), ITGA2B, integrins s a_{IIb}b₃, a₂b₁, a₅b₁ and a₆b₁. Accordingly, in some embodiments the genes encoding a protein involved in recognition of primary stimuli of thrombus formation include GPIb/V/IX and GPVI (GP6), ITGA2B, CLEC2, integrins s a_{II}b₃, a₂b₁, a₅b₁ and a₆b₁.

Once platelets have made contact with the exposed endothelium, for example via the interactions discussed above, the platelets release secondary messengers such as ADP, thrombin and TxA₂ which are detected by other platelets and which cause platelet aggregation at the wound site. In some embodiments, it is preferred if the ability of the platelets to recognize the secondary messengers is disrupted. It is not desirable if a platelet of the invention is targeted to wound site for example, rather than the intended target. Accordingly, in preferred embodiments the ability of the platelets to recognize the secondary messengers is disrupted. Receptors that are involved in this function include Par1, Par4, P2Y12, GPIb/V/IX, the Thromboxane receptor (TBXA2R), P2Y1, P2X1 and integrin a_{IIb}b₃.

As mentioned above, once platelets have recognized the exposed tissue, they release secondary messengers to recruit other platelets to the site. Once a platelet of the invention has bound to a target, for example to a tumour antigen, it is not desirable for the platelet of the invention to then recruit other platelets to a target site and form a thrombus, for example a thrombus at a tumour site. Accordingly, in preferred embodiments, the pathway by which the activated platelet releases the secondary messengers is disrupted. The pathway can include those proteins that are involved in the production and/or storage and/or release of the secondary mediators. Genes involved in this pathway include Cox1, Cox2, HPS, TMEM16F, prothrombin, PDGF, EGF, von Willebrand Factor and thromboxane-A synthase (TBXAS1).

Alternatively, the deletion or modification is introduced to genes that mediate platelet signal transduction, such as HPS (biogenesis of lysosomal organelles complex 3 subunit) genes, which are vital to ADP, serotonin, and ATP release from dense granules; and mitochondrially encoded cytochrome C oxidase II (COX2), which generates inflammatory and prothrombogenic mediators and is a target of aspirin. Alternatively, the deletion or modification is introduced to genes expressing thrombotic mediators, such as prothrombin (major protein thrombotic inducer); PDGF which is a pro-angiogenic factor; EGF (elongation growth factor); and von Willebrand Factor (collagen adaptor protein).

The combinatorial loss of thrombin and ADP signaling has been observed to abrogate vessel occlusion, but ITAM receptors can still be activated (See, Boulaftali et al. “Platelet ITAM signaling is critical for vascular integrity in inflammation”. JCI, 2013 and Cornelissen et al. “Roles and interactions among protease-activated receptors and P2ry12 in hemostasis and thrombosis”, PNAS. 2010, each of which is hereby incorporated by reference in its entirety). This work demonstrates that disruption of crucial endogenous platelet signaling pathways does not abrogate a platelet's ability to signal through ITAM receptors, indicating that the engineered CPRs described herein are likely to function on a non-thrombogenic platelet background.

For example, thrombin activates platelets through cleavage of PARs (protease activated receptors). Platelet signaling is also driven by protease activated GPCRs, namely PAR1 and PAR4 which are cleaved by thrombin. Signaling is potent and acts to recruit platelets and facilitate positive feedback between platelets after platelet activation. The thrombin cleavage sequence on PAR1 and PAR4 is well defined.

In some embodiments, the engineered platelets described herein may comprise at least one deletion or modification introduced into or replacing domains of endogenous platelet receptors, such as, but not limited to, PAR4 (protease activated receptor 4), which is a primary thrombin receptor; GP1b-1X-V (Glycoprotein Ib complexed with glycoprotein IX), which is a primary anchor receptor; P2Y12 (purinergic receptor P2Y12), which is an ADP (adenosine diphosphate) receptor and target of clopidogrel inhibition; GPVI (glycoprotein deletiontein VI platelet), which is a collagen receptor; or a thromboxan receptor to prevent activation of the engineered platelet.

It is clear to the skilled person that by a protein involved in recognition of primary stimuli of thrombus we include the meaning of any protein that is involved in this process, for example includes the protein that is directly involved in contact with or recognition of primary stimuli of thrombus, and also genes that for example lead to the expression of those proteins that are directly involved in contact with or recognition of the primary stimuli of thrombus. The skilled person understands which proteins are considered to be involved in recognition of primary stimuli.

The key feature is that disruption of the proteins are that their disruption leads to a defect in the recognition of primary stimuli of thrombus. However, in some embodiments a protein involved in recognition of primary stimuli of thrombus includes only those proteins that directly make contact with the primary stimuli of thrombus.

By a protein involved in recognition of secondary mediators of thrombus formation we include those proteins that are directly involved in the contact with or recognition of secondar mediators of thrombus formation, as well as proteins that are indirectly involved in those processes, for example those proteins that are involved in the production of the proteins that are directly involved in the contact with or recognition of secondar mediators of thrombus formation. The skilled person understands what is mean by proteins involved in recognition of secondary mediators of thrombus formation. The key feature of the proteins are that their disruption leads to a defect in the recognition of secondary mediators of thrombus formation. However, in some embodiments a protein involved in recognition of secondary mediators of thrombus formation includes only those proteins that make direct contact with the secondary mediators of thrombus formation.

By a protein involved in the release of secondary mediators of thrombus formation we include those proteins that are involved in the production and/or storage and/or release of the secondary mediators. The key feature of the proteins are that their disruption leads to a defect in the ultimate release of the secondary mediators. The defect may be in the production of the secondary mediators, the storage of the secondary mediators, and/or the actual release process.

In some embodiments any one or more of the following three pathways are disrupted in the progenitor, producer or effector-chassis: recognition of primary stimuli of thrombus formation; recognition of secondary stimuli of thrombus formation; and release of secondary mediators of thrombus formation. In preferred embodiments, all three of the pathways are disrupted. Engineered platelets stripped of all thrombogenic potential by disrupting the thrombogenic pathways as described herein in the progenitor, producer or effector-chassis, for example engineered iPSC, engineered adipocyte, engineered adipose-derived mesenchymal stromal/stem cell line (ASCL), engineered megakaryocytes or engineered platelet alleviate potential thrombotic safety concerns.

The skilled person appreciates that a single gene can be involved in one, two or three of the above functions.

Examples of genes that may be deleted or disrupted (i.e. expression of the ultimate gene product is prevented) from the engineered progenitor, producer or effector-chassis for example an engineered iPSC or engineered megakaryocyte genome or engineered platelet that are considered to disrupt the thrombogenic response are shown in Table 3 on page 33 of PCT/GB2020/053247 which is hereby incorporated by reference.

In some embodiments, the engineered progenitor, producer or effector-chassis, for example engineered myeloid stem cell, a megakaryoblast, a megakaryocyte, a megakaryocyte-like cell, an iPSC, a platelet, or a platelet-like membrane-bound cell fragment comprises a disruption of a least one gene, at least one, two, three, four, five, six, seven, eight, nine, or at least ten genes encoding an endogenous receptor, mediator protein, and/or signaling transduction protein.

In some embodiments the engineered progenitor, producer or effector-chassis comprises a disruption or deletion of at least:
one gene that encodes a protein involved in recognition of primary stimuli of thrombus formation;
one gene that encodes a protein involved in recognition of secondary mediators of thrombus formation; and
one gene that encodes a protein involved in the release of secondary mediators of thrombus formation;

In some embodiments the engineered progenitor, producer or effector-chassis comprises a disruption or deletion of at least:
two genes that encode a protein involved in recognition of primary stimuli of thrombus formation;
two genes that encode a protein involved in recognition of secondary mediators of thrombus formation; and
two genes that encode a protein involved in the release of secondary mediators of thrombus formation;

In some embodiments the engineered progenitor, producer or effector-chassis comprises a disruption or deletion of at least:
three genes that encode a protein involved in recognition of primary stimuli of thrombus formation;
three genes that encode a protein involved in recognition of secondary mediators of thrombus formation; and
three genes that encode a protein involved in the release of secondary mediators of thrombus formation.
Genes that are considered to encode a protein involved in recognition of primary stimuli of thrombus formation include GPIb/V/IX and GPVI (GP6), ITGA2B, CLEC2, integrins s a_{IIb}b₃, a₂b₁, a₅b₁ and a₆b₁, or optionally include GPVI and ITGA2B.
Genes that are considered to encode a protein involved in recognition of secondary stimuli of thrombus formation include Par1, Par4, P2Y12, GPIb/V/IX, the Thromboxane receptor (TBXA2R), P2Y1, P2X1 and integrin aribb3 or optionally include Par1, Par4 and P2Y12.
Genes that are considered to a protein involved in release of secondary mediators of thrombus formation include Cox1, HPS and thromboxane-A synthase (TBXAS1), or optionally include Cox1 and HPS.

In some embodiments
the at least one, two or three genes that encode a protein involved in recognition of primary stimuli of thrombus formation are selected from the group consisting of: GPIb/V/IX and GPVI (GP6), ITGA2B, CLEC2, integrins s a_{IIb}b₃, a₂b₁, a₅b₁ and a₆b₁, or from the group consisting of GPVI and ITGA2B;
the at least one, two or three that encode a protein involved in recognition of secondary mediators of thrombus formation are selected from the group consisting of Par1, Par4, P2Y12, GPIb/V/IX, the Thromboxane receptor (TBXA2R), P2Y1, P2X1 and integrin a_{IIb}b₃ or from the group consisting of Par1, Par4 and P2Y12; and/or
the at least one, two or three genes that encode a protein involved in the release of secondary mediators of thrombus formation are selected from the group consisting of Cox1, Cox2, HPS, prothrombin, PDGF, EGF, von Willebrand Factor and thromboxane-A synthase (TBXAS1).

In some embodiments, the engineered progenitor, producer or effector-chassis has a disruption or deletion in at least each of the following genes:
ITGA2B, HPS and PSY12.

In some embodiments, the engineered progenitor, producer or effector-chassis has a disruption or deletion in at least each of the following genes:
ITGA2B, HPS1 and PAR1.

In a preferred embodiment, the engineered progenitor, producer or effector-chassis has a disruption or deletion in each of the following genes:
GPVI, ITGA2B, Par1, Par4, P2Y12, Cox1 and HPS.

For example the engineered progenitor, producer or effector-chassis may comprise a knockout of each of GPVI, ITGA2B, Par1, Par4, P2Y12, Cox1 and HPS.

The effects of knock-out of a gene in a megakaryocyte on the resulting engineered platelet may be varied. For example, RAB27a (RAS oncogene) and HPS (haptoglobin) genes function in dense granule loading and formation, respectively. Knock-out or deletion of Rab27a may result in engineered platelets with no dense granule mediators but with otherwise normal platelet biology.

Knock-out or deletion of HPS genes may result in engineered platelets containing no dense granules. Knock-out or deletion of AIIbB3 or GP1b-IX-V may result in failure of the platelets to aggregate with each other by decreasing interaction between the platelet and von Willebrand factors (vWF) after activation. Further, AIIbB3 is also involved in inside-out signaling to increase the affinity of the integrin for fibrinogen (See, Durrant, Blood. 2017 Oct. 5; 130(14): 1607-1619). Knock-out or deletion of IP (PGI2R or prostaglandin I2 receptor) may result in negative regulation of prostaglandin. Knock-out or deletion of TP (TxA2R or Thromboxane A2 Receptor) may result in reduction of recruitment of additional platelets on activation to stimulate clotting.

GPVI (ITAM receptor) has been observed to still be stimulated in G-protein alpha-q (Galphaq) knockout mice. Conversely, ITAM agonists, such as collagen, induce release of G-protein-coupled receptors (GPCR agonists), such as ADP and thromboxane A2 receptor (TXA2), thus indirectly activating phospholipase C (PLC) through the Gq pathway. Further, Galphaq is active for proper function for thrombin, ADP, 5-hydroxytryptamine (5HT), PAF, and thromboxane A (TXA). Knock-out or deletion of P-selectin, thromboxane synthase, and platelet activating factor (PAF) results in failure of platelet aggregation once activated. Knock-out or deletion of LIM Domain Kinase 1 (LIMK1) reduces TxA2 synthesis. CXCL4 (C-X-C motif chemokine ligand 4) and CXCL7 (C-X-C motif chemokine ligand 7) are chemokines; therefore, knock-out or deletion of the gene would interfere in at least one signaling pathway. Talin1 and kindlins function in signal transduction to allow integrins to enter a sensitive state.

Knock-out or deletion of ANO6/TMEM16F disrupts the platelets ability to expose phosphatidylserine on platelet activation. Phosphatidlyserine is a membrane lipid which is usually kept on the cytoplasmic face of the platelet. On platelet activation, calcium influx triggers phosphatidylserine exposure on the outside of the platelet via ANO6/TMEM16F, where it acts to catalyse the production of active thrombin in combination with clotting factors. Thus, knockout of TMEM16F prevents phosphatidylserine exposure and thus would decrease platelet thrombogenicity. This is exemplified by Scott's syndrome patients, who feature ANO6 mutations and clinically have increased risk of bleeding.

It is clear to the skilled person that in addition to, or instead of disrupting the thrombogenic pathway, it is considered advantageous to also modulate the immunogenic and/or inflammatory properties of the engineered progenitor, producer or effector-chassis, for example the engineered iPSC progenitor, megakaryocyte or platelet.

In some embodiments, the engineered progenitor, producer or effector-chassis of the invention, for example the engineered myeloid stem cell, a megakaryoblast, a megakaryocyte, a megakaryocyte-like cell, an iPSC, a platelet, or a platelet-like membrane-bound cell fragment is, or produces platelets that are, less immunogenic than platelets produced from a “natural” engineered myeloid stem cell, a megakaryoblast, a megakaryocyte, a megakaryocyte-like cell, an iPSC, a platelet, or a platelet-like membrane-bound cell fragment—i.e. are less immunogenic than platelets or platelet-like membrane-bound cell fragments produced from a progenitor, producer or effector-chassis for example from a myeloid stem cell, a megakaryoblast, a megakaryocyte, a megakaryocyte-like cell, or an iPSC that has not been intentionally engineered to have reduced immunogenicity—for example the engineered progenitor, producer or effector-chassis of this embodiment is less immunogenic than the corresponding iPSC, megakaryocyte or platelet that is found in vivo, (e.g., platelets from a human donor). By an engineered progenitor, producer or effector-chassis that is less immunogenic than a non-engineered progenitor, producer or effector-chassis we include the meaning that the progenitor, producer or effector-chassis is a hypoimmunogenic producer or effector-chassis.

The engineered progenitor, producer or effector-chassis of the invention, for example the engineered iPSC, engineered megakaryocyte or engineered platelet may be made universal through deletion of the β2 microglobulin gene (See, Feng et al. “Scalable Generation of Universal Platelets from Human Induced Pluripotent Stem Cells”. Stem Cell Reports, 2014, which is hereby incorporated by reference in its entirety). Even without this deletion, platelets with ABO matching are generally used in clinical practice without adverse effects. O-type platelets from humans are not universal donors as they are contaminated with anti-A/B antibodies, but contamination would not be an issue with in vitro platelets. Therefore, in certain embodiments, the inventions described herein may use these technologies to mass produce gene-edited platelets, which are also easily stored, transported, and do not require patient matching.

Additional strategies that are considered to be suitable for the generation of a hypo-immune progenitor, producer or effector-chassis, i.e. a progenitor, producer or effector-chassis such as an engineered myeloid stem cell, a megakaryoblast, a megakaryocyte, a megakaryocyte-like cell, an iPSC, a platelet, or a platelet-like membrane-bound cell fragment with reduced immunogenicity. See for example Sugimoto, N & Eto, K. Cellular and Molecular Life Sciences (2021) doi.org/10.1007/s00018-020-03749-8).

In some embodiments there are 3 strategies that can be used to make a progenitor, producer or effector-chassis for example an engineered myeloid stem cell, a megakaryoblast, a megakaryocyte, a megakaryocyte-like cell, an iPSC, a platelet, or a platelet-like membrane-bound cell fragment that is less immunogenic than a non-engineered engineered myeloid stem cell, a megakaryoblast, a megakaryocyte, a megakaryocyte-like cell, an iPSC, a platelet, or a platelet-like membrane-bound cell fragment:
1. Generation of a universal progenitor, producer or effector-chassis by disruption of HLA-class Ia expression on cell surface
2. Generation of a universal progenitor, producer or effector-chassis overexpression of HLA class Ib genes
3. Generation of universal progenitor, producer or effector-chassis by overexpression of immune-modulatory genes.

It is clear to the skilled person that these strategies can be combined for potentiation of hypo-immune effects. Each strategy is discussed and exemplified below.

### Strategy #1: Generation of a Universal Engineered Progenitor, Producer or Effector-Chassis by Disruption of HLA-Class Ia Expression on Cell Surface

1.1 Disruption of Expression Beta 2 Microglobulin (b2M)

Several investigators have designed hPSCs in which B2M is knocked out. This is because the B2M protein forms a heterodimer with HLA class I proteins and is required for HLA class I expression on the cell surface. Knocking out the B2M gene can restrict an immune response from cytotoxic CD8+ T cells by depleting all HLA class I molecules (HLA-A, -B, -C, -E, -F and -G). Disruption of the B2M gene can be achieved using CRISPR, TALEN or RNA interference or other methods of engineering described herein. This has been demonstrated in CD34+ HSC progenitor cells and in hiPSCs (Borger A K, Eicke D, Wolf C, et al. Generation of HLA-universal iP-SC-derived megakaryocytes and platelets for survival under refractoriness conditions. Mol Med. 2016; 22:2742-2785; Norbnop P, Ingrungruanglert P, Israsena N, Suphapeetiporn K, Shotelersuk V. Generation and characterization of HLA-universal platelets derived from induced pluripotent stem cells. Sci Rep. 2020; 10(1):8472; Figueiredo C, Goudeva L, Horn P A, Eiz-Vesper B, Blasczyk R, Seltsam A. Generation of HLA-deficient platelets from hematopoietic progenitor cells. Transfusion. 2010; 50(8):1690-1701; Gras C, Schulze K, Goudeva L, Guzman C A, Blasczyk R, Figueiredo C. HLA-universal platelet transfusions prevent platelet refractoriness in a mouse model. Hum Gene Ther. 2013; 24(12):1018-1028.).

Norbnop et al generated HSCs, MKs and platelets differentiated from B2M-knockout hiPSCs using CRISPR gene editing. HLA-universal hiPSC-derived platelets were found to be functional: activated by enhanced CD62P (activated platelet) expression and enhanced aggregation by stimulation with thrombin and arachidonic acid (classic platelet agonists). Interestingly, Suzuki et al (Suzuki D, Flahou C, Yoshikawa N, Stirblyte I, Hayashi Y, Sawaguchi A, Akasaka M, Nakamura S, Higashi N, Xu H, Matsumoto T, Fujio K, Manz M G, Hotta A, Takiåzawa H, Eto K, Sugimoto N (2020) iPSC-derived platelets depleted of HLA class I are inert to anti-HLA class I and natural killer cell immunity. Stem Cell Rep 14(1):49-59) found that B2M-KO iPSC-PLTs, which are completely devoid of HLA-I expression, do not elicit an NK cell response in vitro. While this observation removes the concern of NK cells rejecting HLA-KO platelets (see Strategies #2 and #3), the reason for the low immunogenicity of platelets remains unknown.

In some embodiments, expression of B2M is knocked-out using any one or more of the following gRNAs (see Example 5):

B2M_1:
[SEQ ID NO: 112]
AAGUCAACUUCAAUGUCGGA
B2M_2:
[SEQ ID NO: 113]
AGUCACAUGGUUCACACGGC
B2M_3:
[SEQ ID NO: 114]
ACUUGUCUUUCAGCAAGGAC
B2M_4:
[SEQ ID NO: 115]
UCACGUCAUCCAGCAGAGAA

Preferably any of:

B2M_2:
[SEQ ID NO: 113]
AGUCACAUGGUUCACACGGC
B2M_4:
[SEQ ID NO: 115]
UCACGUCAUCCAGCAGAGAA

Preferably:

B2M_4:
[SEQ ID NO: 115]
UCACGUCAUCCAGCAGAGAA.

Even significant but not total abolition of B2M expression can provide the desired hypo-immune phenotype. Using a lentiviral delivered B2M short hairpin RNA (shRNA), Karabekian et al (Karabekian Z, Ding H, Stybayeva G, et al. HLA class I depleted hESC as a source of hypoimmunogenic cells for tissue engineering applications. Tissue Eng Pt A. 2015; 21(19-20):2559-2571) reported that the mRNA levels of B2M were decreased by 90%. The continuous suppression of B2M expression by shRNA was effective in suppressing not only immune reactions by T-cell activation but also NK cell responses. The latter effect might be due to the fact that shRNA expression in the cells did not lead to a complete elimination of B2M expression in hESCs and this conferred the advantage of escaping NK cell-mediating cell killing.

Accordingly, in some embodiments the engineered progenitor, producer or effector-chassis of the invention has been engineered to have reduced immunogenicity with respect to a non-engineered progenitor, producer or effector-chassis. In some embodiments the engineered progenitor, producer or effector-chassis that has been engineered to have reduced immunogenicity with respect to a non-engineered chassis has been engineered so as to disrupt the function of endogenous MHC Class 1 has been disrupted; and/or disrupt expression from the 02 microglobulin gene has been disrupted. In some embodiments the β2 microglobulin gene has been knocked out. In some embodiments the β2 microglobulin gene has been knocked out through the use of CRISPR gene editing, or shRNA, optionally lentiviral delivery of shRNA.

1.2 Disrupting Expression from HLA Genes

In addition to, or instead of to B2M disruption, is the knocking out of HLA-A, B and C molecules: Deuse et al (Deuse T, Seifert M, Tyan D, et al. Immunobiology of naive and genetically modified HLA-class-I-knockdown human embryonic stem cells. J Cell Sci. 2011; 124(Pt 17):3029-3037) prepared hESCs in which HLA class I molecules were knocked down using intrabody technology to generate hypoimmunogenic hESCs. The engineered hESCs induced an extensively reduced immune response from T cells, NK cells and macrophages thus extended the survival of the engineered hESCs.

Xu et al (Xu H, Wang B, Ono M, et al. Targeted disruption of HLA genes via CRISPR-Cas9 generates iPSCs with enhanced immune compatibility. Cell Stem Cell. 2019; 24(4):566-578) also prepared hiPSCs in which HLA-A and HLA-B were knocked out, but only a single allele of HLA-C was knocked out by CRISPR. The hiPSCs-C cells with HLA-C expression but no HLA-A and -B expression suppressed NK cell activity.11 They also evaluated the graft survival of hiPSCs-C in vivo using humanized mice. The number of B2M-knockout hiPSCs quickly decreased after NK cells transplantation, whereas hiPSCs-C cells survived extensively in vivo.

Accordingly in some embodiments the engineered progenitor, producer or effector-chassis according to any of the preceding claims wherein the progenitor, producer or effector-chassis has been engineered to have disrupted expression from one or more HLA genes. In some embodiments the engineered progenitor, producer or effector-chassis has been engineered to have disrupted expression from any one or more of HLA-A, HLA-B and/or HLA-C. In some embodiments expression from both alleles of HLA-A, HLA-B and HLA-C has been disrupted. In some embodiments expression of HLA-A and HLA-B has been entirely disrupted but wherein expression of HLA-C has been partially disrupted. In some embodiments expression from both alleles of HLA-A and HLA-B have been disrupted but wherein expression from only one allele of HLA-C has been disrupted.

Strategy #2: Generation of Progenitor, Producer or Effector-Chassis with Reduced Immunogenicity by Overexpression of the HLA Class Ib Genes.

It is generally anticipated that knocking out B2M in hPSCs makes them become sensitive to natural killer (NK) cell-mediated killing because they lack the missing-self response. HLA-I molecules are inhibitory ligands of killer immunoglobulin-like receptors (KIRs) and CD94/NKG2 on NK cells.

In a number of physiological and pathological states, immunomodulatory molecules occur naturally. Such molecules include HLA-G, HLA-E, CD47 and PD-L1. Over the last few years, these four molecules have emerged as the top contenders in the engineering of universal cells. They fall into 2 groups: ‘nonclassical HLA class Ib (HLA-G and E)’ and ‘immune checkpoint’ strategies (PDL-1 and CD47).

Accordingly in some embodiments the engineered progenitor, producer or effector-chassis has been engineered to overexpress anyone or more of HLA-G, HLA-E, CD47 and PD-L1. In some embodiments the engineered progenitor, producer or effector-chassis has been engineered so as to have inhibited expression from the Beta 2 microglobulin and has also been engineered to overexpress any one or more of HLA-G, HLA-E, CD47 and PD-L1.

### 2.1. Overexpression of HLA-G

HLA-G is unique among immunomodulatory molecules in that it has a potent immunosuppressive action on virtually all arms of the innate and adaptive immune systems, through inhibitory receptors such as ILT2, ILT4 and KIR2DL4, one or more of which is expressed on cytotoxic CD8+ T cells, CD4+ T helper cells, Treg cells, B cells, NK cells, macrophages, dendritic cells and monocytes. HLA class Ia-negative, HLA-G-positive iPSC-derived NK cells have reduced immunogenicity, leading to increased survival in vitro (Bjordahl R, Clarke R, Gaidarova S et al. Multi-functional genetic engineering of pluripotent cell lines for universal off-the-shelf natural killer cell cancer immunotherapy. Blood 130(Suppl. 1), 3187 (2017).

However, perhaps most importantly, it appears HLA-G is capable of promoting tolerance to allogeneic cells, in which HLA class Ia (HLA-A, B and C) has been left intact, such as in the following three reports (Zhao L, Teklemariam T, Hantash B M. Heterologous expression of mutated HLA-G decreases immunogenicity of human embryonic stem cells and their epidermal derivatives. Stem Cell Res. 13(2), 342-354 (2014); Teklemariam T, Zhao L, Hantash B M. Heterologous expression of mutated HLA-G1 reduces alloreactivity of human dermal fibroblasts. Regen. Med. 9(6), 775-784 (2014); Zhao H X, Jiang F, Zhu Y J et al. Enhanced immunological tolerance by HLA-G1 from neural progenitor cells (NPCs) derived from human embryonic stem cells (hESCs). Cell. Physiol. Biochem. 44(4), 1435-1444 (2017)). Hence, to date, HLA-G1 is the only immunomodulatory molecule that has been shown to single-handedly induce tolerance to allogeneic cells in which genetic engineering of HLA class Ia molecules has not taken place.

### 2.2. Overexpression of HLA-E

HLA-E, like HLA-G, is a nonclassical HLA class Ib molecule; it is minimally polymorphic. At a simplistic level, HLA-E has a dual role, being an Immune Inhibitor via receptor CD94/NKG2A on NK and CD8+ T cells, or an immune activator via receptor CD94/NKG2C on NK and CD8+ T cells and via T-cell receptors on T cells.

Gornalusse et al (Gornalusse G G, Hirata R K, Funk S E, et al. HLA-E-expressing pluripotent stem cells escape allogeneic responses and lysis by NK cells. Nat Biotechnol. 2017; 35(8):765-772) also reported that a deficiency in the missing-self response could be prevented not only by CD47 overexpression (see below) but also by the forced expression of HLA-E. HLA-E was knocked in into hESCs at the B2M locus, where the HLA-edited hESCs showed no surface expression of HLA-A, -B or -C. The HLA-edited hESCs and their differentiated cells (RPE cells and HSCs) did not show an allogeneic response by CD8+ T cells and resisted lysis by NK cells. This study demonstrated that HLA-E expression in hESCs and their differentiated cells that do not express polymorphic HLA class I molecules except for that HLA-E can prevent the inhibition of the missing-self response by NK cells.

Strategy #3: Generation of Progenitor, Producer or Effector-Chassis with Reduced Immunogenicity by Overexpression of Immune-Modulatory Genes.

### 3.1. Overexpression of CD47

CD47 μlays a key role in self-recognition by acting as a ‘don't eat me’ signal to macrophages to protect cells from phagocytosis. This is achieved through interaction of CD47 with SIRPα/CD172a, an inhibitory receptor, found on macrophages. CD47 is extensively upregulated in solid and hematological malignancies for immune escape. Also, the interface between foetal-maternal blood and foetal tissues, which are composed of cytotrophoblast cells, expresses a low level of HLA class I and II molecules and a high level of CD47. Therefore, Deuse et al (Deuse T, Hu X, Gravina A, et al. Hypoimmunogenic derivatives of induced pluripotent stem cells evade immune rejection in fully immunocompetent allogeneic recipients. Nat Biotechnol. 2019; 37(3):252-258) designed hiPSCs (BCC-hiPSCs) in which B2M and CIITA were both knocked out using CRISPR, and subsequently, the CD47 gene was knocked in into hiPSCs. Cardiomyocytes and endothelial cells derived from BCC-hiPSCs escaped the immune response in allogeneic recipients. In particular, the overexpression of CD47 on BCC-hiPSCs inhibited NK cell activity and killing potential in vitro and in vivo.

### 3.2. Overexpression of PD-L

PD-L1, also known as CD274 or B7-H1, delivers a ‘don't find me’ signal to T cells, whereby it binds to the PD-1 receptor located on T cells to inhibit them. PD-L1 has high binding affinity to programmed cell death 1 (PD-1), which is displayed on T-cell surfaces where the interaction between PD-L1 and PD-1 leads to the inhibition of T-cell activities. Rong et al (Rong Z, Wang M, Hu Z, et al. An effective approach to prevent immune rejection of human ESC-derived allografts. Cell Stem Cell. 2014; 14(i):121-130) generated gene-edited hESCs (PC-hESCs) that constructively express PD-L1 and cytotoxic T lymphocyte antigen 4 (CTLA4)-immunoglobulin (Ig). CTLA4 has high binding affinity to CD86 and CD80, which are the primary signaling pathways involved in the activation of T cells. Then, a fusion protein of CTLA4 and Ig was designed to inhibit the T cell-mediated immune response. The differentiated cells from PC-hESCs did not generate an immune response when transplanted into humanized mice, whereas the genetically non-edited original hESCs were extensively rejected in humanized mice.

It is apparent to the skilled person that various combinations of disruptions and overexpressions of the genes described herein can be made. For example Han et al (Han X, Wang M, Duan S, et al. Generation of hypoimmunogenic human pluripotent stem cells. Proc Natl Acad Sci USA. 2019; 116(21):10441-10446) generated hypoimmunogenic hESCs using CRISPR/Cas9 gene editing in which HLA-A, —B and -C were knocked out. Subsequently, PD-L1, HLA-G and CD47 were knocked in into the safe harbour locus of AAVS1 in these HLA-deficient KO cells.

Accordingly, in some embodiments the invention provides an engineered progenitor, producer or effector-chassis wherein the progenitor, producer or effector-chassis has been engineered to have reduced immunogenicity with respect to a non-engineered chassis and wherein the progenitor, producer or effector-chassis has been engineered to:
a) have disrupted function of MHC Class 1 genes or proteins;
b) have disrupted expression from the β2 microglobulin gene, optionally to knock out the 02 microglobulin gene;
c) have disrupted expression from one or more HLA genes;
d) have disrupted expression from any one or more of HLA-A, HLA-B and/or HLA-C, optionally wherein expression of HLA-A and HLA-B has been entirely disrupted but wherein expression of HLA-C has been partially disrupted, optionally wherein expression from both alleles of HLA-A and HLA-B have been disrupted but wherein expression from only one allele of HLA-C has been disrupted;
e) overexpress any one or more of the HLA class Ib genes, optionally any one or more of HLA-G, HLA-E, CD47 and PD-L1;
f) engineered to overexpress any one or more of HLA-G, HLA-E, CD47 and PD-L1, and has optionally been engineered to have disrupted expression from the beta 2 microglobulin gene; and/or
g) overexpress one or more immunomodulatory genes, optionally wherein the one or more immunomodulatory genes is selected from the group comprising CD47 and PD-L1.

In addition to any of the above gene modulations and/or overexpressions, the engineered progenitor, producer or effector-chassis described herein can be further engineered into a more advantageous progenitor, producer or effector-chassis, for example a 2nd gen progenitor, producer or effector-chassis. These further engineering steps are aimed at eliminating one or more genes of which the product(s) could negatively affect the potency of a cargo that is in some embodiments contained within the progenitor, producer or effector-chassis, for example contained with the engineered platelet or engineered platelet-like membrane-bound cell fragment. Native properties of the progenitor, producer or effector-chassis can be tuned up or down with regards the innate/adaptive response. Described below are two exemplary genes that can be engineered, but the concept can be applied for other genes (listed in the appendixes). This approach could be extended to modify the baseline property of platelets in angiogenesis desirable.

There has been a gradual realization that—beyond their well-characterised role as primary cellular mediators of haemostasis—platelets have important roles in modulating innate and adaptive immune responses. For example, platelets have been shown to have roles in the initiation of inflammation, angiogenesis, atherosclerosis, lymphatic development and tumour growth.

Proteomic analysis has demonstrated that platelets have the ability to secrete more than 300 different proteins following activation with thrombin, some of which (such as IL-1, TLRs and CD154 (aka CD40L)) are clearly involved in processes other than blood clotting.

Whether they are resting or activated, platelets present on their surfaces (or on the surface of exosomes and micro-vesicles) a range of adhesive proteins which facilitate both homo-typic interactions between platelets and heterotypic interactions between platelets and different immune cell populations. Upon activation, they also release the content of their granules which contain various pro-inflammatory and anti-inflammatory cytokines and chemokines.

Numerous reports have studied the interaction of resting and activated platelets in contact with cells from the innate and adaptive systems as well as with pathogens or cell infected by pathogens. The picture emerging is that platelets combine pro-inflammatory and immunosuppressive properties which are entirely dependent on context (micro-environment, cell status, tissue integrity, etc).

It is considered that the progenitor, producer or effector-chassis described herein, for example the engineered progenitor, producer or effector-chassis, for example the engineered progenitor, producer or effector-chassis that has reduced thrombogenic potential, for example the engineered platelets or platelet-like membrane-bound cell fragments that have reduced thrombogenic potential have broadly similar properties to human platelets in modulating innate and adaptive responses, since the gene disruptions of the progenitor, producer or effector-chassis are exclusively targeted to remove the thrombogenic program.

As described herein, in some embodiments the progenitor, producer or effector-chassis comprises a cargo that is to be released on degranulation, and in preferred embodiments the progenitor, producer or effector-chassis also comprises one or more CPR, universal CPR, complex of universal CPR and tagged targeting peptide, SAPR or ePAR of the invention so as to result in localised triggering of degranulation and release of the cargo at the site of the target. It is expected that upon degranulation, the standard contents of the granules are also released alongside the cargo.

While this may bear no negative effect on the biological and thus therapeutic potential of an engineered platelets given the vast amount of circulating wild-type platelets, it is conceivable that—in some therapeutic settings—some adhesive proteins and/or cargo entities naturally produced by engineered platelets may indirectly counter the localised biological action of the engineered platelet, thereby reducing the therapeutic efficacy.

To address this, it is considered to be advantageous to disrupt one or more genes encoding adhesive proteins and/or cargo entities which are likely to indirectly counter the biological action of the engineered cargo, potentially leading to a greater net therapeutic effect.

The concept outlined here has been applied with other cell therapies, such as CAR T cells: see for example McGowan et al 2020 Biomedicine and Pharmacotherapy PD-1 disrupted CAR-T cells in the treatment of solid tumors: Promises and challenges https://doi.org/10.1016/j.biopha.2019.109625)

Accordingly, in one embodiment the engineered progenitor, producer or effector-chassis, for example engineered myeloid stem cell, a megakaryoblast, a megakaryocyte, a megakaryocyte-like cell, an iPSC, a platelet, or a platelet-like membrane-bound cell fragment has been engineered so as to disrupt or knockout the expression of one or more genes encoding adhesive proteins and/or cargo entities which are likely to indirectly counter the biological action of the engineered cargo, potentially leading to a greater net therapeutic effect.

### Downmodulation of CD40L

Upon activation platelets release CD40L (aka CD154). Although CD40L was first described on T-cells and is a critical co-stimulatory signal development and function of the immune system, the sheer number of blood platelets makes them the predominant source of CD40L in the circulation.

CD40L binds to CD40 which is a receptor predominantly found on antigen-presenting cells (B-cells, macrophages, dendritic cells, monocytes). CD40 is also present on non-immune cells such as epithelial and endothelial cells, fibroblasts, myofibroblasts, stellate cells, and resting platelets. The CD40/CD40L system allows interactions between immune cells, and between immune and non-immune cells.

CD40 can engage with CD40L that is presented as cell surface receptor (as on T helper cells and activated platelets), on the surface of platelet micro-vesicles (PMV) that are released into the circulation for dissemination and distal control of immunity, or as soluble trimeric sCD40L.

Engagement of CD40L with CD40 results in activation of the immune response program of CD40+ cells. For example, (1) endothelial cells (EC) secrete chemokines (IL-8, TNF alpha and MCP-1) and express adhesion molecules (E-selectin, VCAM and ICAM-1); (2) DC produce Il-6 and IL-12 as well as increase their expression of CD80 and CD86; (3) B cells undergo isotype switching, maturation to a memory phenotype, and proliferation.

CD40 is an important activation receptor for inflammation response, humoral and cellular immunity. Since CD40 is present on a wide range of cell types and since activated platelets are the main contributors of CD40L (membrane bound and soluble) alongside T helper cells, it can be deduced that in therapeutic indications where immune system activation is sought to promote a therapeutic response, it is beneficial to maintain CD40L level in platelets.

However, in other therapeutic settings, the presence of CD40L in engineered platelets may counteract/overwhelm the therapeutic potency of orthogonal endogenous/exogenous cargos if these are selected to block immune, inflammatory and/or proliferation events. Such therapeutic settings are for example: some forms of blood-borne cancers or autoimmune diseases (e.g. RA, Chron's, Sjogren's syndrome) which are driven or supported by antigen-presenting CD40+ cells. For example CD40+ mononuclear cells (SFMC) are abundant in the synovial fluid of biopsies from patients with rheumatoid arthritis (RA). TNF-alpha secretion from RA SFMC is enhanced by CD40 stimulation, whereas spontaneous secretion of TNF-alpha from RA SFMC is inhibited by anti-CD40 antibody. Thus, an engineered platelet directed at delivering payload to SFMC to treat RA may show greater disease modifying effect if the platelet was devoid of CD40L. (Expression and function of CD40 in rheumatoid arthritis synovium J Rheumatol. 1998 June; 25(6):1048-53.) In another example, CD40 is expressed on the surface of many B-cell malignancies (i.e., chronic lymphocytic leukemia (CLL) and multiple myeloma (MM), non-Hodgkin lymphoma, Hodgkin disease, and acute lymphoblastic leukemia) and certain solid malignancies (e.g., renal cell carcinoma, breast carcinoma, melanoma, pancreatic carcinoma). CD40L engagement by follicular T helper cells induces strong pro-survival signaling in these malignant cells. It also induces resistance to apoptosis-inducing small molecule agents such as fludarabine and the Bcl-2 antagonist venetoclax. Approaches at blocking CD40 with a non-agonistic antibody have progressed in the clinic (Leuk Lymphoma. 2012 November; 53(11) Phase I study of the anti-CD40 humanized monoclonal antibody lucatumumab (HCD122) in relapsed chronic lymphocytic leukemia.). Thus, it is expected that an engineered platelet directed at delivering payload to malignant B-cells may show greater disease modifying effect if the platelet was devoid of CD40L.

In conclusion, a progenitor, producer or effector-chassis of the invention that is devoid of CD40L would open the possibility of greater therapeutic efficacy of engineered platelets in settings where local release of CD40L would counteract orthogonal therapeutic effects aimed at blocking inflammation, immune response or cell proliferation.

Accordingly, in some embodiments the invention provides engineered progenitor, producer or effector-chassis, for example engineered myeloid stem cell, a megakaryoblast, a megakaryocyte, a megakaryocyte-like cell, an iPSC, a platelet, or a platelet-like membrane-bound cell fragment that has been engineered so as to disrupt or knockout the expression the CD40L gene.

By extension, other immune activators/effectors presented at the surface of activated platelets or released from activated platelets (either as soluble proteins, receptors anchored on exosomes or PMVs) could be targeted to gene disruption/silencing. The efficiency and multiplex capacity of genome engineering would allow to target multiple immune activators in one progenitor, producer or effector-chassis. A non-exclusive list of anti-inflammatory immune effectors that it is considered to be beneficial, in some embodiments, to disrupt or inhibit the expression of are: CD36, NOD2, SRB1, TLR1, TLR2, TLR3, TLR4, TLR6, TLR9, CD40L, CD93 (C1qRp), C3aR, CD88 (C5aR), CD89 (FcαR1), CD23 (FcεR1), CD32 (FcγRIIa), MHC class1, CD191 (CCR1), CD193 (CCR3), CD194 (CCR4), CD184 (CXCR4), CX3CR1, CD102 (ICAM-2), JAM-C/JAM-3, CD62P (P-selectin), CD31 (PECAM-1), CD150 (SLAMF1), CCL2, CCL3, CCL5, CXCL1, CXCL12, CXCL4/PF4, CXCL5, CXCL8, NAP2 (CXCL7), IL-1β. See FIG. 12.

Accordingly, in some embodiments the invention provides an engineered progenitor, producer or effector-chassis, for example engineered myeloid stem cell, a megakaryoblast, a megakaryocyte, a megakaryocyte-like cell, an iPSC, a platelet, or a platelet-like membrane-bound cell fragment that has been engineered so as to disrupt or knockout the expression of any one or more of the following genes: CD36, NOD2, SRB1, TLR1, TLR2, TLR3, TLR4, TLR6, TLR9, CD40L, CD93 (C1qRp), C3aR, CD88 (C5aR), CD89 (FcαR1), CD23 (FcεR1), CD32 (FcγRIIa), MHC class1, CD191 (CCR1), CD193 (CCR3), CD194 (CCR4), CD184 (CXCR4), CX3CR1, CD102 (ICAM-2), JAM-C/JAM-3, CD62P (P-selectin), CD31 (PECAM-1), CD150 (SLAMF1), CCL2, CCL3, CCL5, CXCL1, CXCL12, CXCL4/PF4, CXCL5, CXCL8, NAP2 (CXCL7), IL-1p.

### Down-Modulation of the GARP-TGFβ Axis

Transforming growth factor-β (TGF-β) is a pleiotropic cytokine expressed by the majority of cells and found in all tissues. It plays important roles in numerous aspects of biological processes such as cell proliferation, development, apoptosis, fibrosis, angiogenesis, wound healing, and cancer.

In acquired immunity, TGF-β1 is required to convert conventional CD4⁺ T (Tconv) cells into induced regulatory T (iTreg) cells that express the transcription factor Foxp3, and to promote Treg proliferation. With regard to innate immunity and cancer, TGF-β inhibits dendritic cell (DC) maturation as well as natural killer (NK) cells through the downregulation of NKG2D ligand. The role of TGF-β is also well studied in the non-resolving inflammation that facilitates cancer initiation. Tumour-derived TGF-β polarizes macrophages into tumour-associated macrophages (TAM). TGF-β derived from TAMs is one of the major drivers of the epithelial to mesenchymal transition (EMT) which leads to metastasis. Furthermore, TGF-β impairs the adaptive anti-tumour immunity by directly inhibiting the clonal expansion and cytotoxicity of the CD8+ cytotoxic T cells (CTLs). Finally, TGF-β indirectly attenuates CTLs by inducing the expression of Foxp3, which confers a regulatory and immune suppressive phenotype to CD4⁺ T cells.

Platelets are the dominant source of functional TGFβ systemically as well as in the tumour microenvironment through constitutive expression of TGFβ-docking GARP rather than secretion of TGFβ per se. GARP (glycoprotein-A repetitions predominant protein) has been identified as a latent TGF-β1 receptor expressed on immune cells, specifically on activated Tregs and constitutively on platelets. Platelet-intrinsic GARP plays the most dominant role in capturing and activating TGFβ and thus contributes significantly to Treg cell homeostasis.

In line with this, several reports have shown that patients with immune thrombocytopenia have deficiencies in CD4+CD25+FOXP3+ regulatory T (TReg) cells. Therapies that increase platelet counts (such as intravenous immunoglobulins, dexamethasone, rituximab or TPO) have been shown to restore Treg cell numbers and functions in ITP patients.

TGFβ signaling has key roles in cancer progression: most carcinoma cells have inactivated their epithelial antiproliferative response and benefit from increased TGFβ expression and autocrine TGFβ signaling through effects on gene expression, release of immunosuppressive cytokines and epithelial plasticity. As a result, TGFβ enables cancer cell invasion and dissemination, stem cell properties and therapeutic resistance. TGFβ released by cancer cells, stromal fibroblasts and other cells in the tumour microenvironment further promotes cancer progression by shaping the architecture of the tumour and by suppressing the antitumour activities of immune cells, thus generating an immunosuppressive environment that prevents or attenuates the efficacy of anticancer immunotherapies.

Thus, the GARP-TGFβ axis is a key immunosuppressive molecular hallmark in the cancer microenvironment (J Hematol Oncol. 2018 Feb. 20; 11(1) Immunoregulatory functions and the therapeutic implications of GARP-TGF-β in inflammation and cancer). Platelets are not bystanders. Indeed, proof that platelets constrain T cell immunity though a GARP-TGFβ axis has been obtained by platelet-specific deletion of GARP-encoding gene Lrrc32 which resulted in blunted TGFβ activity at the tumour site and potentiated protective immunity against both melanoma and colon cancer in animal models (Platelets subvert T cell immunity against cancer via GARP-TGFbeta axis. Sci Immunol. 2017 May 5; 2(11).

The GARP-TGFβ axis is also engaged on platelets which ‘cloak’ metastatic cells: they inhibit Natural Killer (NK) cells, by inducing the release of soluble NKG2D ligands from the tumour cell to mask detection (‘immune decoy’) and by actively suppressing NK cell degranulation and inflammatory cytokine (IFNγ) production, concomitantly.

Thus, a progenitor, producer or effector-chassis of the invention that is devoid of GARP-TGFβ axis (either by disruption of the GARP or TGFβ genes at iPSC level, or by expression of silencing RNAs) would open the possibility of greater therapeutic efficacy of engineered platelets in many settings where activation and local concentration of TGFβ would counteract orthogonal therapeutic effects aimed at blocking cancer cell proliferation, and EMT.

In some embodiments, the GARP LRRC32 gene is knocked out using any of the following gRNAs:

gRNA99:
[SEQ ID NO: 116]
CCUGAGCUGCAACAGCAUCG
gRNA100:
[SEQ ID NO: 117]
GCCACCAGCACUCAGCGCAG

By extension, other immune modulators presented at the surface of activated platelets or released from activated platelets (either as soluble proteins, receptors anchored on exosomes or PMVs) could be targeted to gene disruption/silencing. The efficiency and multiplex capacity of genome engineering would allow to target multiple immune down-modulators in one progenitor, producer or effector-chassis. A non-exclusive list of anti-inflammatory immune effectors that it is considered to be beneficial, in some embodiments, to disrupt or inhibit the expression of are: Siglec-7, Siglec-9, Siglec-11, TGFβ. See FIGS. 14 and 15.

Accordingly, in some embodiments the invention provides an engineered progenitor, producer or effector-chassis, for example engineered myeloid stem cell, a megakaryoblast, a megakaryocyte, a megakaryocyte-like cell, an iPSC, a platelet, or a platelet-like membrane-bound cell fragment that has been engineered so as to disrupt or knockout the expression of any one or more of the following genes: Siglec-7, Siglec-9, Siglec-11, TGFβ.

It is to be noted, and as described herein, that a progenitor, producer or effector-chassis that comprises any one or more of the gene disruptions or overexpressions may or may not comprise anyone or more of a CPR, universal CPR, complex of universal CPR and tagged targeting peptide, SAPR or ePAR of the invention, i.e. the invention provides a progenitor, producer or effector-chassis as described herein that does not comprise any one or more of CPR, universal CPR, complex of universal CPR and tagged targeting peptide, SAPR or ePAR; and also provides a progenitor, producer or effector-chassis as described herein that does comprise one or more CPR, universal CPR, complex of universal CPR and tagged targeting peptide, SAPR or ePAR of the invention.

In some embodiments, the progenitor, producer or effector-chassis may be engineered to express one or more additional ITAM receptors to enhance T cell signaling and stimulate an immune response. T cell receptors (TCRs) recognize antigens bound in the major histocompatibility complex (MHC) (See, James et al. Sci. Signal. 11, eaan1088 (2018), which is hereby incorporated by reference in its entirety). ITAMs on the TCRs convert the action of binding and recognition into an intracellular signal (Ibid). Inserting additional ITAMs into chimeric TCRs was observed to scale linearly with the number of ITAM receptors and decreasing or knocking-out the number of ITAM receptors was observed to inhibit T cell development by impairing thymocyte lineage commitment (Ibid). Accordingly in one embodiment the invention provides a progenitor, producer or effector-chassis or engineered progenitor, producer or effector-chassis as described herein that has been engineered to express one or more additional ITAM receptors to enhance T cell signaling and stimulate an immune response.

It is clear then from the above that the engineered progenitor, producer or effector-chassis may comprise any number of different gene disruptions, gene deletions, or gene overexpressions, or other modifications.

In some embodiments, the engineered progenitor, producer or effector-chassis, for example engineered myeloid stem cell, a megakaryoblast, a megakaryocyte, a megakaryocyte-like cell, an iPSC, a platelet, or a platelet-like membrane-bound cell fragment comprises a disruption of a least one gene, at least one, two, three, four, five, six, seven, eight, nine, or at least ten genes, for example a disruption of a least one gene, at least one, two, three, four, five, six, seven, eight, nine, or at least ten genes wherein the genes are involved in a the thrombogenic pathway, are involved in immunogenicity and/or are involved in inflammation,

In some embodiments, the engineered progenitor, producer or effector-chassis been engineered to synthesise a protein in response to a particular signal. For example, in Weyrich et al., BCL-3 was specifically upregulated in activated platelets through a mechanistic target of rapamycin (mTOR) dependent signaling mechanism (See, Weyrich et al. “Signal-dependent translation of a regulatory protein, Bcl-3, in activated human platelets”. PNAS, 1998, which is hereby incorporated by reference in its entirety). Therefore, knock-in of a gene into the BCL-3 locus or identification of the minimal 5′ UTR region that mediates activation dependent translation would allow synthetic gene expression regulation in platelets. Therefore, the engineered progenitor, producer or effector-chassis described herein may in some embodiments have an altered signaling pathway resulting in signaling induced protein translation. For example, the progenitor, producer or effector-chassis may be engineered to as to express a cargo protein upon platelet activation; and/or may be engineered to express a toxic protein upon activation, either with a view to destroying the platelet, or with a view to delivering a toxic payload to the target cell/tissue.

In some embodiments, the engineered progenitor, producer or effector-chassis, for example engineered platelets or engineered platelet-like membrane-bound cell fragments can synthesize protein in response to an activation signal. For example, in Weyrich et al., BCL-3 was specifically upregulated in activated platelets through a mechanistic target of rapamycin (mTOR) dependent signaling mechanism (See, Weyrich et al. “Signal-dependent translation of a regulatory protein, Bcl-3, in activated human platelets”. PNAS, 1998, which is hereby incorporated by reference in its entirety). Therefore, knock-in of a gene into the BCL-3 locus or identification of the minimal 5′ UTR region that mediates activation dependent translation would allow synthetic gene expression regulation in platelets. Therefore, platelets described herein may have an altered signaling pathway resulting in signaling induced protein translation. For example, expressing a toxic protein once activated or triggering downstream events following target cell recognition. Accordingly in some embodiments the progenitor, producer or effector-chassis has been engineered to synthesise a protein or RNA of interest in response to activation of the platelet or platelet-like membrane-bound cell fragment, optionally wherein the protein or RNA of interest is expressed from the BCL-3 locus.

A progenitor, producer or effector-chassis as described herein, for example an engineered progenitor, producer or effector-chassis that lacks thrombogenic potential, and/or has reduced immunogenicity and/or reduced inflammatory potential that expresses one or more CPRs, universal CPRs, complexes of universal CPRs and tagged targeting peptides, SAPRs or ePARs of the invention can be considered to be a targeted delivery system. Accordingly the invention provides a targeted delivery system comprising a progenitor, producer or effector-chassis or engineered progenitor, producer or effector-chassis as described herein that expresses any one or more CPRs, universal CPRs, complexes of universal CPRs and tagged targeting peptides, SAPRs or ePARs of the invention.

It is clear to the skilled person that the effector-chassis that actually does the targeted delivery of the cargo is the platelet or platelet-like membrane-bound cell fragment, or Synlet—however, the platelet or platelet-like membrane-bound cell fragment, or Synlet is derived from a precursor progenitor or producer-chassis, for example an iPSC or a megakaryocyte.

In some instances, the progenitor, producer or effector-chassis, for example a progenitor, or producer or effector-chassis of the targeted delivery system, comprises more than one CPRs, universal CPRs, complexes of universal CPRs and tagged targeting peptides, SAPRs or ePARs of the invention. For example, the progenitor, producer or effector-chassis in some instances comprises at least 2, 3, 4, 5, 6, 7, 8, 9, or at least 10 different CPRs, universal CPRs, complexes of universal CPRs and tagged targeting peptides, SAPRs or ePARs of the invention.

In some instances this allows the progenitor, producer or effector-chassis to be targeted to at least two different targets—for example where the progenitor, producer or effector-chassis expresses at least two CPRs, universal CPRs, complexes of universal CPRs and tagged targeting peptides, SAPRs or ePARs of the invention and wherein the target binding domains of the at least two CPRs, universal CPRs, complexes of universal CPRs and tagged targeting peptides, SAPRs or ePARs of the invention are directed to different targets.

In some instances, the platelet modulation domains of the at least two CPRs, universal CPRs, complexes of universal CPRs and tagged targeting peptides, SAPRs or ePARs that have different target binding domains are both platelet activating domains, such that upon binding of one or both of the CPRs, universal CPRs, complexes of universal CPRs and tagged targeting peptides, SAPRs or ePARs to the respective target, the platelet degranulates. This can be considered to be an OR system—i.e. the platelet activates upon binding to a first target OR a second target. A situation in which this may be useful is, for example, where a particular cancer is known to express two different cell surface tumour specific antigens.

In some instances, the platelet modulation domains of the at least two CPRs, universal CPRs, complexes of universal CPRs and tagged targeting peptides, SAPRs or ePARs that have different target binding domains have opposing functions, i.e. one platelet modulation domain is a platelet activation domain, and the second platelet modulation domain is a domain that inhibits activation of the platelet. In this situation for example the platelet modulation domain of a first CPR, universal CPR, complex of universal CPR and tagged targeting peptide, SAPR or ePAR that has a first target binding domain may be a platelet activation domain for example may be an ITAM containing domain, and the platelet modulation domain of a second CPR, universal CPR, complex of universal CPR and tagged targeting peptide, SAPR or ePAR that has a second target binding domain may be a domain that inhibits activation of platelet degranulation, for example may be an ITIM containing domain. Where the progenitor, producer or effector-chassis of this embodiment only binds to the first target, the ITAM domain results in platelet activation and degranulation. However, where both the first and second target are present, and the first and second CPR, universal CPR, complex of universal CPR and tagged targeting peptide, SAPR or ePAR binds to the first and second target, the ITIM domain of the second CPR, universal CPR, complex of universal CPR and tagged targeting peptide, SAPR or ePAR represses activation of the platelet that would otherwise be triggered by binding of the first target to the first CPR, universal CPR, complex of universal CPR and tagged targeting peptide, SAPR or ePAR. In this way it is possible to build up complex logic networks such as AND/OR/NOR. In the above example, activation would only occur in the presence of the first target and the absence of the second target. When the second target is present and also bound by the second CPR, universal CPR, complex of universal CPR and tagged targeting peptide, SAPR or ePAR, platelet activation through the first CPR, universal CPR, complex of universal CPR and tagged targeting peptide, SAPR or ePAR is inhibited.

These types of logic networks can incorporate any of the CPR, universal CPR, complex of universal CPR and tagged targeting peptide, SAPR or ePAR described herein, for example including those based on ITAM domains, those based on ITIM domains, and those based on GPCRs.

In some embodiments, where the progenitor, producer or effector-chassis is an engineered progenitor, producer or effector-chassis that has been engineered so as to render it non-thrombogenic, for example an engineered iPSC, engineered megakaryocyte or engineered platelet that has been engineered so as to be non-thrombogenic, and the progenitor, producer or effector-chassis expresses one or more CPRs of the invention, the combination of the engineered progenitor, producer or effector-chassis and one or more CPRs, universal CPRs, complexes of universal CPRs and tagged targeting peptides, SAPRs or ePARs can be considered to be a non-thrombogenic delivery system. Accordingly, the invention provides a non-thrombogenic delivery system comprising an engineered progenitor, producer or effector-chassis according to the invention wherein the engineered progenitor, producer or effector-chassis is non-thrombogenic and expresses one or more CPRs, universal CPRs, complexes of universal CPRs and tagged targeting peptides, SAPRs or ePARs of the invention. Such a system may be referred to a Synlet delivery system.

The non-thrombogenic delivery system, or Synlet delivery system, may be for the delivery of a therapeutic cargo in which case the system can be considered to be a non-thrombogenic therapeutic delivery system—or the cargo may be a non-therapeutic cargo, for example may be a cosmetic-cargo or an imaging agent, in which case the system can be considered to be a non-thrombogenic non-therapeutic delivery system.

Accordingly, the invention provides a targeted delivery system comprising a progenitor, producer or effector-chassis—preferably an effector chassis—as defined in any of the preceding claims that expresses one or more CPRs, universal CPRs, complexes of universal CPRs and tagged targeting peptides, SAPRs or ePARs according to any of the preceding claims, optionally wherein the targeted delivery system is a therapeutic targeted delivery system or a non-therapeutic delivery system.

The invention also provides a non-thrombogenic targeted delivery system comprises a progenitor, producer or effector-chassis—preferably an effector chassis—as defined in any of the preceding claims that expresses one or more CPRs, universal CPRs, complexes of universal CPRs and tagged targeting peptides, SAPRs or ePARs according to any of the preceding claims and wherein the progenitor, producer or effector-chassis has been engineered to disrupt the thrombogenic pathway targeted delivery system is a non-thrombogenic therapeutic targeted delivery system or a non-thrombogenic non-therapeutic delivery system.

As mentioned, the progenitor, producer or effector-chassis of the invention or an engineered progenitor, producer or effector-chassis of the invention may comprise a cargo. The progenitor, producer or effector-chassis of the invention or engineered progenitor, producer or effector-chassis of the invention that comprises a cargo may comprise one or more CPRs, universal CPRs, complexes of universal CPRs and tagged targeting peptides, SAPRs or ePARs of the invention, or may not comprise one or more CPRs, universal CPRs, complexes of universal CPRs and tagged targeting peptides, SAPRs or ePARs of the invention.

Reference to a cargo herein is intended to refer to any cargo that can be delivered using a platelet or platelet-like membrane-bound cell fragment, or engineered platelet or engineered platelet-like membrane-bound cell fragment. To be able to be delivered, the cargo can be located in the cytoplasm, in the granules such as the alpha-granules, or in the plasma membrane or on the plasma membrane surface.

The cargo may be a therapeutic cargo or may be a non-therapeutic cargo, for example may be an imaging agent or a cosmetic agent.

It is clear to the skilled person which agents are suitable for use as a cargo. A cargo can be any entity that can either be endogenously expressed by the chassis, or which can be exogenously loaded in to a chassis. For example in some embodiments the cargo is selected from the group comprising or consisting of:
a) a protein or peptide—in some embodiments the protein or peptide is:
   i) an antibody or antigen binding fragment thereof, for example an antibody or antigen binding fragment thereof binds to a tumor antigen or a neoantigen;
   ii) an enzyme, such as a nuclease for example a TALEN;
   iii) a cytokine for example IL-10; or
   iv) a CRISPR associated protein, for example Cas9;
   v) a bispecific protein, for example a bispecific antibody or a T-cell engager (BiTE)
b) a nucleic acid—in some embodiments the nucleic acid is:
   i) an RNA, for example selected from mRNA, a miRNA, shRNA, and a clustered regularly interspaced short palindromic repeats (CRISPR) sequence; or
   ii) a DNA vector;
c) a toxin;
d) a small molecule drug or imaging agent;
e) a viral vector such as AAV;
f) a virus such as oncolytic virus;
g) agents for performing CRISPR mediated gene editing;
or any combination thereof;
h) radionucleotide drugs;
i) radionucleotide tagged antibodies, or conjugate any thereof.

In some embodiments the cargo is an antibody or antigen binding fragment thereof. As used herein, the terms “antibody” or “antibodies” refer to molecules that contain an antigen binding site, e.g. immunoglobulin molecules and immunologically active fragments of immunoglobulin molecules that contain an antigen binding site. Immunoglobulin molecules can be of any type (e.g. IgG, IgE, IgM, IgD, IgA and IgY), class (e.g. IgG1, IgG2, IgG3, IgG4, IgA1 and IgA2) or a subclass of immunoglobulin molecule. Antibodies include, but are not limited to, synthetic antibodies, monoclonal antibodies, single domain antibodies, single chain antibodies, recombinantly produced antibodies, multi-specific antibodies (including bispecific antibodies), human antibodies, humanized antibodies, chimeric antibodies, intrabodies, scFvs (e.g. including mono-specific and bi-specific, etc.), Fab fragments, F(ab′) fragments, disulfide-linked Fvs (sdFv), anti-idiotypic (anti-Id) antibodies, and epitope-binding fragments of any of the above.

As used herein, the term “antibody fragment” is a portion of an antibody such as F(ab′)₂, F(ab)₂, Fab′, Fab, Fv, scFv and the like. Regardless of structure, an antibody fragment binds with the same antigen that is recognized by the intact antibody. For example, an anti-OX40 antibody fragment binds to OX40. The term “antibody fragment” also includes isolated fragments consisting of the variable regions, such as the “Fv” fragments consisting of the variable regions of the heavy and light chains and recombinant single chain polypeptide molecules in which light and heavy variable regions are connected by a peptide linker (“scFv proteins”). As used herein, the term “antibody fragment” does not include portions of antibodies without antigen binding activity, such as Fc fragments or single amino acid residues.

By “Fab fragment”, we include Fab fragments (comprising a complete light chain and the variable region and CH1 region of a heavy chain) which are capable of binding the same antigen that is recognized by the intact antibody. Fab fragment is a term known in the art, and Fab fragments comprise one constant and one variable domain of each of the heavy and the light chain.

In some embodiments, the progenitor, producer or effector-chassis for example an engineered platelet may be loaded with toxin, which would be cloaked from the immune system. The progenitor, producer or effector-chassis for example an engineered platelet may also be loaded with chemokines and/or selectins to mediate transfer of an agent across the blood brain barrier (BBB). Other embodiments of the progenitor, producer or effector-chassis may have platelet secretory granules loaded with membrane and/or soluble proteins. In certain embodiments, a toxin may be encoded with an α-granule localization signal attached to direct its uptake into secretory granules, which would be released on platelet receptor activation.

Platelet expression of programmed cell death protein (PD-1) and loading of an engineered platelet with cyclophosphamide has been observed to function as a potent anti-melanoma agent (See, Zhang et al. “Engineering PD-1-Presenting Platelets for Cancer Immunotherapy.” Nano Letters, 2018, which is hereby incorporated by reference in its entirety). Specifically, megakaryocytes were engineered to express PD-1, then the resulting engineered platelets were passively loaded with cyclophosphamide. Platelet targeting to the melanoma was driven by surgical wounding of the tumor in vivo (i.e. using the natural thrombogenic properties of the platelet), not a synthetic receptor, resulting in Tre depletion in the tumor and increased CD8⁺ T cell mediated killing. Tumor volume was observed to be significantly less 20 days after the beginning treatment for animals in the group with both PD-1 and cyclophosphamide compared to animals treated with platelets either expressing PD-1 or loaded with cyclophosphamide.

In some embodiments, the cargo of the engineered platelets of the invention may be a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo. Such mRNA molecules may have the structural components or features of any of those taught in International Publication No. WO 2013/151666, which is incorporated herein by reference in its entirety.

In some embodiments, a CRISPR/Cas gene editing system may be used to alter the genome of a megakaryocyte to produce the engineered platelets described herein. Alternatively, a CRISPR/Cas system may be packaged in a vesicle to be released on activation of the platelet by an antigen recognized by the CPR. CRISPR/Cas systems are bacterial adaptive immune systems that utilize RNA-guided endonucleases to target specific sequences and degrade target nucleic acids. They have been adapted for use in various applications in the field of genome editing and/or transcription modulation. Any of the enzymes or orthologs known in the art or disclosed herein may be utilized in the methods herein for genome editing.

In certain embodiments, the CRISPR/Cas system may be a Type II CRISPR/Cas9 system. Cas9 is an endonuclease that functions together with a trans-activating CRISPR RNA (tracrRNA) and a CRISPR RNA (crRNA) to cleave double stranded DNAs. The two RNAs can be engineered to form a single-molecule guide RNA by connecting the 3′ end of the crRNA to the 5′ end of tracrRNA with a linker loop. Jinek et al., Science, 337(6096):816-821 (2012), which is hereby incorporated by reference in its entirety, showed that the CRISPR/Cas9 system is useful for RNA-programmable genome editing, and international patent application WO 2013/176772 provides numerous examples and applications of the CRISPR/Cas endonuclease system for site-specific gene editing, which are incorporated herein by reference in their entirety. Exemplary CRISPR/Cas9 systems include those derived from Streptococcus pyogenes, Streptococcus thermophilus, Neisseria meningitidis, Treponema denticola, Streptococcus aureas, and Francisella tularensis.

In certain embodiments, the CRISPR/Cas system may be a Type V CRISPR/Cpf1 system. Cpf1 is a single RNA-guided endonuclease that, in contrast to Type II systems, lacks tracrRNA. Cpf1 produces staggered DNA double-stranded break with a 4 or 5 nucleotide 5′ overhang. Zetsche et al. Cell. 2015 Oct. 22; 163(3):759-71, which is hereby incorporated by reference in its entirety, provides examples of Cpf1 endonuclease that can be used in genome editing applications, which is incorporated herein by reference in its entirety. Exemplary CRISPR/Cpf1 systems include those derived from Francisella tularensis, Acidaminococcus sp., and Lachnospiraceae bacterium.

In certain embodiments, nickase variants of the CRISPR/Cas endonucleases that have one or the other nuclease domain inactivated may be used to increase the specificity of CRISPR-mediated genome editing. Nickases have been shown to promote HDR versus NHEJ. HDR can be directed from individual Cas nickases or using pairs of nickases that flank the target area.

In certain embodiments, catalytically inactive CRISPR/Cas systems may be used to bind to target regions (e.g., gene encoding an antigen, such as a receptor) and interfere with their function. Cas nucleases such as Cas9 and Cpf1 encompass two nuclease domains. Mutating critical residues at the catalytic sites creates variants that only bind to target sites but do not result in cleavage.

In certain embodiments, a CRISPR/Cas system may include additional functional domain(s) fused to the CRISPR/Cas endonuclease or enzyme. The functional domains may be involved in processes including but not limited to transcription activation, transcription repression, DNA methylation, histone modification, and/or chromatin remodeling. Such functional domains include but are not limited to a transcriptional activation domain (e.g., VP64 or KRAB, SID or SID4X), a transcriptional repressor, a recombinase, a transposase, a histone remodeler, a DNA methyltransferase, a cryptochrome, a light inducible/controllable domain or a chemically inducible/controllable domain.

In certain embodiments, a CRISPR/Cas endonuclease or enzyme may be administered to a cell or a patient as one or a combination of the following: one or more polypeptides, one or more mRNAs encoding the polypeptide, or one or more DNAs encoding the polypeptide.

In certain embodiments, guide nucleic acids may be used to direct the activities of an associated CRISPR/Cas enzymes to a specific target sequence within a target nucleic acid. Guide nucleic acids provide target specificity to the guide nucleic acid and CRISPR/Cas complexes by virtue of their association with the CRISPR/Cas enzymes, and the guide nucleic acids thus can direct the activity of the CRISPR/Cas enzymes.

In one aspect, guide nucleic acids may be RNA molecules. In one aspect, guide RNAs may be single-molecule guide RNAs. In one aspect, guide RNAs may be chemically modified. In certain embodiments, more than one guide RNAs may be provided to mediate multiple CRISPR/Cas-mediated activities at different sites within the genome.

In some embodiments, the cargo in the vesicles of an engineered platelets described herein is a small molecule drug such as, but not limited to those described in paragraph [0190] as presented on pages 98-123 of PCT PCT/GB2020/053247 which is hereby incorporated by reference.

As mentioned previously, although the actual delivery tool is the platelet or platelet-like membrane-bound cell fragment, these are fragments of precursor cells and so it is appropriate that the precursor cells are in some embodiments able to also carry the cargo, for example in instances where the cargo is endogenously produced, in some embodiments the cargo may be produced by any one or more of the progenitor, producer or effector-chassis as defined herein, for example any of the myeloid stem cell, a megakaryoblast, a megakaryocyte, a megakaryocyte-like cell, an iPSC, a platelet, or a platelet-like membrane-bound cell fragment. In this way it is considered that cargo expressed by, for example a megakaryocyte, is also found in the platelet or platelet-like membrane-bound cell fragment.

Accordingly, in some embodiments the cargo is endogenously produced by the progenitor, producer or effector-chassis of the invention, for example the engineered myeloid stem cell, a megakaryoblast, a megakaryocyte, a megakaryocyte-like cell, an iPSC, a platelet, or a platelet-like membrane-bound cell fragment platelet (or Synlet) or precursor cells such as a megakaryocyte.

The skilled person is well able to further engineer any of the progenitor, producer or effector-chassis as described herein to comprise the necessary constructs, promoters and coding sequences so as to express cargo, for example a cargo that is a:
a) a protein or peptide—in some embodiments the protein or peptide is:
   i) an antibody or antigen binding fragment thereof, for example an antibody or antigen binding fragment thereof binds to a tumor antigen or a neoantigen;
   ii) an enzyme, such as a nuclease for example a TALEN;
   iii) a cytokine for example IL-10; or
   iv) a CRISPR associated protein, for example Cas9;
   v) a bispecific protein, for example a bispecific antibody or optionally a T-cell engager (BiTE)
b) a nucleic acid—in some embodiments the nucleic acid is:
   i) an RNA, for example selected from mRNA, a miRNA, shRNA, and a clustered regularly interspaced short palindromic repeats (CRISPR) sequence.

As described above, it is possible to engineered the progenitor, producer or effector-chassis so as to place expression of the cargo under the regulation of a promoter that is only induced upon platelet activation. Such a strategy is particularly useful in situations where the cargo may be toxic to the progenitor, producer or effector-chassis or to the subject.

Any of the progenitor, producer or effector-chassis as described herein may be engineered so as to express a cargo from a genomic location, i.e. where the nucleic acid encoding the cargo and associated regulatory sequences are targeted to a locus within the genome. The nucleic acid may encode a fusion protein of a cargo protein or peptide, fused to an exosome targeting sequence, as described elsewhere herein.

It is clear that a nucleic acid encoding a cargo as described herein can be introduced in to a progenitor, producer or effector-chassis in a variety of ways. For example, in some embodiments the nucleic acid that encodes the cargo is introduced in to the genomic nucleic acid. For example, a nucleic acid encoding a cargo can be introduced to a first allele of a first locus, and/or the nucleic acid can be introduced to a second allele of a first locus. Additionally or alternatively, the nucleic acid can be introduced into a first allele of a first locus and a second nucleic acid (for example encoding a second cargo) can be introduced in to a first allele of a second locus. Additionally or alternatively, a first nucleic acid can be introduced into a first allele of a first locus and a second nucleic acid can be introduced into a second allele of the first locus.

In some embodiments the nucleic acid that encodes the cargo is introduced in to the progenitor, producer or effector-chassis and maintained in the progenitor, producer or effector-chassis episomally, for example as a circular nucleic acid, for example a vector.

In some embodiments the nucleic acid that encodes a cargo of the invention is introduced in to a progenitor, producer or effector-chassis via nucleofection.

The invention also provides a nucleic acid that encodes a cargo as described herein.

Also as described herein, the cargo can be exogenously loaded into the cargo.

In some embodiments it is preferred if the cargo is targeted to the alpha-granules. Exemplary alpha-granule targeting signals include PF4 and vWf. Accordingly in one embodiment the progenitor, producer or effector-chassis comprises a cargo as described herein that comprises an alpha-granule localisation signal, optionally a PF4 or vWf peptide sequence. In some embodiments the progenitor, producer or effector-chassis comprises a nucleic acid that encodes the cargo in-frame with an alpha-granule localisation signal, optionally wherein the alpha-granule localisation signal is selected from PF4 of vWf. The cargo comprising the alpha-granule localisation signal may be endogenously expressed, or exogenously loaded.

As described above, a means of targeting the delivery of cargo-loaded exosomes to a particular cell, tissue, organ or marker within the body is considered to be advantageous. Accordingly, in some embodiments, it is preferred if the cargo (endogenously generated or exogenously loaded) is directed towards the exosomes. Exosomes are typically stored in the alpha granules and are one of the most significant agents that are exported from the cell via degranulation.

The progenitor, producer or effector-chassis as described herein (whether non-engineered and expressing one or more of the receptors of the invention; or engineered progenitor, producer or effector-chassis expressing one or more receptors of the invention), which allow target engagement dependent activation of the platelet or platelet-like membrane-bound cell fragment, allow for target specific, localised exosome release from α-granules, avoiding the issues currently faced by systemic exosome delivery strategies. It is expected that exosome loading strategies developed in other cell types for a variety of cargoes can be applied to the targeting of cargo to the exosomes in the progenitor, producer or effector-chassis of the invention, since the constituent proteins of the platelet exosome are similar to classical exosomes.

Described below are exemplary means for targeting various cargo types to the exosome, for example for targeting protein or peptide cargo; RNA cargo; and/or Cas9 gene editing tools; to the exosome. Other means and combinations are also available for targeting these and other cargo types. The skilled person is therefore in a position to target cargo to the exosome.

There are two main classes of exosome loading strategy. The first requires no additional modification to the progenitor, producer or effector-chassis, for example to the megakaryocyte or megakaryocyte-like cell, since the targeting components are part of the cargo itself (i.e. where the progenitor, producer or effector-chassis is engineered to express the cargo endogenously, the exosome targeting motifs are incorporated within the cargo and a separate engineering step is not required in order to target the cargo to the exosomes). The second strategy requires some further engineering of the progenitor, producer or effector-chassis, for example a megakaryocyte or megakaryocyte-like cell or precursor thereof, so as to put in place something to which the cargo can be targeted, for example an engineered e.g. the TAMEL system described below.

### Strategy 1—Exosome Targeting Inherent in the Cargo

### Protein Cargo—Exosome Protein Loading

Exosomes feature distinct and specific membrane proteins which can be modified at their N or C-terminus to load cargo. By e.g. fusing a cargo protein or peptide to the C terminus of a tetraspanin (such as CD63) or non-tetraspanin such as PTGFRN or BASP1, that cargo protein of peptide is expected to localise within the lumen of an exosome as the homing protein would still be specifically targeted to the exosome compartment (Dooley, K et al. (2021). Molecular Therapy, 29(5), 1729-1743; Fu, S. et al (2020) 20(September), 100261).

Other genes that are considered to be located in the platelet exosome include the list of genes in Table A, derived from an ExoCarta search for proteins localised to the platelet.

TABLE A

| | | | | | | | | | |
|---|---|---|---|---|---|---|---|---|---|
| STEAP3 | CMIP | ATP1A3 | DENND3 | VPS4A | APOA4 | FCGR2C | VAV1 | PA2G4 | WASF3 |
| SERPINB1 | IGKV1-5 | LRRC57 | NHLRC2 | SEC22B | RDX | PTPN6 | IQGAP2 | MLEC | ANK1 |
| ATP2B1 | IGLC2 | VTN | ORMDL3 | DERA | TYMP | FN3K | ACVR1 | CCT6B | CLIC4 |
| GLIPR2 | DCD | CASP3 | CANX | GSN | APOD | DSP | ESAM | TMED2 | ST13P4 |
| NCKAP1 | FAM129A | CEACAM1 | ARPC3 | TUBA4A | ITGA2 | TARS | GPX1 | CTTN | JAK2 |
| SUSD3 | ATP9B | LMAN1 | SKAP2 | DNM2 | ACLY | FCN3 | CYFIP1 | WIPF1 | GP1BA |
| RAB6A | ARL15 | UNC13D | USP5 | BIN2 | HLA-E | WDR1 | HBD | SLC7A1 | CISD2 |
| TUBB2B | F2RL3 | C4BPA | ST13 | LIMS1 | PTPRJ | RAB27A | TC2N | PARK7 | BCAP31 |
| ATP6V0A2 | CAPNS1 | PGRMC1 | UBE2D2 | ZC3HAV1 | FYN | PRKACA | ITGA6 | CHMP2A | EEF1A2 |
| RAB8B | C1R | HAL | IGKC | FERMT3 | LIMS2 | M6PR | BLMH | VAMP8 | CNDP2 |
| BLVRB | CYFIP2 | MME | C8G | DAD1 | SLC12A6 | GNAS | PRKCA | LAT | PGM1 |
| PRDX6 | TLN1 | ATP5B | LTF | ST13P5 | C8A | RAB30 | MYLK | C1QA | PECAM1 |
| MYL6 | TRPC6 | ATP2C1 | GANAB | TSG101 | RAP2B | PFKP | STX7 | VAT1 | NCKAP1L |
| FCGBP | SNX18 | EGF | HLA- | CLTC | ALDH16A1 | S100A7 | EXOC3L4 | DSC1 | GRAP2 |
| | | | DRA | | | | | | |
| INPPL1 | LPCAT3 | SLC4A1 | MTHFD1 | GDI1 | ANXA3 | RASA3 | TCP1 | SLC2A1 | TUBB1 |
| EEF1A1P5 | FGG | CTSD | IGHG4 | PDLIM7 | CXCR4 | MYO1C | ABCB4 | FHL1 | RAN |
| PTPN11 | MGLL | MYL3 | ICAM3 | ITIH3 | SNAP23 | PIGR | RHOC | F5 | ELMO1 |
| PYGB | YES1 | NEDD4L | ARPC5 | C2orf88 | ENO2 | VPS36 | TRIP10 | RDH11 | TXNL1 |
| S100A7A | LRRC59 | IGHM | EFNB1 | CAPN7 | TBXAS1 | FRYL | ABI1 | GCA | SCAMP2 |
| TMEM40 | RAB32 | STRAP | TESC | HRG | TAPBP | CAMP | LDHB | PRKAR1A | RASA1 |
| FTL | ATP1A1 | AP1G1 | CETP | HPSE | C16orf54 | ADD1 | YIF1B | PDIA3 | YWHAH |
| RHBDF2CA2 | | | | | | | | | |
| TAP1 | ALB | ADD2 | PARVB | CAT | CORO1A | CLDN5 | HSPA1A | S100A8 | TUBB |
| NUDT5 | ATP6AP1 | UBAP1 | RAB5B | C9 | DBNL | TUBA3E | LST1 | Sep-11 | ALAD |
| AKR7A2 | GAPDH | RENBP | ACTR2 | NCK2 | TFG | TPM1 | WASF2 | SFN | PTP4A2 |
| SLA2 | RAB2A | LTBP1 | PVRL2 | TMC8 | SERPING1 | FCGR2A | TPST2 | ITGA4 | DSTN |
| RALB | ARHGAP18 | LRBA | HSPB1 | PEBP1 | AP1B1 | SLC6A4 | HLA- | RGS18 | CD63 |
| | | | | | | | DRB1 | | |
| CCT4 | ARHGAP6 | TMEM104 | EMILIN1 | PLXNA4 | WDR44 | CRKL | UBE2V2 | IDH1 | SND1 |
| APOC3 | MPP1 | P2RX1 | IGKV4-1 | MTPN | IDE | IGHG1 | WASF1 | PRKACB | LCN1 |
| C1QB | ADH1B | STIM1 | CA1 | MRVI1 | FGR | FLOT1 | NT5E | ACTA1 | SLC43A3 |
| LAP3 | RAB33A | GSDMA | HARS2 | EIF5A | UBE2N | DAAM1 | PFKL | PLXNB2 | BROX |
| PTP4A1 | MAPRE2 | APOA2 | CLIC1 | EPS15 | IGHG2 | FAM177A1 | UBA52 | PGD | RPN1 |
| CHMP2B | ANGPT1 | PGAM1 | VCL | ATP6VOD1 | SNCB | ARPC1B | INF2 | PZP | MYADM |
| RASGRP2 | DNM1L | TMC6 | IGHA2 | ENO1 | AP2B1 | PPP3CA | RAB18 | ALPL | PFN1 |
| ARF6 | TNPO1 | MMRN1 | CAND1 | TMED8 | MSN | CPNE1 | TALDO1 | IGSF8 | PGRMC2 |
| SLC25A4 | LCP2 | PSMB6 | STT3A | HLA- | TUBB4B | SLC25A6 | MYL12A | ERLIN2 | GDI2 |
| | | | | DRB5 | | | | | |
| DOK1 | RPN2 | LNPEP | LGALSL | DAB2 | TOR4A | TNIK | APOE | SLC44A1 | TSPAN15 |
| LMAN2 | F13A1 | APOA1 | NEDD4 | DHRS7 | ATP7A | TAOK1 | PGLS | LRSAM1 | VAPA |
| LHFPL2 | LCK | CPN2 | FGA | BGN | PKP2 | IGHA1 | ITGA2B | EPB41 | CORO1C |
| USP9X | ATP2B2 | HSD17B4 | LTK | PSMA7 | LTB4R | UBA1 | TGFB1 | ANXA1 | PTPRC |
| CD9 | STX12 | SLC7A5 | TJP2 | CD226 | CCT2 | PTTG1IP | ARAP1 | ADH1C | HSPA6 |
| NAPA | VTA1 | FAM65C | TOM1L2 | RAB7A | SPN | LDHA | HLA-A | TUBB4A | TM9SF4 |
| DARS | CDSN | ABCB6 | ROCK1 | TKT | TEC | CLCN3 | SELP | SRC | RPS27A |
| PCMT1 | EIF4A2 | CAPZA1 | HLA-B | CASP14 | ARL8B | RAP1B | DUSP3 | PSMA3 | HBA1 |
| PLXNB3 | PEF1 | SNCA | TSPAN14 | ARPC4 | EPS15L1 | ATP2B3 | PSTPIP2 | ILK | PLSCR3 |
| IGHG3 | GNAI3 | ST3GAL6 | P4HB | CFL2 | SLC25A5 | INPP5D | SERPIND1 | ATP2B4 | PTPRB |
| GP5 | ENDOD1 | STAM2 | ICAM2 | PRKCB | CD40LG | VWF | ITCH | SYTL4 | ACTR3 |
| C1S | BTN3A2 | CBR3 | Sep-06 | TUBA3C | CCT3 | SELL | CD82 | TREML1 | RHOG |
| BLVRA | PSMA8 | GGCT | FCER1G | RAC3 | HSPA5 | LY6G6F | NME2 | ERLIN1 | TPI1 |
| GGT2 | TUBA8 | PCBP3 | HPCAL1 | LAMTOR1 | CHMP6 | EPB42 | ATP1B3 | S100A4 | STK10 |
| CAB39 | ITGAV | PON1 | ACTN4 | ANKFY1 | B2M | DNM1 | PNP | NRAS | CCT7 |
| RAP1GDS1 | PPP1CC | UBE2V1 | CPNE3 | ITGAL | CDC42BPB | YWHAG | COLEC10 | RAB11B | DOCK5 |
| ANPEP | TAOK3 | PLCB3 | STOM | PRKAG1 | VAMP2 | PLXNA3 | DPYSL2 | RAB5A | TWF2 |
| HPRT1 | FAM151A | SRI | CD36 | ATP5A1 | CKAP5 | VPS28 | CAPZB | FAM49B | STT3B |
| HBB | PRDX1 | PLXDC2 | SLC6A6 | GNAQ | HNRNPK | GNA11 | SIT1 | ERBB2IP | USO1 |
| SERPINB4 | ESYT2 | RNF11 | DOK2 | TPP2 | SLC12A4 | KNG1 | PIP4K2A | VDAC2 | BRK1 |
| SLC3A2 | SERPINB12 | TOLLIP | OLA1 | PDIA5 | ESD | RAB4A | TM9SF2 | SDPR | GP1BB |
| SLC29A1 | FLNA | ERMAP | GRB2 | CDC37 | MTMR12 | PI4KA | CAPZA2 | TMEM63B | AK1 |
| CNP | TMED9 | TMEM30A | SLC44A2 | RALA | IGLL5 | ACTB | NAPG | APOB | SPCS3 |
| STAM | GNB2 | PMVK | ARPC2 | TMBIM1 | PLCG2 | ATP2A2 | TMED4 | PACSIN2 | CD5L |
| CHMP4A | VASP | AQP1 | SPCS2 | CAPN1 | NAP1L1 | AKR7L | IMPA1 | NCSTN | FTH1 |
| EPHX2 | RAP2A | FAH | PRKCQ | LPA | HSP90AA1 | MYL1 | PCBP1 | SERPINA1 | SLC16A3 |
| PRDX2 | CHMP5 | VAPB | TUBB2A | UBASH3B | YWHAE | ATP6V1B2 | DDOST | PSMA1 | SDCBP |
| PEAR1 | VPS13C | UBE2NL | KRT6B | TUBA1C | WARS | BAIAP2 | GSTP1 | PLTP | TPM4 |
| GYPC | GNAI2 | RAB27B | ITGB2 | PPIB | SERPINB6 | EHD1 | ATP6V0A1 | SNAP29 | DOCK10 |
| GOLGA7 | SYNGR2 | RSU1 | JAM3 | CD109 | KPNB1 | S100A9 | VCP | PYGL | YWHAB |
| SUSD1 | RRAS | MYL12B | PDE5A | BTK | CAP1 | RGS6 | CD37 | ECE1 | PLEKHO2 |
| GNB1 | YWHAQ | CCM2 | RAB35 | SELPLG | EIF5A2 | MFGE8 | C4B | LAMP1 | RRAS2 |
| ARL6IP5 | BSG | MLC1 | C3 | SNX3 | ABI2 | IGLC3 | C10orf54 | S100A14 | RHOA |
| PDCD10 | ATP2A3 | NBEAL2 | STXBP5 | SHANK3 | CSTA | PGK1 | TMEM55A | HP | LASP1 |
| CTDSPL | SLC1A5 | DOK3 | TMEM63A | WBP2 | MOB1B | PTPRCAP | APOA5 | TBXA2R | C1QC |
| UNC45A | YWHAZ | MYH9 | MTSS1 | PLG | USP9Y | GGT3P | PDLIM1 | F2 | TUBA1A |
| GSR | AP2A1 | ITGA1 | FHOD1 | CP | RAB1A | MYH14 | ATIC | FGB | ANXA4 |
| ARHGDIB | SRGAP2 | EFR3A | HLA- | VAMP7 | FYB | RAB1C | STX4 | ACSL4 | RAB39A |
| | | | DPB1 | | | | | | |
| ARHGAP1 | RAB11A | PGK2 | IST1 | CLEC1B | STXBP3 | TUBA1B | PPP1CA | ANXA2 | DIP2B |
| DNAJB6 | EHD3 | PDCD6 | NCCRP1 | GP9 | BPIFB1 | ABCB11 | IGLC1 | RAB1B | FKBP1A |
| LAPTM5 | LGALS3BP | SPTB | ALOX12 | ARRDC1 | JUP | SLC2A14 | PPP2R1A | C6 | LGALS7 |
| ARF4 | VAMP3 | WWP1 | EIF4A1 | C5 | ITIH4 | RUVBL1 | MYL9 | PDCD6IP | DEF6 |
| PKHD1L1 | IGLC6 | ACTC1 | TSPAN33 | PSMA6 | SOD1 | P2RY12 | TRIM58 | ASAP1 | LRP1 |
| ESYT1 | ARF1 | HPR | GSTO1 | CHMP1A | APOL1 | FASN | KALRN | SCAMP3 | NPEPPS |
| SLC9A3R1 | TTR | ATG9A | ERP44 | F11R | STXBP2 | LYPLA1 | EHD4 | ADAM10 | TMED10 |
| TTC7B | NME1 | ANXA11 | LRRC32 | A2M | GTPBP2 | GP6 | ITGB3 | LYZ | MOB1A |
| ALDOA | G6PD | CHMP4B | TPM2 | UBC | ASAP2 | HMHA1 | PPBP | BTN3A1 | TPM3 |
| RAB8A | VPS25 | BCAM | MAPK14 | ACTG2 | AZGP1 | GNAZ | STK38 | RAP1A | NSF |
| ANXA6 | NCK1 | ACTG1 | MAGT1 | HSPA8 | SACM1L | GNAI1 | ENPEP | CCT5 | RAB3C |
| MAPK1 | HEG1 | TTYH3 | VNN1 | ABCC4 | LCN1P1 | RAB6B | ABHD16A | ORMDL2 | MVP |
| MAPRE1 | UBB | HPCA | EEF1A1 | GABARAPL2 | RTN4 | EMD | DAPP1 | SEC11A | RAB10 |
| ROCK2 | CFL1 | ATP6V1A | SCAMP1 | ABCC1 | TF | CSK | PRKAR2B | SERPINA3 | LYN |
| CDC42 | THBS1 | ITIH2 | CD44 | RTN2 | YKT6 | LSR | IQGAP1 | PCBP2 | PPIA |
| PPM1A | DSG1 | RAC1 | ARF3 | ZYX | WAS | VPS4B | TFRC | ENG | DPP4 |
| RAB14 | FAM63A | RAB37 | PRKCD | EEF1G | EIF5AL1 | COTL1 | DSC3 | PITPNB | SORT1 |
| ITIH1 | ARG1 | C4A | OSTF1 | HLA- | EEF2 | VPS45 | GPRC5C | ATP8A1 | GRHPR |
| | | | | DRB3 | | | | | |
| MINK1 | RAP1GAP2 | CHMP1B | STK24 | AHCY | SLC2A3 | MAP4K4 | CCT6A | PTPRA | CD2AP |
| MFSD2B | STAT3 | ARHGDIA | HGS | KIF2A | ADH1A | ANXA7 | PSMD9 | UGP2 | CTNNB1 |
| TSPAN9 | CCT8 | ANXA5 | TAGLN2 | CPNE2 | PROS1 | MYO1G | APRT | ORMDL1 | TMX1 |
| UBE2D3 | AGT | PRDX4 | EZR | PLEK | RAB13 | SERPINB3 | ADCY6 | SLC9A9 | GNB4 |
| MYCT1 | PDIA6 | RAB2B | HSPA7 | UBA7 | KIAA0513 | OIT3 | AMPD2 | ARHGAP4 | IGLC7 |
| STX11 | CLDN3 | DOCK11 | DIAPH1 | PTGS1 | ACTN1 | RHEB | MPL | GGT1 | ALDOC |
| CLU | CIB1 | CAPNS2 | AHSG | ACTA2 | RAB21 | GNA13 | SYK | PRDX5 | XPNPEP2 |
| APPL2 | CDS2 | GPI | ARL8A | ANXA2P2 | ANO6 | VTI1B | INPP5A | ITGB1 | EPS8 |
| ITGA5 | CD84 | CD47 | LBP | PODXL | RAB5C | VPS37B | SLC9A1 | BTN3A3 | FN1 |
| HARS | HSP90AB1 | HSP90B1 | SERPINB9 | CYB5R3 | FLOT2 | C1orf198 | TGM3 | C1orf198 | RHOF |
| RAC2 | | | | | | | | | |

The expression of a fusion protein comprising a cargo protein or peptide and a membrane resident exosome protein in a megakaryocyte expression, is expected to result in platelet exosome loading with that cargo protein or peptide.

Soluble protein can be targeted to the exosome by fusion of targeting sequences to a cargo protein or peptide. One such approach is fusion of the WW domain of Nedd4 ubiquitin ligases to a therapeutic protein (Sterzenbach, U. et al (2017) 25(6), 1269-1278). This strategy results in the uptake of the chimeric WW domain—cargo protein/peptide fusion into the lumen of the exosome by an Ndfip1 dependent mechanism.

Ubiquitinated proteins are also often trafficked to the exosome. Tagging a cargo protein or peptide with a ubiquitin tag can also be used to direct trafficking of the cargo protein or peptide to the exosome lumen as a soluble protein (Cheng, Y., & Schorey, J. S. (2016) Biotechnology and Bioengineering, 113(6), 1315-1324; Giovannone, A. et al (2017) Molecular Biology of the Cell, 28(21), 2843-2853).

The expression of a fusion protein comprising a cargo protein or peptide and an an exosome loading tag in a megakaryocyte expression, is expected to result in platelet exosome loading with that cargo protein or peptide.

Accordingly, in some embodiments where the cargo is a protein or peptide, the cargo protein or peptide is expressed as a fusion protein comprising:
a) the cargo protein or peptide; and
b) an exosome targeting domain, for example where the exosome targeting domain is selected from the group comprising or consisting of:
   i) an exosome specific membrane protein or exosome membrane targeting portion thereof, for example:
      a tetraspanin, for example CD63; or
      a non-tetraspanin such as PTGFRN or BASP1
   ii) an exosome targeting sequence from a soluble protein, for example the WW domain of Nedd4 ubiquitin ligases; and/or
   iii) a ubiquitin tag; and/or
   iv) a protein selected from the proteins listed in Table A.

As stated elsewhere, although the preferred effector-chassis is a platelet or platelet-like membrane-bound cell fragment (or engineered version thereof), the skilled person appreciates that when expressing a cargo endogenously, that expression typically occurs in one or more of the upstream progenitor cells, for example in the progenitor or producer-chassis e.g. a megakaryocyte of megakaryocyte-like cell.

Accordingly, in some embodiments the invention provides a progenitor, producer or effector-chassis as described herein that expresses any one or more cargo proteins or peptides wherein the cargo protein or peptide is expressed as a fusion protein comprising:
a) the cargo protein or peptide; and
b) an exosome targeting domain, for example where the exosome targeting domain is selected from the group comprising or consisting of:
   i) an exosome specific membrane protein or exosome membrane targeting portion thereof, for example:
      a tetraspanin, for example CD63; or
      a non-tetraspanin such as PTGFRN or BASP1
   ii) an exosome targeting sequence from a soluble protein, for example the WW domain of Nedd4 ubiquitin ligases; and/or
   iii) a ubiquitin tag; and/or
   iv) a protein selected from the proteins listed in Table A.

As described above, the progenitor, producer or effector-chassis may be engineered so as to express the cargo fusion protein or peptide from a genomic location, and/or so as to express the cargo fusion protein or peptide episomally.

Also as described elsewhere herein, the cargo may be loaded in to the progenitor, producer or effector-chassis exogenously. Accordingly the invention also provides a progenitor, producer or effector-chassis as described herein that comprises one or more cargo proteins or peptides and wherein the cargo protein or peptide is a fusion protein comprising:
a) the cargo protein or peptide; and
b) an exosome targeting domain, for example where the exosome targeting domain is selected from the group comprising or consisting of:
   i) an exosome specific membrane protein or exosome membrane targeting portion thereof, for example:
      a tetraspanin, for example CD63; or
      a non-tetraspanin such as PTGFRN or BASP1
   ii) an exosome targeting sequence from a soluble protein, for example the WW domain of Nedd4 ubiquitin ligases; and/or
   iii) a ubiquitin tag; and/or
   iv) a protein selected from the proteins listed in Table A

and wherein the cargo protein or peptide has been exogenously loaded into the progenitor, producer or effector-chassis.

### RNA Cargo—Exosome RNA Loading

As described elsewhere, the cargo that is to be delivered in a targeted (e.g. through a targeted means using a progenitor, producer or effector-chassis or engineered progenitor, producer or effector-chassis as described herein that comprises one or more of the receptors of the invention) or non targeted (e.g. via an engineered progenitor, producer or effector-chassis as described herein that does not comprise one or more of the receptors of the invention) fashion can be an RNA. It is also considered advantageous to target the RNA cargo to the exosome for delivery.

Exosomes naturally contain a variety of RNA cargoes, including mRNA and miRNA. Platelets have been previously shown to deliver RNA cargo to cells on activation, (miRNA transfer (Laffont et al., 2013 Blood, 122(2), 253-261; Michael et al., (2017) Blood, 130(5), 567-580), mRNA transfer (Kirschbaum, M., (2015). Blood, 126(6), 798-806; Risitano, A et al (2012) Blood, 119(26), 6288-6295).

### RNA Targeting Motifs

Studies of exosome resident RNAs have elucidated a variety of sequences which can increase exosomal RNA localisation in exogenous RNAs (Batagov, A. O. et al (2011) 10th Int. Conference on Bioinformatics—1st ISCB Asia Joint Conference 2011, InCoB 2011/ISCB-Asia 2011: Computational Biology—Proceedings from Asia Pacific Bioinformatics Network (APBioNet), 12(SUPPL. 3) https://doi.org/10.1186/1471-2164-12-S3-S18). Different engineering strategies have been proposed to increase exosome RNA localisation. Some of these sequences mediate interactions with RNA binding proteins, resulting in exosomal RNA sorting, via hnRNPagihA2B1 (Villarroya-Beltri et al (2013) Nature Communications, 4, 1-10), SYNCRIP (Santangelo, L. et al (2016) Cell Reports, 17(3)) or Annexin A2 (Hagiwara, K. et al (2015) FEBS Letters, 589(24), 4071-4078). Some pre-miRNA backbones feature specific hairpin structures (such as pre-miR-451) that target them to the exosome which can be repurposed to facilitate targeting of alternate mRNAs when used as a packaging scaffold (Reshke, R. et al (2020) Nature Biomedical Engineering, 4(1), 52-68). Finally, some viral RNAs contain exosome targeting motifs which can be repurposed for therapeutic RNA re-targeting (Lévesque et al., 2006 Traffic, 7(9), 1177-1193).

### Strategy 2—Co-Engineering the Progenitor, Producer or Effector-Chassis to Facilitate Exosome Targeting

In addition to taking advantage of endogenous RNA exosome trafficking systems, new, orthogonal targeting systems have been developed to drive the exosome specific accumulation of RNA. The TAMEL system involves the fusion of a the bacteriophage coat protein MS2 was fused to Lamp2b, VSVG and CD63 (exosome membrane proteins). Target therapeutic RNA was engineered to express MS2 binding stem-loops. The engineered stem-loops present in the cargo mRNA drive association with the engineered MS2-exosome membrane protein fusions, driving cargo RNA accumulation within the exosome (Hung & Leonard, 2016 Journal of Extracellular Vesicles, 5(1), 1-13). The MS2 loading system has been further modified to drive RNA association with the exosomal membrane in a blue light-dependent manner. By fusing the MS2 coat protein and an exosomal membrane protein to light dependent dimerization proteins, MS2 stem-loop containing RNA can be specifically loaded into exosomes in the presence of blue-light only. On blue light shut off, the MS2 coat protein/RNA dissociate from the exosomal membrane protein. This facilitates cytoplasmic translation of the mRNA upon its uptake by a target cell (Huang, L., et al (2019) Advanced Functional Materials, 29(9), 1-8; Yim, N. et al (2016) Nature Communications, 7, 1-9). Finally, a similar strategy has been employed by fusing the archeal ribosomal protein L7Ae to CD63, facilitating L7Ae targeting to the exosome. L7Ae binds to an RNA structure known as the C/D box. Engineering of the C/D box into the 3′ UTR of an mRNA, in cells co-expressing the L7Ae-CD63 fusion, resulted in C/D box containing mRNA enrichment within the exosome compartment. These strategies could be employed to target mRNA to platelet exosome compartment (Kojima et al., 2018 Nature Communications, 9(1)).

In some embodiments, any of the progenitor, producer or effector-chassis of the invention described herein has been engineered to fuse the bacteriophage coat protein MS2 to an exosome membrane protein, optionally to Lamp2b, VSVG and/or CD63. The presence of a cargo RNA comprising the corresponding MS2 binding stem-loops within the progenitor, producer or effector-chassis (either expressed endogenously or exogenously loaded) results in the targeting of the cargo RNA to the exosome.

In some embodiments, any of the progenitor, producer or effector-chassis of the invention described herein has been engineered to fuse the bacteriophage coat protein MS2 to:
an exosome membrane protein, optionally to Lamp2b, VSVG and/or CD63; and
a light dependent dimerisation protein.

A cargo RNA comprising the corresponding MS2 binding stem-loops present within the progenitor, producer or effector-chassis (either expressed endogenously or exogenously loaded) is only loaded into the exosome in the presence of blue light.

In some embodiments, any of the progenitor, producer or effector-chassis of the invention described herein has been engineered to fuse the archeal ribosomal protein L7Ae to an exosome membrane protein, optionally to Lamp2b, VSVG and/or CD63; in some embodiments fused to CD63. The presence of a cargo RNA comprising the corresponding C/D box binding partner within the progenitor, producer or effector-chassis (either expressed endogenously or exogenously loaded) results in the targeting of the cargo RNA to the exosome.

As described above, the progenitor, producer or effector-chassis (i.e. any progenitor, producer or effector-chassis as described herein) may be engineered so as to express the cargo RNA from a genomic location, and/or so as to express the cargo fusion RNA epiosmally.

Also as described elsewhere herein, the cargo RNA may be loaded in to the progenitor, producer or effector-chassis exogenously.

Accordingly the invention also provides a progenitor, producer or effector-chassis as described herein that has been engineered to express any one or more of:
a) a cargo RNA that comprises an exosome targeting motif, optionally a hairpin or a viral exosome targeting RNA or exosome targeting fragment thereof;
b) a cargo RNA that comprises an aptamer domain, optionally wherein the aptamer domain is selected from:
   i) a MS2 binding stem-loops; and/or
   ii) a C/D box; and/or
   iii) an AU rich element, optionally wherein the RNA is an mRNA that encodes Cas9.

The invention also provides a progenitor, producer or effector-chassis as described herein that comprises any one or more of:
a) a cargo RNA that comprises an exosome targeting motif, optionally a hairpin or a viral exosome targeting RNA or exosome targeting fragment thereof;
b) a cargo RNA that comprises an aptamer domain, optionally wherein the aptamer domain is selected from:
   i) a MS2 binding stem-loops; and/or
   ii) a C/D box; and/or
   iii) an AU rich element, optionally wherein the RNA is an mRNA that encodes Cas9;

and wherein the cargo RNA has been exogenously loaded.

The skilled person appreciates that where the cargo RNA comprises an MS2 binding stem-loop, the progenitor, producer or effector-chassis should typically be engineered to express a fusion of the bacteriophage coat protein MS2 to an exosome membrane protein, optionally to Lamp2b, VSVG and/or CD63. In some instances the progenitor, producer or effector-chassis may be engineered to express a fusion protein comprising:
a) the bacteriophage coat protein MS2
b) an exosome membrane protein, optionally to Lamp2b, VSVG and/or CD63; and
c) a light dependent dimerization protein.

The skilled person also appreciates that where the cargo RNA comprises a C/D box, the progenitor, producer or effector-chassis should typically be engineered to express a fusion of the archeal ribosomal protein L7Ae to an exosome membrane protein, optionally to Lamp2b, VSVG and/or CD63; in some embodiments fused to CD63. In some instances the progenitor, producer or effector-chassis may be engineered to express a fusion protein comprising
a) the archeal ribosomal protein L7Ae
b) an exosome membrane protein, optionally to Lamp2b, VSVG and/or CD63; and
c) a light dependent dimerization protein.

The skilled person appreciates that there are other combinations of RNA sequence (i.e. aptamer) and RNA-binding protein that can be used, in addition to or instead of the C/D box:L7Ae and stem-loop:MS2 combinations described above.

Accordingly in some embodiments the progenitor, producer or effector-chassis may be engineered to express a fusion protein comprising:
a) an exosome specific protein
   for example a tetraspanin, for example CD63; or
   a non-tetraspanin such as PTGFRN or BASP1; or Lamp2b or VSVG;
   or a protein selected from those listed in Table A; and
b) a protein or fragment thereof capable of binding to a specific RNA aptamer.

It is clear to the skilled person that in such an embodiment, the corresponding cargo RNA should comprise the specific aptamer to which the protein in (b) above binds.

### Cas9 RNP Delivery

The above mentioned strategies focus on RNA and protein exosomal loading in isolation. To facilitate genome engineering by Cas9/guide RNA delivery, both guide and protein must be delivered. A number of engineering strategies have been proposed to drive Cas9 RNP uptake in exosomes, and thus facilitate gene editing in target cells exposed to the exosome on release. One such approach is through the engineering of a CD9-HuR. CD9 is an exosomal membrane protein, and HuR is an RNA binding protein that specifically targets AU rich elements (AREs). Engineering of AREs into the 3′ UTR of an mRNA encoding for Cas9, and its addition to an guide RNA, drive their accumulation within exosomes, through their association with HuR and thus CD9 (Li, Z. (2019) Nano Letters, 19(1), 19-28). Another approach for loading Cas9 into exosomes involves fusion of GFP to a exosomal membrane protein (such as CD9 or CD63). Cas9 can then be fused to a GFP binding nanobody. This results in the binding of Cas9 protein itself to the GFP of bound to the exosome membrane protein, driving its accumulation with the exosome compartment. As this strategy is driven by targeting of the Cas9 protein, sgRNAs expressed in the producer cell bind to the soluble Cas9 taken up within the exosome, facilitating complete RNP transfer). Finally, functional Cas9 systems have also been delivered to exosomes in the form of a plasmid. When exosome producer cells (in this case—the megakaryocyte) is nucleofected with a plasmid, some of this plasmid is stochastically packaged within exosomes. This plasmid can then be transferred to target cells upon exosome release (Kim, S. M. et al (2017) Journal of Controlled Release, 266(July), 8-16).

Accordingly, in some embodiments any of the progenitor, producer or effector-chassis of the invention described herein has been engineered to express a CD9-HuR fusion protein. A cargo RNA comprising AU rich elements is targeted to the exosome. The cargo RNA comprising AU rich elements may be expressed endogenously (i.e. the progenitor, producer or effector-chassis may also be engineered so as to express the cargo RNA), or loaded exogenously. This method may be used to target any cargo RNA to the exosome.

In some embodiments, any of the progenitor, producer or effector-chassis of the invention described herein may have been engineered to express a fusion protein comprising GFP fused to an exosomal membrane protein such as CD9 or CD63. A cargo protein fused to an GFP binding nanobody is targeted to the exosome membrane driving its accumulation within the exosome. The methods is considered to be suitable for the targeting of any cargo protein or peptide to the exosome. In some embodiments the progenitor, producer or effector-chassis has been engineered to also express the corresponding sgRNA—the sgRNA bind to the soluble Cas9 and so are both targeted to the exosome. The cargo protein (e.g. Cas90 and/or sgRNAs may be expressed endogenously, i.e. the progenitor, producer or effector-chassis may have been engineered so as to express the cargo protein (e.g Cas9) and/or sgRNAs; or the cargo protein (e.g. Cas9) and/or sgRNAs may have been loaded exogenously.

It is clear from the above that there are many appropriate means by which a cargo (e.g. protein, peptide, RNA etc) can be targeted to the exosome. Engineered progenitor, producer or effector-chassis that are engineered megakaryocytes of megakaryocyte-like cells that have been engineered to express any one or more cargo and/or corresponding components required for exosome targeting as described above are expressed to produce platelets or platelet-like cell fragments that comprise the cargo in the exosomes.

Embodiments where the cargo is targeted to the exosome are considered to be particularly advantageous when the progenitor, producer or effector-chassis comprises one or more of the receptors of the invention, (i.e. one or more CPR, universal CPR, SAPR or ePAR)—the presence of the receptor of the invention means that the progenitor, producer or effector-chassis is targeted to a particular site, or activates in response to a specific signal. Due to the limited half life, and potential for extravasation of exosomes, local release of exosomes at the site of a specific target is expected to have a massive impact on the therapeutic potential of exosome based therapies.

The invention provides a nucleic acid that encodes any one or more of the cargos (including any exosome target binding domains), suitable for expression of the cargo from the genomic locus, or episomally.

Various methods are provided for delivering a cargo to a subject in need thereof. As described herein, the cargo may be a therapeutic drug or a toxin. Preferences for the cargo are as described elsewhere herein, for example the cargo may be located within exosomes and exported from the platelet/Synlet in an exosome, for example from an alpha granule.

Other targeting signals may be used in a similar manner, for example to target the cargo to the exosome, or other granule. Accordingly in some embodiments the cargo (whether it is endogenously or exogenously loaded) is attached to a targeting signal, for example an α-granule localization signal and/or an exosome targeting signal.

In other embodiments, the cargo is exogenously loaded into or onto the progenitor, producer or effector-chassis, for example into or onto an engineered myeloid stem cell, a megakaryoblast, a megakaryocyte, a megakaryocyte-like cell, an iPSC, a platelet, or a platelet-like membrane-bound cell fragment or Synlet of the invention. Platelets naturally absorb drugs and antibodies in their environment through endocytosis and the open canalicular system and the skilled person is aware of techniques for exogenously loading platelets with cargo, see for example Wu et al 2020 J Biomed Sci 27 discusses the loading of platelets with doxorubicin by incubation with doxirubicin. Following platelet activation doxirubicin was found in the platelet extracellular vesicles that are normally released upon platelet activation. Verheul et al 2007 Clin Cancer Res 15: 5341-5347 demonstrates the loading of platelets with Bevacizumab and the release of the antibody upon platelet activation; Xu et al 2019 Biometer Sci 7: 4568-4577 and shows loading of platelets with vincristine, a chemotherapy medication. Accordingly, platelets are able to be exogenously loaded with a wide range of cargo, which upon platelet activation is released from the platelet.

In some embodiments then the invention provides any of the chassis of the invention that has been exogenously loaded with a cargo, for example where the cargo is selected from:
a) a protein or peptide—in some embodiments the protein or peptide is:
   i) an antibody or antigen binding fragment thereof, for example an antibody or antigen binding fragment thereof binds to a tumor antigen or a neoantigen;
   ii) an enzyme, such as a nuclease for example a TALEN;
   iii) a cytokine for example IL-10; or
   iv) a CRISPR associated protein, for example Cas9;
   v) a bispecific protein, for example a bispecific antibody or optionally a T-cell engager (BiTE)
b) a nucleic acid—in some embodiments the nucleic acid is:
   i) an RNA, for example selected from mRNA, a miRNA, shRNA, and a clustered regularly interspaced short palindromic repeats (CRISPR) sequence; or
   ii) a DNA vector;
c) a toxin;
d) a small molecule drug, imaging agent, radionucleotide drug, radionucleotide tagged antibody, or any conjugate thereof;
e) a viral vector such as AAV;
f) a virus such as oncolytic virus;
g) agents for performing CRISPR mediated gene editing;
h) an exosome, for example an exosome pre-loaded with a second cargo; and/or
i) a nanoparticle or nanoparticles.
or any combination thereof.

Preferences for the cargo are as described elsewhere herein. For example in some embodiments the cargo is a protein that comprises an exosome targeting domain, as described above. In some embodiments the cargo is soluble. In some embodiments the cargo is membrane-bound.

The cargo may also be an imaging agent.

It is also considered that incubating the chassis of the invention with a composition comprising exosomes or other lipid bound vesicles such as synthetic exosomes allows the exogenous loading of the exosomes into the chassis that can be targeted for delivery using the chassis described herein. In some embodiments the composition comprises exosomes that comprise one or more secondary cargo such as any of the cargo described herein.

In some preferred embodiments the cargo is not an agent that is naturally found within the platelet, i.e. the cargo is an exogenous cargo rather than an endogenous cargo with respect to the platelet. The skilled person appreciates that a cargo can be exogenous to the platelet but endogenous to the subject.

In some preferred embodiments the cargo is not an agent that is naturally found within the platelet α-granule. For example the cargo may be an agent that is naturally found within the platelet, but not naturally found within the α-granule.

In some embodiments the cargo may be an agent that is endogenously found within the platelet but is found at a higher concentration or amount within the platelet, or within the α-granule of the platelet than in a platelet not of the invention.

In some embodiments the cargo comprises an α-granule localization signal wherein the α-granule localization signal directs the cargo to uptake into 0-granule vesicles of the engineered platelet.

For example in some embodiments a therapeutic agent or an imaging agent comprises or is conjugated to an α-granule localization signal.

Cargo that is endogenously expressed or exogenously loaded may be stored with the progenitor, producer or effector-chassis in various places. For example in some embodiments the cargo is stored in the cytoplasm and/or cargo is stored in the plasma membrane, and/or the cargo is stored on the external surface of the plasma membrane and/or the cargo is stored in one or more granules, preferably is stored in the alpha-granule.

In some embodiments, the cargo agent for example therapeutic agent is stored within an exosome within the progenitor, producer or effector-chassis for example within the platelet or platelet-like membrane-bound cell fragment. In some embodiments the at least one cargo agent for example therapeutic agent is within a granule, for example within an alpha granule within the progenitor, producer or effector-chassis for example within the platelet or platelet-like membrane-bound cell fragment. In some further embodiments, the at least one cargo agent for example therapeutic agent is within an exosome that itself is within a granule, for example an alpha granule.

Platelet α-granules contain protein effectors and loading of soluble proteins is performed through a simple signal peptide. A minimal targeting sequence for directing proteins into platelet secretory α-granules has been previously defined (See, Golli et al. “Evidence for a Granule Targeting Sequence within Platelet Factor 4.”, JBC, 2004, which is hereby incorporated by reference in its entirety) and in any of these embodiments, the cargo may be attached to an α-granule localization signal. For example, where the cargo is endogenously produced, the cargo may be expressed by the platelet (or Synlet) or precursor cell such as a megakaryocyte in frame with an α-granule localization signal. In fact,

Exogenously loaded cargo may also be attached to an α-granule localization signal so that once the cargo enters the platelet or the progenitor cell, it is then subsequently targeted to the α-granule.

In instances where the cargo is endogenously produced by the progenitor, producer or effector-chassis or engineered progenitor, producer or effector-chassis, the cargo can be encoded by a nucleic acid that is expressed to produce the cargo protein or peptide, or RNA that is to be targeted to the specific target site, tissue or cell; or to produce enzymes or other active entities that produce the cargo within the platelet (or Synlet) or precursor cell such as a megakaryocyte.

The engineered progenitor, producer or effector-chassis according to any of the preceding claims wherein the progenitor, producer or effector-chassis has been engineered to:
a) have disrupted function of MHC Class 1 genes or proteins;
b) have disrupted expression from the β2 microglobulin gene, optionally to knock out the 02 microglobulin gene;
c) have disrupted expression from one or more HLA genes;
d) have disrupted expression from any one or more of HLA-A, HLA-B and/or HLA-C, optionally wherein expression of HLA-A and HLA-B has been entirely disrupted but wherein expression of HLA-C has been partially disrupted, optionally wherein expression from both alleles of HLA-A and HLA-B have been disrupted but wherein expression from only one allele of HLA-C has been disrupted;
e) overexpress any one or more of the HLA class Ib genes, optionally any one or more of HLA-G, HLA-E, CD47 and PD-L1;
f) overexpress any one or more of HLA-G, HLA-E, CD47 and PD-L1 and optionally been engineered to have disrupted expression from the Beta 2 microglobulin gene; and/or
g) overexpress one or more immunomodulatory genes, optionally wherein the one or more immunomodulatory genes is selected from the group comprising CD47 and PD-L1;
h) eliminate one or more genes or gene products for which the product(s) could negatively affect the potency of a cargo;
i) tune up or down the innate/adaptive response;
j) reduce inflammation, angiogenesis, atherosclerosis, lymphatic development and tumour growth;
k) have disrupted expression of one or more genes encoding adhesive proteins and/or cargo entities which are likely to indirectly counter the biological action of the engineered cargo, potentially leading to a greater net therapeutic effect;
l) downregulate or inhibit expression of CD40L;
n) downregulate or inhibit expression of any one or more of CD36, NOD2, SRB1, TLR1, TLR2, TLR3, TLR4, TLR6, TLR9, CD40L, CD93 (C1qRp), C3aR, CD88 (C5aR), CD89 (FcαR1), CD23 (FcεR1), CD32 (FcγRIIa), MHC class1, CD191 (CCR1), CD193 (CCR3), CD194 (CCR4), CD184 (CXCR4), CX3CR1, CD102 (ICAM-2), JAM-C/JAM-3, CD62P (P-selectin), CD31 (PECAM-1), CD150 (SLAMF1), CCL2, CCL3, CCL5, CXCL1, CXCL12, CXCL4/PF4, CXCL5, CXCL8, NAP2 (CXCL7), IL-1p
o) disrupt or inhibit expression of GARP and/or TGFb;
q) disrupt or inhibit expression of any one or more of Siglec-7, Siglec-9, Siglec-11 or TGFβ
s) disrupt or inhibit expression of any one or more of GPIb/V/IX and GPVI (GP6), ITGA2B, CLEC2, integrins s aIIbb3, a2b1, a5b1 and a6b1, GPVI and ITGA2B;
t) disrupt or inhibit expression of any one or more of Par1, Par4, P2Y12, GPIb/V/IX, the Thromboxane receptor (TBXA2R), P2Y1, P2X1 and integrin aIIbb3 or from the group consisting of Par1, Par4 and P2Y12;
u) disrupt or inhibit expression of any one or more of Cox1, Cox2, HPS, prothrombin, PDGF, EGF, von Willebrand Factor and thromboxane-A synthase (TBXAS1);
v) synthesise a protein or RNA of interest in response to activation of the platelet or platelet-like membrane-bound cell fragment, optionally wherein the protein or RNA of interest is expressed from the BCL-3 mRNA untranslated regions, optionally 5′UTR;
z) express one or more cargo proteins or cargo RNAs, optionally wherein the cargo protein or cargo RNA comprises an alpha-granule targeting signal, optionally comprises a platelet factor 4 (PF4) or von Willebrand factor (vWf);
aa) express at least two CPRs, universal CPRs, complexes of universal CPRs and tagged targeting peptides, SAPRs or ePARs, optionally express at least 3, 4, 5, 6, 7, 8, 9 or at least 10 different CPRs, universal CPRs, complexes of universal CPRs and tagged targeting peptides, SAPRs or ePARs;
bb) express at least two CPRs, universal CPRs, complexes of universal CPRs and tagged targeting peptides, SAPRs or ePARs, and wherein the target binding domain of the at least two CPRs, universal CPRs, complexes of universal CPRs and tagged targeting peptides, SAPRs or ePARs are directed towards different targets;
cc) express at least two CPRs, universal CPRs, complexes of universal CPRs and tagged targeting peptides, SAPRs or ePARs, and wherein the target binding domain of the at least two CPRs, universal CPRs, complexes of universal CPRs and tagged targeting peptides, SAPRs or ePARs are directed towards different targets, and wherein:
   i) the platelet modulation domain of a first CPR, universal CPR, complex of universal CPR and tagged targeting peptide, SAPR or ePAR is a platelet activation domain optionally a degranulation triggering domain optionally an ITAM containing domain, and wherein the platelet modulation domain of a second CPR, universal CPR, complex of universal CPR and tagged targeting peptide, SAPR or ePAR is a platelet inhibition domain, optionally is a domain that prevents triggering of platelet degranulation, optionally is an ITAM containing domain;
   ii) the platelet modulation domain of a first CPR, universal CPR, complex of universal CPR and tagged targeting peptide, SAPR or ePAR is a platelet activation domain optionally a degranulation triggering domain optionally an ITAM containing domain, and wherein the platelet modulation domain of a second CPR, universal CPR, complex of universal CPR and tagged targeting peptide, SAPR or ePAR is a platelet activation domain optionally a degranulation triggering domain optionally an ITAM containing domain;
dd) express at least two CPRs, universal CPRs, complexes of universal CPRs and tagged targeting peptides, SAPRs or ePARs that operate together to form a logic circuit;
ee) express one or more cargo, optionally wherein the cargo is selected from the group comprising:
   a) a protein or peptide—optionally wherein the protein or peptide is:
      i) an antibody or antigen binding fragment thereof, for example an antibody or antigen binding fragment thereof binds to a tumor antigen or a neoantigen;
      ii) an enzyme, such as a nuclease for example a TALEN;
      iii) a cytokine for example IL-10; or
      iv) a CRISPR associated protein, for example Cas9;
      v) a bispecific protein, for example a bispecific antibody or optionally a T-cell engager (BiTE);
      vi) a fusion protein comprising an exosome targeting domain, optionally wherein the fusion protein comprises:
         a) the cargo protein or peptide; and
         b) an exosome targeting domain, optionally wherein the exosome targeting domain is selected from the group comprising or consisting of:
         i) an exosome specific membrane protein or exosome membrane targeting portion thereof, for example:
         a tetraspanin, for example CD63; or a non-tetraspanin such as PTGFRN or BASP1
         ii) an exosome targeting sequence from a soluble protein, optionally the WW domain of Nedd4 ubiquitin ligases;
         iii) a ubiquitin tag; and/or
         iv) a tag binding domain, optionally a nanobody directed against a tag, optionally a nanobody directed against GFP.
   b) a nucleic acid, optionally wherein the nucleic acid is:
      i) an RNA, for example selected from mRNA, a miRNA, shRNA, and a clustered regularly interspaced short palindromic repeats (CRISPR) sequence; and/or
      ii) an RNA that comprises an exosome targeting domain, optionally wherein the exosome targeting domain is selected from the group comprising or consisting of:
         a) an exosome targeting hairpin or linear motif;
         b) a viral exosome targeting RNA or exosome targeting fragment thereof;
   iii) an RNA that comprises an aptamer domain, optionally wherein the aptamer domain is selected from:
      a) a MS2 binding stem-loop;
      b) a C/D box; and/or
      c) an AU rich element, optionally wherein the RNA is an mRNA that encodes Cas9;
ff) express a fusion protein wherein the fusion protein comprises:
   i) the bacteriophage coat protein MS2 fused to an exosome membrane protein, optionally wherein the exosome membrane protein is selected from the group comprising or consisting of Lamp2b, VSVG, CD63; and/or
   ii) the archaeal ribosomal protein L7Ae fused to an exosome membrane protein, optionally wherein the exosome membrane protein is selected from the group comprising or consisting of Lamp2b, VSVG, CD63; and/or
   iii) a CD9-HuR fusion protein;
optionally wherein the fusion protein further comprises a light activated dimerization protein;
gg) translate one or more cargo from an mRNA only upon binding of one or more CPRs, universal CPRs, complexes of universal CPRs and tagged targeting peptides, SAPRs or ePARs to the target, optionally wherein the cargo is selected from the group comprising:
   a) a protein or peptide, optionally
      i) an antibody or antigen binding fragment thereof, for example an antibody or antigen binding fragment thereof binds to a tumor antigen or a neoantigen;
      ii) an enzyme, such as a nuclease for example a TALEN;
      iii) a cytokine for example IL-10; or
      iv) a CRISPR associated protein, for example Cas9;
      v) a bispecific protein, for example a bispecific antibody or optionally a T-cell engager (BITE)
   b) a nucleic acid—in some embodiments the nucleic acid is:
      i) an RNA, for example selected from mRNA, a miRNA, shRNA, and a clustered regularly interspaced short palindromic repeats (CRISPR) sequence,
      optionally wherein the cargo is expressed from the Bcl-3 mRNA untranslated regions, optionally 5′UTR.

Accordingly in some embodiments the invention also provides a nucleic acid encoding a cargo protein or peptide or cargo RNA. In some embodiments the protein or peptide is selected from an antibody or antigen binding fragment thereof, an enzyme (such as a nuclease for example a TALEN), a cytokine, or a CRISPR associated protein 9 (Cas9.) In some embodiments the cargo RNA is selected from mRNA, a miRNA, shRNA, and a clustered regularly interspaced short palindromic repeats (CRISPR) sequence). In preferred embodiments the nucleic acid. comprises sequences suitable for driving expression in a megakaryocyte and/or platelet. For example, in some embodiments the nucleic acid encoding the cargo protein, cargo peptide or cargo RNA is operatively linked to a heterologous expression control sequence such as a promoter. In some embodiments the nucleic acid encodes a cargo protein or peptide or cargo RNA and also comprises a megakaryocyte specific promoter or a platelet specific promoter. In some embodiments the nucleic acid encodes a cargo protein or peptide or cargo RNA and comprises a heterologous sequence, such as a megakaryocyte specific promoter or a platelet specific promoter. In some embodiments the nucleic acid is DNA that encodes a cargo protein or peptide or cargo RNA.

The invention provides a method of delivering a cargo comprising administering an effective amount of any one or more of an engineered megakaryocyte, engineered platelet, and/or CPR according to any of the preceding claims.

The invention provides a targeted delivery system comprising a progenitor, producer or effector-chassis of the invention that expresses one or more CPRs, universal CPRs, complexes of universal CPRs and tagged targeting peptides, SAPRs or ePARs of the invention. In some embodiments the targeted delivery system is a therapeutic targeted delivery system. In some embodiments the targeted-delivery system is a non-therapeutic delivery system. In preferred embodiments the system comprises an effector-chassis.

The invention also provides a non-thrombogenic targeted delivery system that comprises a producer or effector-chassis of the invention that expresses one or more CPRs, universal CPRs, complexes of universal CPRs and tagged targeting peptides, SAPRs or ePARs according the invention and wherein the progenitor, producer or effector-chassis has been engineered to disrupt the thrombogenic pathway targeted delivery system. In some embodiments the non-thrombogenic targeted delivery system is a non-thrombogenic therapeutic targeted delivery system. In some embodiments the non-thrombogenic targeted delivery system is a non-thrombogenic non-therapeutic delivery system. In preferred embodiments the system comprises an effector-chassis.

In preferred embodiments, the targeted delivery system or the non-thrombogenic targeted delivery system further comprises one or more cargo. Preferences for the cargo are as described herein.

It is clear that the various components, progenitor, producer or effector-chassis, CPR, universal CPR, complex of universal CPR and tagged targeting peptide, SAPR or ePAR and systems described herein can be used to treat or prevent a range of diseases through the delivery of particular cargo to target sites; and/or through the activation of T cells towards a particular antigen. Accordingly, various embodiments of the invention described herein provide a method of treating a disease, disorder, or condition in a subject, the method comprising: administering to the subject one or more progenitor, producer or effector-chassis of the invention, one or more CPR, universal CPR, complex of universal CPR and tagged targeting peptide, SAPR or ePAR of the invention, preferably administration of the previously described therapeutic delivery system.

The skilled person is able to design the CPR, universal CPR, complex of universal CPR and tagged targeting peptide, SAPR or ePAR of the invention so as to be directed towards an appropriate target for a given disease.

Any of the progenitor, producer or effector-chassis as described herein, for example an engineered progenitor, producer or effector-chassis expressing one of more CPRs, universal CPRs, complexes of universal CPRs and tagged targeting peptides, SAPRs or ePARs of the invention can be used to deliver cargo to a particular cell or tissue, for example to a tumour. Where the progenitor, producer or effector-chassis has been loaded with a therapeutic cargo, binding of the CPR, universal CPR, complex of universal CPR and tagged targeting peptide, SAPR or ePAR to the target results in localised delivery of the therapeutic cargo. For example, targeting a progenitor, producer or effector-chassis that has been loaded with a toxin, for example a toxin stored in the alpha-granule, to a tumour using a CPR results in degranulation and local delivery of the toxin to the tumour.

In addition, or alternatively, rather than delivering a particular cargo, the progenitor, producer or effector-chassis may comprise a CPR, universal CPR, complex of universal CPR and tagged targeting peptide, SAPR or ePAR with the appropriate target binding domain so as to bind to antigen specific T cells to upregulate their function to clear tumours expressing defined antigens. Conversely, antigen specific T Cells that mediate autoimmune diseases could be targeted for destruction, with defined antigens known in a variety of common diseases including Hashimoto's thyroiditis, type 1 diabetes and multiple sclerosis.

As described above, where the progenitor, producer or effector-chassis for example platelet or platelet-like membrane-bound cell fragment retains thrombogenic potential it can be targeted to a tumour via the CPR, starving the tumour of oxygen.

The progenitor, producer or effector-chassis described herein may be engineered to kill cancerous cells. For example, CD19 targeted TRAIL expressing platelets that treat cancerous B cell leukemias (BCL). CD19 targeted CAR-T cells have shown great promise in the clinic versus BCL. TNF Superfamily Member (TRAIL) and Fas ligand (FASL) have been shown to induce BCL death via apoptosis upon CD40 stimulation (See, Dicker et al. “Fas-ligand (CD178) and TRAIL synergistically induce apoptosis of CD40-activated chronic lymphocytic leukemia B cells”. Blood, 2005, which is hereby incorporated by reference in its entirety). CD40L is naturally exposed on activated platelets (see, Henn et al. “CD40 ligand on activated platelets triggers an inflammatory reaction of endothelial cells”. Nature, 1998, which is hereby incorporated by reference in its entirety) and could thus activate FASL/TRAIL dependent cell death pathways when bound to BCL. FASL is naturally exposed on activated platelets (See, Schleicher et al. “Platelets induce apoptosis via membrane-bound FasL”. Blood, 2015, which is hereby incorporated by reference in its entirety). TRAIL expressing platelets have been used to decrease prostate cancer metastasis in mice (See, Li et al. “Genetic engineering of platelets to neutralize circulating tumor cells”. Journal of Controlled Release, 2016, which is hereby incorporated by reference in its entirety). In one embodiment, a resting platelet presenting a CD19-single-chain variable fragment(scFv)-ITAM and containing TRAIL, CD40L, and FASL ligands is activated by binding of the CD19-scFv-ITAM with CD19 on a B cell. Activation results in the presentation of TRAIL, CD40L, and FASL on the platelet surface. Platelet-induced death of leukemia cells is mediated by binding of CD40L to the CD40 receptor of the B cell to activate the FASL/TRAIL-dependent cell death pathways.

In certain embodiments, the progenitor, producer or effector-chassis may be engineered to direct expansion of neoantigen specific T cells in vivo. Neoantigens are presented in many human tumors and can be computationally identified. Expansion of T cells ex vivo and reinfusion results in targeted tumor killing. Immune checkpoint inhibition allows for T cells to kill tumors expressing neoantigens (however non-specificity results in severe side effects). Megakaryocytes can be loaded with MHC class 1 molecules with exogenous peptides and transfer these to platelets. Neoantigens may be expressed in megakaryocytes, and an MHC class 1-ITAM fusion protein is able to stimulate checkpoint inhibitors. This would allow in vivo expansion of neoantigen specific T cells. For example, a platelet may be engineered to express MHC1-Neoantigen-ITAM. Both the engineered platelets and the T cell are activated by interaction of the MHC1-Neoantigen-ITAM with a neoantigen specific T cell receptor (TCR). Activation results in presentation of cytotoxic T-lymphocyte associated protein 4 (CTLA4) and programmed cell death 1 (PD-1) on the surface of the platelet and interaction with CTLA4 inhibitor (CTLA4i) and PD-1 inhibitor (PD-li), respectively, on the T cell. Maximum T cell activation and expansion is reached by checkpoint blockade.

Accordingly in one embodiment the invention provides a progenitor, producer or effector-chassis as described herein, or a therapeutic delivery system or a therapeutic targeted delivery system or a non-thrombogenic therapeutic delivery system for use in the treatment or prevention of disease.

A range of diseases may be treated or prevented using the components described herein, and the skilled person is aware that the target binding domain of the one or more CPRs, universal CPRs, complexes of universal CPRs and tagged targeting peptides, SAPRs or ePARs need to be designed to bind to an appropriate target depending on the disease to be treated/prevented. In addition, the cargo that is loaded in to the progenitor, producer or effector-chassis depends on the disease that is to be treated or prevented.

In some embodiments, the progenitor, producer or effector-chassis as described herein, or a therapeutic delivery system or a therapeutic targeted delivery system or a non-thrombogenic therapeutic delivery system can be used to vaccinate against a particular disease.

In some embodiments, the disease, disorder, or condition may be, but is not limited to, a cancer, an autoimmunity disease or disorder, genetic disease, cardiovascular disease and an infection, for example a bacterial or viral infection, for example an infections with SARS-COV-2.

In some embodiments, the cancer is selected from any of the cancers described in paragraph [0019] on page 9-11 of PCT/GB2020/053247 which is hereby incorporated by reference.

In some embodiments, the engineered platelets described herein may be used to treat autoimmunity conditions.

In some embodiments, the autoimmunity disease or disorder is selected from any of the autoimmunity diseases or disorders described in paragraph [0020] on page 11-12 of PCT/GB2020/053247 which is hereby incorporated by reference.

In some embodiments, the progenitor, producer or effector-chassis described herein may be used to suppress autoantigen specific T cells to treat autoimmune disease. In some embodiments, CPRs, universal CPRs, complexes of universal CPRs and tagged targeting peptides, SAPRs or ePARs in the progenitor, producer or effector-chassis (for example engineered platelet) may include a region specific to a tissue associate with the autoantigen. For example, the tissue is selected from the group consisting of: adipose tissue, adrenal gland, ascites, bladder, blood, bone, bone marrow, brain, cervix, connective tissue, ear, embryonic tissue, esophagus, eye, heart, intestine, kidney, larynx, liver, lung, lymph, lymph node, mammary gland, mouth, muscle, nerve, ovary, pancreas, parathyroid, pharynx, pituitary gland, placenta, prostate, salivary gland, skin, stomach, testis, thymus, thyroid, tonsil, trachea, umbilical cord, uterus, vascular, and spleen.

Table 12 on page 129-130 of PCT/GB2020/053247 which is hereby incorporated by reference shows the molecular target and/or tissue target for a non-exhaustive list of neurological system autoimmunity disorders from Hayter, et al; and Table 13 on page 130 of PCT/GB2020/053247 which is hereby incorporated by reference shows the molecular target and/or tissue target for a non-exhaustive list of endocrine system autoimmunity disorders from Hayter, et al.; Table 14 on page 131 of PCT/GB2020/053247 which is hereby incorporated by reference shows the molecular target and/or tissue target for a non-exhaustive list of gastrointestinal system autoimmunity disorders from Hayter, et al; Table 15 on page 131 of PCT/GB2020/053247 which is hereby incorporated by reference shows the molecular target and/or tissue target for a non-exhaustive list of hematopoietic autoimmunity disorders from Hayter, et al.; Table 16 on page 132 of PCT/GB2020/053247 which is hereby incorporated by reference shows the molecular target and/or tissue target for a non-exhaustive list of musculoskeletal system autoimmunity disorders from Hayter, et al.; Table 17 on page 132-133 of PCT/GB2020/053247 which is hereby incorporated by reference shows the molecular target and/or tissue target for a non-exhaustive list of cutaneous and mucous autoimmunity disorders from Hayter, et al.; Table 18 on page 133 of PCT/GB2020/053247 which is hereby incorporated by reference shows the molecular target and/or tissue target for a non-exhaustive list of cutaneous autoimmunity disorders from Hayter, et al.; Table 19 on page 133 of PCT/GB2020/053247 which is hereby incorporated by reference shows the molecular target and/or tissue target for a non-exhaustive list of cardiovascular autoimmunity disorders from Hayter, et al.; Table 20 on page 134 of PCT/GB2020/053247 which is hereby incorporated by reference shows the molecular target and/or tissue target for a non-exhaustive list of other autoimmunity disorders from Hayter, et al.

Various embodiments of the invention described herein provide a method of reducing activity in the immune system of a subject, the method comprising: administering to the subject a platelet or engineered platelet that expresses at least one SAPR, wherein the target binding domain of the SAPR comprises a major histocompatibility complex (MHC) molecule bound to a peptide derived from a tumor antigen, a neoantigen, or an autoantigen. In some embodiments the engineered platelet comprises an anti-inflammatory cytokine, for example IL-10. The skilled person is aware of other suitable anti-inflammatory cytokines.

In some embodiments, the SAPR expresses a MHC class I molecule. In some embodiments, the SAPR expresses a MHC class II molecule. In some embodiments, the MHC molecule stimulates an immune response to an antigen. In some embodiments, the antigen is associated with at least one disease, disorder, or condition selected from the group consisting of: a cancer, an autoimmunity, genetic disease, cardiovascular disease and an infection.

Various embodiments of the invention described herein provide a method of in vivo gene editing or gene therapy in a subject, the method comprising: administering to the subject an engineered platelet comprising a chimeric platelet receptor described herein specific to a tissue to be edited, wherein the engineered platelet is cloaking a viral particle such as an adenovirus or Sendai virus loaded with genome engineering machinery; and releasing the genome machinery at the tissue. In some embodiments, the genome machinery is a CRISPR/Cas gene editing system.

Various embodiments of the invention described herein provide a use of the therapeutic delivery system previously described, wherein the chimeric receptor is specific to an antigen associated with the disease, disorder, or condition in treating a disease, disorder, or condition in a subject. In some embodiments of the use described herein, the disease, disorder, or condition is selected from the group consisting of: a cancer, an autoimmunity, genetic disease, cardiovascular disease and an infection.

In some embodiments of the use described herein, the cancer may be, but is not limited to any of the cancers described in paragraph [0030] on page 14-16 of PCT/GB2020/053247 which is hereby incorporated by reference.

In some embodiments of the use described herein, the disease, disorder, or condition is an autoimmunity such as, but not limited to, any of the autoimmunity diseases, disorders or conditions described in paragraph [0031] on page 16 of PCT/GB2020/053247 which is hereby incorporated by reference.
Various embodiments of the invention herein provide a therapeutic delivery system comprising: (a) an engineered platelet presenting the chimeric platelet receptor, wherein the engineered platelet has been produced through genetic modification of a progenitor megakaryocyte to be non-thrombogenic and non-immunogenic, and optionally has been engineered to have a reduced pro-inflammatory effect; and (b) at least one therapeutic agent selected from the group consisting of: a cargo as defined herein, a toxin, a protein, a small molecule drug, imaging agent, radionucleotide drugs, radionucleotide tagged antibodies, or conjugate any thereof; and a nucleic acid packaged within a vesicle inside the platelet, i) wherein the therapeutic agent is the nucleic acid or the protein, loading occurs through expression in a progenitor megakaryocyte, or ii) wherein the therapeutic agent is loaded by incubation of the engineered platelet with the therapeutic agent.

As described above, it is possible to produce platelets directly from iPSCs. In some embodiments, the platelets or platelet-like membrane-bound cell fragments, or engineered platelets or platelet-like membrane-bound cell fragments described herein may be produced using the technique described in Ito et al. (Cell, 174(3): 636-648.e18, 2018, which is hereby incorporated by reference in its entirety). Ito provides a method of clinical scale production of platelets from iPSC progenitors. Turbulence was observed to activate platelet biogenesis for clinical scale ex vivo production of platelets from human-induced pluripotent stem cells (iPSCs) (Ibid.). iPSCs derived from immortalized megakaryocyte progenitor cell lines (imMKCLs) were combined with soluble factors insulin Like Growth Factor Binding Protein 2 (IGFBP2), macrophage migration inhibitory factor (MIF), and nardilysin convertase (NRDC) in a bioreactor with control over the physical parameters of turbulent energy and shear stress (Ibid.). Production of greater than 10¹¹ μlatelets were observed (Ibid.). Platelets were observed to function analogously to those derived from donors (Ibid.).

In certain embodiments of the invention herein, the imMKCL may be established by introducing cancer-derived MYC (c-MYC)/polycomb ring finger proto-oncogene (BMI-1) and BCL2 I like 1 (BCL-XL) genes into the an iPSC of the invention using a lentivirus (for example wherein the iPSC may comprise one or more of the engineering modifications described here). Additional genes may be introduced or deleted resulting in an edited megakaryocyte, in fact even platelet specific promoters have been previously characterized. These genes provide inducible gene expression in the presence of an agent, such as doxorubicin (DOX). The imMKCL may be cyropreserved until cultivation is desired. Megakaryocyte expansion is stimulated by contacting the cell line with the agent resulting expression of the inserted genes. The agent is removed to halt gene expression and allow platelet production.

Current Federal Drug Administration (FDA)-approved rules for storage of platelets for transfusion require storage at 22° C. and must be used within 6 days. Slichter et al. “Treatment of Bleeding in Severely Thrombocytopenic Patients with Transfusion of Dimethyl Sulfoxide (DMSO) Cryopreserved Platelets (CPP) Is Safe—Report of a Phase 1 Dose Escalation Safety Trial”. Blood, 2016, which is hereby incorporated by reference in its entirety, hypothesizes cryopreservation is possible for two years when frozen with DMSO. After a positive phase 1 trial, phase 2 and 3 trials are underway. Infusion of up to three sequential units of cryopreserved platelets (CPP) in patients with severe thrombocytopenia and active bleeding appeared to be “safe and without any evidence of thrombotic complications despite CPP having a procoagulant phenotype resulting from the cryopreservation process.” Therefore, cryopreserved platelets likely have efficacy for stabilizing, reducing, or stopping bleeding in thrombocytopenic patients as measured using the World Health Organization (WHO) bleeding grades. No evidence was found to undermine the hypothesis that cryopreserved platelets used for non-clotting purposes would be as effective as platelets stored according to the present FDA rules.

Various embodiments of the invention described herein provide a method of in vitro production of platelets or platelet-like membrane-bound cell fragments, the method comprising: transfecting a plurality immortal progenitor cells, for example induced pluripotent stem cell (iPSC) progenitors with an expression system, wherein the expression system is induced by an agent not found in an iPSC; establishing a megakaryocyte progenitor cell line by contacting the expression system with the agent to expand megakaryocytes; and engineering the megakaryocyte to have at least one of the following:
insertion of a nucleic sequence encoding a chimeric platelet receptor previously described;
insertion of a nucleic acid sequence encoding a toxin;
insertion of a nucleic acid encoding a cargo, for example a cargo that is a protein or peptide, or an RNA for example an mRNA;
insertion of a nucleic acid encoding a therapeutic agent or imaging agent, for example a therapeutic agent of imaging agent that is a protein or peptide, or an RNA for example an mRNA for example a therapeutic agent or imaging agent;
deletion of or mutation in a nucleic acid sequence encoding a platelet receptor, mediator, and/or signal transduction protein; and/or
deletion of or mutation in a nucleic acid sequence that results in the platelet being less immunogenic than a platelet without the deletion or mutation;

and removing the agent from the expression system to induce differentiation of the megakaryocytes into platelets.

In some embodiments, the iPSC already comprises an expression system that has been introduced into the iPSC and which is induced by an agent not found in the iPSC. Accordingly, in some embodiments the invention provides a method for the in vitro production of platelets (or Synlets as described herein) wherein the method comprises establishing a megakaryocyte progenitor cell line from an iPSC that comprises an expression system that had been introduced into the iPSC (i.e. is a non-native expression system) wherein the expression system is induced by an agent not found in the iPSC, wherein said establishing comprises contacting the expression system with the said agent to expand megakaryocytes; engineering the megakaryocyte to have at least one of the following;
insertion of a nucleic sequence encoding a chimeric platelet receptor previously described;
insertion of a nucleic acid sequence encoding a toxin;
insertion of a nucleic acid encoding a cargo, for example a cargo that is a protein or peptide, or an RNA for example an mRNA;
insertion of a nucleic acid encoding a therapeutic agent or imaging agent, for example a therapeutic agent of imaging agent that is a protein or peptide, or an RNA for example an mRNA for example a therapeutic agent or imaging agent;
deletion of or mutation in a nucleic acid sequence encoding a platelet receptor, mediator, and/or signal transduction protein; and/or
deletion of or mutation in a nucleic acid sequence that results in the platelet being less immunogenic than a platelet without the deletion or mutation;
and removing the agent from the expression system to induce differentiation of the megakaryocytes into platelets.

In some embodiments, the method comprises incubating the megakaryocyte progenitor cell line with an exogenous cargo to be loaded into the megakaryocyte progenitor cell line. The exogenous cargo may be any exogenous cargo where it is considered to be beneficial to load the cargo into the megakaryocyte progenitor cell line, for example a protein or peptide; a nucleic acid such as an RNA or an mRNA, or a vector such as a DNA vector; a viral vector; a small molecule; a therapeutic agent and/or an imaging agent, or an exosome, for example an exosome pre-loaded with a second cargo; or a nanoparticle or nanoparticles. Preferences for the cargo, and for methods of loading the cargo, are described elsewhere herein.

In some embodiments, the method comprises incubating the platelets produced from the megakaryocyte progenitor cell line with an exogenous cargo to be loaded into the platelets. The exogenous cargo may be any exogenous cargo where it is considered to be beneficial to load the cargo into the platelets, for example a protein or peptide; a nucleic acid such as an RNA or an mRNA, or a vector such as a DNA vector; a viral vector; a small molecule; a therapeutic agent and/or an imaging agent and/or an exosome, for example an exosome pre-loaded with a second cargo; or a nanoparticle or nanoparticles. Preferences for the cargo, and for methods of loading the cargo, are described elsewhere herein.

Gene symbols are used herein, along with ENSEMBL Gene IDs, to refer to genes from humans. Unless otherwise noted, the gene name and ENSEMBL Gene (ENSG) IDs corresponding to each gene symbol are shown in Table 1 on pages 19-23 of PCT/GB2020/053247 which is hereby incorporated by reference. The unique identifiers for each ENSEMBL entry in this table has been modified to remove the first five leading zeros (0) of the identifier after the ENSG label.

Symbols and names are used herein, along with ENSEMBL protein IDs, to refer to proteins from humans. Unless otherwise noted, the protein name (if used to refer to the protein herein) and symbol and ENSEMBL protein (ENSP) IDs corresponding to each symbol are shown in Table 2 on pages 23-31 of PCT/GB2020/053247 which is hereby incorporated by reference. The unique identifiers for each ENSEMBL entry in this table has been modified to remove the first five leading zeros (0) of the identifier after the ENSP label.

CD3 or CD3 is also known as Cluster of differentiation 2 (multiple subunits). FCER2 or CD23 is also known as (IgE receptor. NT5E is also known as 5′-nucleotidase. F9, F10 is also known as activated F9, F10. ACVRL1 is also known as activin receptor-like kinase 1. AFP is also known as alpha-fetoprotein. ANGPTL3 is also known as angiopoietin 3. BSG or CD147 is also known as basigin. APP or N/a is also known as beta-amyloid. CALCA is also known as calcitonin gene-related peptide. CA9 is also known as carbonic anhydrase 9 (CA-IX). MYH7 is also known as cardiac myosin. MET is also known as c-Met. F3 is also known as coagulation factor III. CLEC6A is also known as dendritic cell-associated lectin 2. EGFR or EGFR is also known as elongating growth factor receptor. ENG is also known as endoglin. EPHA3 is also known as ephrin receptor A3. FGB or is also known as fibrin II, beta chain. FN1 is also known as fibronectin extra domain-B. FOLH1 is also known as folate hydrolase. FOLR2 is also known as folate receptor 2. FOLR1 is also known as folate receptor alpha. FZD1 is also known as Frizzled receptor. B4GALNT1 is also known as GD2 ganglioside. ST8SIA1 is also known as GD3 ganglioside. MMP9 is also known as gelatinase B. TYRP1 or TYRP1 is also known as glycoprotein 75. GPC3 is also known as glypican 3. CSF2RA is also known as GMCSF receptor α-chain. IGF1R or CD221 is also known as IGF-1 receptor. IL31RA is also known as IL31RA. ITGA2B or CD41 is also known as integrin alpha-IIb. ITGA5 is also known as integrin α5. ITGB3 is also known as integrin αIIbβ3. ITGB7 is also known as integrin β7. IFNG is also known as interferon gamma. IFNAR1, IFNAR2 is also known as interferon α/β receptor. CXCL10 is also known as interferon gamma-induced protein. IL12A or IL-12 is also known as interleukin 12. IL13 or IL-13 is also known as interleukin 13. IL17A or IL17A is also known as interleukin 17 alpha. IL17F or IL17F is also known as interleukin 17 F. IL2 or IL2 is also known as interleukin 2. IL22 or IL-22 is also known as interleukin 22. IL23A or IL23 is also known as interleukin 23. IL6 or IL6 is also known as interleukin 6. SELL or CD62L is also known as L-selectin. MSLN is also known as mesothelin. MUC1 is also known as mucin CanAg. MADCAM1 is also known as mucosal addressin cell adhesion molecule. MAG is also known as myelin-associated glycoprotein. NECTIN4 is also known as nectin-4. CASP2 is also known as neural apoptosis-regulated proteinase 2. PTDSS1 is also known as phosphatidylserine. PDGFRB is also known as platelet-derived growth factor receptor beta. RHD, RHCE is also known as Rhesus factor. RSPO3 is also known as root plate-specific spondin 3. SELP is also known as selectin P. SAA1 or SAA2 is also known as serum amyloid A protein. APCS is also known as serum amyloid P component. S1PR1 is also known as sphingosine-1-phosphate. MAPT is also known as tau protein. TNC is also known as tenascin C. TNFRSF12A is also known as TWEAK receptor. VIM is also known as vimentin. VWF is also known as von Willebrand factor. IL2RA or CD25 is also known as a chain of IL-2receptor.

It is clear to the skilled person that the invention provides various compositions comprising any one or more of the progenitor, producer or effector-chassis, CPRs, universal CPRs, complexes of universal CPRs and tagged targeting peptides, SAPRs or ePARs, or delivery systems as described herein.

The present teachings further comprise pharmaceutical compositions comprising one or more of the progenitor, producer or effector-chassis of the invention, and optionally at least one pharmaceutically acceptable excipient or inert ingredient. Further, a pharmaceutical may comprise the therapeutic delivery system described herein.

Preferences for the pharmaceutical compositions and dosage are as set out in paragraphs [0197]-[237] of PCT/GB2020/053247 which is hereby incorporated by reference.

At various places in the present specification, features or functions of the compositions of the present disclosure are disclosed in groups or in ranges. It is specifically intended that the present disclosure include each and every individual sub combination of the members of such groups and ranges. The following is a non-limiting list of term definitions.

As used herein, the term “antigen” is defined as a molecule that provokes an immune response when it is introduced into a subject or produced by a subject such as tumor antigens which arise by the cancer development itself. This immune response may involve either antibody production, or the activation of specific immunologically-competent cells such as cytotoxic T lymphocytes and T helper cells, or both.

As used herein, the term “approximately” or “about,” as applied to one or more values of interest, refers to a value that is similar to a stated reference value. In certain embodiments, the term “approximately” or “about” refers to a range of values that fall within 25, 20, 19, 18, 17, 16, 15, 14, 13, 12, 11, 10, 9, 8, 7, 6, 5, 4, 3, 2, 1, or less in either direction (greater than or less than) of the stated reference value unless otherwise stated or otherwise evident from the context (except where such number would exceed 100 of a possible value).

As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

As used herein, the term “cancer” refers a broad group of various diseases characterized by the uncontrolled growth of abnormal cells in the body. Unregulated cell division and growth results in the formation of malignant tumors that invade neighboring tissues ultimately metastasize to distant parts of the body through the lymphatic system or bloodstream.

As used herein, the term “cytokines” refers to a family of small soluble factors with pleiotropic functions that are produced by many cell types that can influence and regulate the function of the immune system.

As used herein, the term “delivery” refers to the act or manner of delivering a compound, substance, entity, moiety, cargo or payload. A “delivery agent” refers to any agent which facilitates, at least in part, the in vivo delivery of one or more substances (including, but not limited to a compound and/or compositions of the present invention) to a cell, subject or other biological system cells.

As used herein, embodiments of the invention described herein are “engineered” when they are designed to have a feature or property, whether structural or chemical, that varies from a starting point, wild type or native molecule.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

As used herein, a “formulation” includes at least a compound and/or composition of the present invention and a delivery agent.

As used herein, a “fragment,” as used herein, refers to a portion. For example, fragments of proteins may comprise polypeptides obtained by digesting full-length protein. In some embodiments, a fragment of a protein includes at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250 or more amino acids. In some embodiments, fragments of an antibody include portions of an antibody.

As used herein, the term “an immune cell” refers to any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Exemplary immune system cells include a CD4+ T cell, a CD8+ T cell, a CD4− CD8− double negative T cell, a T γδ cell, a Tαβ cell, a regulatory T cell, a natural killer cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, a “linker” or “targeting domain” refers to a portion of a chimeric platelet receptor that recognizes and binds a desired antigen.

As used herein, a “checkpoint factor” is any moiety or molecule whose function acts at the junction of a process. For example, a checkpoint protein, ligand or receptor may function to stall or accelerate the cell cycle.

As used herein, the term “messenger RNA” (mRNA) refers to any polynucleotide which encodes a polypeptide of interest and which is capable of being translated to produce the encoded polypeptide of interest in vitro, in vivo, in situ, or ex vivo.

As used herein, the term “mutation” refers to a change and/or alteration. In some embodiments, mutations may be changes and/or alterations to proteins (including peptides and polypeptides) and/or nucleic acids (including polynucleic acids). In some embodiments, mutations comprise changes and/or alterations to a protein and/or nucleic acid sequence. Such changes and/or alterations may comprise the addition, substitution and or deletion of one or more amino acids (in the case of proteins and/or peptides) and/or nucleotides (in the case of nucleic acids and or polynucleic acids e.g., polynucleotides). In some embodiments, wherein mutations comprise the addition and/or substitution of amino acids and/or nucleotides, such additions and/or substitutions may comprise 1 or more amino acid and/or nucleotide residues and may include modified amino acids and/or nucleotides. The resulting construct, molecule or sequence of a mutation, change or alteration may be referred to herein as a mutant.

As used herein, the term “neoantigen”, as used herein, refers to a tumor antigen that is present in tumor cells but not normal cells and do not induce deletion of their cognate antigen specific T cells in thymus (i.e., central tolerance). These tumor neoantigens may provide a “foreign” signal, similar to pathogens, to induce an effective immune response needed for cancer immunotherapy.

A neoantigen may be restricted to a specific tumor. A neoantigen be a peptide/protein with a missense mutation (missense neoantigen), or a new peptide with long, completely novel stretches of amino acids from novel open reading frames (neoORFs). The neoORFs can be generated in some tumors by out-of-frame insertions or deletions (due to defects in DNA mismatch repair causing microsatellite instability), gene-fusion, read-through mutations in stop codons, or translation of improperly spliced RNA (e.g., Saeterdal et al., Proc Natl Acad Sci USA, 2001, 98: 13255-13260, which is hereby incorporated by reference in its entirety).

As used herein, the term “pharmaceutically acceptable excipient,” as used herein, refers to any ingredient other than active agents (e.g., as described herein) present in pharmaceutical compositions and having the properties of being substantially nontoxic and non-inflammatory in subjects. In some embodiments, pharmaceutically acceptable excipients are vehicles capable of suspending and/or dissolving active agents. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Pharmaceutically acceptable salts include the conventional non-toxic salts, for example, from non-toxic inorganic or organic acids.

In some embodiments, a pharmaceutically acceptable salt is prepared from a parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

As used herein, the term “T cell” refers to an immune cell that produces T cell receptors (TCRs). As used herein, the term “T cell receptor” (TCR) refers to an immunoglobulin superfamily member having a variable antigen binding domain, a constant domain, a transmembrane region, and a short cytoplasmic tail, which is capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). The extracellular portion of TCR chains (e.g., α-chain, β-chain) contains two immunoglobulin domains, a variable domain (e.g., α-chain variable domain or Vα, β-chain variable domain or Vβ) at the N-terminus, and one constant domain (e.g., α-chain constant domain or Ca and β-chain constant domain or Cβ,) adjacent to the cell membrane. Similar to immunoglobulin, the variable domains contain complementary determining regions (CDRs) separated by framework regions (FRs).

As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art appreciate that in some embodiments, a unit dosage form may be considered to comprise a therapeutically effective amount of a particular agent or entity if it comprises an amount that is effective when administered as part of such a dosage regimen.

As used herein, the terms “treatment” or “treating” denote an approach for obtaining a beneficial or desired result including and preferably a beneficial or desired clinical result. Such beneficial or desired clinical results include, but are not limited to, one or more of the following: reducing the proliferation of (or destroying) cancerous cells or other diseased, reducing metastasis of cancerous cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of individuals.

As used herein, the term “therapeutic agent” refers to a biological, pharmaceutical, or chemical compound. Non-limiting examples include simple or complex organic or inorganic molecule, a peptide, a protein, an oligonucleotide, an antibody, an antibody derivative, antibody fragment, a receptor, and a soluble factor.

### EQUIVALENTS AND SCOPE

Those skilled in the art recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

Described herein are compositions and methods for the design, production, administration, and/or formulation of engineered platelets described herein. In some embodiments, the engineered platelets may carry cargo in the vesicles for delivery on activation by a target, which does not activate wild-type platelets. In some embodiments then the engineered platelets of the invention carry cargo in the vesicles for delivery on activation by a target, wherein the target does not activate wild-type platelets. For example the target to which the CPR, universal CPR, complex of universal CPR and tagged targeting peptide, SAPR or ePAR binds is not a target that would typically activate wild-type platelets, but which does activate the engineered platelet through the interaction with the target-binding CPR, universal CPR, complex of universal CPR and tagged targeting peptide, SAPR or ePAR.

The present invention is further illustrated by the following non-limiting examples. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

### FIGURE LEGENDS

FIG. 1—genome editing optimization/guide ID. A) Schematic of CRISPR guide selection and screening procedure. B) Guide KO generation efficiency (as predicted by Synthego ICE algorithm) within a pools of iPSCs. C) Summary and repetition of highest efficiency guide nucleofection. N=2 per result, error bars indicate standard deviation.

FIG. 2—Sequential editing process->7xKO. A) Schematic of sequential knock-out approach. B) Quantification of viable cell number during sequential KO approach. Viable cells identified based on exclusion of PI stain. C) Pooled knock-out efficiencies throughout sequential KO approach. At each Cas9 RNP nucleofection event, half the cells were taken for genomic DNA extraction and amplicons for all previous target sites were amplified and screened for their KO level using Synthego ICE.

FIG. 3—7xKO clone identification. A) Table showing Synthego ICE results for gene KOs within clones produced from a single cell sort of the 7xKO pool. B) Repetition of Synthego ICE analysis on amplicons generated from further expanded clones where results were absent in (A).

FIG. 4—7xKO pool of cells forward programs towards a megakaryocyte like phenotype. A) Flow cytometry based MK differentiation marker panel and viability analysis on 7xKO pool 10 days post forward programming induction using doxycycline. Performed in both unedited and 7xKO pool. B) As in (A), however 13 days post forward programming induction.

FIG. 5—7xKO pool of cells is not activated by standard agonist. A) Microscopy images of unedited MKs and 7xKO pools, stained for P-Selectin at day 13 post doxycycline addition, following fixation. B) As in (A), however after the addition of TRAP6 (10 uM) and CRP (10ug/ml) for 30 minutes, followed by fixation. C) Flow cytometry assay of P-Selectin exposure in MKs stimulated with 300 ng/mL of PMA. Vehicle control or PMA was added to live MKs and histograms shown are of P-Selectin staining 7-10 minutes post agonist/vehicle addition. This assay was performed on a 7xKO clone (not pool) and done on day 15 post doxycycline addition.

FIG. 6—Receptor design and lentiviral transduction. A) CPR receptor design IDs. B) Schematic of CPR expression vectors packaged within in lentivirus. CPRs listed in A and mCherry expressed as a multicistronic transcript through the use of T2A sequence. Expression is driven by the EFla promoter. C) Microscopy images of iPSCs transduced with lentivirus expressing CPR sequences in (A), 2 days post transduction

FIG. 7—Receptor expression on iPSC cell surface. A) CPR receptor design IDs. B) mCherry expression and CPR surface localisation as assayed by CD19-FITC based staining for CPR expression. 10 days post transduction with lentivirus.

FIG. 8—Receptor expressing cells FoP and retain expression. A) Flow cytometry based MK differentiation marker panel and viability analysis on CPR3 expressing cells 10 and 16 days post forward programming induction using doxycycline. B) CPR3 surface expression quantified using FMC63-FITC staining of CPR3 expressing MKs and unstained MKs 10 days post doxycycline addition.

FIG. 9—Receptor expressing MKs activate/degranulate in response to CD19+ve cells. A) Microscopy images of P-Selectin staining on fixed MKs expressing CPR3 or untransduced controls following 30 minutes of incubation with either BJABs (CD19+ve B cells) or Jurkats (CD19 negative T cells). B) Flow cytometry quantification of P-Selectin staining of samples imaged in (A). C) MFI fold change of P-Selectin staining in indicated comparisons. MFI calculated following background subtraction, and performed within CD42 positive MK cell population.

FIG. 10—schematic demonstrating the reduced thrombogenic potential of the platelets of the invention.

FIG. 11—taken from MALIK, N, JENKINS A M, MELLOM J, BAILEY G. Regen.Med.(2019)14(11),983-989

FIG. 12—taken from Ye, Q et al (2020) Cell Proliferation, DOI: 10.1111/cpr.12946. Strategic gene editing of hPSCs to suppress the immune response. (A) Schematic structure of HLA class I and class I molecules. (B) Strategic gene editing of hPSCs to suppress the immune response.

FIG. 13. Pleiotropic effects resulting from the interaction of CD40+ T cells, activated by CD40L+ platelets, or soluble CD40L with immune and non-immune cells. From: The CD40/CD40L costimulatory pathway in inflammatory bowel disease Danese et al 2004 Gut 53: 1035-1043

FIG. 14: TGF-β-mediated escape from NKG2D-mediated tumour immunorecognition by cytotoxic lymphocytes. NKG2D down-regulation on cytotoxic lymphocytes impairs their immunosurveillance of NKG2DL-expressing malignant cells and subsequent tumour elimination. Tumour cells release both soluble TGF-β and TGF-β-containing exosomes locally and systemically acting on NK cells and cytotoxic T lymphocytes (CTL), thereby inducing downregulation of NKG2D. In addition, tumour-derived exosomes may contain NKG2DLs and miRNA with the capacity to down-regulate NKG2D surface expression. TGF-β also acts on tumour cells in an autocrine or paracrine manner thereby reducing NKG2DL expression and further subverting cancer immunosurveillance by the NKG2D-NKG2DL axis. Other major source of TGF-β are platelets as well as regulatory T cells (Tregs) and myeloid derived suppressor cell. From: Impairment of NKG2D-Mediated Tumor Immunity by TGF-β; Front. Immunol., 15 Nov. 2019 https://doi.org/10.3389/fimmu.2019.02689

FIG. 15: The intimate crosstalk between platelets and cancer: TGFb. (A) EMT induction in cancer cells is a key mechanism involved in platelet-mediated metastasis formation and is characterized by reduced levels of typical epithelial markers and increased expression of many mesenchymal markers with prothrombotic properties. This leads to the activation of platelets by cancer cells and the release of TXA2, which binds to the platelet receptor TP, allowing the amplification of the platelet response. PGE2, PDGF, and TGF-β are platelet-derived mediators that mediate the induction of EMT, thus leading to tumour invasion and metastasis formation. (B) Platelets promote metastasis by providing cancer cells with protection from immune surveillance due to the so-called “platelet mimicry” phenomenon, characterized by the transferring of platelet proteins to cancer cells, including the MHC-I. The resulting “phenotype of false pretenses” disrupts recognition of tumour cell missing self, thereby impairing cytotoxicity and IFN-γ production by NK cells. Also, GARP activates latent TGF-β, promoting the suppression of immune response to cancer cells mediated by regulatory T cells. Platelet release of TGF-β impairs interferon-γ production and NK cell cytotoxicity.

FIG. 16: 3xKO Pool frequency determination.

Frequency was determined using the ICE analysis software (Synthego) as described in Example 5.

FIG. 17: 3xKO pool Forward Programming efficiency

A) Flow cytometry based MK differentiation marker panel and viability analysis of 3xKO pool 10 days post forward programming induction using doxycycline. Performed in both unedited and 7xKO pool. Further method details on forward programming and markers can be found in exmaple 6. Performed on (A) 3xKO ITGA2B/HPS1/PAR1 pool, (B) 3xKO ITGA2B/HPS1/P2Y12 pool and (C) wildtype, unedited.

FIG. 18: B2M guide screening

Frequency was determined using the ICE analysis software (Synthego) as described in Example 5.

FIG. 19: GARP/LRCC32 knock-out guide screening

Frequency was determined using the ICE analysis software (Synthego) as described in Example 5.

FIG. 20: CAR expression on surface of PLPs derived from PBMKs. Anti-CD19 targeted with anti-FMC63 antibody

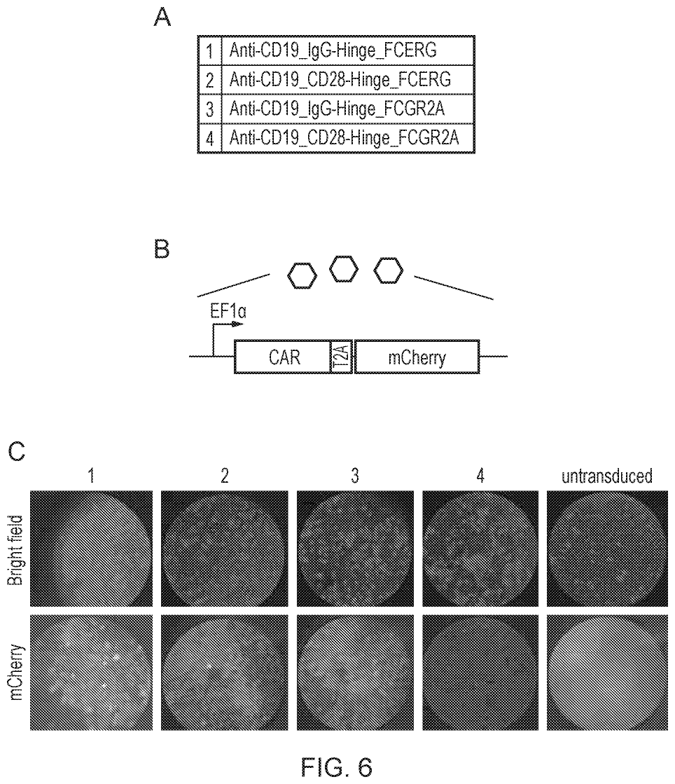

Adult, peripheral blood derived HSCs were driven to differentiate into megakaryocytes using a cocktail of cytokines in Stemspan SFEM2 (Stemspan 100× MK Supplement, Stem Cell Technologies, Catalog Number 02696). At day 7 post differentiation induction, cells were transduced with lentivirus 1 particles (FIG. 6A,B). At day 10, surface expression of the CAR was assayed using an anti-CAR antibody. In addition, PLPs were generated from these now differentiated PBMKs and show to contain both soluble mCherry and surface CAR expression. PLPs were generated from MKs by culture in RPMI high-glucose for a period of 6 hours.

FIG. 21: CAR expression on surface of PLPs derived from iPSC-MKs transduced with lentivirus Schematic of iPSC forward programming and lentivirus addition protocol (at day 10) for FIGS. 22, 24 and 25.

FIG. 22: CAR expression on surface of PLPs derived from iPSC-MKs

Following lentiviral transduction of iPSCs undergoing forward programming towards the megakaryocyte lineage, cells were assayed at day 15 for their expression of surface level CAR using a CAR binding antibody. iPSC-MKs expressed surface level CAR dose-responsively with amount of virus used at day 10 for transduction (as measured by MOI). PLPs derived from these iPSC-MKs exhibited similar MOI dependent dose responsiveness.

FIG. 23: RNA loading in MKs and PLPs. (A) Schematic design of RNA loading strategy. BASP1 is a luminal exosome protein, and so fusion of it to L7Ae a hairpin binding protein should permit loading of RNA into PLP exosomes. (B) FoP iPSC megakaryocytes were nucleofected with plasmid expressing minimal BASP1-mScarlet-L7Ae fusion protein, plated on Fibrinogen three days later (at D16 post differentiation), and imaged following fixation (2% formaldehyde) on Zeiss Cell Discoverer 7 at 50× objected with 0.5× Tube lens. Prior to imaging cells were permeabilized (PBS+0.3% TWEEN20) subsequently stained with Mouse anti-CD62P (AbCam ab255822)+Goat anti Mouse Alexa Fluor Plus 647 (Invitrogen).

FIG. 24: CD34/41 KO iPSC derived MKs exhibit robust CAR expression Following lentiviral transduction of CD34 and CD41 KO iPSCs at day 10 undergoing forward programming towards the megakaryocyte lineage, cells were assayed at day 13 for their expression of surface level CAR using a CAR binding antibody.

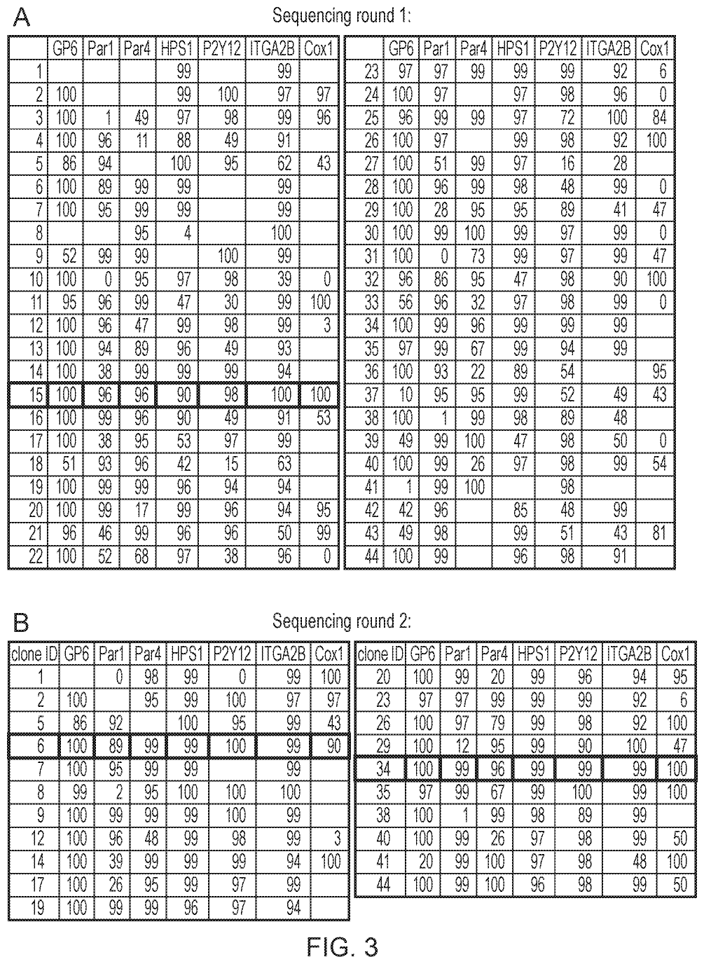

FIG. 25: 7xKO iPSC derived MKs exhibit robust CAR expression Following lentiviral transduction of clonal 7xKO iPSCs at day 10 undergoing forward programming towards the megakaryocyte lineage, cells were assayed at day 13 for their expression of surface level CAR using a CAR binding antibody. This 7xKO iPSC clone corresponds to clone 34 identified in FIG. 3.

### EXAMPLES

### Example 1. Establishing Platelet Production in a Laboratory

iPSC-iMKCL are obtained from the Koji Eto Lab at Megakaryon Corporation (Kyoto office/Kyoto Lab: Kyoto Research Park, 93,Awatacho,Chudoji, Shimogyo-ku,Kyoto, 600-8815, JAPAN and the Tokyo office: 337 Bldg #1, The University of Tokyo Institute of Medical Science 4-6-1,Shirokanedai, Minato-ku,Tokyo, 108-8639, JAPAN, in addition to a VERMES™ bioreactor (Satake Multimix) to allow rapid, high-quality platelet production.

Alternatively, a megakaryocyte line of choice, chosen after consultation with key opinion leaders (KOLs) are obtained and cultured. Back-up cell lines are established and stored at −80° C. Platelet production may take place in a VERMES™ bioreactor, or in a shaking flask with the six factors identified Ito et al., Cell, 174(3): 636-648.e18, 2018, which is hereby incorporated by reference in its entirety. The method is hypothezied to yield about 2.4×10⁶ μlatelets/ml in three days). A hybrid approach combining the techniques described herein also may be used. For example, Meg01 cells (ATCC® CRL-2021” from Sigma Aldrich) may be combined with the six factors in a bioreactor with turbulence to result in less clinical translation.

An in vitro assay for CD62 (specifically displayed on platelets on activation) may be performed to ensure the platelets are active. For example, platelet CD62 is measured using flow cytometry prior to activation. Adenosine diphosphate (ADP), thrombin, or collagen is added to activate platelets, then percent of surface exposure of CD62 is measured.

### Example 2. Generating Non-Thrombogenic Platelets

Once the progenitor cell line is established, it can be edited before platelet production. Genes may be knocked out, such as genes that affect the thrombogenicity of a platelet. Cas9 may be introduced to the megakaryocytes using a retrovirus to assist the editing process. Then, guide RNA (gRNA) electroporation is performed. A tracking of indels by decomposition (TIDE) analysis is performed to confirm the knockout of desired regions.

The cloning efficiency of cells also is measured to ensure the cells can be singly plated and grown up. In some embodiments of the invention described herein, the function of the edited platelets is measured using in vitro assays of platelet function, for example, microfluidic chips are commercially available to test aggregation.

Then, the platelets are moved to in vivo function testing. A mouse model as shown in Boulaftali et al. 2013, where endogenous mouse platelets can be depleted, may be used (See, Boulaftali et al. “Platelet ITAM signaling is critical for vascular integrity in inflammation”. JCI, 2013, which is hereby incorporated by reference in its entirety). A line of CLEC-2 knock-out (KO) human platelets is generated to act as a control line.

The non-thrombogenic platelets (CLEC-2 and vascular endothelial cadherin (ve)) are combined with a dye or beta-gal (0-Gal). Each mouse is transfused with a mix of control (CLEC-2) human platelets and non-thrombogenic edited platelets. The mouse is injured according to the protocol of an assay, such as hemoglobin (Hb) skin accumulation or tail vein bleeding time.

Any clot formed as a result of the assay is observed for the presence of edited platelets. The mice are treated with rhodocytin (a snake venom component that acts through CLEC-2) to trigger CLEC-2 dependent platelet aggregation of the edited platelets. Mice are examined for the presence of a clot. If no clot is present, the edited platelets are truly non-thrombogenic.

### Example 3. Generating CPR-Expressing Platelets

To test whether the edited platelets can be activated by an engineered stimulus using a CPR, CPRs were designed between known ITAM containing platelet receptors (GPVI, CLEC-2, and FCgR2A) and a model single chain antibody specific to an antigen (e.g. CD19). The construct is introduced either as an additional copy or by knock-in to the endogenous platelet receptor locus to replace the cognate extracellular domain of the receptor. The CPR expressing platelets aregenerated in vitro and exposed to a cell line expressing CD19 (e.g., NALM-6 cell line) and a control CD19 negative cell line (e.g., B16 melanoma cell line).

The ability of the CPR expressing platelets to subsequently activate in response to the presence of CD19 is assayed in vitro through microscopy. In some embodiments, a gene (e.g., TRAIL) is expressed to increase cytotoxicity by the engineered platelets.

Using a similar technique, the CPR is engineered to include portions of known ITAM containing platelet receptors (GPVI, CLEC-2, and FCgR2A) and single chain MHC class 1 and MHC class 2 receptors. The variant of MHC receptor used depends on the model used, e.g. New York esophageal squamous cell carcinoma 1 (NY-ESO-1) from Astarte Biologics. The construct is introduced as either an additional copy or by knock-in to the endogenous platelet receptor locus replacing its cognate extracellular domain. These CPR-expressing platelets are produced in vitro, and a peptide antigen is added to the sample. The CPR-expressing platelets are exposed to a T-cell line responsive to peptide-MHC (or to a naïve batch of mixed T cells), and T cell response to exposure is observed. The platelets are loaded with different cytokine cocktails to determine whether the T cell response can be modified.

### Example 4. Testing Non-Thrombogenic CPR-Expressing Platelets In Vivo

Non-thrombogenic platelets derived from a CD19 expressing melanoma cell line (or other melanoma cell line) are engineered to contain CTLA4 and PD-1 antibodies either passively or through retroviral transduction. Immunocompetent mice are treated with these platelets and checked for melanoma treatment.

Using the CD19 Nalm-6 B Cell leukemia model, TRAIL is expressed in non-thrombogenic platelets. FASL and CD40L are already present, which synergize with TRAIL to induce B Cell leukemia death. NOD scid gamma mice (NSG) mice having a tumor are treated with the engineered platelets. The mice are observed for a therapeutic benefit to validate the approach.

Alternatively, experimental autoimmune encephalomyelitis (EAE) is induced in mice using previously described protocol (vaccinated with maltose binding protein (MBP)). Human platelets with mouse MHC and/or L8057 mouse cells with mouse MHC are loaded with MBP peptide used for immunization. Further, platelets are loaded with at least one of cytotoxic components (to kill off specific cells) and TGF-β and other anti-inflammatories. A well-defined clinical score system is used to establish whether the above is an effective model system for testing the efficacy of non-thrombogenic CPR-expressing platelets in vivo.

### Example 5—Materials and Methods for Example 6 and 7

### CRISPR Guide Design

Guides were designed by identifying the first common exon of the target exon of a gene. This exon was used as input to the CRISPOR algorithm for guide selection. Four guides per target gene were chosen based on their distribution across the exon and their specificity score, listed in table 21.

Lentiviral iPSC Transduction

Replication deficient lentiviral particles containing CPR constructs and mCherry were produced by Flash Therapeutics. hiPSC lines were routinely transduced by 18-24 h single exposure to LVPs using multiplicity of infection of 100 in presence of 10 μg ml⁻¹ Protamine Sulfate (Sigma) in routine culture medium.

iPSC Cloning

HiPSCs were cloned by single cell sorting into 96 well plates. The day prior to sorting, iPSCs were treated with CloneR (Stem Cell Technologies). 96 well plates were coated with Biolaminin 521 LN (Biolamina). CloneR was kept in the media until day 2 post sorting. Colonies were harvested 15-20 days post sorting, by treating wells with ReLeSr and replating colonies into 24 well plates.

### Flow Cytometry and Staining

Single-cell suspensions were stained for 20 min at room temperature using combinations of FITC-, PE-, PE-Cy7-, APC-, and APC-H7-conjugated antibodies. Background fluorescence were set against fluorochrome-matched isotype control antibodies and compensation matrices defined using single colour-stained cells.

### CRISPR Editing—Screening

24 hours prior to nucleofection media was swapped for CloneR containing media. On the day of nucleofection, 1 μl of 61 pmol/μL of Alt-R HiFi Cas9 V3 (Integrated DNA Technologies) was mixed with 2 μl of 91.5 pmol/μL of sgRNA in TE (Synthego) (a 1:3 molar ratio) directly and incubated for at least 1 hour at room temperature. 100,000-500,000 HiPSCs per nucleofection were harvested with GCDR (Stem Cell Technologies). Harvested cells were spun down and resuspended in 20 μL nucleofection buffer P3 (Lonza). Cas9/gRNA mix was then added to the 20 μL cell/buffer P3 mix, then nucleofection was performed using 16-well Nucleocuvette Strip with 4D Nucleofector system (Lonza). Following nucleofection, 80 μL of media was added to the nucleocuvette well, and cells were replated into a single well of a 24 well plate, in CloneR containing media. Media was changed two days later for mTeSR Plus.

### CRISPR Editing—Sequential

24 hours prior to nucleofection media was swapped for CloneR containing media. On the day of nucleofection, 5 μl of 61 pmol/μL of Alt-R HiFi Cas9 V3 (Integrated DNA Technologies) was mixed with 10 μl of 91.5 pmol/μL of sgRNA in TE (Synthego) (a 1:3 molar ratio) directly and incubated for at least 1 hour at room temperature. 1-2.5 million HiPSCs per nucleofection were harvested with GCDR (Stem Cell Technologies). Harvested cells were spun down and resuspended in 100 μL nucleofection buffer P3 (Lonza). Cas9/gRNA mix was then added to the 100 μL cell/buffer P3 mix, then nucleofection was performed using the 100 μL Nucleocuvette with 4D Nucleofector system (Lonza). Following nucleofection, 400 μL of media was added to the nucleocuvette well, and cells were replated into two wells of a 6 well plate and one well of a 24 well plate, in CloneR containing media. Media was changed two days later for mTeSR Plus. Cells were given 3-4 days total to recover, before the subsequent nucleofection was performed.

### CRISPR KO Quantification

Genotyping was performed by first harvesting HiPSC cells using GCDR or ReLeSr. Genomic DNA was extracted using Kapa Express Extract Kit (Roche) following manufacturers instructions. Following genomic DNA extraction, the targeted genomic region was amplified using target locus specific primers (See table 2). PCR fragments were PCR purified and submitted for Sanger Sequencing (Source Bioscience). These sequences were then input into the ICE analysis software (Synthego) and thus editing efficiencies were quantified.

iPSC Cell Culture and Forward Programming to MK

The iPSC cell line RCIB-10 was forward programmed to megakaryocytes by the concurrent expression of TAL1, FLI1 and GATA1 from a doxycycline inducible promoter (see for example Dalby thesis, University of Cambridge “Forward programming of human pluripotent stem cells to a megakaryocyte-erythrocyte bi-potent progenitor population”; and Moreau 14 Sep. 2017 “Forward Programming Megakaryocytes from Human Pluripotent Stem Cells” BBTS Annual Conference Glasgow 2017). The parental RCIB-10 line was originally derived by episomal vector mediated expression of human OCT4, SOX2, KLF4 and MYC reprogramming factors from the donor cell line.

Cells were cultured under standard conditions with doxycycline for 10 days at which point the cells were harvested.

### HSC Differentiation to Meagkaryocytes

Adult, peripheral blood derived HSCs were driven to differentiate into megakaryocytes using a cocktail of cytokines in Stemspan SFEM2 (Stemspan 100× MK Supplement, Stem Cell Technologies, Catalog Number 02696). Protocols are available on the manufacturers website. Peripheral blood HSCs become competent for PLP production at D10-D13 of differentiation.

### PLP Production

PLPs were produced from megakaryocytes by moving them from standard growth media to RPMI 1640+25 mM glucose containing media. This step resulted in PLP production, however is not essential for PLPs production and PLPs can be harvested from standard culture media.

### P-Selectin Based Activation Assay (CRP/TRAP-6/PMA)

To assay the activation of MKs in response to mixing with known agonists, 100,000-500,000 MKs were first harvested by centrifugation at 100G for 8 minutes and resuspended in 100 μL of Tyrode's buffer (134 mM NaCl, 12 mM NaHCO₃, 2.9 mM KCl, 0.34 mM Na2HPO4, 1 mM MgCl2, 10 mM HEPES, pH 7.4) containing anti P-Selectin antibody (Biolegend, clone AK4, variable fluorophore at 1 μL/100 μL of cells). Where live cells were assayed by flow, this was performed by direct sampling from the tube without resuspension of cells. Agonists were subsequently added and incubated with MKs for 40 minutes, before fixation with 1% PFA for 15 minutes. Following PFA fixation, cells were resuspended in 300 μL Tyrode's buffer containing anti-CD42 antibody (1 μL/100 μL) was added to allow for mature MK identification. MKs were analysed either by imaging using confocal microscopy, or by flow cytometry. CRP (Cambcol) was added to cells at a concentration of 10 μg/ml, TRAP-6 (Abcam) at a concentration of 10 μM, PMA (Sigma) at a concentration of 300 ng/mL. When cells were used as agonists (Jurkats, DSMZ cat no: ACC 282 and BJABs—B Cell lymphoma line, Ghevaert lab stock) they were added in 1:1 number vs. MKs.

Table 21-23 presented on pages 154-161 of PCT/GB2020/053247 and which present gRNA primer sequences; amplicon primers; and media recipes are hereby incorporated by reference. Table 21 and 22 are reproduced below for convenience:

TABLE 21
gRNA primer sequences:

| | | PAM |
|---|---|---|
| | | se- |
| Name | Sequence | quence |
| HPS1 | | |
| Exon 7 | | |
| grna1_ | GGGGTGAATCAGTCGCTCCA | GGG |
| HPS1_1r | [SEQ ID NO: 56] | |
| grna2_ | GTCAACACCAGCCCCGAGCG | GGG |
| HPS1_2 | [SEQ ID NO: 57] | |
| grna3_ | GCTGGAGCGGCACGTCATCC | AGG |
| HPS1_3 | [SEQ ID NO: 58] | |
| grna4_ | CTTGGAGTGCACGAGCAGGA | AGG |
| HPS1_4r | [SEQ ID NO: 59] | |
| ITGA2B | | |
| Exon 7 | | |
| grna5_ | CAGTAGCCGTCGAAGTACTC | TGG |
| ITGA2B_1r | [SEQ ID NO: 60] | |
| grna6_ | ATTTTCTCGAGTTACCGCCC | AGG |
| ITGA2B_2 | [SEQ ID NO: 61] | |
| grna7_ | CTCGAGAAAATATCCGCAAC | TGG |
| ITGA2B_3r | [SEQ ID NO: 62] | |
| grna8_ | GGGAGGACACGTGCCACAAA | AGG |
| ITGA2B_4r | [SEQ ID NO: 63] | |
| GP6 | | |
| Exon 3 | | |
| grna9_ | GGGCGTGGACCTGTACCGCC | TGG |
| GP6_1 | [SEQ ID NO: 64] | |
| grna10_ | ACGAGCTCCAGCTGGTCGCT | GGG |
| GP6_2r | [SEQ ID NO: 65] | |
| grna11_ | CGGAGGTCCCTGGCACCGGA | GGG |
| GP6_3r | [SEQ ID NO: 66] | |
| grna12_ | CCAGTGACCCTCCGGTGCCA | GGG |
| GP6_4 | [SEQ ID NO: 67] | |
| Par1 | | |
| Exon 2 | | |
| grna13_ | GGAGCTGGTCAAATATCCGG | AGG |
| Par1_1r | [SEQ ID NO: 68] | |
| grna14_ | TTCCTGAGAAGAAATGACCG | GGG |
| Par1_2r | [SEQ ID NO: 69] | |
| grna15_ | ACACTCCGGTGTACACAGAT | GGG |
| Par1_3r | [SEQ ID NO: 70] | |
| grna16_ | ACGATGGCCATGATGTTTAG | TGG |
| Par1_4r | [SEQ ID NO: 71] | |
| Par4 | | |
| Exon 2 | | |
| grna17_ | ACTTGGCCTGGGTAGCCGCG | GGG |
| Par4_1r | [SEQ ID NO: 72] | |
| grna18_ | GGTGCCCGCCCTCTATGGGC | TGG |
| Par4_2 | [SEQ ID NO: 73] | |
| grna19_ | TGGTGGGGCTGCCGGCCAAT | GGG |
| Par4_3 | [SEQ ID NO: 74] | |
| grna20_ | AGCAGTGCCCGTGAGCTGTC | CGG |
| Par4_4r | [SEQ ID NO: 75] | |
| Cox1 | | |
| Exon 7 3′ and exon 8 | | |
| grna21_ | ACTTCTGGCAAGATGGGTCC | TGG |
| Cox1_1 | [SEQ ID NO: 76] | |
| grna22_ | TCACCAAGGCCTTGGGCCAT | GGG |
| Cox1_2 | [SEQ ID NO: 77] | |
| grna23_ | TGTCTCCATAAATGTGGCCG | AGG |
| Cox1_3r | [SEQ ID NO: 78] | |
| grna24_ | AACTGCGGCTCTTTAAGGAT | GGG |
| Cox1_4 | [SEQ ID NO: 79] | |
| P2Y12 | | |
| Exon 3 | | |
| grna29_ | GTAGTCTCTGGTGCACAGAC | TGG |
| P2Y12_1r | [SEQ ID NO: 80] | |
| grna30_ | GAAAGAAAATCCTCATCGCC | AGG |
| P2Y12_2r | [SEQ ID NO: 81] | |
| grna31_ | ATTCTTAGTGATGCCAAACT | GGG |
| P2Y12_3 | [SEQ ID NO: 82] | |
| grna32_ | GATCGATAGTTATCAGTCCC | AGG |
| P2Y12_4r | [SEQ ID NO: 83] | |
| B2M | | |
| Exon 2 | | |
| grna40_ | AAGTCAACTTCAATGTCGGA | TGG |
| B2M_1r | [SEQ ID NO: 84] | |
| grna41_ | AGTCACATGGTTCACACGGC | AGG |
| B2M_2r | [SEQ ID NO: 85] | |
| grna42_ | ACTTGTCTTTCAGCAAGGAC | TGG |
| B2M_3 | [SEQ ID NO: 86] | |
| grna43_ | TCACGTCATCCAGCAGAGAA | TGG |
| B2M_4 | [SEQ ID NO: 87] | |

As mentioned elsewhere herein, the skilled person will appreciate that it is conventional to provide the sequence of a RNA molecule using the nucleotides AGTC. However the skilled person knows that in RNA the T is replaced with Uracil. Accordingly, any sequence described herein that relates to an RNA molecule can be written with a T or a U, though in practice the RNA molecule will contain U rather than T.

TABLE 22
amplicon primers:

| | Sequencing primers | |
|---|---|---|
| HPS1 | | |
| RocO1_ | F | ATCTGGTGCAGAGTCCAAGC |
| sHPS1_F1 | | [SEQ ID NO: 88] |
| RocO2_ | R | TGGAGGAGGTGATTCTTGGC |
| sHPS1_R1 | | [SEQ ID NO: 89] |
| | Product | 387 |
| | size: | |
| ITGA2B | | |
| RocO3_ | F | GGCTCCTGGCGGCTATTATT |
| ITGA2B_F1 | | [SEQ ID NO: 90] |
| RocO4_ | R | CTTAGGCGGTGGGTTGGC |
| ITGA2B_R1 | | [SEQ ID NO: 91] |
| | Product | 360 |
| | size: | |
| GP6 | | |
| RocO5_ | F | AGCAGCGGGGTCCAGG |
| GP6_F1 | | [SEQ ID NO: 92] |
| RocO6_ | R | CGTGGCACCACCACCC |
| GP6_R1 | | [SEQ ID NO: 93] |
| | Product | 462 |
| | size: | |
| Par1 | | |
| RocO7_ | F | ACCCACTCTCCTAGTAAGAAAACAT |
| Par1_F1 | | [SEQ ID NO: 94] |
| RocO8_ | R | CAAACTGCCAATCACTGCCG |
| Par1_R1 | | [SEQ ID NO: 95] |
| | Product | 541 |
| | size: | |
| Par4 | | |
| RocO9_ | F | ATGTCCAGCTGTTTCCCACC |
| Par4_F1 | | [SEQ ID NO: 96] |
| RocO10_ | R | GCAGGTGGTAGGCGATCC |
| Par4_R1 | | [SEQ ID NO: 97] |
| | Product | 415 |
| | size: | |
| Cox1 | | |
| RocO11_ | F | CCAACCAGGGAAGAAGCAGT |
| Cox1_F1 | | [SEQ ID NO: 98] |
| RocO12_ | R | TGGCACAAGCTTCCCACTC |
| Cox1_R1 | | [SEQ ID NO: 99] |
| | Product | 514 |
| | size: | |
| P2Y12 | | |
| RocO15_ | F | GAGGAGGCTGTGTCCAAAAA |
| P2Y12_F1 | | [SEQ ID NO: 100] |
| RocO16_ | R | GGCTGCCTGTTGGTCAGAAT |
| P2Y12_R1 | | [SEQ ID NO: 101] |
| | Product | 607 |
| | size: | |
| B2M | | |
| RocO58_ | F | TGACACCAAGTTAGCCCCAA |
| B2M_F1 | | [SEQ ID NO: 102] |
| RocO59_ | R | GGGATGGGACTCATTCAGGG |
| B2M_R1 | | [SEQ ID NO: 103] |
| | Product | 463 |
| | size: | |

### Example 6

To generate a non-thrombogenic, iPSC derived platelet-like progenitor, producer or effector-chassis, genes encoding key components of the endogenous thrombotic process must be deleted.

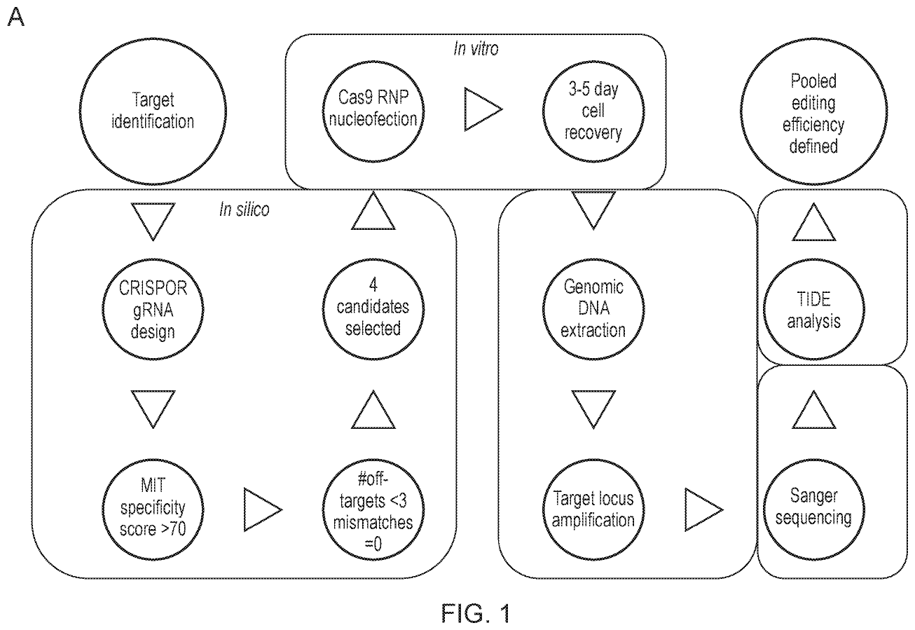

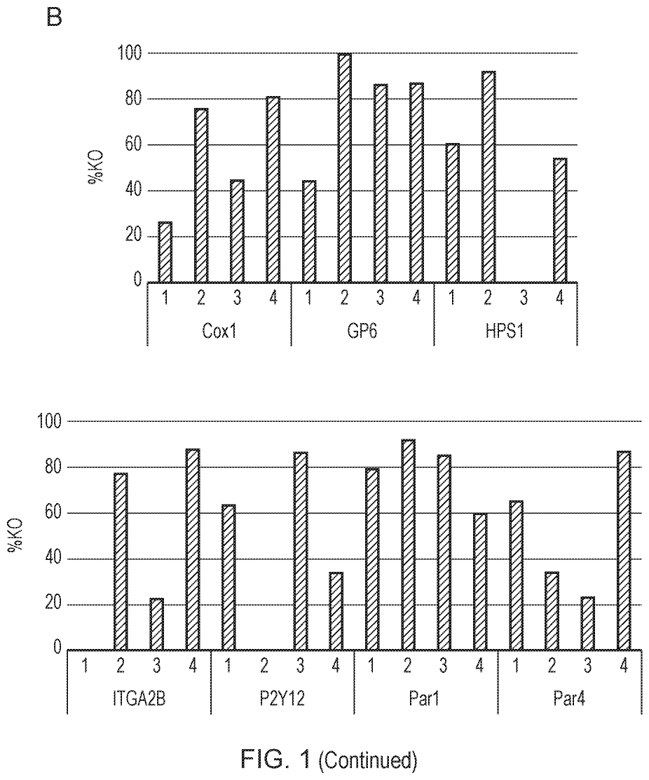

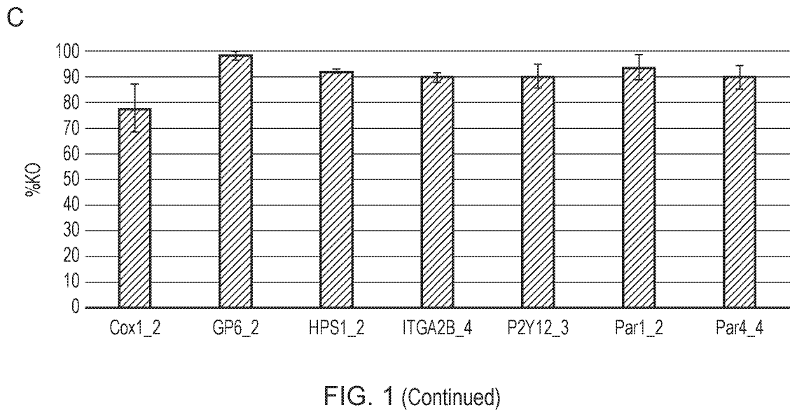

In this instance, the genes targeted were Cox1, GPVI, HPS1, ITGA2B, P2Y12, Par and Par4. CRISPR/Cas9 mediated IN/DEL generation was chosen as the method for gene knock-out (KO). First, guides were designed to target Cas9 nuclease to the above mentioned targets (FIG. 1A). Four guides were designed per target, and nucleofected as complex with the Cas9 protein into iPSCs, and their gene editing efficiency within the pool measured by Sanger sequencing and TIDE or the Synthego ICE algorithm. High efficiency guides resulting in >80% KO of each target were identified in the guide screen (FIG. 1B). These guides generated reproducibly high editing efficiency (FIG. 1C).

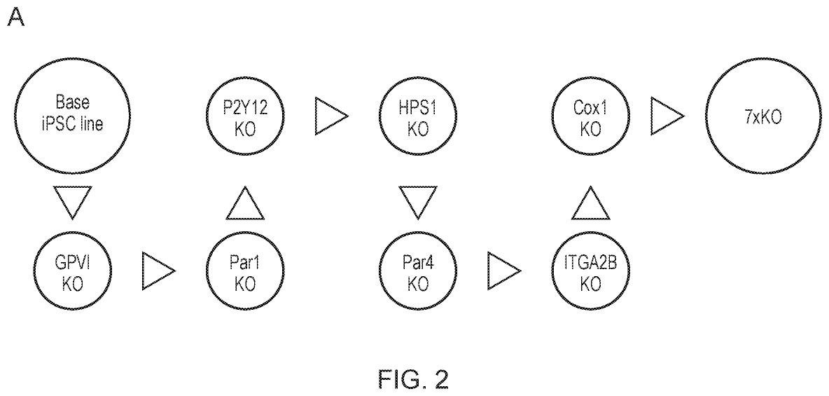

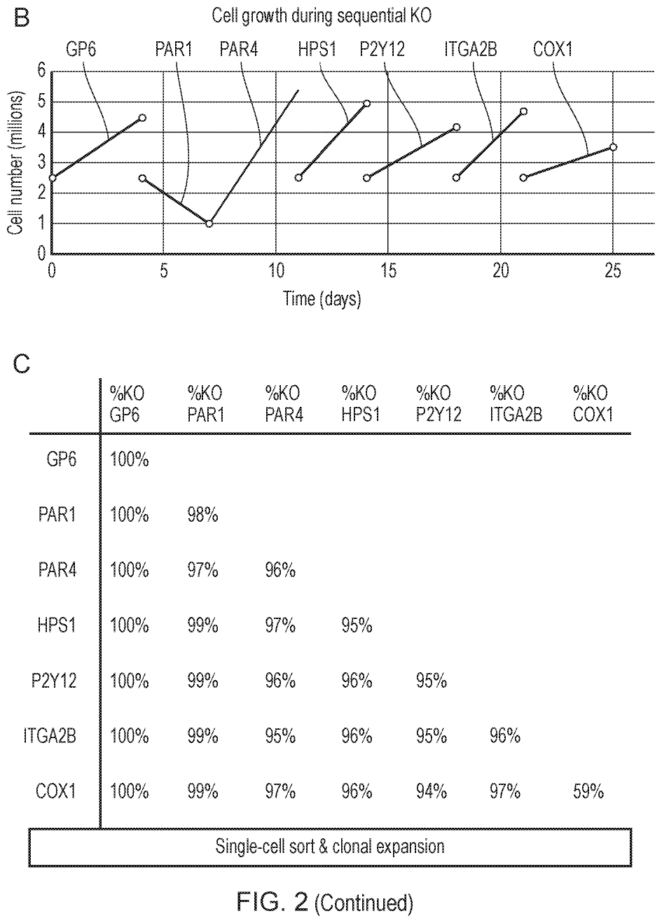

To generate the non-thrombogenic progenitor iPSC line, these KOs must all be introduced into the same cell. To achieve this, a sequential editing protocol was designed (FIG. 2A). In brief, Cas9 RNP complexes featuring the high efficiency guides identified previously were nucleofected into the same population of iPSCs sequentially, with 3-4 days rest between each nucleofection. This protocol did not produce an adverse effect on cell viability or growth throughout the ˜3.5 week process (FIG. 2B). Gene KO was quantified for each target hit previously throughout the sequential nucleofection protocol. No gene KO dilution was observed (as might occur if the KO itself was detrimental), and surprisingly high gene editing efficiencies were observed for all targets (>94% for all targets except COX1) (FIG. 2C). Following the sequential KO protocol, single cells were sorted into a 96 well plate and allowed to grow up forming clonal colonies. These colonies were subsequently isolated and sequenced. Three 7xKO clones were identified (FIG. 3).

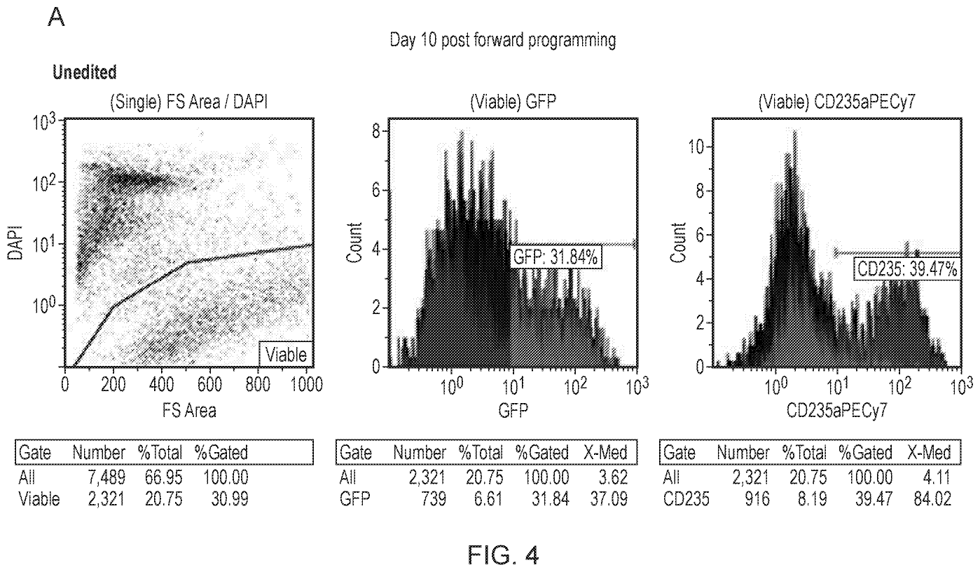

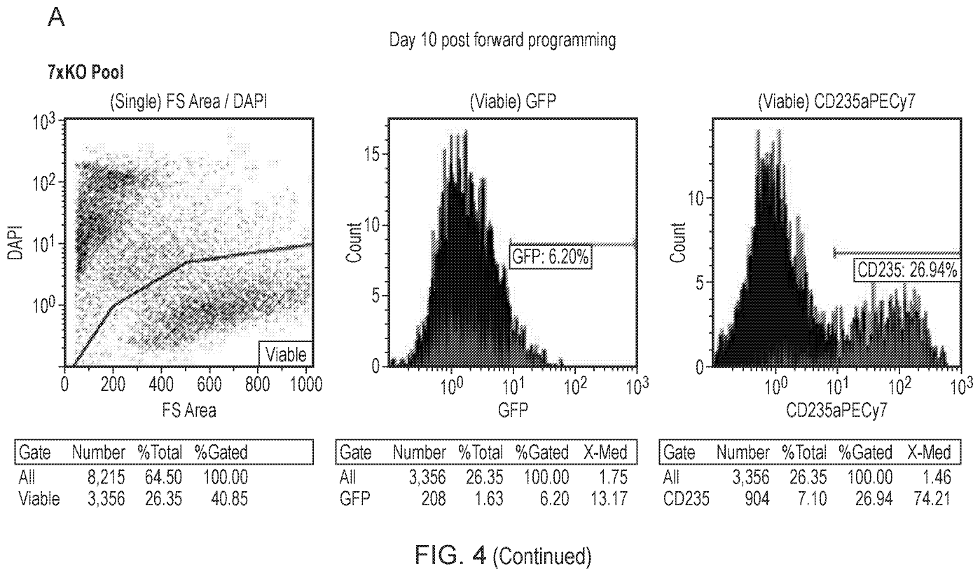

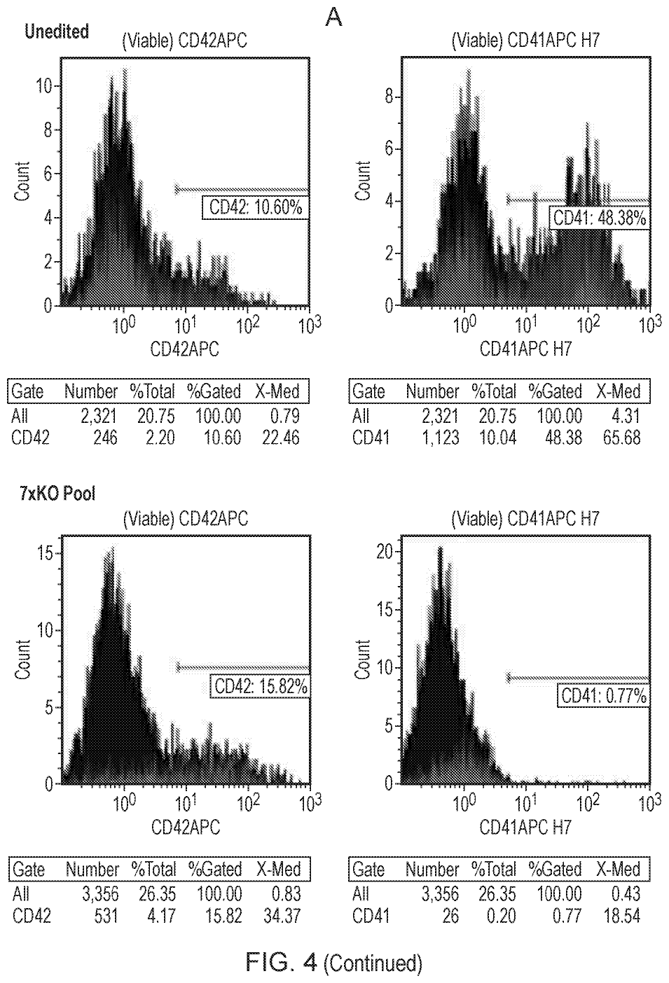

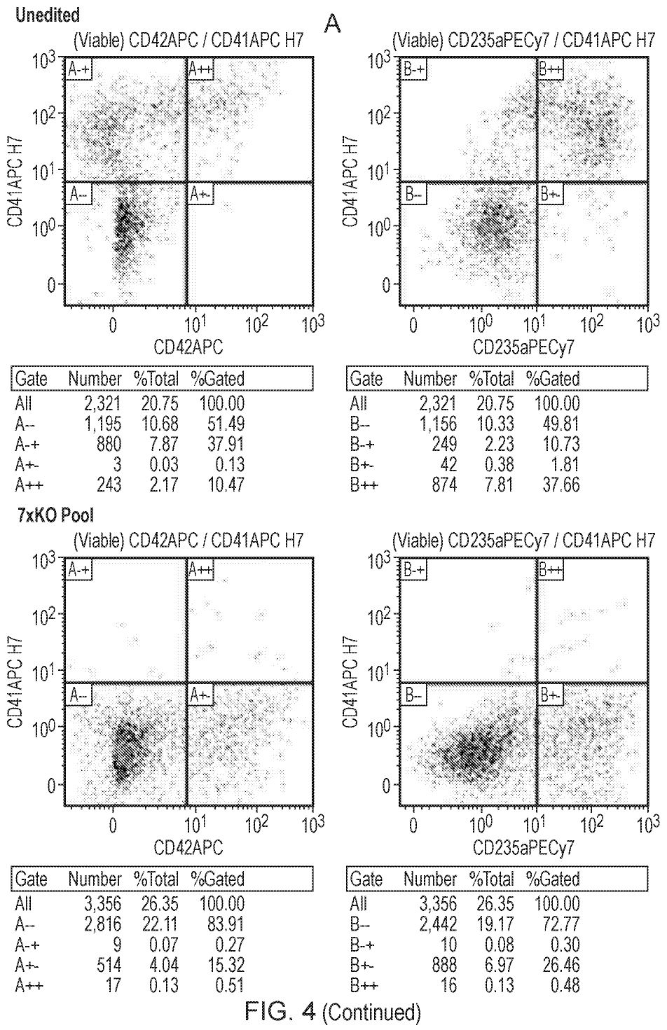

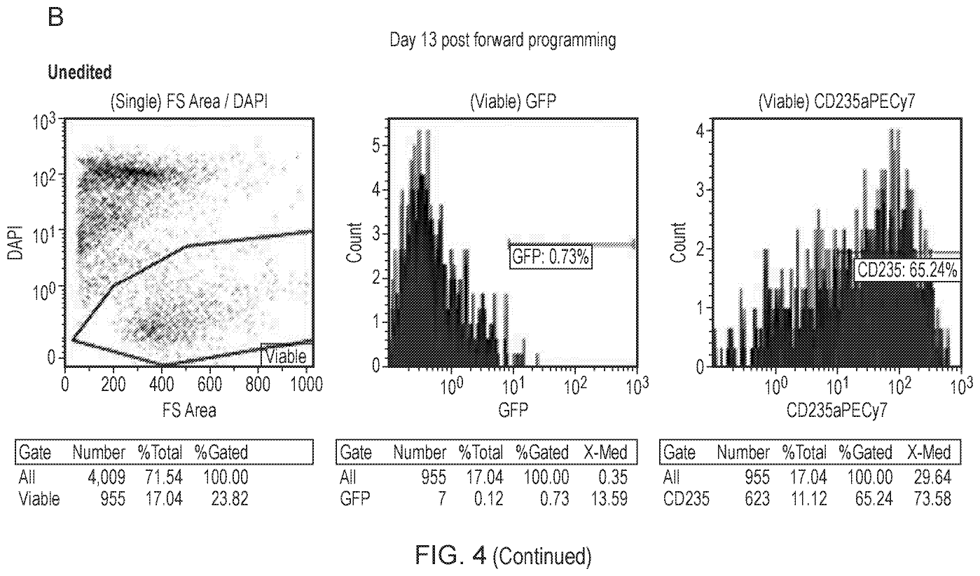

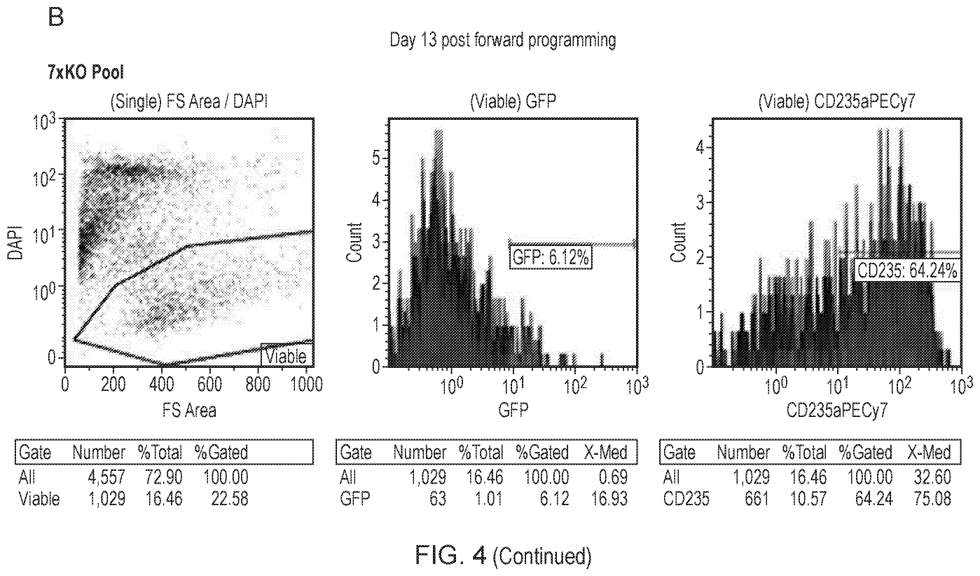

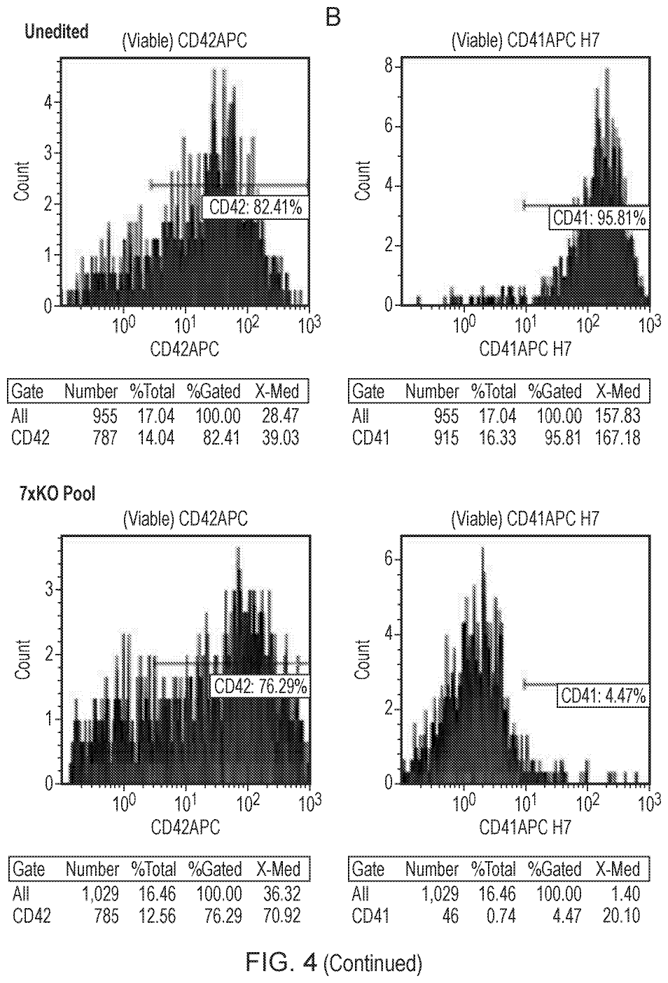

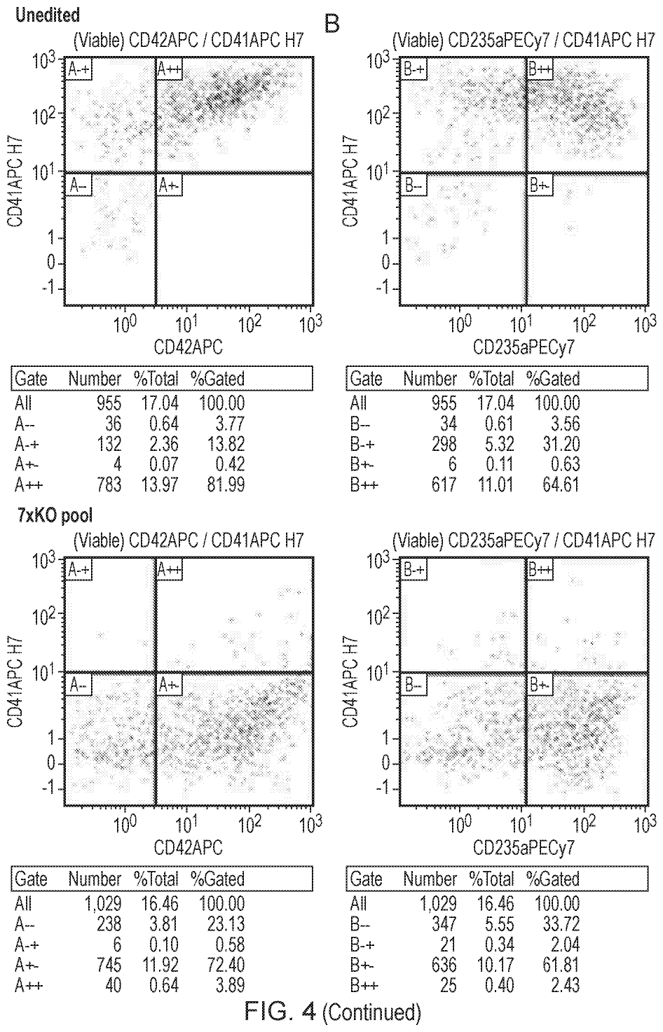

Given the number of megakaryocyte (MK) specific genes KO'd within these iPSCs, it remained unclear as to whether these iPSCs would still be able to differentiate into MK like cells. To understand this, iPSCs were forward programmed into MKs by doxycycline mediated induction of MK specific transcription factors GATA1, TAL1 and FLI1. Cell surface expression of known, well defined MK markers and viability was assayed during the forward programming process (FIGS. 4A and B). This study was performed in the pool of 7xKO MKs, but given the exceedingly high editing efficiencies within the pool it is likely >90% of cells feature at least 6 KOs. We observed no effect on forward programming efficiency or MK viability during the forward programming process. CD41 is ITGA2B, one of our target genes. Thus the lack of CD41 expression within the 7xKO population validated the protein level KO of this gene as predicted by our sequencing based approach.

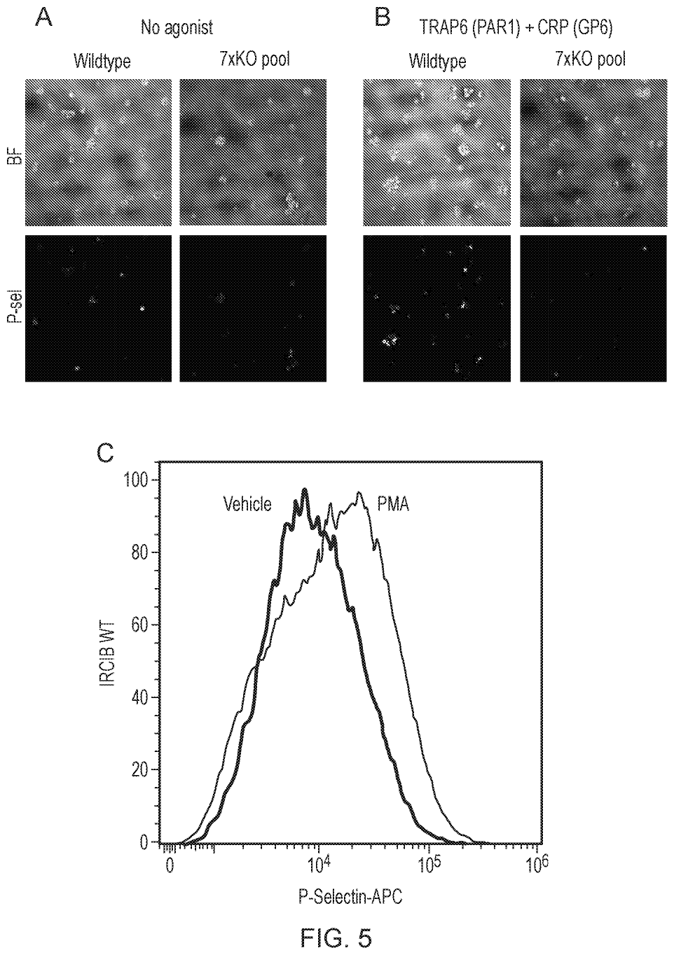

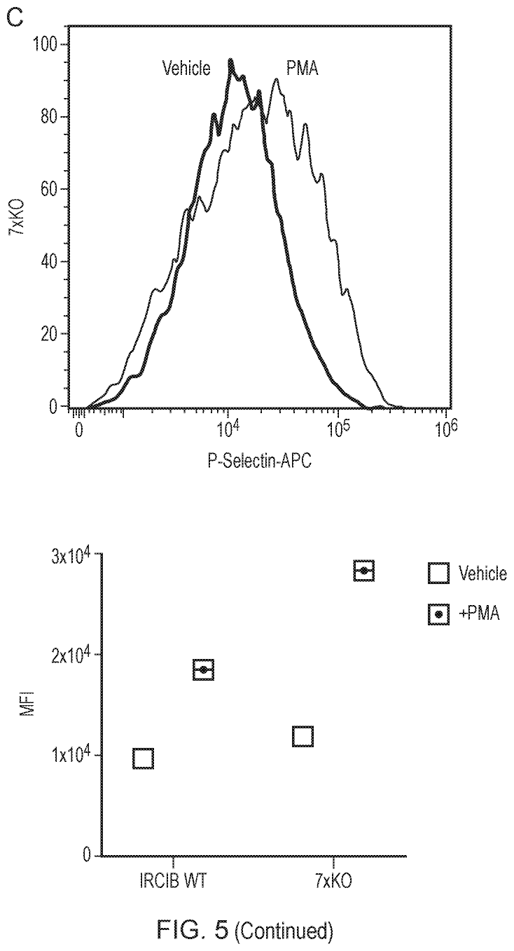

To validate the non-thrombogenicity of our 7xKO MKs, and also their retained function, we studied their degranulation response to known platelet agonists. MKs contain the same core signal transduction machinery, plasma membranes and components as platelets (given platelets are fragments of MKs), and thus MKs were used here as a surrogate for actual platelets. It is expected that the results seen in MKs would translate directly to platelets. To assay for degranulation, cell surface P-Selectin exposure was used as a marker. P-Selectin is an alpha-granule membrane protein, and is not usually present on the platelet surface. Upon platelet activation, alpha-granules fuse with the plasma membrane and exocytose their contents (degranulation), and their membrane components mix with the plasma membrane. P-Selectin thus becomes exposed and detectable by fluorescent antibody mediated staining. Resting 7xKO MKs feature lower basal levels of P-Selectin exposure than unedited wildtype MKs (FIG. 5A). Upon stimulation with two classical platelet agonists, CRP and TRAP6 (which signal through GPVI and PAR1 respectively—both KO'd in the 7xKO pool), no increase in P-Selectin staining was observed in the 7xKO MK pool. This is in contrast to the unedited MKs, which increased their P-Selectin and also appeared began to form small aggregates of cells (FIG. 5B). Importantly, upon stimulation of the 7xKO MKs with PMA, an agonist that bypasses the signaling pathways removed within the 7xKO line, 7xKO MKs exposed P-Selectin as well if not better than unedited MKs (FIG. 5C). Taken together, these activation experiments and the cell surface marker experiments discussed previously demonstrate that deletion of our candidate non-thrombogenic genes in iPSCs does not perturb their ability to differentiate into MK like cells, and does not disrupt the ability of MKs to degranulate in response to non-deleted signal transduction mechanisms.

Platelets contain ITAM domain containing receptors—specifically CLEC2, FCERG and FCGR2A. CLEC2 is a type-II membrane protein, whilst FCERG and FCGR2A are type-I membrane proteins. Type-I membrane proteins are amenable to fusion with scFV fab domains (and other N-terminal targeting mechanisms). Chimeric platelet receptors (CPRs) were thus designed as fusions between an scFV targeting the B cell antigen CD19 derived from the FMC63 antibody, a hinge domain, and the transmembrane and cytoplasmic domains of FCERG and FCGR2A. This yielded four potential receptor designs (FIG. 6A). These designs were inserted into lentiviral expression vectors as a multicistronic construct, with an mCherry fluorescent protein linked by a T2A peptide splitting sequence (FIG. 6B). Viral particles were transduced onto iPSCs, and transduction efficiency examined by mCherry expression. Notable mCherry expression was detected across all four lentiviral expression vectors, and was not present in the untransduced control (FIG. 6C).

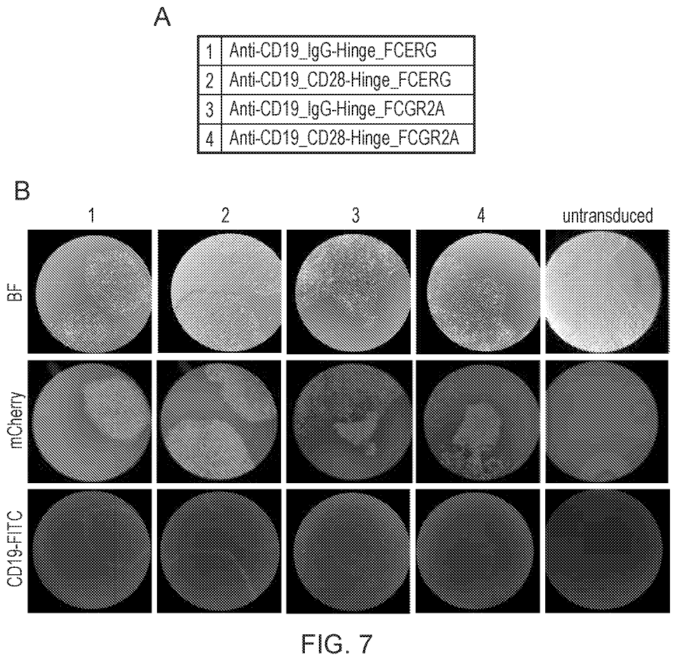

To validate that the receptor itself was expressed and cell surface localised, virally transduced iPSCs were stained with recombinant CD19 fluorescently labelled with FITC. CD19-FITC should only label iPSCs if they express the anti-CD19 scFV on their cell surface, in the correct orientation. Notably, colonies positive for transduction (i.e. mCherry positive) were also positive for CD19-FITC, indicating that the designed CPRs fold and correctly localised to the plasma membrane of the cells expressing them (FIG. 7).

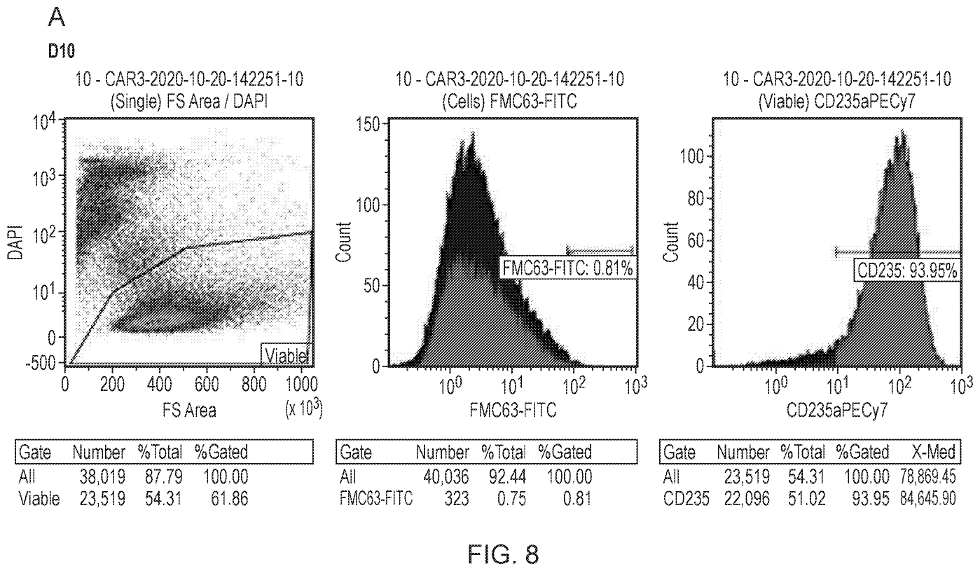

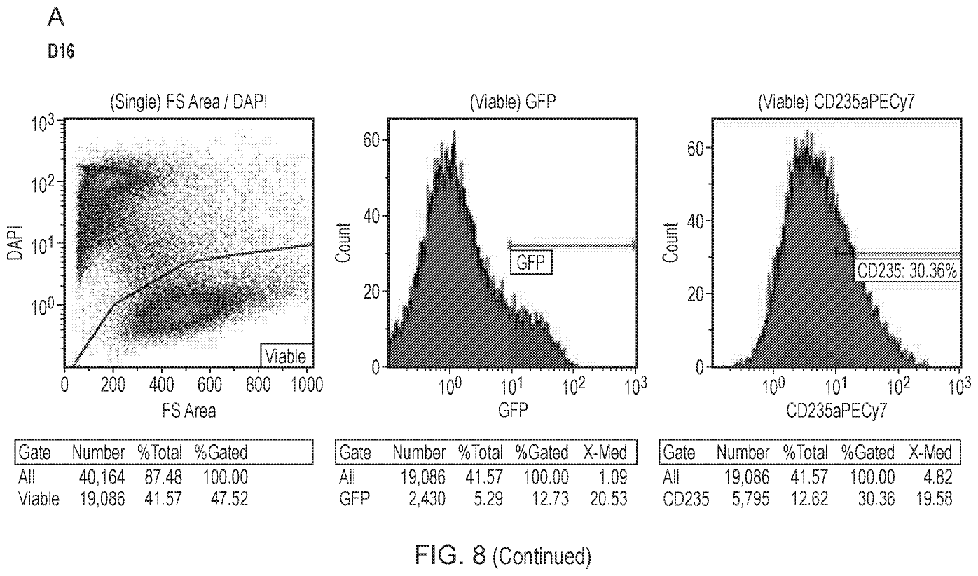

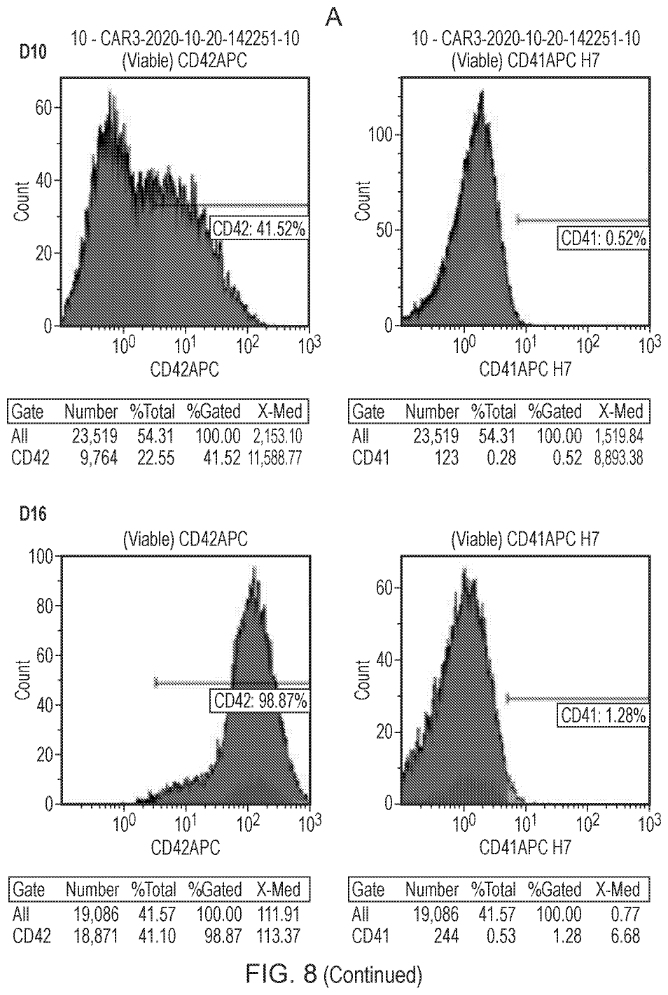

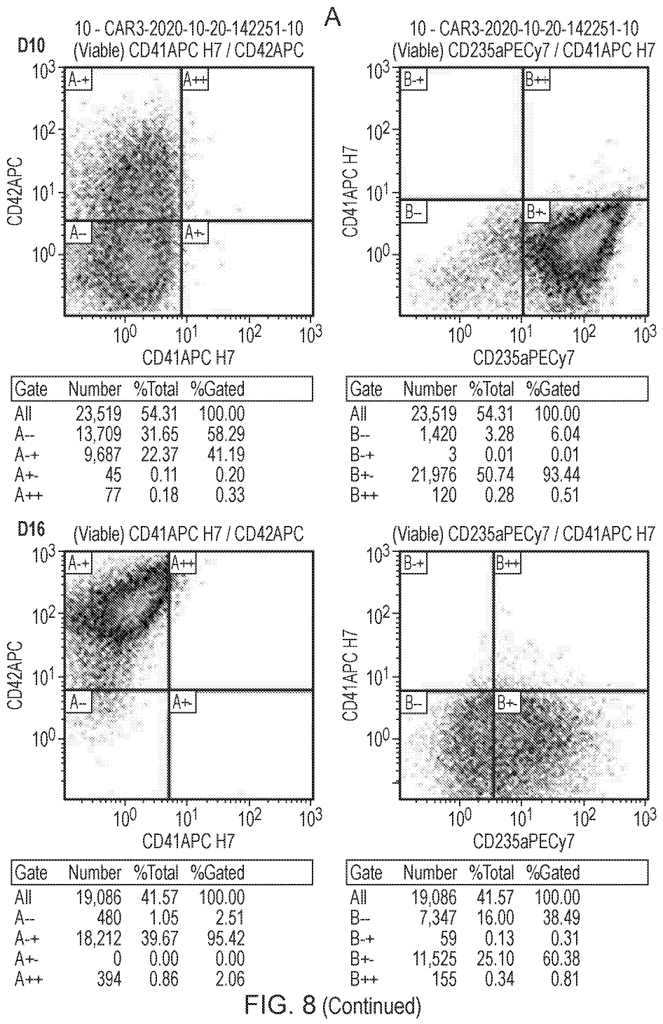

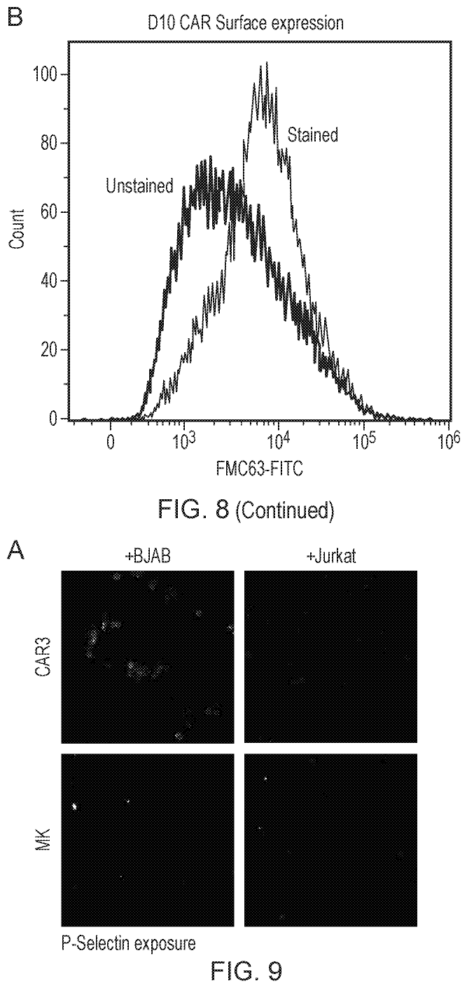

A clonal high CPR3 expressing iPSC line was forward programmed into MKs. Expression of the CPR3 construct did not impact the ability for iPSCs to forward program, as all classical MK specific markers were expressed within these cells. MK viability was not impacted by CPR3 expression either (FIG. 8A). Note that CD41 is clonally KO'd within these cells, and thus the lack of its expression is expected. To validate that CPR3 was expressed and that this expression was maintained on the MK cell surface, CD19-FITC staining was conducted (FIG. 8B). CPR surface expression was observed, indicating MK differentiation did not silence the lentiviral expression construct, or somehow alter receptor localisation.

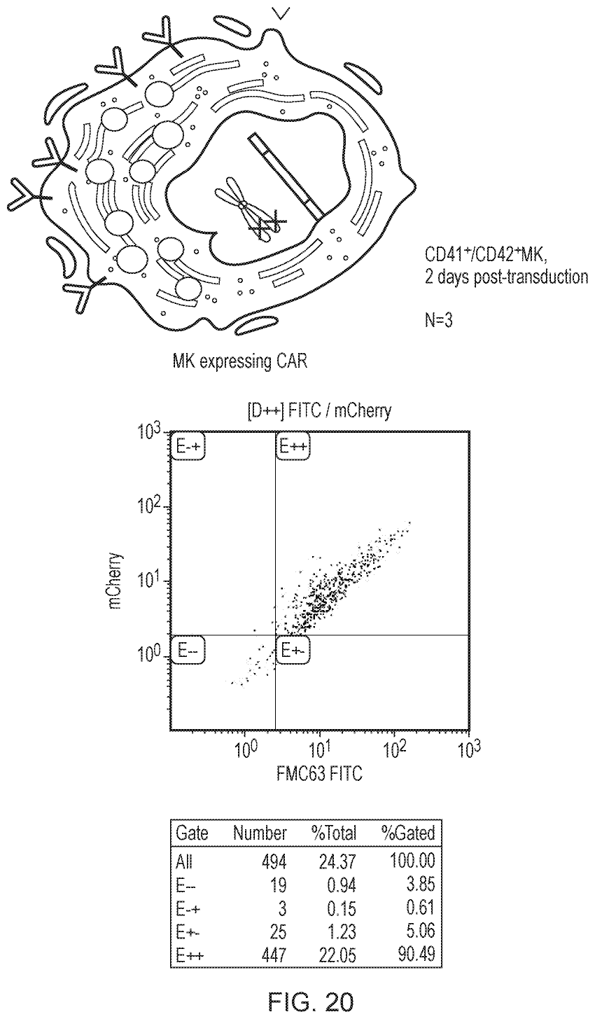

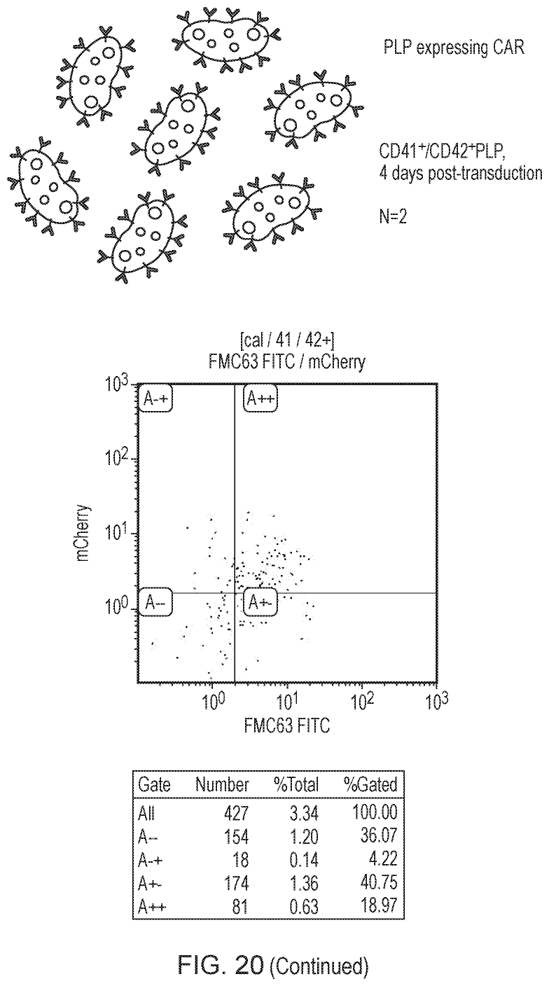

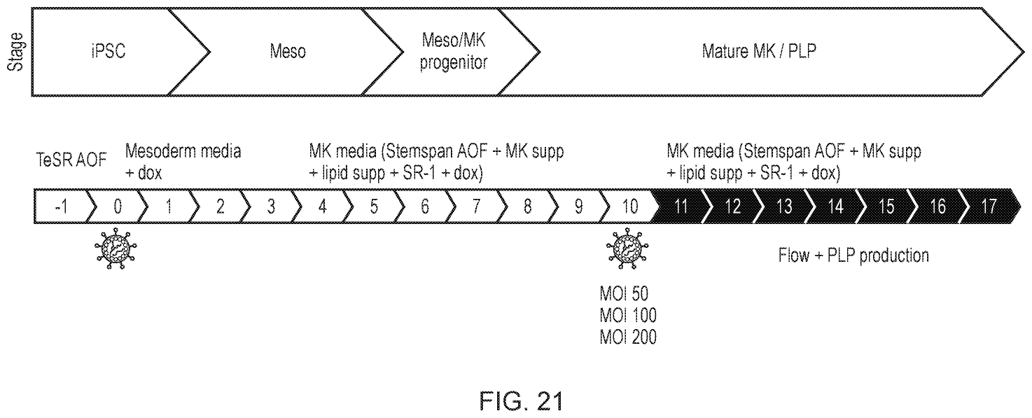

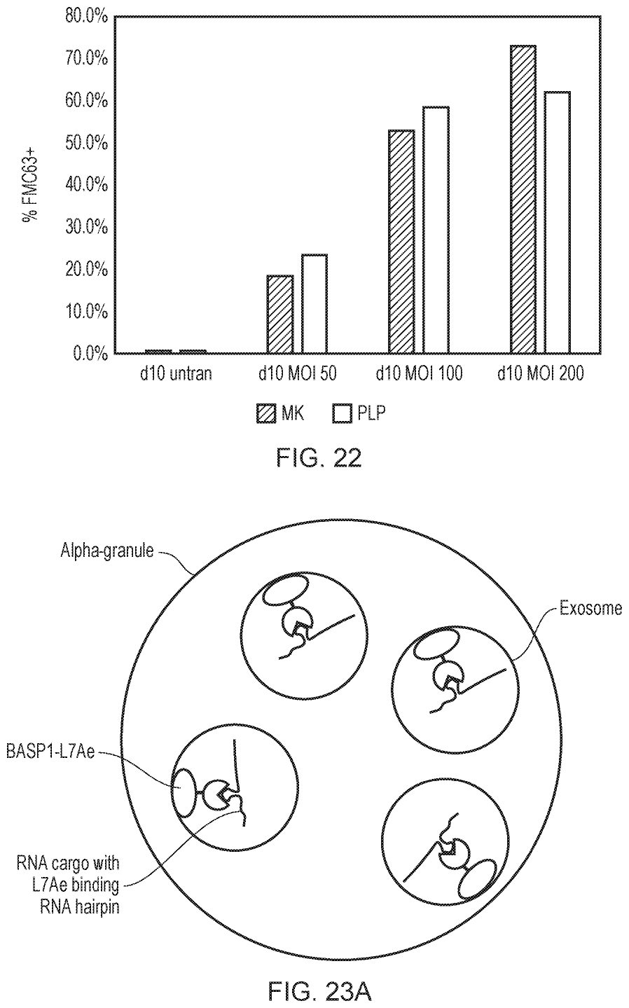

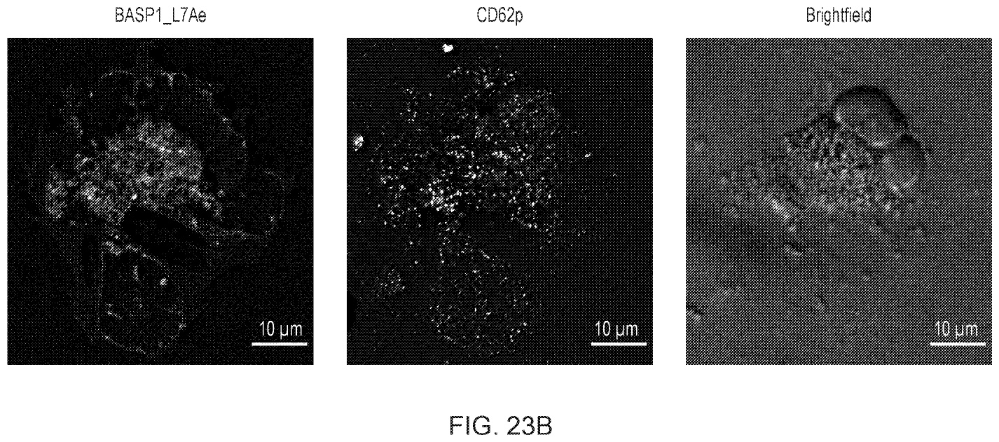

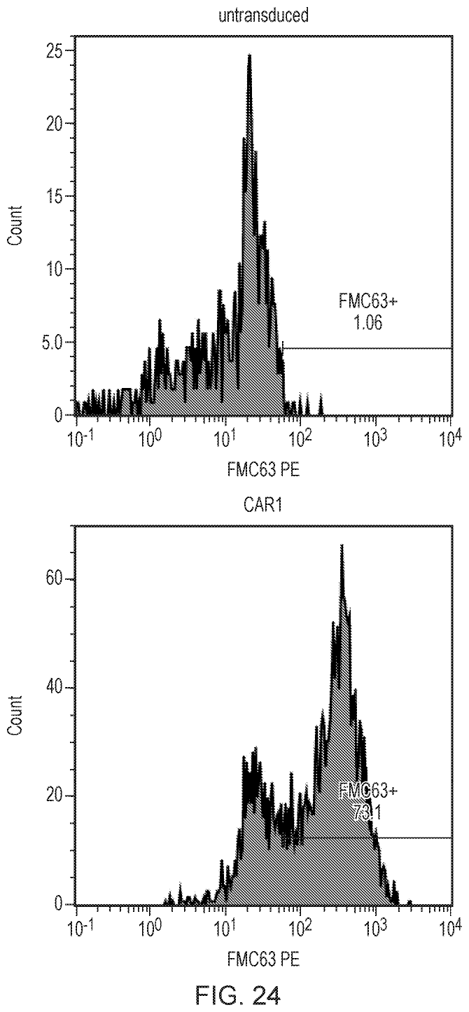

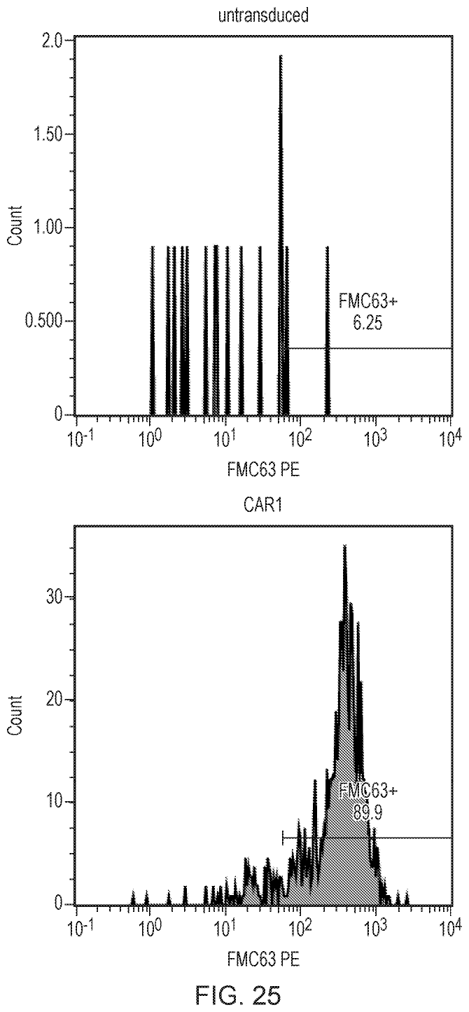

To further confirm the correct surface localisation of platelet specific CARs, CPR1 was expressed in haematopoetic stem cell derived megakaryocytes. Following CPR1 lentivirus transduction (featuring coupled CPR and mCherry expression), there was a srong positive correlation between mCherry levels and surface scFV expression levels. This demonstrates that CPR1 can fold and localise well to the surface of a megakaryocyte (FIG. 20 part 1). Importantly, following PLP production from these megakaryocytes, PLPs were also shown to express surface resident, correctly oriented CPR1. These results have also been subsequently repeated in iPSC derived megakaryocytes and PLPs derived from them (FIG. 22). Importantly, CPR1/CAR1 localised to the surface in non-thrombogenic 7xKO clone 34 line (FIG. 25), and in a control CD34/CD41 KO line (FIG. 24). This demonstrates experimental evidence that non-thrombogenic platelet “chasses” can be functionalised with a CPR.

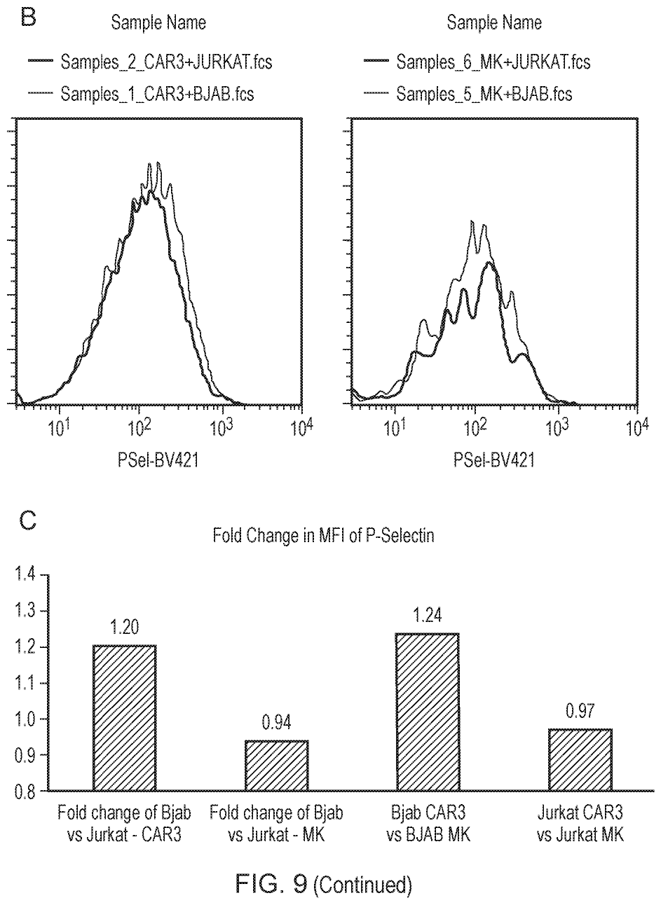

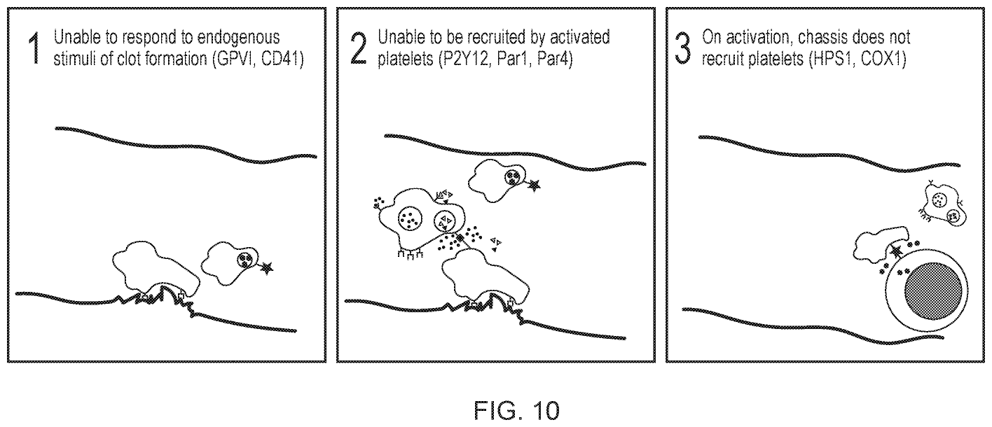

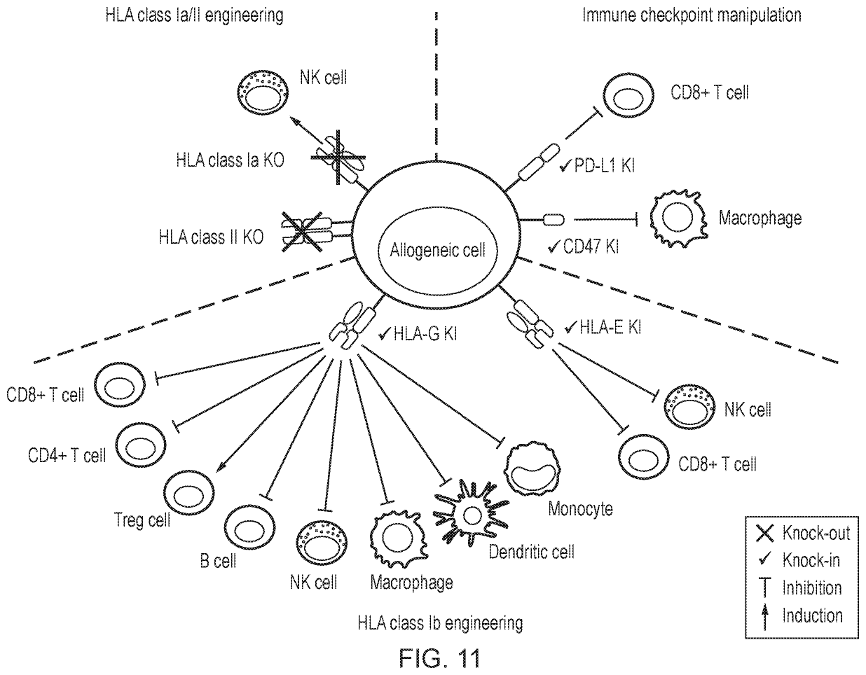

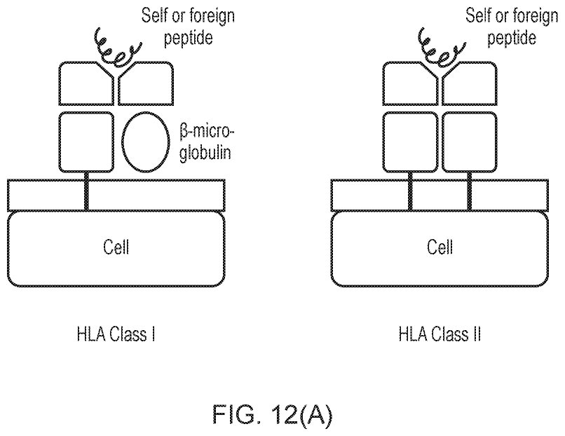

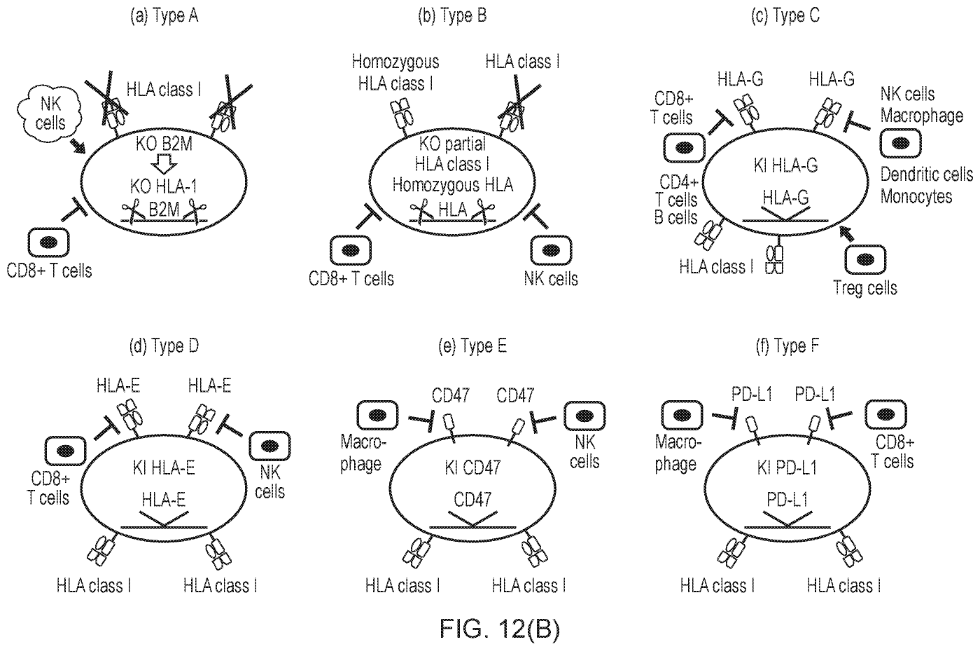

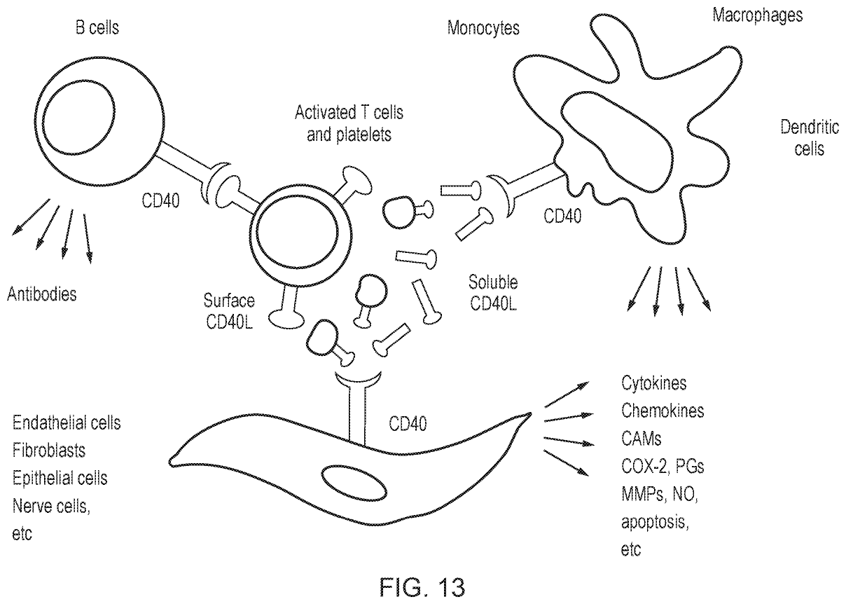

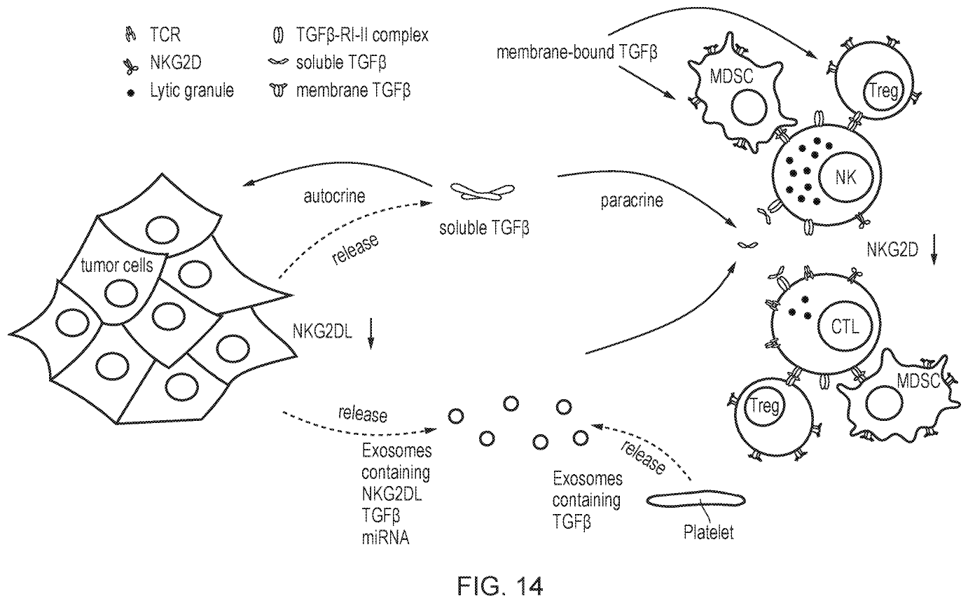

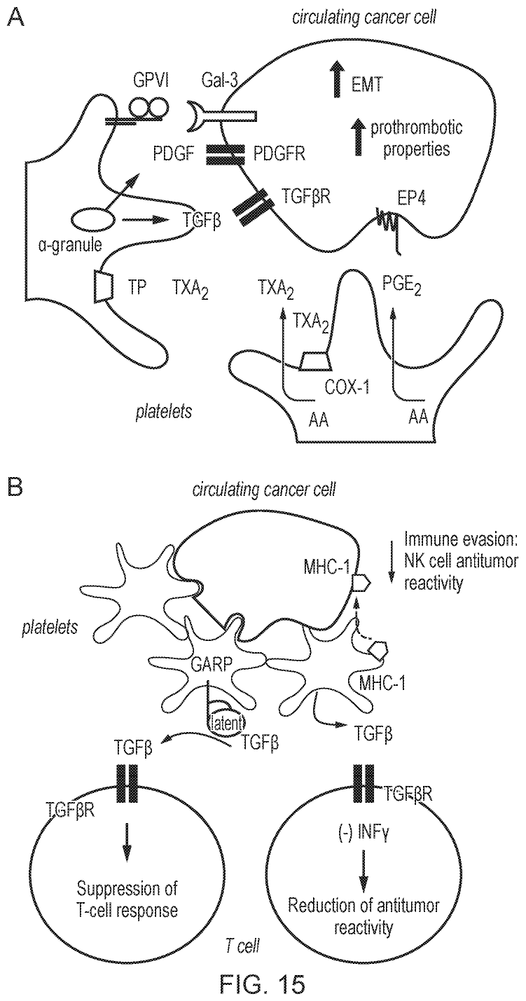

To study the functionality of the CPR, CPR3 expressing MKs and control untransduced MKs were mixed with a CD19 expressing B cell leukemia line (BJABs) or CD19 negative T cell leukemia line (Jurkats) and P-Selectin exposure was measured as before. Microscopy imaging of mixed cell populations demonstrated increased P-Selectin exposure specifically within CPR3 expressing MKs when mixed with the CD19+ve BJABs (FIG. 9A). This was result was confirmed quantitatively by FACS based measurement of P-Selectin exposure (FIGS. 9B and C). BJAB cells do not activate untransduced MKs, and CD19 negative Jurkats do not activate CPR3 expressing MKs. These results demonstrate that the CPR3 construct specifically stimulates MK degranulation in response to triggering by CD19 positive BJAB cells. Given that platelets are cytoplasmic fragments of MKs and the core signaling machinery is shared between them (given the shared cytoplasm), it is expected that these results should translate into platelets when produced from CPR3 expressing MKs. Additionally, given our observation that 7xKO MKs retain the ability to activate and degranulate in response to agonists that have not had their cognate receptors deleted, it is expected that CPR3 expression within a 7xKO line should trigger its degranulation upon mixing with CD19 positive cells. Given the swappable nature of the external CPR targeting domain, target specificity could be altered by swapping the anti-CD19 scFV for alternative targeting mechanisms, while retaining the same internal signaling domain that has been shown here to trigger MK degranulation on target engagement.

### Example 7—Testing of IPSC Knock-Out Lines Designed for Non-Thrombogenic Activity

Two different iPSC cell lines were generated, each having three gene knockouts:
Clone 1: iTGA2b KO, PAR1 KO, HPS1 KO
Clone 2: iTGA2b KO, P2Y12 KO, HPS1 KO

Each clone has been designed to become phenotypically defective in 3 key processes involved in thrombogenesis.
Inactivation of iTGA2b inactivates the GPIIb/IIIa receptor which is essential for recognition of stimuli (fibrinogen) resulting in the exposure of basement membrane under the damaged endothelium
Inactivation of PAR1 or P2Y12 inactivates the receptors for thrombin and ADP, respectively. These agonists are platelet-derived secondary messengers which are released by activated platelets to recruit further platelets to a growing thrombus
Inactivation of HPS1 prevents the formation of dense granules in platelets. Dense granules contain and release secondary mediators of platelet activation such as ADP and serotonin. Thus, the removal of dense granules in platelets prevents normal, wild-type platelet recruitment to iPSC-derived knock out platelets.

iPSCs featuring knock-outs of the above mentioned genes were generated using methodologies described in Example 5. Guide RNAs used to target genes were as described in Example 5 for each gene. Megakaryocytes and platelets derived from these iPSC KO lines are described as “chassis” platelets or megakaryocyte. Where an experiment describes the use of a chassis platelet, a chassis megakaryocyte could be substituted.

Below are described a set of experiments (in vitro and in vivo) designed to demonstrate the absence of thrombogenic activity in engineered platelets. These assays are well described in the literature and are adapted to the study of chassis platelets.

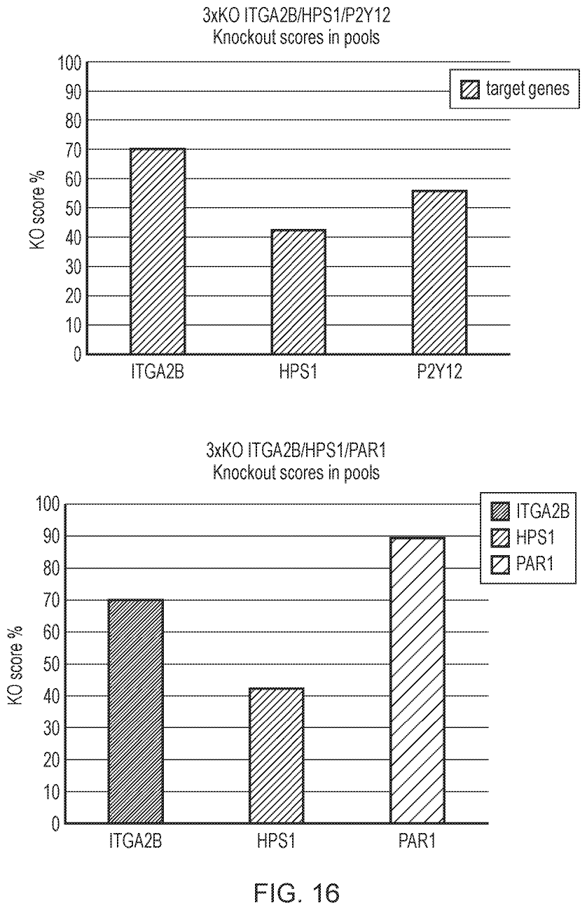

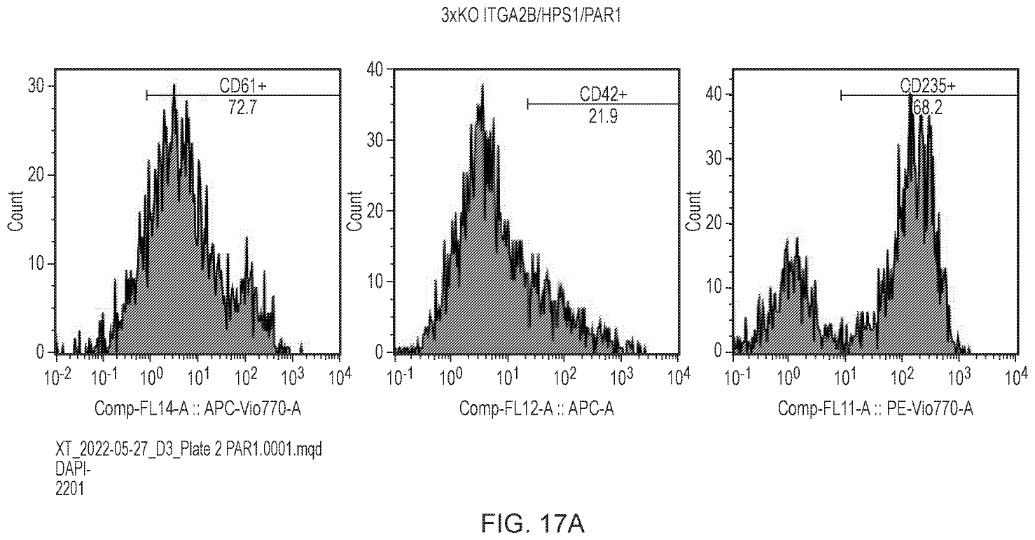

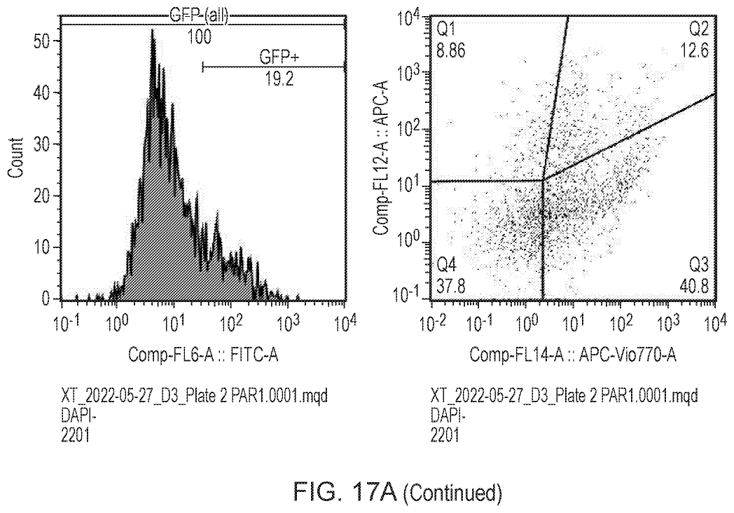

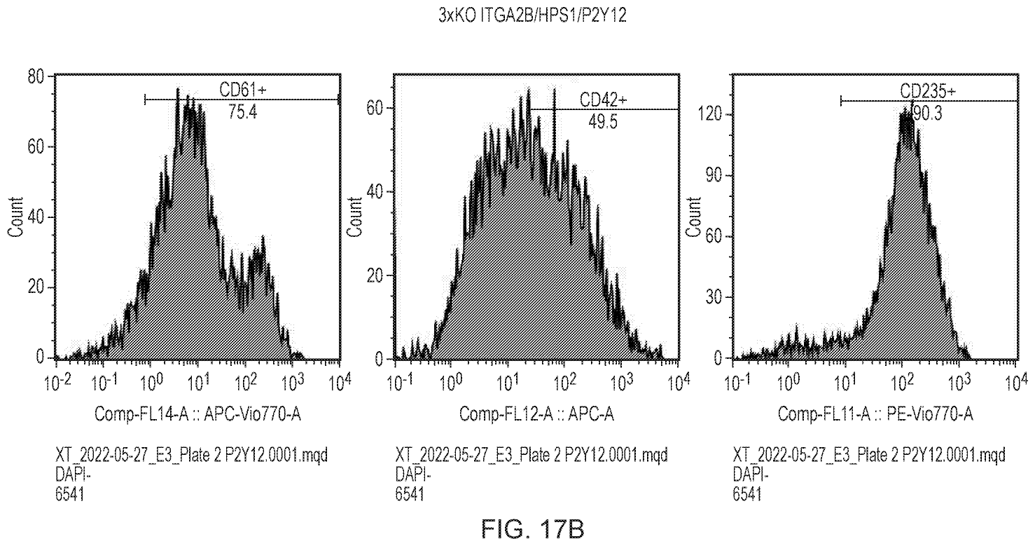

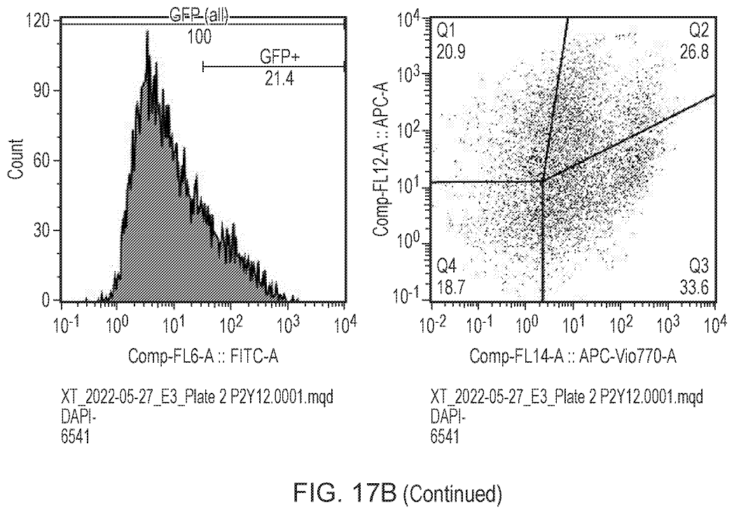

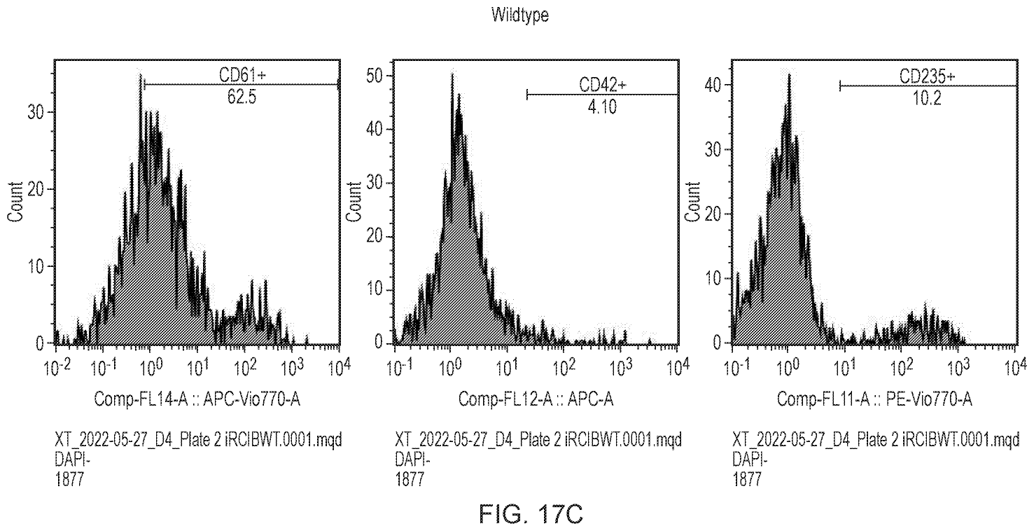

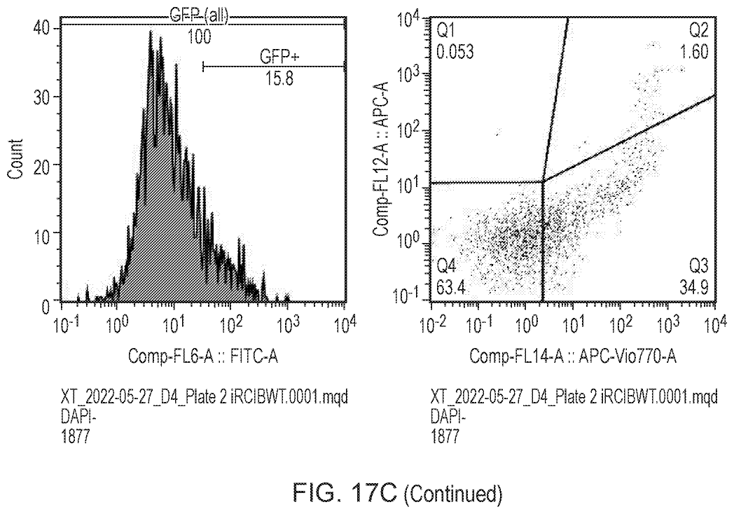

Generation of iPSC pools featuring KOs of the above genes have been generated using previously identified guides (Example 5) (FIG. 16). These knock-out pools have been differentiated using a forward programming approach towards the megakaryocyte phenotype, and their ability to differentiate based on expression of known MK surface markers has been confirmed (FIG. 17).

A. Demonstration that Chassis Platelets do not Respond to Primary/Major Platelet Activation Stimuli In Vitro

Flow chambers coated with either collagen or fibrinogen represent a well-validated method to observe platelet adhesion and activation. Fibrinogen is bound by the gpIIb/IIIa complex, which is disrupted through ITGA2B KO. Platelet containing samples are passed through the chamber and examined for a range of features: (i) visual inspection/counting of binding events by microscopy, (ii) platelet activation status by IF staining of any bound platelets with a P-selectin targeting antibody or by calcium flux, CD40L and annexin V staining. A positive outcome in this experiment is a lack of and/or reduction in chassis platelet binding to the flow chamber in the first instance, and any bound chassis platelets should not be activated.

B. Demonstration that Chassis do not Respond to Agonists/Secondary Messengers In Vitro

In this assay, platelets in suspension are treated with defined dose-escalating platelet mediators (thrombin, ADP) and platelet activation status is measured by staining/flow cytometry for the following markers: CD62p/P-selectin, PF4 release and other markers of activation such as calcium flux, CD40L and annexin V staining if necessary. A positive outcome in this assay is a reduction in and/or absence of activation in chassis platelets. In a control experiment, the capacity for platelet activation (independently of receptor PAR1/P2Y12 activation) is confirmed by treatment of platelets with rhodocytin or podoplanin. These two molecules signal through the platelet Clec2 receptor, which is not a vital receptor for platelet mediated haemostasis, and as such can be left intact on the chassis.

C. Demonstration that Chassis Platelets do not Respond to Platelet Activation Stimuli when Mixed with Normal (Donor) Platelets In Vitro

In this assay, chassis platelets are incubated in the presence of donor derived platelets and the mix is incubated with dose-escalating platelet mediators such as thrombin and ADP. This assay represents a more stringent test for chassis platelets as they are exposed to a combination of soluble mediators of activation and activated normal platelets, hence representing more physiologically relevant conditions. This assay is run in flow chambers, coated with fibrinogen or collagen, and with a flow of 50:50 mix of differentially stained donor derived platelets & chassis platelets through chamber.

Features to be analysed are: (i) fluorescence microscopy for platelet counting, (ii) activation status of bound platelets (e.g. CD62p/P-selectin, PF4 release and other markers of activation such as calcium flux, CD40L and annexin V staining if necessary). The same experimental setup is extended further by co-incubation of chassis platelets with whole blood and flowing the mix through the chamber coated with fibrinogen or collagen.

Fluorescence microscopy of the thrombus demonstrates whether any stained engineered platelets are present and if chassis platelets are entrapped, their activation status is ascertained by IF using P-selectin is the primary marker, or calcium flux, CD40L and annexin V staining as alternative/additional markers.

A positive outcome in this assay is the following:
No significant change in thrombus size when chassis platelet concentration is varied
Absence of or reduced chassis platelet incorporation within the thrombus
If any chassis platelets are captured by the thrombus, they should not be activated
   D. Demonstration that Chassis Platelets do not Contribute to Thrombus Formation In Vivo

The objective of this experiment is to test the phenotypic performance of chassis platelets in an in vivo model of thrombus formation. This provides a deeper level of validation of the approach taken to engineer out the thrombogenic program in chassis platelets.

A mouse model devoid of most of its immune system (e.g. NSG) is required as human platelets are rapidly degraded by mouse macrophages. In addition, antibody mediated depletion of endogenous mouse platelets using a mouse specific anti-CD41 antibody is included to ensure lack of contaminating effect by endogenous platelets. Labelled chassis platelets are injected IV into the mouse, after which laser-mediated cremaster muscle vessel damage is applied locally to stimulate thrombus formation. In vivo fluorescence imaging at the site of injury is used to: (i) measure thrombus size, (ii) chassis platelet incorporation within thrombus.

A positive result will include the following:
The size of the thrombus is not significantly altered by the number of engineered platelets administered
Engineered platelets are not part of the growing thrombus
If engineered platelets do become part of the thrombus, they are not activated (as assayed ex vivo on thrombus removal).
   E. Demonstration that Chassis Platelets do not Impact Normal Hemostatic Capacity

As above, this experiment involves the use of NSG mice and a mouse platelet depletion technique (e.g. infusion with murine CD42 binding monoclonal antibody). Here mice are inoculated with various proportions of chassis platelets and donor platelets. The mouse tail is nicked and the time until the mouse stops bleeding is assayed.

A positive result in this experiment is the following:
Infusion of donor platelets prevents bleeding within a short time frame
Infusion of engineered platelets does not prevent bleeding or prevents bleeding less than donor platelets
Infusion of engineered platelets in the presence of donor platelets does not impact bleeding time vs. donor platelets alone at the same concentration

### 2. Testing of P2M Knock-Out Line Designed for Reduction of Allo-Immunity

As described above (see 1.1 Disruption of expression Beta 2 microglobulin (b₂M) disruption of the β2M gene is expected to be advantageous in some situations. β2M knockout does not impact the differentiation and production of MKs and PLPs from iPSC cells, nor does it impact the phenotype or function.

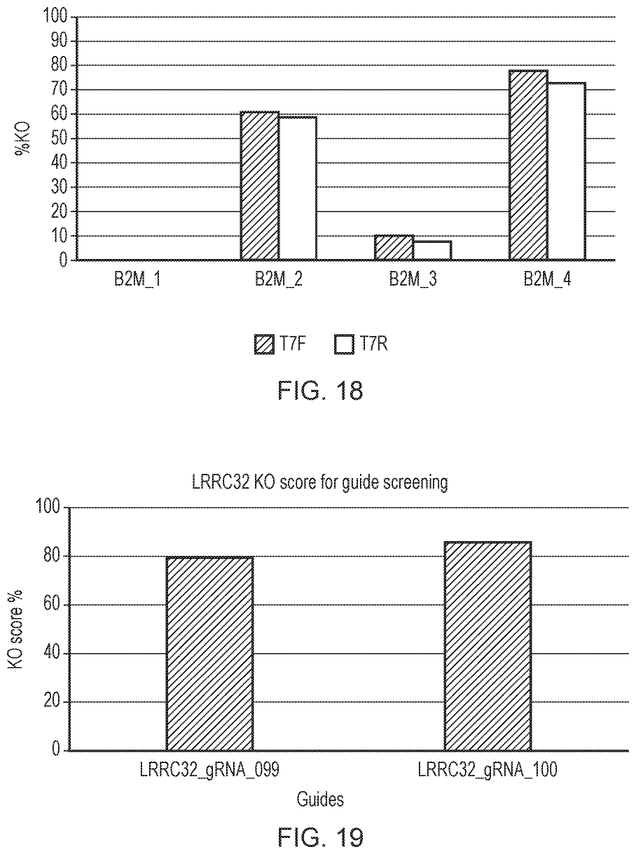

iPSCs featuring knock-outs of the above mentioned genes were generated using methodologies described in Example 5. Guide RNA targeting B2M was designed as described in Example 5, and a high efficiency guide was selected through screening (FIG. 18).

To characterise this β2M knockout, flow cytometry is used to assess absence of β2M and HLA, where the specific HLA expressed on our cells has been determined by genotyping. Antibodies targeted against the specific HLA of our cells, and against β2M, are used to characterise the absence of these proteins after a knockout (Stem Cell Reports 14:49-59).

Characterising reduction of HLA activity is performed by assessing complement dependent cytotoxicity, and antibody-mediated cellular cytotoxicity, where HLA KO is characterised by the reduction of lysis (Mol Med 22: 274-285). Cells are incubated with complement-binding donor-specific anti-HLA antibodies. A non-specific (specific for an HLA not expressed on the cell) is used as a control. Addition of complement only demonstrates lysis of HLA expressing MKs. Alternatively, an antibody-dependent cellular cytotoxicity kit is used with specific antibodies targeted against the HLA of our iPSC. β2M KO reduces the lysis potential of ADCC by reduction of HLA expression.

### 3. Testing of GARP Knock-Out Line Designed for Reduction of Mature TGF1 Release Upon Chassis Platelet Activation

Platelets are the dominant source of the pro-mitogenic TGFβ protein systemically as well as in the tumour microenvironment. Platelets express surface Glycoprotein-A Repetitions Predominant Protein (GARP), a receptor for Latent Transforming Growth Factor Beta (LTGFβ). In LTGFβ, the mature TGFβ protein is bound to the latency-associated peptide (LAP) and is thereby prevented from binding to the TGF-beta receptor. Upon degranulation, activated platelets dramatically upregulate GARP and convert bound LTGFβ into the mature TGFβ. It has been shown that platelet-specific deletion of GARP blunted TGFβ activity in the tumour microenvironment and boosted protective immunity against pre-established cancers (Metelli, A. et al. (2017) J Immunol May 1, 198 (1 Supplement) 126.17)

iPSCs featuring knock-outs of the above mentioned genes were generated using methodologies described in Example 5. Guide RNA targeting LRRC32 was designed as described in Example 5, and a high efficiency guide was selected through screening (FIG. 19).

LRRC32 knockout does not impact the differentiation and production of MKs and PLPs from iPSC cells, nor does it impact the phenotype or function (PLoS ONE 12(3): e0173329).

LRRC32 knockout is characterised by a reduction of GARP, assessed by flow cytometry using a GARP specific antibody to demonstrate absence of GARP protein on the MK and platelet surface.

The function of a GARP knockout manifests by reduction of TGFB binding. This is determined by measurement of TGFbeta bound to WT and KO platelets. TGFbeta is incubated with WT and KO platelets, with successful KO characterised by a reduction of TGFB binding. Binding of TGFbeta to platelets is characterised by flow cytometry or ELISA.

To assess whether the GARP KO can reduce TGFbeta immune cell inhibition, platelets are incubated with T-cells to demonstrate a reduction of activity caused by bound TGFbeta. Reduction of activation is measured by cytokine release such as IFNg (Sci Immunol 2(11):eaai7911). Knockout of GARP reduces TGFbeta binding capacity, thus reducing the inhibition observed from platelets.

### 4. Testing of Exosomal RNA Targeting

Platelet alpha-granules contain exosomes. Exosomes are small vesicles, ˜50-200 nm in size, and can contain nucleic acids as cargo. Exosome producing cells have been generated previously.

To drive exogenous RNA loading to the megakaryocyte and thus platelet exosome, an exosome resident protein can be engineered as fusions with RNA hairpin binding proteins. Two examples of RNA hairpin binding proteins are L7Ae and MS2 (schematic shown in FIG. 23A). Initially, correct, granular localisation of a known exosome resident protein (BASP1) fused to L7Ae was confirmed in megakaryocytes (FIG. 23B. To show RNA co-localisation, two different RNA constructs are tested—one featuring a set of hairpins and one not featuring hairpins. Localisation of the RNA is confirmed by RNA FISH, where co-localisation of hairpin containing RNA to the fluorescently labelled BASP1-L7Ae protein occurs.

Minimal BASP1_mCherry_L7Ae
[SEQ ID NO: 118]
MGGKLSKKKGGSGGGSGGGSG
VSKGEAVIKEFMRFKVHMEGSMNGHEFEIEGEG
EGRPYEGTQTAKLKVTKGGPLPFSWDILSPQFM
YGSRAFTKHPADIPDYYKQSFPEGFKWERVMNF
EDGGAVTVTQDTSLEDGTLIYKVKLRGTNFPPD
GPVMQKKTMGWEASTERLYPEDGVLKGDIKMAL
RLKDGGRYLADFKTTYKAKKPVQMPGAYNVDRK
LDITSHNEDYTVVEQYERSEGRHSTGGMDELYK
GGSGGGSGGGSGMYVRFEVPEDMQNEALSLLEK
VRESGKVKKGTNETTKAVERGLAKLVYIAEDVD
PPEIVAHLPLLCEEKNVPYIYVKSKNDLGRAVG
IEVPCASAAIINEGELRKELGSLVEKIKGLQK
Minimal BASP1
(GGSG)x3 Linker
mScarlet
(GGSG)x3 Linker
L7Ae

Once correct localisation of the RNA and exosome directed loading construct is shown in platelets and megakaryocytes, release of exosomes from platelets and megakaryocytes upon activation investigated. Platelets and megakaryocytes are activated using known agonists (e.g. PMA, CRP/TRAP-6/ADP), which drives alpha-granule exocytosis (otherwise referred to as degranulation). Exosomes are collected through either commercially available kits or centrifugation based approaches. RNA i extracted from the exosomes, and is assayed by qPCR for the presence or absence of exogenous RNA. As negative controls, untargeted (i.e. hairpin negative) RNA i used, and hairpin containing RNA in a megakaryocyte/platelet background not expressing BASP-L7Ae is also tested. This confirms exosome specific loading of RNA into megakaryocyte and platelet alpha-granules, and the ability of platelets and megakaryocytes to conditionally release these loaded exosomes in response to some agonist single.

To demonstrate the ability of these loaded exosomes to drive gene expression in some nearby cell, the RNA loaded by our hairpin targeting approach features a reporter gene ORF (e.g. GFP). Upon activation of platelets or megakaryocytes in the vicinity of a target cell, the reporter RNA containing exosomes are taken up by the nearby cell after their release from platelet or megakaryocyte alpha-granules. By performing flow cytometric analysis, target cells are gated based on specific cell surface marker expression, and then the level of reporter gene expression is measured. Reporter gene expression only increases in target cells if the exogenous RNA was appropriately targeted to the megakaryocyte or platelet exosome compartment, and only upon activation of the platelet or megakaryocyte.

5. Antigen Specific T Cell Targeting with Platelet MHC-B2M-CAR

Fusion of MHC, B2M, antigen derived peptide and internal platelet CAR signalling domain into a single protein allows for targeting of cargo containing synlets to antigen specific T Cells. To test this hypothesis, proof of concept peptide-MHC-B2M-CAR constructs (MHC-CAR) were engineered, targeting MART1 and NY-ESO-1 in the context HLA-A*02. Different variants were generated, utilising distinct transmembrane domain regions and internal signalling regions.

MART1_B2M_HLA-A*0201_FCERG-TM_FCERG-CYTO
[SEQ ID NO: 104]
MIPAVVLLLLLLVEQAAAELAGIGILTVGCGGSGGGGSGGGGS
IQRTPKIQVYSRHPAENGKSNFLNCYVSGFHPSDIEVDLLKNG
ERIEKVEHSDLSFSKDWSFYLLYYTEFTPTEKDEYACRVNHVT
LSQPKIVKWDRDMGGGGSGGGGSGGGGSGGGGSGSHSMRYFFT
SVSRPGRGEPRFIAVGYVDDTQFVRFDSDSAAQRMEPRAPWIE
QEGPEYWDGETRKVKAHSQTHRVDLGTLRGCYNQSEAGSHTVQ
RMYGCDVGSDWRFLRGYHQYAYDGKDYIALKEDLRSWTAADMA
AQTTKHKWEAAHVAEQLRAYLEGTCVEWLRRYLENGKETLQRT
DAPKTHMTHHAVSDHEATLRCWALSFYPAEITLTWQRDGEDQT
QDTELVETRPAGDGTFQKWAAVVVPSGQEQRYTCHVQHEGLPK
PLTLRWEPSSQPTIPILGEPQLCYILDAILFLYGIVLTLLYCR
LKIQVRKAAITSYEKSDGVYTGLSTRNQETYETLKHEKPPQ
FCERG Signal Peptide
MART1 peptide antigen
GCGGS(G4S)2 Linker
B2M
(G4S)4 Linker
HLA-A*0201
FCERG-TM
FCERG-CYTO
MART1 B2M HLA-A*0201 FCGR2A-TM FCGR2A-CYTO
[SEQ ID NO: 105]
MIPAVVLLLLLLVEQAAAELAGIGILTVGCGGSGGGGSGGGGS
IQRTPKIQVYSRHPÅENGKSNFLNCYVSGFHPSDIEVDLLKNG
ERIEKVEHSDLSFSKDWSFYLLYYTEFTPTEKDEYACRVNHVT
LSQPKIVKWDRDMGGGGSGGGGSGGGGSGGGGSGSHSMRYFFT
SVSRPGRGEPRFIAVGYVDDTQFVRFDSDAASQRMEPRAPWIE
QEGPEYWDGETRKVKAHSQTHRVDLGTLRGCYNQSEAGSHTVQ
RMYGCDVGSDWRFLRGYHQYAYDGKDYIALKEDLRSWTAADMA
AQTTKHKWEAAHVAEQLRAYLEGTCVEWLRRYLENGKETLQRT
DAPKTHMTHHAVSDHEATLRCWALSFYPAEITLTWQRDGEDQT
QDTELVETRPAGDGTFQKWAAVVVPSGQEQRYTCHVQHEGLPK
PLTLRWEPSSQPTIPISSPMGIIVAVVIATAVAAIVAAVVALI
YCRKKRISANSTDPVKAAQFEPPGRQMIAIRKRQLEETNNDYE
TADGGYMTLNPRAPTDDDKNIYLTLPPNDHVNSNN
FCERG Signal Peptide
MART1 peptide antigen
GCGGS(G4S)2 Linker
B2M
(G4S)4 Linker
HLA-A*0201
FCGR2A-TM
FCGRZA-CYTO
MART1 B2M HLA-A*0201 HLA-TM FCERG-CYTO
[SEQ ID NO: 106]
MIPAVVLLLLLLVEQAAAELAGIGILTVGCGGSGGGGSGGGGS
IQRTPKIQVYSRHPAENGKSNFLNCYVSGFHPSDIEVDLLKNG
ERIEKVEHSDLSFSKDWSFYLLYYTEFTPTEKDEYACRVNHVT
LSQPKIVKWDRDMGGGGSGGGGSGGGGSGGGGSGSHSMRYFFT
SVSRPGRGEPRFIAVGYVDDTQFVRFDSDAASQRMEPRAPWIE
QEGPEYWGETRKVKAHSQTHRVDLGTLRGCYNQSEAGSHTVQR
MYGCDVGSDDWRFLRGYHQYAYDGKDYIALKEDLRSWTAADMA
AQTTKHKWEAAHVAEQLRAYLEGTCVEWLRRYLENGKETLQRT
DAPKTHMTHHAVSDHEATLRCWALSFYPAEITLTWQRDGEDQT
QDTELVETRPAGDGTFQKWAAVVVPSGQEQRYTCHVQHEGLPK
PLTLRWEPSSQPTIPIVGIIAGLVLFGAVITGAVVAAVMWRRK
SSGGEGVKDRKGGSYTQAASSDSAQGSDVSLTACKVRLKIQVR
KAAITSYEKSDGVYTGLSTRNQETYETLKHEKPPQ
FCERG Signal Peptide
MART1 peptide antigen
GCGGS(G4S)2 Linker
B2M
(G4S)4 Linker
HLA-A*0201
HLA-A*0201-TM
FCERG-CYTO
MART1 B2M HLA-A*0201 HLA-TM FCGR2A-CYTO
[SEQ ID NO: 107]
MIPAVVLLLLLLVEQAAAELAGIGILTVGCGGSGGGGSGGGGS
IQRTPKIQVYSRHPAENGKSNFLNCYVSGFHPSDIEVDLLKNG
ERIEKVEHSDLSFSKDWSFYLLYYTEFTPTEKDEYACRVNHVT
LSQPKIVKWDRDMGGGGSGGGGSGGGGSGGGGSGSHSMRYFFT
SVSRPGRGEPRFIAVGYVDDTQFVRFDSDAASQRMEPRAPWIE
QEGPEYWDGETRKVKAHSQTHRVDLGTLRGCYNQSEAGSHTVQ
RMYGCDVGSDWRFLRGYHQYAYDGKDYIALKEDLRSWTAADMA
AQTTKHKWEAAHVAEQLRAYLEGTCVEWLRRYLENGKETLQRT
DAPKTHMTHHAVSDHEATLRCWALSFYPAEITLTWQRDGEDQT
QDTELVETRPAGDGTFQKWAAVVVPSGQEQRYTCHVQHEGLPK
PLTLRWEPSSQPTIPIVGIIAGLVLFGAVITGAVVAAVMWRRK
SSGGEGVKDRKGGSYTQAASSDSAQGSDVSLTACKVCRKKRIS
ANSTDPVKAAQFEPPGRQMIAIRKRQLEETNNDYETADGGYMT
LNPRAPTDDDKNIYLTLPPNDHVNSNN
FCERG Signal Peptide
MART1 peptide antigen
GCGGS(G4S)2 Linker
B2M
(G4S)4 Linker
HLA-A*0201
HLA-A*0201-TM
FCGR2A-CYTO
NYESO1 B2M HLA-A*0201 FCERG-TM FCERG-CYTO
[SEQ ID NO: 108]
MIPAVVLLLLLLVEQAAASLLMWITQCGCGGSGGGGSGGGGSI
QRTPKIQVYSRHPAENGKSNFLNCYVSGFHPSDIEVDLLKNGE
RIEKVEHSDLSFSKDWSFYLLYYTEFTPTEKDEYACRVNHVTL
SQPKIVKWDRDMGGGGSGGGGSGGGGSGGGGSGSHSMRYFFTS
VSRPGRGEPRFIAVGYVDDTQFVRFDSDAASQRMEPRAPWIEQ
EGPEYWDGETRKVKAHSQTHRVDLGTLRGCYNQSEAGSHTVQR
MYGCDVGSDWRFLRGYHQYAYDGKDYIALKEDLRSWTAADMAA
QTTKHKWEAAHVAEQLRAYLEGTCVEWLRRYLENGKETLQRTD
APKTHMTHHAVSDHEATLRCWALSFYPAEITLTWQRDGEDQTQ
DTELVETRPAGDGTFQKWAAVVVPSGQEQRYTCHVQHEGLPKP
LTLRWEPSSQPTIPILGEPQLCYILDAILFLYGIVLTLLYCRL
KIQVRKAAITSYEKSDGVYTGLSTRNQETYETLKHEKPPQ
FCERG Signal Peptide
NY-ESO-1 peptide antigen
GCGGS(G4S)2 Linker
B2M
(G4S)4 Linker
HLA-A*0201
FCERG-TM
FCERG-CYTO
NYESO1 B2M HLA-A*0201 FCGR2A-TM FCGR2A-CYTO
[SEQ ID NO: 109]
MIPAVVLLLLLLVEQAAASLLMWITQCGCGGSGGGGSGGGGSI
QRTPKIQVYSRHPAENGKSNFLNCYVSGFHPSDIEVDLLKNGE
RIEKVEHSDLSFSKDWSFYLLYYTEFTPTEKDEYACRVNHVTL
SQPKIVKWDRDMGGGGSGGGGSGGGGSGGGGSGSHSMRYFFTS
VSRPGRGEPRFIAVGYVDDTQFVRFDSDAASQRMEPRAPWIEQ
EGPEYWDGETRKVKAHSQTHRVDLGTLRGCYNQSEAGSHTVQR
MYGCDVGSDWRFLRGYHQYAYDGKDYIALKEDLRSWTAADMAA
QTTKHKWEAAHVAEQLRAYLEGTCVEWLRRYLENGKETLQRTD
APKTHMTHHAVSDHEATLRCWALSFYPAEITLTWQRDGEDQTQ
DTELVETRPAGDGTFQKWAAVVVPSGQEQRYTCHVQHEGLPKP
LTLRWEPSSQPTIPISSPMGIIVAVVIATAVAAIVAAVVALIY
CRKKRISANSTDPVKAAQFEPPGRQMIAIRKRQLEETNNDYET
ADGGYMTLNPRAPTDDDKNIYLTLPPNDHVNSNN
FCERG Signal Peptide
NY-ESO-1 peptide antigen
GCGGS(G4S)2 Linker
B2M
(G4S)4 Linker
HLA-A*0201
FCGR2A-TM
FCGR2A-CYTO
NYESO1 B2M HLA-A*0201 HLA-TM FCERG-CYTO
[SEQ ID NO: 110]
MIPAVVLLLLLLVEQAAASLLMWITQCGCGGSGGGGSGGGGSI
QRTPKIQVYSRHPAENGKSNFLNCYVSGFHPSDIEVDLLKNGE
RIEKVEHSDLSFSKDWSFYLLYYTEFTPTEKDEYACRVNHVTL
SQPKIVKWDRDMGGGGSGGGGSGGGGSGGGGSGSHSMRYFFTS
VSRPGRGEPRFIAVGYVDDTQFVREDSDAASQRMEPRAPWIEQ
EGPEYWDGETRKVKAHSQTHRVDLGTLRGCYNQSEAGSHTVQR
MYGCDVGSDWRFLRGYHQYAYDGKDYIALKEDLRSWTAADMAA
QTTKHKWEAAHVAEQLRAYLEGTCVEWLRRYLENGKETLQRTD
APKTHMTHHAVSDHEATLRCWALSFYPAEITLTWQRDGEDQTQ
DTELVETRPAGDGTFQKWAAVVVPSGQEQRYTCHVQHEGLPKP
LTLRWEPSSQPTIPIVGIIAGLVLFGAVITGAVVAAVMWRRKS
SGGEGVKDRKGGSYTQAASSDSAQGSDVSLTACKVRLKIQVRK
AAITSYEKSDGVYTGLSTRNQETYETLKHEKPPQ
FCERG Signal Peptide
NY-ESO-1 peptide antigen
GCGGS(G4S)2 Linker
B2M
(G4S)4 Linker
HLA-A*0201
HLA-A*0201-TM
FCERG-CYTO
NYESO1 B2M HLA-A*0201 HLA-TM FCGR2A-CYTO
[SEQ ID NO: 111]
MIPAVVLLLLLLVEQAAASLLMWITQCGCGGSGGGGSGGGGSI
QRTPKIQVYSRHPAENGKSNFLNCYVSGFHPSDIEVDLLKNGE
RIEKVEHSDLSFSKDWSFYLLYYTEFTPTEKDEYACRVNHVTL
SQPKIVKWDRDMGGGGSGGGGSGGGGSGGGGSGSHSMRYFFTS
VSRPGRGEPRFIAVGYVDDTQFVRFDSDAASQRMEPRAPWIEQ
EGPEYWDGETRKVKAHSQTHRVDLGTLRGCYNQSEAGSHTVQR
MYGCDVGSDWRFLRGYHQYAYDGKDYIALKEDLRSWTAADMAA
QTTKHKWEAAHVAEQLRAYLEGTCVEWLRRYLENGKETLQRTD
APKTHMTHHAVSDHEATLRCWALSFYPAEITLTWQRDGEDQTQ
DTELVETRPAGDGTFQKWAAVVVPSGQEQRYTCHVQHEGLPKP
LTLRWEPSSQPTIPIVGIIAGLVLFGAVITGAVVAAVMWRRKS
SGGEGVKDRKGGSYTQAASSDSAQGSDVSLTACKVCRKKRISA
NSTDPVKAAQFEPPGRQMIAIRKRQLEETNNDYETADGGYMTL
NPRAPTDDDKNIYLTLPPNDHVNSNN
FCERG Signal Peptide
NY-ESO-1 peptide antigen
GCGGS(G4S)2 Linker
B2M
(G4S)4 Linker
HLA-A*0201
HLA-A*0201-TM
FCGR2A-CYTO

To initially assay for expression of the MHC-CARs, they are inserted into B2M KO iPSC and MK cell lines. Surface expression of the MHC-CAR is confirmed through FACS based measurement of B2M surface expression (which, in the absence of MHC-CAR containing B2M is absent in the B2M KO background). To further confirm functional expression of the MHC-CAR, recombinant TCRs and antibodies raised specifically against the peptide-MHC are used too.

Following introduction of MHC-CAR and confirmation of expression in iPSCs, megakaryocytes and platelets, functionality of the car is investigated. Jurkat or some other T cell line, expressing a TCR which is known to target the MHC-CAR is mixed with platelets or megakaryocytes expressing MHC-CAR. Platelet activation is assayed upon binding to T cells expressing the target TCR, through exposure of P-selectin (or some other degranulation dependent surface marker) on platelets surface as measured by FACS or through ELISA based detection of platelet cargo release.

MHC-CAR expressing Synlets are loaded with different cargoes, and then assayed for the ability of those cargoes to stimulate TCR expressing T cell activation status (through e.g. ELISA based measurement of T cell cytokine production, T cell proliferation, T cell FACs for known markers of activation and genetic approaches such as measurement of T cell NFAT reporter gene activation as measured by microscopy or FACs).

The invention also provides a number of specific embodiments, described in paragraph [0287]-[353] of PCT/GB2020/053247 which is hereby incorporated by reference.

The invention also provides the following embodiments presented as numbered paragraphs.

1. A chimeric platelet receptor (CPR) wherein the receptor comprises:
a) an intracellular domain that is a platelet modulation domain; and
b) a heterologous target binding domain that recognizes and binds a target.

2. The CPR of paragraph 1 wherein the target binding domain binds to a target that is endogenous to a subject, optionally wherein the target is a human target.

3. The CPR of any of the preceding paragraphs wherein the target is present on a cell surface or a tissue surface.

4. The CPR of any of the preceding paragraphs wherein the target is a target such that when the CPR is present in a platelet membrane, after binding of the target to the target binding domain the CPRs cluster on the plasma membrane.

5. The CPR according to any the preceding paragraphs wherein when the CPR is present in a platelet membrane, after binding of the target to the target binding domain the platelet modulation domain is activated.

6. The CPR according to any the preceding paragraphs wherein when the CPR is present in a platelet plasma membrane, after binding of the target binding domain to the target the CPRs cluster on the surface of the platelet plasma membrane, wherein said clustering is sufficient to activate the platelet modulation domain.

7. The CPR according to any of the preceding paragraphs wherein the target binding domain comprises a human target binding domain sequence or a sequence that has at least 75%, 80%, 85%, 90%, 92%, 94%, 96%, 98% or 100% sequence identity to a human target binding domain sequence.

8. The CPR according to any of the preceding paragraphs wherein the target binding domain comprises a non-human target binding domain sequence, optionally:
a humanised sequence; or
a sequence from a mouse.

9. The CPR according to any of the preceding paragraphs wherein said target binding domain comprises a target-binding ligand or fragment thereof that binds specifically to said target.

11. The CPR according to any of the preceding paragraphs wherein said target binding domain comprises an antibody or antibody fragment that binds specifically to said target.

12. The CPR according to any of the preceding paragraphs wherein said target binding domain comprises a variable heavy chain domain and/or a variable light chain domain, optionally an scFV.

13. The CPR according to any of the preceding paragraphs wherein the target is a tumor antigen, neoantigen or autoantigen.

14. The CPR according to any of the preceding paragraphs wherein the target is:
a) an antigen associated with a disease, disorder or condition; and/or
b) on a target tissue or cell in the body of a subject, optionally wherein the target tissue or cell is a cancer tissue or cell; and/or
c) an autoimmune B cell.

15. The CPR according to any of the preceding paragraphs wherein the target binding domain comprises at least one of:
a) FCERG EC domain, CLEC1 EC domain, FCGR2 EC domain, GPVIA EC domain, CEACAM1 EC domain, G6b-B EC domain, LILRB2 EC domain, PECAM1 EC domain TLT1 EC domain and/or a sequence that has at least 75%, 80%, 85%, 90%, 92%, 94%, 96%, 98% or 100% sequence identity to a FCERG EC domain, CLEC1 EC domain,FCGR2 EC domain, GPVIA EC domain, CEACAM1 EC domain, G6b-B EC domain, LILRB2 EC domain, PECAM1 EC domain and/or TLT1 EC domain; and/or
b) the target binding domain comprises any one or more of the domains or portions thereof set out on page 46 to 49 of PCT/GB2020/053247 which is hereby incorporated by reference, or a sequence that has at least 75%, 80%, 85%, 90%, 92%, 94%, 96%, 98% or 100% sequence identity to any one or more of the domains or portions thereof set out on page 46 to 49 of PCT/GB2020/053247 which is hereby incorporated by reference.

16. The CPR according to any of the preceding paragraphs wherein the target binding domain comprises a peptide associated with autoimmunity, optionally:
a peptide or portion of any one or more of the following proteins: MOG, GAD65, MAG, PMP22, TPO, VGKC, PLP, AChR, TRIB2, NMDA, GluR, GAD2, ARMC9, CYP21A2, CASR, NASP, insulin, TSHR, thyroperoxidase, asioglycoprotein receptor, CYP2D6, LF, TTG, H/K ATP-ase, Factor XIII, Beta2-GPI, ITGB2, G-CSF, GP IIb/IIa, COLII, FBG beta alpha, MPO, CYO, PRTN3, TGM, COLVII, COIL, DSG1, DSG3, SOX10, 70SNRNP70, SAG and a3(IV)NC1 collagen; or
a peptide or portion that has at least 75%, 80%, 85%, 90%, 92%, 94%, 96%, 98% or 100% sequence identity to any one or more of the following proteins: MOG, GAD65, MAG, PMP22, TPO, VGKC, PLP, AChR, TRIB2, NMDA, GluR, GAD2, ARMC9, CYP21A2, CASR, NASP, insulin, TSHR, thyroperoxidase, asioglycoprotein receptor, CYP2D6, LF, TTG, H/K ATP-ase, Factor XIII, Beta2-GPI, ITGB2, G-CSF, GP IIb/IIa, COLII, FBG beta alpha, MPO, CYO, PRTN3, TGM, COLVII, COIL, DSG1, DSG3, SOX10, 70SNRNP70, SAG and a3(IV)NC1 collagen.

17. The CPR according to any of the preceding paragraphs wherein the target binding domain binds to a target that is:
a) an endogenous target that is found on a tissue in the body of a subject or on a cell or in a particular location of a subject;
b) present on tissue, or on a particular subset of tissue, or in plasma or blood of a subject, optional in a human subject optionally in the blood;
c) only presented during one or more disease states, optionally the target is a neoantigen that arises in a tumour cell;
d) only present in significant amounts optionally present in abnormal levels on a tissue or cell that does not normally express the target and/or is only present in a localised manner during or more disease states;
e) an antigen, optionally a tumour neoantigen or a tumour specific antigen;
f) CD19;
g) a cytokine receptor;
h) not collagen;
i) an artificial or exogenous target;
j) a designer drug;
k) a drug that has been designed using DREADD;
l) a protein selected from Table 2 on pages 23-31 of PCT/GB2020/053247 which is hereby incorporated by reference;
m) CD276; and/or
n) I12, KLK, amyloid, a Notch receptor and/or OLR1.

18. The CPR according to any of the preceding paragraphs wherein the target binding domain:
a) is an antibody or antigen binding fragment thereof;
b) comprises a variable heavy chain domain of an antibody and/or a variable light chain domain of an antibody; and/or
c) comprises a kappa light chain or a fragment thereof targeting.

19. The CPR according to any of the preceding paragraphs wherein the target binding domain comprises a portion of a protein or peptide associated with autoimmunity

20. The CPR according to any of the preceding paragraphs wherein the platelet modulation domain is a platelet activation domain, optionally an ITAM comprising domain, optionally a platelet ITAM comprising domain, optionally is domain that has at least 75%, 80%, 85%, 90%, 92%, 94%, 96%, 98% or 100% sequence identity to an ITAM comprising domain optionally a platelet ITAM comprising domain.

21. The CPR of any of the preceding paragraphs wherein when the CPR is present in the membrane of a platelet, when activated, the platelet activation domain:
a) results in degranulation of the platelet;
b) results in the release of contents from the platelet;
c) results in the presence of intraplatelet contents on the plasma membrane of the platelet;
d) results in the release of extracellular vesicles via blebbing from the plasma membrane; and/or
e) results in a change of shape of the platelet from a biconcave disk to fully spread cell fragments.

22. The CPR of any of the preceding paragraphs wherein the platelet activation domain is a platelet degranulation triggering domain.

23. The CPR according to any of the preceding paragraphs wherein the platelet modulation domain is an inhibition of platelet activation domain that prevents activation of a platelet, optionally wherein the inhibition of platelet activation domain is an mM comprising domain, optionally is a domain that has at least 75%, 80%, 85%, 90%, 92%, 94%, 96%, 98% or 100% sequence identity to an MM comprising domain.

24. The CPR of any of the preceding paragraphs wherein when the CPR is present in the membrane of a platelet, and when the inhibition of platelet activation domain is activated, the platelet inhibition of activation domain:
a) prevents degranulation of the platelet;
b) prevents the release of contents from the platelet;
c) prevents the presence of intracellular contents on the plasma membrane of the platelet; d) prevents the release of extracellular vesicles via blebbing from the plasma membrane; and/or
e) prevents a change of shape of the platelet from a biconcave disk to fully spread cell fragments.

25. The CPR of any of the preceding paragraphs wherein the platelet activation domain is a platelet degranulation triggering domain.

26. The CPR according to any of the preceding paragraphs wherein the platelet modulation domain comprises a human modulation domain sequence.

27. The CPR according to any of the preceding paragraphs wherein the platelet modulation domain comprises a non-human modulation domain sequence, optionally a sequence from a mouse.

28. The CPR according to any of the preceding paragraphs wherein the platelet modulation domain is endogenous to the progenitor, producer or effector-chassis that the CPR is to be used with, optionally wherein the platelet modulation domain is endogenous to an iPSC, a megakaryocyte or a platelet.

29. The CPR according to any of the preceding paragraphs wherein the platelet modulation domain does not comprise domains from an immunoreceptor tyrosine based activation motif (ITAM) receptor, optionally does not comprise one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2).

30. The CPR according to any of the preceding paragraphs wherein when the CPR is localised to a platelet plasma membrane, after binding of the target to the target binding domain, the platelet modulation domain triggers degranulation of the platelet.

31. The CPR according to any the preceding paragraphs wherein the platelet modulation domain is a platelet degranulation triggering domain and comprises:
one or more domains from an immunoreceptor tyrosine based activation motif (ITAM) receptor, optionally comprises one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2); or
a domain that has at least 75%, 80%, 85%, 90%, 92%, 94%, 96%, 98% or 100% sequence identity to an ITAM comprising domain, optionally a platelet ITAM comprising domain, optionally has at least 75%, 80%, 85%, 90%, 92%, 94%, 96%, 98% or 100% sequence identity to one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2).

32. The CPR according to any the preceding paragraphs wherein the platelet modulation domain is an inhibition of platelet activation domain that inhibits triggering of platelet degranulation and comprises one or more mM motifs, optionally wherein the one or more ITIM motifs is an mM motif from PECAM1, TLT1, LILRB2, CEACAM1 or G6b-B, optionally wherein the ITIM domain from:
LILRB2 is SEQ ID NO: 34 shown in Table 5 on page 44 of PCT/GB2020/053247 which is hereby incorporated by reference
PECAM1 is SEQ ID NO: 38 shown in Table 5 on page 44 of PCT/GB2020/053247 which is hereby incorporated by reference
CEACAM1 is SEQ ID NO: 24 shown in Table 5 on page 44 of PCT/GB2020/053247 which is hereby incorporated by reference.

33. The CPR according to any the preceding paragraphs wherein the platelet modulation domain comprises one or more mutations, insertions or deletions relative to a native platelet modulation domain sequence.

34. The CPR according to any the preceding paragraphs wherein the one or more mutations, insertions or deletions relative to the native modulation domain sequence increases the sensitivity of the CPR relative to a CPR that comprises a platelet modulation domain that does not comprise the one or more mutations.

35. The CPR according to any the preceding paragraphs wherein the one or more mutations, insertions or deletions relative to the native modulation domain sequence decreases the sensitivity of the CPR relative to a CPR that comprises a platelet modulation domain that does not comprise the one or more mutations.

36. The CPR according to any the preceding paragraphs wherein the platelet modulation domain comprises at least 75%, 80%, 85%, 90%, 92%, 94%, 96%, 98% or 100% sequence identity to a naturally occurring platelet modulation domain.

37. The CPR according to any the preceding paragraphs further comprising a signal peptide and/or linker sequence, optionally wherein;
the signal peptide comprises or consists of a portion of the sequences set out in Table 1; and/or
the signal peptide comprises or consists of a portion of any of the sequences in Table 7 on page 46 of PCT/GB2020/053247 which is hereby incorporated by reference; and/or optionally wherein the linker comprises or consists of the linkers or portions thereof as set out on page 51 of PCT/GB2020/053247 which is hereby incorporated by reference.

38. The CPR according to any the preceding paragraphs further comprising a transmembrane domain, optionally wherein the transmembrane domain comprises or consists of any one or more of the transmembrane domains or portions thereof as set out on page 49-50 of PCT/GB2020/053247 which is hereby incorporated by reference.

39. The CPR according to any the preceding paragraphs wherein the CPR comprises an intracellular domain that comprises or consists of the intracellular domains or a portion thereof as set out on page 50 and 51 of PCT/GB2020/053247 which is hereby incorporated by reference.

40. The CPR according to any the preceding paragraphs wherein the CPR, comprises or consists of a combination of domains as set out on pages 41-63 of PCT/GB2020/053247 which is hereby incorporated by reference.

41. A universal chimeric platelet receptor wherein the receptor comprises:
a) an intracellular domain that is a platelet modulation domain; and
b) a heterologous tag binding domain.

42. A tagged targeting peptide comprising a tag and a target binding domain, optionally wherein the tagged targeting peptide is a soluble peptide.

43. The tagged targeting peptide of paragraph 43 wherein the tag is a leucine zipper.

44. The universal CPR of paragraph 41 wherein the heterologous tag binding domain binds to a tag present on a tagged targeting peptide, wherein the tagged targeting peptide comprises the tag and a target binding domain, and
wherein when the Universal CPR is located in a platelet plasma membrane, binding of the targeting peptide to the universal CPR in the absence of simultaneous binding of the target binding domain to the target is not sufficient to activate the platelet modulation domain.

45. The universal CPR of paragraph 41 or 44 wherein the heterologous tag binding domain binds to a tag present on a targeting peptide, wherein when the Universal CPR is located in a platelet plasma membrane binding of the targeting peptide to the universal CPR in the absence of simultaneous binding of the tagged target binding domain to the target does not cause sufficient receptor clustering to lead to activation of the platelet modulation domain.

46. The universal CPR of any of the preceding paragraphs 2 wherein the tagged targeting peptide is a soluble peptide.

47. The Universal CPR of any of the preceding paragraphs wherein the heterologous tag binding domain comprises a leucine zipper.

48. A complex comprising the universal CPR according to any of paragraphs 41, and 44-47 and the targeting peptide according to any of paragraphs 42 or 43, wherein the universal CPR is bound to the corresponding tag on the tagged targeting peptide via the heterologous tag binding domain.

49. The tagged targeting peptide according to any of paragraphs 42 or 43 or complex according to paragraph 48 wherein the target binding domain binds to a target that is endogenous to the intended subject, optionally wherein the target is a human target.

50. The tagged targeting peptide according to any of paragraphs 42, 43 or 49, or complex according to any of paragraphs 48 or 49 wherein the target is present on a cell surface or a tissue surface.

51. The complex according to any of paragraphs 48-50 wherein where the complex is located in the plasma membrane of a platelet, the target is a target such that target binding by the complex results in complex clustering on the plasma membrane, optionally wherein said clustering is sufficient to activate the platelet modulation domain.

52. The complex according to any of paragraphs 48-51 wherein where the complex is located in the platelet plasma membrane binding of the complex to the target activates the platelet modulation domain.

53. The tagged targeting peptide according to any of paragraphs 42, 43, 49 or 50 or the complex according to any of paragraphs 48-52 wherein the target binding domain comprises a human target binding domain sequence or a sequence that has at least 75%, 80%, 85%, 90%, 92%, 94%, 96%, 98% or 100% sequence identity to a human target binding domain sequence.

54. The tagged targeting peptide according to any of paragraphs 42, 43, 49, 50 or 53 or the complex according to any of paragraphs 48-53 wherein the target binding domain comprises a non-human target binding domain sequence, optionally:
a humanised sequence; or
a sequence from a mouse.

55. The tagged targeting peptide according to any of paragraphs 42, 43, 49, 50, 53 or 54 or the complex according to any of paragraphs 48-54 wherein said target binding domain comprises a target-binding ligand or fragment thereof that binds specifically to said target.

56. The tagged targeting peptide according to any of paragraphs 42, 43, 49, 50, or 53-55 54 or the complex according to any of paragraphs 48-55, wherein said target binding domain comprises an antibody or antibody fragment that binds specifically to said target.

57. The tagged targeting peptide according to any of paragraphs 42, 43, 49, 50, or 53-56 or the complex according to any of paragraphs 48-56 wherein said target binding domain comprises a variable heavy chain domain and/or a variable light chain domain, optionally an scFV.

58. The tagged targeting peptide according to any of paragraphs 42, 43, 49, 50, or 53-57 or the complex according to any of paragraphs 48-57 wherein the target is a tumor antigen, neoantigen or autoantigen.

59. The tagged targeting peptide according to any of paragraphs 42, 43, 49, 50, or 53-58 or the complex according to any of paragraphs 48-58 wherein the target is:
a) an antigen associated with a disease, disorder or condition; and/or
b) on a target tissue or cell in the body of a subject. optionally wherein the target tissue or cell is a cancer tissue or cell.

60. The tagged targeting peptide according to any of paragraphs 42, 43, 49, 50, or 53-59 or the complex according to any of paragraphs 48-59 wherein the target binding domain comprises at least one of:
a) FCERG EC domain, CLEC1 EC domain,FCGR2 EC domain, GPVIA EC domain, CEACAM1 EC domain, G6b-B EC domain, LILRB2 EC domain, PECAM1 EC domain and/or TLT1 EC domain or a sequence that has at least 75%, 80%, 85%, 90%, 92%, 94%, 96%, 98% or 100% sequence identity to a FCERG EC domain, CLEC1 EC domain, FCGR2 EC domain, GPVIA EC domain, CEACAM1 EC domain, G6b-B EC domain, LILRB2 EC domain, PECAM1 EC domain and/or TLT1 EC domain; and/or
b) the target binding domain comprises any one or more of the domains or portions thereof set out on page 46 to 49 of PCT/GB2020/053247 which is hereby incorporated by reference, or a sequence that has at least 75%, 80%, 85%, 90%, 92%, 94%, 96%, 98% or 100% sequence identity to any one or more of the domains or portions thereof set out on page 46 to 49 of PCT/GB2020/053247 which is hereby incorporated by reference.

61. The tagged targeting peptide according to any of paragraphs 42, 43, 49, 50, or 53-60 or the complex according to any of paragraphs 48-60 wherein the target binding domain comprises a peptide associated with autoimmunity, optionally:
a peptide or portion of any one or more of the following proteins: MOG, GAD65, MAG, PMP22, TPO, VGKC, PLP, AChR, TRIB2, NMDA, GluR, GAD2, ARMC9, CYP21A2, CASR, NASP, insulin, TSHR, thyroperoxidase, asioglycoprotein receptor, CYP2D6, LF, TTG, H/K ATP-ase, Factor XIII, Beta2-GPI, ITGB2, G-CSF, GP IIb/IIa, COLII, FBG beta alpha, MPO, CYO, PRTN3, TGM, COLVII, COIL, DSG1, DSG3, SOX10, 70SNRNP70, SAG and a3(IV)NC1 collagen; or
a peptide or portion that has at least 75%, 80%, 85%, 90%, 92%, 94%, 96%, 98% or 100% sequence identity to any one or more of the following proteins: MOG, GAD65, MAG, PMP22, TPO, VGKC, PLP, AChR, TRIB2, NMDA, GluR, GAD2, ARMC9, CYP21A2, CASR, NASP, insulin, TSHR, thyroperoxidase, asioglycoprotein receptor, CYP2D6, LF, TTG, H/K ATP-ase, Factor XIII, Beta2-GPI, ITGB2, G-CSF, GP IIb/IIa, COLII, FBG beta alpha, MPO, CYO, PRTN3, TGM, COLVII, COIL, DSG1, DSG3, SOX10, 70SNRNP70, SAG and a3(IV)NC1 collagen.

62. The tagged targeting peptide according to any of paragraphs 42, 43, 49, 50, or 53-61 or the complex according to any of paragraphs 48-61 according to any of the preceding paragraphs wherein the target binding domain binds to a target that is:
a) an endogenous target that is found on a tissue in the body of a subject or on a cell or in a particular location of a subject;
b) present on tissue, or on a particular subset of tissue, or in plasma or blood of a subject, optional in a human subject optionally in the blood;
c) only presented during one or more disease states, optionally the target is a neoantigen that arises in a tumour cell;
d) only present in significant amounts optionally present in abnormal levels on a tissue or cell that does not normally express the target and/or is only present in a localised manner during or more disease states;
e) an antigen, optionally a tumour neoantigen or a tumour specific antigen;
f) CD19;
g) a cytokine receptor;
h) not collagen;
i) an artificial or exogenous target;
j) a designer drug;
k) a drug that has been designed using DREADD;
l) a protein selected from Table 2 on pages 23-31 of PCT/GB2020/053247 which is hereby incorporated by reference;
m) CD276; and/or
n) I12, KLK, amyloid, a Notch receptor and/or OLR1.

63. The tagged targeting peptide according to any of paragraphs 42, 43, 49, 50, or 53-62 or the complex according to any of paragraphs 48-62 wherein the target binding domain:
a) is an antibody or antigen binding fragment thereof;
b) comprises a variable heavy chain domain of an antibody and/or a variable light chain domain of an antibody; and/or
c) comprises a kappa light chain or a fragment thereof targeting.

64. The tagged targeting peptide according to any of paragraphs 42, 43, 49, 50, or 53-63 or the complex according to any of paragraphs 48-63 wherein the target binding domain comprises a portion of a protein or peptide associated with autoimmunity

65. The universal CPR according to any of paragraphs 41 and 44-47, or the complex according to any of paragraphs 48-64 wherein the platelet modulation domain is a platelet activation domain, optionally an ITAM comprising domain, optionally a platelet ITAM comprising domain, optionally is domain that has at least 75%, 80%, 85%, 90%, 92%, 94%, 96%, 98% or 100% sequence identity to an ITAM comprising domain, optionally a platelet ITAM comprising domain.

66. The universal CPR according to any of paragraphs 41, 44-47 and 65, or the complex according to any of paragraphs 48-65 wherein the platelet modulation domain is a platelet activation domain, optionally wherein the platelet activation domain is a degranulation triggering domain.

67. The universal CPR according to any of paragraphs 41, 44-47, 65 and 66, or the complex according to any of paragraphs 48-66 wherein when the universal CPR or complex is present in the membrane of a platelet, and when activated, the platelet activation domain:
a) results in degranulation of the platelet;
b) results in the release of contents from the platelet;
c) results in the presence of intracellular contents on the plasma membrane of the platelet; d) results in the release of extracellular vesicles via blebbing from the plasma membrane; and/or
e) results in a change of shape of the platelet from a biconcave disk to fully spread cell fragments.

68. The universal CPR according to any of paragraphs 41, 44-47, and 65-67, or the complex according to any of paragraphs 48-67 wherein the platelet activation domain is a platelet degranulation triggering domain.

69. The universal CPR according to any of paragraphs 41, 44-47, and 65-68, or the complex according to any of paragraphs 48-68 wherein the platelet modulation domain is an inhibition of platelet activation domain that prevents activation of a platelet, optionally wherein the inhibition of platelet activation domain is an MM comprising domain, optionally is a domain that has at least 75%, 80%, 85%, 90%, 92%, 94%, 96%, 98% or 100% sequence identity to an ITIM comprising domain.

70. The universal CPR according to any of paragraphs 41, 44-47, and 65-69, or the complex according to any of paragraphs 48-69 wherein when the universal CPR or complex is present in the membrane of a platelet, and when the inhibition of platelet activation domain is activated, the platelet inhibition of activation domain:
a) prevents degranulation of the platelet;
b) prevents the release of contents from the platelet;
c) prevents the presence of intracellular contents on the plasma membrane of the platelet;
d) prevents the release of extracellular vesicles via blebbing from the plasma membrane; and/or
e) prevents a change of shape of the platelet from a biconcave disk to fully spread cell fragments.

71. The universal CPR according to any of paragraphs 41, 44-47, and 65-70, or the complex according to any of paragraphs 48-70 wherein the platelet modulation domain comprises a human modulation domain sequence.

72. The universal CPR according to any of paragraphs 41, 44-47, and 65-71, or the complex according to any of paragraphs 48-71 wherein the platelet modulation domain comprises a non-human modulation domain sequence, optionally a sequence from a mouse.

73. The universal CPR according to any of paragraphs 41, 44-47, and 65-72, or the complex according to any of paragraphs 48-72 wherein the platelet modulation domain is endogenous to the progenitor, producer or effector-chassis that the universal CPR or complex is to be used with, optionally wherein the platelet modulation domain is endogenous to an iPSC, a megakaryocyte or a platelet.

74. The universal CPR according to any of paragraphs 41, 44-47, and 65-73, or the complex according to any of paragraphs 48-73 wherein the platelet modulation domain does not comprise domains from an immunoreceptor tyrosine based activation motif (ITAM) receptor, optionally does not comprise one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2).

75. The universal CPR according to any of paragraphs 41, 44-47, and 65-74, or the complex according to any of paragraphs 48-74 wherein when the universal CPR or complex is localised to a platelet plasma membrane, upon binding of the target to the target binding domain the platelet modulation domain triggers degranulation of the platelet.

76. The universal CPR according to any of paragraphs 41, 44-47, and 65-75, or the complex according to any of paragraphs 48-75 wherein the platelet modulation domain is a platelet degranulation triggering domain and comprises:
one or more domains from an immunoreceptor tyrosine based activation motif (ITAM) receptor, optionally comprises one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2); or
a domain that has at least 75%, 80%, 85%, 90%, 92%, 94%, 96%, 98% or 100% sequence identity to an ITAM comprising domain, optionally a platelet ITAM comprising domain, optionally has at least 75%, 80%, 85%, 90%, 92%, 94%, 96%, 98% or 100% sequence identity to one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2).

77. The universal CPR according to any of paragraphs 41, 44-47, and 65-76, or the complex according to any of paragraphs 48-76 wherein the platelet modulation domain is a domain that inhibits triggering of platelet degranulation and comprises one or more ITIM motifs, optionally wherein the one or more ITIM motifs is an ITIM motif from PECAM1, TLT1, LILRB2, CEACAM1 or G6b-B, optionally wherein the ITIM domain from:
LILRB2 is SEQ ID NO: 34 shown in Table 5 on page 44 of PCT/GB2020/053247 which is hereby incorporated by reference
PECAM1 is SEQ ID NO: 38 shown in Table 5 on page 44 of PCT/GB2020/053247 which is hereby incorporated by reference
CEACAM1 is SEQ ID NO: 24 shown in Table 5 on page 44 of PCT/GB2020/053247 which is hereby incorporated by reference.

78. The universal CPR according to any of paragraphs 41, 44-47, and 65-77, or the complex according to any of paragraphs 48-77 wherein the platelet modulation domain comprises one or more mutations, insertions or deletions relative to the native platelet modulation domain sequence.

79. The universal CPR according to paragraph 78 or the complex according to paragraph 78 wherein the one or more mutations, insertions or deletions relative to the native modulation domain sequence increases the sensitivity of the universal CPR or complex of universal CPR and tagged targeting peptide relative to a universal CPR or complex of universal CPR and tagged targeting peptide that comprises a platelet modulation domain that does not comprise the one or more mutations.

80. The universal CPR according to paragraph 78 or complex according to paragraph 78 wherein the one or more mutations, insertions or deletions relative to the native modulation domain sequence decreases the sensitivity of the universal CPR or complex of universal CPR and tagged targeting peptide relative to a universal CPR or complex of universal CPR and tagged targeting peptide that comprises a platelet modulation domain that does not comprise the one or more mutations.

81. The universal CPR according to any of paragraphs 41, 44-47, and 65-80, or the complex according to any of paragraphs 48-80 wherein the platelet modulation domain comprises at least 75%, 80%, 85%, 90%, 92%, 94%, 96%, 98% or 100% sequence identity to a naturally occurring platelet modulation domain.

82. The universal CPR according to any one of paragraphs 41, 44-47 and 65-81 or the complex according to any of paragraphs 48-81 further comprising a signal peptide and/or linker sequence, optionally wherein;
the signal peptide comprises or consists of a portion of the sequences set out in Table 1; and/or
the signal peptide comprises or consists of a portion of any of the sequences in Table 7 on page 46 of PCT/GB2020/053247 which is hereby incorporated by reference; and/or optionally wherein the linker comprises or consists of the linkers or portions thereof as set out on page 51 of PCT/GB2020/053247 which is hereby incorporated by reference.

83. The universal CPR according to any of paragraphs 41, 44-47 and 65-82 or complex according to any of paragraphs 48-82 further comprising a transmembrane domain, optionally wherein the transmembrane domain comprises or consists of any one or more of the transmembrane domains or portions thereof as set out on page 49-50 of PCT/GB2020/053247 which is hereby incorporated by reference.

84. The universal CPR according to any of paragraphs 41, 44-47 and 65-83 or complex according to any of paragraphs 48-83 wherein the CPR comprises an intracellular domain that comprises or consists of the intracellular domains or a portion thereof as set out on page 50 and 51 of PCT/GB2020/053247 which is hereby incorporated by reference.

85. The universal CPR according to any of paragraphs 41, 44-47 and 65-84 or complex according to any of paragraphs 48-84 wherein the CPR comprises or consists of a combination of domains as set out on pages 41-63 of PCT/GB2020/053247 which is hereby incorporated by reference.

86. A synthetic antigen presenting receptor (SAPR) comprising a heterologous target binding domain wherein the target binding domain comprises:
a) an extracellular domain comprising:
   i) the MHC-1 protein or fragment thereof, or a protein or fragment thereof that has at least 75%, 80%, 85%, 90%, 92%, 94%, 96%, 98% or 100% sequence identity to a human MHC-1 protein or fragment thereof; or
   ii) the MHC-2 protein or fragment thereof or a protein or fragment thereof that has at least 75%, 80%, 85%, 90%, 92%, 94%, 96%, 98% or 100% sequence identity to a human MHC-2 protein or fragment thereof; and
b) an intracellular platelet modulation domain,
   wherein said:
MHC-1 protein or fragment thereof or a protein or fragment thereof that has at least 75%, 80%, 85%, 90%, 92%, 94%, 96%, 98% or 100% sequence identity to a human MHC-1 protein or fragment thereof; or
MHC-2 protein or fragment thereof or a protein or fragment thereof that has at least 75%, 80%, 85%, 90%, 92%, 94%, 96%, 98% or 100% sequence identity to a human MHC-2 protein or fragment thereof;
   is able to bind to a T Cell Receptor (TCR).

87. The SAPR according to paragraph 86 wherein said extracellular domain comprises: heterologous target binding domain comprises:
a) the MHC-1 protein or fragment thereof, or a protein or fragment thereof that has at least 75%, 80%, 85%, 90%, 92%, 94%, 96%, 98% or 100% sequence identity to a human MHC-1 protein or fragment thereof. and an antigenic peptide, wherein said MHC-1 protein or fragment thereof, or a protein or fragment thereof that has at least 75%, 80%, 85%, 90%, 92%, 94%, 96%, 98% or 100% sequence identity to a human MHC-1 protein or fragment thereof and antigenic peptide is able to bind to a TCR; and/or
b) the MHC-2 protein or fragment thereof, or a protein or fragment thereof that has at least 75%, 80%, 85%, 90%, 92%, 94%, 96%, 98% or 100% sequence identity to a human MHC-1 protein or fragment thereof. and an antigenic peptide, wherein said MHC-2 protein or fragment thereof, or a protein or fragment thereof that has at least 75%, 80%, 85%, 90%, 92%, 94%, 96%, 98% or 100% sequence identity to a human MHC-1 protein or fragment thereof and antigenic peptide is able to bind to a TCR.

88. The SAPR of any of 86 or 87 wherein the antigenic peptide comprises a peptide or antigenic portion thereof:
a) associated with cancer, an autoimmune condition, genetic disease, cardiovascular disease and/or an infection; and/or
b) selected from:
   i) the antigenic peptides listed in Table F on page 206-207; Table G on page 208; Table H on page 208-209; Table I on page 209-211; Table J page 212; Table 4 page 219-221; Table 5 page 221-230; Table 6 page 231-234; Table 7 page 235-242 and Table 89 page 243 of WO 2015153102 these sections of which are hereby incorporated by reference;
   ii) a sequence that has at least 75%, 80%, 85%, 90%, 92%, 94%, 96%, 98% or 100% sequence identity to the antigenic peptides listed in Table F on page 206-207; Table G on page 208; Table H on page 208-209; Table I on page 209-211; Table J page 212; Table 4 page 219-221; Table 5 page 221-230; Table 6 page 231-234; Table 7 page 235-242 and Table 89 page 243 of WO 2015153102 these sections of which are hereby incorporated by reference; and/or
c) selected from:
   i) the antigenic peptides listed in Table 1 page 47-86; Table 14 page 321; Table 15 page 321; Table 16 page 327; Table 17 page 328; Table 18 page 328-329; Table 19 page 221-223; Table 20 page 333-334; Table 21 page 340-342; Table 22 page 344-347; Table 23 page 347-348; and Table 24 page 349-352 of WO 2019/126818, these sections of which are hereby incorporated by reference; or
   ii) a sequence that has at least 75%, 80%, 85%, 90%, 92%, 94%, 96%, 98% or 100% sequence identity to the antigenic peptides listed the antigenic peptides listed in Table 1 page 47-86; Table 14 page 321; Table 15 page 321; Table 16 page 327; Table 17 page 328; Table 18 page 328-329; Table 19 page 221-223; Table 20 page 333-334; Table 21 page 340-342; Table 22 page 344-347; Table 23 page 347-348; and Table 24 page 349-352 of WO 2019/126818, these sections of which are hereby incorporated by reference;
d) selected from:
   i) any one or more of the following proteins: MOG, GAD65, MAG, PMP22, TPO, VGKC, PLP, AChR, TRIB2, NMDA, GluR, GAD2, ARMC9, CYP21A2, CASR, NASP, insulin, TSHR, thyroperoxidase, asioglycoprotein receptor, CYP2D6, LF, TTG, H/K ATP-ase, Factor XIII, Beta2-GPI, ITGB2, G-CSF, GP IIb/IIa, COLII, FBG beta alpha, MPO, CYO, PRTN3, TGM, COLVII, COIL, DSG1, DSG3, SOX10, 70SNRNP70, SAG and a3(IV)NC1 collagen; or
   ii) a sequence that has at least 75%, 80%, 85%, 90%, 92%, 94%, 96%, 98% or 100% sequence identity to any one or more of the following proteins: MOG, GAD65, MAG, PMP22, TPO, VGKC, PLP, AChR, TRIB2, NMDA, GluR, GAD2, ARMC9, CYP21A2, CASR, NASP, insulin, TSHR, thyroperoxidase, asioglycoprotein receptor, CYP2D6, LF, TTG, H/K ATP-ase, Factor XIII, Beta2-GPI, ITGB2, G-CSF, GP IIb/IIa, COLII, FBG beta alpha, MPO, CYO, PRTN3, TGM, COLVII, COIL, DSG1, DSG3, SOX10, 70SNRNP70, SAG and a3(IV)NC1 collagen.

89. The SAPR according to any of paragraphs 86-88 wherein the extracellular domain is able to bind to a T Cell Receptor (TCR).

90. The SAPR according to any of paragraphs 86-89 wherein the extracellular domain comprises a human target binding domain sequence.

91. The SAPR according to any of paragraphs 86-90 wherein the extracellular domain comprises a non-human target binding domain sequence, optionally:
a humanised sequence; or
a sequence from a mouse.

92. The SAPR according to any of paragraphs 86-91 wherein the platelet modulation domain is a platelet activation domain, optionally an ITAM comprising domain, optionally a platelet ITAM comprising domain, optionally is domain that has at least 75%, 80%, 85%, 90%, 92%, 94%, 96%, 98% or 100% sequence identity to an ITAM comprising domain, optionally a platelet ITAM comprising domain.

93. The SAPR according to any of paragraphs 86-92 wherein the platelet modulation domain is a platelet activation domain, optionally wherein the platelet activation domain is a degranulation triggering domain.

94. The SAPR of any of the paragraphs 86-93 wherein when the SAPR is present in the membrane of a platelet, and when activated, the platelet activation domain:
a) results in degranulation of the platelet;
b) results in the release of contents from the platelet;
c) results in the presence of intracellular contents on the plasma membrane of the platelet; d) results in the release of extracellular vesicles via blebbing from the plasma membrane; and/or
e) results in a change of shape of the platelet from a biconcave disk to fully spread cell fragments.

95. The SAPR of any of paragraphs 86-94 wherein the platelet activation domain is a platelet degranulation triggering domain.

96. The SAPR according to any paragraphs 86-95 wherein the platelet modulation domain is an inhibition of platelet activation domain that prevents activation of a platelet, optionally wherein the inhibition of platelet activation domain is an MM comprising domain, optionally is a domain that has at least 75%, 80%, 85%, 90%, 92%, 94%, 96%, 98% or 100% sequence identity to an MM comprising domain.

97. The SAPR of any of paragraphs 86-96 wherein when the SAPR is present in the membrane of a platelet, and when the inhibition of platelet activation domain is activated the inhibition of platelet activation domain:
a) prevents degranulation of the platelet;
b) prevents the release of contents from the platelet;
c) prevents the presence of intracellular contents on the plasma membrane of the platelet;
d) prevents the release of extracellular vesicles via blebbing from the plasma membrane; and/or
e) prevents a change of shape of the platelet from a biconcave disk to fully spread cell fragments.

98. The SAPR of any of paragraphs 86-97 wherein the platelet activation domain is a platelet degranulation triggering domain.

99. The SAPR according to any of paragraphs 86-98 wherein the platelet modulation domain comprises a human modulation domain sequence.

100. The SAPR according to any of paragraphs 86-99 wherein the platelet modulation domain comprises a non-human modulation domain sequence, optionally a sequence from a mouse.

101. The SAPR according to any of paragraphs 86-100 wherein the platelet modulation domain is endogenous to the progenitor, producer or effector-chassis that the SAPR is to be used with, optionally wherein the platelet modulation domain is endogenous to an iPSC, a megakaryocyte or a platelet.

102. The SAPR according to any of paragraphs 86-101 wherein the platelet modulation domain does not comprise domains from an immunoreceptor tyrosine based activation motif (ITAM) receptor, optionally does not comprise one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2).

103. The SAPR according to any of paragraphs 86-102 wherein when the SAPR is localised to a platelet plasma membrane, upon binding of the target to the target binding domain, the platelet modulation domain triggers degranulation of the platelet.

104. The SAPR according to any of paragraphs 86-104 wherein the platelet modulation domain is a platelet degranulation triggering domain and comprises:
one or more domains from an immunoreceptor tyrosine based activation motif (ITAM) receptor, optionally comprises one or more domains, portions or fragments thereof from Glycoprotein VI (GPVI), C-type lectinlike receptor 2 (CLEC-2), Fc Fragment of IgG Receptor Ha (FCgR2A), high affinity immunoglobulin epsilon receptor subunit gamma (FCERG), C-Type lectin domain family 1 (CLEC1), or Fc fragment of IgG receptor II (FCGR2); or
a domain that has at least 75%, 80%, 85%, 90%, 92%, 94%, 96%, 98% or 100% sequence identity to an ITAM comprising domain, optionally a platelet ITAM comprising domain, optionally has at least 75%, 80%, 85%, 90%, 92%, 94%, 96%, 98% or 100% sequence identity to one or more domains, portions or fragments thereof from Glycoprotein VI (GPVI), C-type lectinlike receptor 2 (CLEC-2), Fc Fragment of IgG Receptor IIa (FCgR2A), high affinity immunoglobulin epsilon receptor subunit gamma (FCERG), C-Type lectin domain family 1 (CLEC1), or Fc fragment of IgG receptor II (FCGR2).

105. The SAPR according to any of paragraphs 86-104 wherein the platelet modulation domain is an inhibition of platelet activation domain that inhibits triggering of platelet degranulation and comprises one or more mM motifs, optionally wherein the one or more MM motifs is an MM motif from PECAM1, TLT1, LILRB2, CEACAM1 or G6b-B, optionally wherein the ITIM domain from: LILRB2 is SEQ ID NO: 34 shown in Table 5 on page 44 of PCT/GB2020/053247 which is hereby incorporated by reference PECAM1 is SEQ ID NO: 38 shown in Table 5 on page 44 of PCT/GB2020/053247 which is hereby incorporated by reference CEACAM1 is SEQ ID NO: 24 shown in Table 5 on page 44 of PCT/GB2020/053247 which is hereby incorporated by reference.

106. The SAPR according to any of paragraphs 86-105 wherein the platelet modulation domain comprises one or more mutations, insertions or deletions relative to a native platelet modulation domain sequence.

107. The SAPR according to any of paragraphs 86-106 wherein the one or more mutations, insertions or deletions relative to the native modulation domain sequence increases the sensitivity of the SAPR relative to a SAPR that comprises a platelet modulation domain that does not comprise the one or more mutations.

108. The SAPR according to any of paragraphs 86-107 wherein the one or more mutations, insertions or deletions relative to the native modulation domain sequence decreases the sensitivity of the SAPR relative to a SAPR that comprises a platelet modulation domain that does not comprise the one or more mutations.

109. The SAPR according to any of paragraphs 86-108 wherein the platelet modulation domain comprises at least 75%, 80%, 85%, 90%, 92%, 94%, 96%, 98% or 100% sequence identity to a naturally occurring platelet modulation domain.

110. The SAPR according to any of paragraphs 86-109 further comprising a signal peptide and/or linker sequence, optionally wherein;
the signal peptide comprises or consists of a portion of the sequences set out in Table 1; and/or
the signal peptide comprises or consists of a portion of any of the sequences in Table 7 on page 46 of PCT/GB2020/053247 which is hereby incorporated by reference; and/or optionally wherein the linker comprises or consists of the linkers or portions thereof as set out on page 51 of PCT/GB2020/053247 which is hereby incorporated by reference.

111. The SAPR according to any of paragraphs 86-110 further comprising a transmembrane domain, optionally wherein the transmembrane domain comprises or consists of any one or more of the transmembrane domains or portions thereof as set out on page 49-50 of PCT/GB2020/053247 which is hereby incorporated by reference.

112. The SAPR according to any of paragraphs 86-111 wherein the SAPR comprises an intracellular domain that comprises or consists of the intracellular domains or a portion thereof as set out on page 50 and 51 of PCT/GB2020/053247 which is hereby incorporated by reference.

113. The SAPR according to any of paragraphs 86-112 wherein the SAPR, comprises or consists of a combination of domains as set out on pages 41-63 of PCT/GB2020/053247 which is hereby incorporated by reference.

114. An engineered protease activated receptor (ePAR) wherein the protease recognition site is engineered to be cleaved by a protease that is not the protease that cleaves the native recognition site.

115. The ePAR of paragraph 114 wherein, when present in the plasma membrane of a platelet, cleavage of the protease recognition site results in:
a) degranulation of the platelet;
b) the release of contents from the platelet;
c) the presence of intracellular contents on the plasma membrane of the platelet;
d) the release of extracellular vesicles via blebbing from the plasma membrane; and/or
e) a change of shape of the platelet from a biconcave disk to fully spread cell fragments.

116. The ePAR of paragraph 113 or 115 wherein cleavage of the protease results in release of a fragment of the ePAR and wherein the fragment of the ePAR is a signalling molecule and effects intracellular signalling.

117. The ePAR of any of paragraphs 113-115 wherein the protease recognition site is engineered to be a protease recognition site for a protease found in the tumour micro-environment, optionally wherein the protease recognition site for a protease found in the tumour micro-environment is selected from the group comprising or consisting matrix metalloproteases, metallopeptidases, Cathepsin B, Urokinases or Caspases.

118. The ePAR of any of paragraphs 113-117 wherein the protease recognition site is engineered to be an orthogonal protease recognition site with respect to the intended subject.

119. The ePAR of any of paragraphs 113-118 wherein the protease recognition site is engineered to be a viral protease recognition site, optionally a Tobacco Etch Virus nuclear-inclusion-a endopeptidase (TEV protease), NS2-3 protease of hepatitis C virus (HCV protease), or tobacco vein mottling virus (TVMV protease).

120. The ePAR according to any of paragraphs 113-119 wherein the ePAR is a GPCR, optionally is an engineered PAR1, PAR2, PAR3 or PAR4.

121. The ePAR according to any of paragraphs 113-120 wherein the ePAR comprises at least 75%, 80%, 85%, 90%, 92%, 94%, 96%, 98% or 100% sequence identity to a naturally occurring PAR.

122. A nucleic acid encoding the CPR according to any of paragraphs 41, 44-47 and 65-85 or complex according to any of paragraphs 48-85, or SAPR according to any of paragraphs 86-113, or ePAR according to any of paragraphs 114-121.

123. The nucleic acid according to paragraph 122 wherein the nucleic acid is DNA.

124. The nucleic acid according to paragraph 122 wherein the nucleic acid is RNA.

125. The nucleic acid according to any of paragraphs 122-124 wherein the nucleic acid is operatively linked to a heterologous expression sequence, optionally a heterologous promoter.

126. The nucleic acid of any of paragraphs 122-125 further comprising a megakaryocyte-specific promoter.

127. The nucleic acid according to any of paragraphs 122-126 wherein the promoter is an inducible promoter, optionally a promoter that is inducible in an intended subject.

128. The nucleic acid according to any paragraphs 122-127 wherein the promoter is a constitutive prompter, optionally a promoter that is constitutive in an intended subject.

129. A vector comprising a nucleic acid according to any of paragraphs 122-127, optionally wherein the vector is a plasmid or circular nucleic acid.

130. A viral vector or viral particle comprising a nucleic acid according to any of paragraphs 122-128 or a vector according to paragraph 129.

131. A chassis comprising:
a) one or more CPRs, universal CPRs, complexes of universal CPRs and tagged targeting peptides, SAPRs, or ePARS according to any of the preceding paragraphs;
b) one or more nucleic acids according to any of the preceding paragraphs that encodes the CPR, universal CPR, SAPR, or ePAR according to any of the preceding paragraphs;
c) one or more vectors according to the previous paragraphs that comprises one or more nucleic acids according to any of the preceding paragraphs that encodes the CPR, universal CPR, SAPR, or ePAR according to any of the preceding paragraphs; and/or
d) one or more viral vectors according to any of the previous paragraphs that comprises one or more nucleic acids according to any of the preceding paragraphs that encodes the CPR, universal CPR, SAPR, or ePAR according to any of the preceding paragraphs.

132. The chassis of paragraph 131 wherein the chassis has not been engineered:
to modulate one or more signaling pathways, optionally engineered to disrupt the thrombogenic pathway and/or engineered to disrupt a platelet inflammatory signaling pathway and/or engineered to make the engineered producer or effector-chassis less immunogenic; and/or
to enhance or disrupt one or more base functions of the progenitor, producer or effector-chassis, optionally wherein the one or more or base functions are involved in the innate and/or adaptive immune response, inflammation, angiogenesis, atherosclerosis, lymphatic development and tumour growth.

133. An engineered chassis, wherein the chassis has been engineered:
to modulate one or more signaling pathways, optionally engineered to disrupt the thrombogenic pathway and/or engineered to disrupt a platelet inflammatory signaling pathway and/or engineered to make the engineered platelet less immunogenic; and/or
to enhance or disrupt one or more base functions of the chassis, optionally wherein the one or more or base functions are involved in the innate and/or adaptive immune response, inflammation, angiogenesis, atherosclerosis, lymphatic development and tumour growth.

134. The engineered chassis of paragraph 66 wherein the chassis has been further engineered to comprise any one or more of:
a) one or more CPRs, universal CPRs, complexes of universal CPRs and tagged targeting peptides, SAPRs, or ePARS according to any of the preceding paragraphs;
b) one or more nucleic acids according to any of the preceding paragraphs that encodes the CPR, universal CPR, SAPR, or ePAR according to any of the preceding paragraphs;
c) one or more vectors according to the previous paragraphs that comprises one or more nucleic acids according to any of the preceding paragraphs that encodes the CPR, d) universal CPR, SAPR, or ePAR according to any of the preceding paragraphs; and/or one or more viral vectors according to any of the previous paragraphs that comprises one or more nucleic acids according to any of the preceding paragraphs that encodes the CPR, universal CPR, SAPR, or ePAR according to any of the preceding paragraphs.

135. The engineered chassis of any of the preceding paragraphs wherein the chassis does not express any one or more of:
a) one or more CPRs, universal CPRs, complexes of universal CPRs and tagged targeting peptides, SAPRs, or ePARS according to any of the preceding paragraphs;
b) one or more nucleic acids according to any of the preceding paragraphs;
c) one or more vectors according to the previous paragraphs; and/or
d) one or more viral vectors according to any of the previous paragraphs.

136. The chassis or engineered chassis according to any of the preceding paragraphs wherein the chassis or engineered chassis is:
a) a progenitor-chassis, optionally is a myeloid stem cell; an iPSC; adipocyte; adipose-derived mesenchymal stromal/stem cell line (ASCL); or cancer cell-line that is capable of producing a producer-chassis; or other immortal cell that is capable of producing a producer-chassis;
b) a producer-chassis, optionally is a megakaryoblast; a megakaryocyte; a megakaryocyte-like cell; a cancer cell line that is capable of forming a platelet, a platelet-like membrane-bound cell fragment or an anucleate cell fragment for example a MEG01 or DAMI cancer cell line; or other immortal cell that is capable of forming a platelet, a platelet-like membrane-bound cell fragment or an anucleate cell fragment; or
c) an effector-chassis, optionally is a platelet, a platelet-like membrane-bound cell fragment or anucleate cell fragment.

137. The chassis or engineered chassis according to any of the preceding paragraphs wherein the chassis has been modified so as to drive differentiation to a producer-chassis, optionally drive differentiation to a megakaryocyte or a megakaryocyte-like cell, optionally has been forward programmed to differentiate into a megakaryocyte or a megakaryocyte-like cell.

138. The chassis or engineered chassis according to any of the preceding paragraphs wherein the chassis or engineered chassis is a producer-chassis or engineered producer-chassis and wherein the producer-chassis or engineered producer-chassis is a megakaryoblast that can produce a platelet, a platelet-like membrane-bound cell fragment or an anucleate cell fragment; a megakaryocyte that can produce a platelet, a platelet-like membrane-bound cell fragment or an anucleate cell fragment; or a megakaryocyte-like cell that can produce a platelet, a platelet-like membrane-bound cell fragment or an anucleate cell fragment.

139. The chassis or engineered chassis according to any of the preceding paragraphs wherein the chassis or engineered chassis is a producer-chassis or engineered producer-chassis that is a megakaryoblast; a megakaryocyte; a megakaryocyte-like cell; a cancer cell line that is capable of forming a platelet, a platelet-like membrane-bound cell fragment or an anucleate cell fragment for example a MEG01 or DAMI cancer cell line; or other immortal cell that is capable of forming a platelet, a platelet-like membrane-bound cell fragment or an anucleate cell fragment and wherein the megakaryoblast; megakaryocyte; megakaryocyte-like cell; cancer cell line that is capable of forming a platelet, a platelet-like membrane-bound cell fragment or an anucleate cell fragment for example a MEG01 or DAMI cancer cell line; or other immortal cell that is capable of forming a platelet, a platelet-like membrane-bound cell fragment or an anucleate cell fragment:
a) can produce pseudopodal extensions; and/or
b) expresses TUBB1.

140. The chassis or engineered chassis according to any of the preceding paragraphs wherein the chassis or engineered chassis is an effector-chassis or engineered effector-chassis and wherein the effector-chassis or engineered effector-chassis is a platelet, platelet-like membrane-bound cell fragment, or anucleate cell fragment and where the platelet, platelet-like membrane-bound cell fragment or anucleate cell fragment has been produced by fragmentation of a producer-chassis or engineered producer-chassis according to any of the preceding paragraphs, optionally wherein the engineered effector-chassis comprises TUBB1 protein.

141. The chassis or engineered chassis according to any of the preceding paragraphs wherein the chassis or engineered chassis is an effector-chassis or engineered effector-chassis and wherein the effector-chassis or engineered effector-chassis is a platelet, platelet-like membrane-bound cell fragment, or anucleate cell fragment that does not aggregate in a platelet aggregation assay.

142. The chassis or engineered chassis according to any of the preceding paragraphs wherein the chassis or engineered chassis comprises one or more nucleic acids according to any of the preceding paragraphs that encodes a CPR, universal CPR, SAPR, or ePAR according to any of the preceding paragraphs and or one or more vectors according to any of the preceding paragraphs that encodes a CPR, universal CPR, SAPR, or ePAR according to any of the preceding paragraphs.

143. The chassis or engineered chassis according to paragraph 142 wherein chassis or engineered chassis is a progenitor or producer-chassis or engineered progenitor or producer-chassis and wherein the one or more nucleic acids are expressed from a position within the genomic nucleic acid of the progenitor or producer-chassis or engineered progenitor or producer-chassis, optionally wherein
1) a nucleic acid encoding a first CPR, universal CPR, complex of universal CPR and tagged targeting peptide, SAPR, or ePAR has been introduced into a first allele of a first locus, and/or a nucleic acid encoding a first CPR, universal CPR, complex of universal CPR and tagged targeting peptide, SAPR, or ePAR has been be introduced to a second allele of a first locus; and/or
2) a nucleic acid encoding a first CPR, universal CPR, complex of universal CPR and tagged targeting peptide, SAPR, or ePAR has been introduced into a first allele of a first locus and a second nucleic acid encoding a second CPR, universal CPR, complex of universal CPR and tagged targeting peptide, SAPR, or ePAR has been introduced in to a first allele of a second locus; and/or
3) a nucleic acid encoding a first CPR, universal CPR, complex of universal CPR and tagged targeting peptide, SAPR, or ePAR has been introduced into a first allele of a first locus and a second nucleic acid encoding a second CPR, universal CPR, complex of universal CPR and tagged targeting peptide, SAPR, or ePAR has been introduced into a second allele of the first locus.

144. The chassis or engineered chassis according to paragraph 142 wherein the one or more nucleic acids are expressed episomally.

145. The chassis or engineered chassis according to any of the preceding paragraphs wherein the chassis or engineered chassis has been engineered so as to have inhibited expression from the beta 2 microglobulin gene, optionally wherein the beta 2 microglobulin gene has been knocked out or deleted.

146. The chassis or engineered chassis according to any of the preceding paragraphs wherein the chassis or engineered chassis is a mammalian chassis, optionally a human chassis, bovine chassis, equine chassis or murine chassis.

147. The engineered chassis according to any of the preceding paragraphs wherein the chassis has been engineered to disrupt a platelet thrombogenic pathway.

148. The engineered chassis according to any of the preceding paragraphs wherein the chassis has been engineered so as to have reduced thrombogenicity relative to a chassis that has not been engineered so as to have reduced thrombogenic potential, optionally wherein the engineered chassis has no thrombogenic potential.

149. The engineered chassis according to any of the preceding paragraphs wherein the chassis comprises a disruption of or deletion of at least two, three, four, five, six, seven, eight, nine, or at least ten genes involved in the thrombogenic pathway, optionally wherein the genes are selected from the group of genes encoding:
a protein involved in recognition of primary stimuli of thrombus formation;
a protein involved in recognition of secondary mediators of thrombus formation; and/or
a protein involved in the release of secondary mediators of thrombus formation.

150. The engineered chassis according to any of the preceding paragraphs wherein the chassis comprises a disruption or deletion of at least:
one gene that encodes a protein involved in recognition of primary stimuli of thrombus formation;
one gene that encodes a protein involved in recognition of secondary mediators of thrombus formation; and
one gene that encodes a protein involved in the release of secondary mediators of thrombus formation;
optionally comprises a disruption of at least:
two genes that encode a protein involved in recognition of primary stimuli of thrombus formation;
two genes that encode a protein involved in recognition of secondary mediators of thrombus formation; and
two genes that encode a protein involved in the release of secondary mediators of thrombus formation;
optionally comprises a disruption of at least:
three genes that encode a protein involved in recognition of primary stimuli of thrombus formation;
three genes that encode a protein involved in recognition of secondary mediators of thrombus formation; and
three genes that encode a protein involved in the release of secondary mediators of thrombus formation.

151. The engineered chassis according any of the preceding paragraphs wherein:
the at least one, two or three genes that encode a protein involved in recognition of primary stimuli of thrombus formation are selected from the group consisting of: GPIb/V/IX and GPVI (GP6), ITGA2B, CLEC2, integrins s a_{IIb}b₃, a₂b₁, a₅b₁ and a₆b₁, or from the group consisting of GPVI and ITGA2B;
the at least one, two or three that encode a protein involved in recognition of secondary mediators of thrombus formation are selected from the group consisting of Par1, Par4, P2Y12, GPIb/V/IX, the Thromboxane receptor (TBXA2R), P2Y1, P2X1 and integrin a_{II}bb₃ or from the group consisting of Par1, Par4 and P2Y12; and/or
the at least one, two or three genes that encode a protein involved in the release of secondary mediators of thrombus formation are selected from the group consisting of Cox1, HPS and thromboxane-A synthase (TBXAS1) or from the group consisting of Cox1 and HPS.

152. The engineered chassis according any of the preceding paragraphs wherein each of the following genes is disrupted or deleted:
GPVI, ITGA2B, Par1, Par4, P2Y12, Cox1 and HPS.

153. The engineered chassis according any of the preceding paragraphs wherein the chassis is an effector-chassis and:
a) does not respond to endogenous stimuli that usual results in clot formation;
b) is not recruited by other activated platelets; and/or
c) on activation, is not able to recruit and activate endogenous platelets in a patient.

154. The engineered chassis according to any of the preceding paragraphs wherein the chassis has been engineered to have reduced immunogenicity relative to a non-engineered chassis.

155. The engineered chassis of any of the preceding paragraphs wherein:
a) the function of endogenous MHC Class 1 and/or MHC Class 2 has been disrupted; and/or
b) expression from the β2 microglobulin gene has been disrupted.

156. The engineered chassis of any of the preceding paragraphs wherein the β2 microglobulin gene has been knocked out.

157. The engineered chassis of any of the preceding paragraphs wherein expression from the β2 microglobulin gene has been disrupted through the use of CRISPR gene editing, or shRNA, optionally lentiviral delivery of shRNA.

158. The engineered chassis according to any of the preceding paragraphs wherein the chassis has been engineered to have disrupted expression from one or more HLA genes.

159. The engineered chassis according to paragraph 158 wherein the chassis has been engineered to have disrupted expression from any one or more of HLA-A, HLA-B and/or HLA-C, optionally wherein expression of HLA-A and HLA-B has been entirely disrupted but wherein expression of HLA-C has been partially disrupted, optionally wherein expression from both alleles of HLA-A and HLA-B have been disrupted but wherein expression from only one allele of HLA-C has been disrupted.

160. The engineered chassis according to any of the preceding paragraphs wherein the chassis has been engineered to overexpress anyone or more of the HLA class Ib genes, optionally any one or more of HLA-G, HLA-E, CD47 and PD-L1.

161. The engineered chassis according to any of the preceding paragraphs wherein the chassis has been engineered to overexpress any one or more of HLA-G, HLA-E, CD47 and PD-L1, and has optionally been engineered to have inhibited expression from the beta 2 microglobulin gene.

162. The engineered chassis according to any of the preceding paragraphs wherein the chassis has been engineered to overexpress on or more immunomodulatory genes, optionally wherein the one or more immunomodulatory genes is selected from the group comprising CD47 and PD-L1.

163. The engineered producer or effector-chassis according to any of the preceding paragraphs wherein the producer or effector-chassis has been engineered to eliminate one or more genes of which the product(s) could negatively affect the potency of a cargo.

164. The engineered chassis according to any of the preceding paragraphs wherein the chassis has been engineered to tune up or down the innate/adaptive response.

165. The engineered chassis according to any of the preceding paragraphs wherein the chassis has been engineered to reduce inflammation, angiogenesis, atherosclerosis, lymphatic development and tumour growth.

166. The engineered chassis according to any of the preceding paragraphs wherein the chassis has been engineered to disrupt one or more genes encoding adhesive proteins and/or cargo entities which are likely to indirectly counter the biological action of an engineered cargo.

167. The engineered chassis according to any of the preceding paragraphs wherein the chassis has been engineered to downregulate or inhibit expression of TGFb and/or GARP and/or CD40L.

168. The engineered chassis according to any of the preceding paragraphs wherein the chassis has been engineered to downregulate or inhibit expression of any one or more of CD36, NOD2, SRB1, TLR1, TLR2, TLR3, TLR4, TLR6, TLR9, CD40L, CD93 (C1qRp), C3aR, CD88 (C5aR), CD89 (FcαR1), CD23 (FcεR1), CD32 (FcγRIIa), MHC class1, CD191 (CCR1), CD193 (CCR3), CD194 (CCR4), CD184 (CXCR4), CX3CR1, CD102 (ICAM-2), JAM-C/JAM-3, CD62P (P-selectin), CD31 (PECAM-1), CD150 (SLAMF1), CCL2, CCL3, CCL5, CXCL1, CXCL12, CXCL4/PF4, CXCL5, CXCL8, NAP2 (CXCL7), IL-1p.

169. The engineered chassis according to any of the preceding paragraphs wherein the chassis has been engineered to disrupt or inhibit expression of TGFb and/or GARP2.

170. The engineered chassis according to any of the preceding paragraphs wherein the chassis has been engineered to disrupt or inhibit expression of any one or more of Siglec-7, Siglec-9, Siglec-11 and TGFβ.

171. The engineered chassis according to any of the preceding paragraphs wherein the chassis has been engineered to express one or more additional ITAM receptors to enhance T cell signaling and stimulate an immune response.

172. The engineered chassis according to any of the preceding paragraphs wherein the chassis has been engineered to have reduced immunogenicity relative to a non-engineered chassis, wherein the chassis has been engineered to:
a) have disrupted function of MHC Class 1 genes or proteins;
b) have disrupted expression from the β2 microglobulin gene, optionally to knock out the β2 microglobulin gene;
c) have disrupted expression from one or more HLA genes;
d) have disrupted expression from any one or more of HLA-A, HLA-B and/or HLA-C, optionally wherein expression of HLA-A and HLA-B has been entirely disrupted but wherein expression of HLA-C has been partially disrupted, optionally wherein expression from both alleles of HLA-A and HLA-B have been disrupted but wherein expression from only one allele of HLA-C has been disrupted;
e) overexpress anyone or more of the HLA class Ib genes, optionally any one or more of HLA-G, HLA-E, CD47 and PD-1;
f) been engineered to overexpress any one or more of HLA-G, HLA-E, CD47 and PD-L1 and optionally has been engineered to have disrupted expression from the beta 2 microglobulin; and/or
g) overexpress one or more immunomodulatory genes, optionally wherein the one or more immunomodulatory genes is selected from the group comprising CD47 and PD-1.

173. The engineered chassis according to any of the preceding paragraphs wherein the chassis has been engineered to:
a) have disrupted function of MHC Class 1 genes or proteins;
b) have disrupted expression from the β2 microglobulin gene, optionally to knock out the 02 microglobulin gene;
c) have disrupted expression from one or more HLA genes;
d) have disrupted expression from any one or more of HLA-A, HLA-B and/or HLA-C, optionally wherein expression of HLA-A and HLA-B has been entirely disrupted but wherein expression of HLA-C has been partially disrupted, optionally wherein expression from both alleles of HLA-A and HLA-B have been disrupted but wherein expression from only one allele of HLA-C has been disrupted;
e) overexpress anyone or more of the HLA class Ib genes, optionally any one or more of HLA-G, HLA-E, CD47 and PD-1;
f) overexpress any one or more of HLA-G, HLA-E, CD47 and PD-L1 and optionally has been engineered to have disrupted expression from the beta 2 microglobulin gene; and/or
g) overexpress one or more immunomodulatory genes, optionally wherein the one or more immunomodulatory genes is selected from the group comprising CD47 and PD-1;
h) eliminate one or more genes or gene products for which the product(s) could negatively affect the potency of a cargo;
i) tune up or down the innate/adaptive response;
j) reduce inflammation, angiogenesis, atherosclerosis, lymphatic development and tumour growth;
k) have disrupted expression of one or more genes encoding adhesive proteins and/or cargo entities which are likely to indirectly counter the biological action of the engineered cargo, potentially leading to a greater net therapeutic effect;
l) downregulate or inhibit expression of TGFb and/or GARP and/or CD40L;
n) downregulate or inhibit expression of any one or more of CD36, NOD2, SRB1, TLR1, TLR2, TLR3, TLR4, TLR6, TLR9, CD40L, CD93 (C1qRp), C3aR, CD88 (C5aR), CD89 (FcαR1), CD23 (FcεR1), CD32 (FcγRIIa), MHC class1, CD191 (CCR1), CD193 (CCR3), CD194 (CCR4), CD184 (CXCR4), CX3CR1, CD102 (ICAM-2), JAM-C/JAM-3, CD62P (P-selectin), CD31 (PECAM-1), CD150 (SLAMF1), CCL2, CCL3, CCL5, CXCL1, CXCL12, CXCL4/PF4, CXCL5, CXCL8, NAP2 (CXCL7), IL-1p
o) disrupt or inhibit expression of TGFb and/or GARP;
q) disrupt or inhibit expression of any one or more of Siglec-7, Siglec-9, Siglec-11 or TGFβ
s) disrupt or inhibit expression of any one or more of GPIb/V/IX and GPVI (GP6), ITGA2B, CLEC2, integrins s aIIbb3, a2b1, a5b1 and a6b1, GPVI and ITGA2B;
t) disrupt or inhibit expression of anyone or more of Par1, Par4, P2Y12, GPIb/V/IX, the Thromboxane receptor (TBXA2R), P2Y1, P2X1 and integrin aIIbb3 or from the group consisting of Par, Par4 and P2Y12;
u) disrupt or inhibit expression of anyone or more of Cox1, Cox2, HPS, prothrombin, PDGF, EGF, von Willebrand Factor and thromboxane-A synthase (TBXAS1);
v) to synthesise a protein or RNA of interest in response to activation of the platelet or platelet-like membrane-bound cell fragment, optionally wherein the protein or RNA of interest is expressed from the BCL-3 mRNA untranslated regions, optionally 5′UTR;
z) to express one or more cargo proteins or cargo RNAs, optionally wherein the cargo protein or cargo RNA comprises an alpha-granule targeting signal, optionally comprises a platelet factor 4 (PF4) or von Willebrand factor (vWf);
aa) express at least two CPRs, universal CPRs, complexes of universal CPRs and tagged targeting peptides, SAPRs or ePARs of any of the preceding paragraphs, optionally express at least 3, 4, 5, 6, 7, 8, 9 or at least 10 different CPRs, universal CPRs, complexes of universal CPRs and tagged targeting peptides, SAPRs or ePARs of any of the preceding paragraphs;
bb) express at least two CPRs, universal CPRs, complexes of universal CPRs and tagged targeting peptides, SAPRs or ePARs of any of the preceding paragraphs, and wherein the target binding domain of the at least two CPRs, universal CPRs, complexes of universal CPRs and tagged targeting peptides, SAPRs or ePARs are directed towards different targets;
cc) express at least two CPRs, universal CPRs, complexes of universal CPRs and tagged targeting peptides, SAPRs or ePARs of any of the preceding paragraphs, and wherein the target binding domain of the at least two CPRs, universal CPRs, complexes of universal CPRs and tagged targeting peptides, SAPRs or ePARs are directed towards different targets, and wherein:
   i) the platelet modulation domain of a first CPR, universal CPR, complex of universal CPR and tagged targeting peptide, SAPR or ePAR is a platelet activation domain optionally a degranulation triggering domain optionally an ITAM containing domain, and wherein the platelet modulation domain of a second CPR, universal CPR, complex of universal CPR and tagged targeting peptide, SAPR or ePAR is a platelet inhibition domain, optionally is a domain that prevents triggering of platelet degranulation, optionally is an ITAM containing domain;
   ii) the platelet modulation domain of a first CPR, universal CPR, complex of universal CPR and tagged targeting peptide, SAPR or ePAR is a platelet activation domain optionally a degranulation triggering domain optionally an ITAM containing domain, and wherein the platelet modulation domain of a second CPR, universal CPR, complex of universal CPR and tagged targeting peptide, SAPR or ePAR is a platelet activation domain optionally a degranulation triggering domain optionally an ITAM containing domain;
dd) express at least two CPRs, universal CPRs, complexes of universal CPRs and tagged targeting peptides, SAPRs or ePARs of any of the preceding paragraphs that operate together to form a logic circuit;
ee) express one or more cargo, optionally wherein the cargo is selected from the group comprising:
   a) a protein or peptide—optionally wherein the protein or peptide is:
      i) an antibody or antigen binding fragment thereof, for example an antibody or antigen binding fragment thereof binds to a tumor antigen or a neoantigen;
      ii) an enzyme, such as a nuclease for example a TALEN;
      iii) a cytokine for example IL-10; or
      iv) a CRISPR associated protein, for example Cas9;
      v) a bispecific protein, for example a bispecific antibody or optionally a T-cell engager (BiTE);
      vi) a fusion protein comprising an exosome targeting domain, optionally wherein the fusion protein comprises:
         a) the cargo protein or peptide; and
         b) an exosome targeting domain, optionally wherein the exosome targeting domain is selected from the group comprising or consisting of:
         i) an exosome specific membrane protein or exosome membrane targeting portion thereof, for example:
         a tetraspanin, for example CD63; or a non-tetraspanin such as PTGFRN or BASP1
         ii) an exosome targeting sequence from a soluble protein, optionally the WW domain of Nedd4 ubiquitin ligases;
         iii) a ubiquitin tag; and/or
         iv) a tag binding domain, optionally a nanobody directed against a tag, optionally a nanobody directed against GFP.
   b) a nucleic acid, optionally wherein the nucleic acid is:
      i) an RNA, for example selected from mRNA, a miRNA, shRNA, and a clustered regularly interspaced short palindromic repeats (CRISPR) sequence; and/or
      ii) an RNA that comprises an exosome targeting domain, optionally wherein the exosome targeting domain is selected from the group comprising or consisting of:
         a) an exosome targeting hairpin or linear motif;
         b) a viral exosome targeting RNA or exosome targeting fragment thereof;
   iii) an RNA that comprises an aptamer domain, optionally wherein the aptamer domain is selected from:
      a) a MS2 binding stem-loop;
      b) a C/D box; and/or
      c) an AU rich element, optionally wherein the RNA is an mRNA that encodes Cas9;
ff) express a fusion protein wherein the fusion protein comprises:
   i) the bacteriophage coat protein MS2 fused to an exosome membrane protein, optionally wherein the exosome membrane protein is selected from the group comprising or consisting of Lamp2b, VSVG, CD63; and/or
   ii) the archaeal ribosomal protein L7Ae fused to an exosome membrane protein, optionally wherein the exosome membrane protein is selected from the group comprising or consisting of Lamp2b, VSVG, CD63; and/or
   iii) a CD9-HuR fusion protein;
optionally wherein the fusion protein further comprises a light activated dimerization protein;
gg) to express one or more cargo only upon binding of one or more CPRs, universal CPRs, complexes of universal CPRs and tagged targeting peptides, SAPRs or ePARs to the target, optionally wherein the cargo is selected from the group comprising:
   a) a protein or peptide, optionally:
      i) an antibody or antigen binding fragment thereof, for example an antibody or antigen binding fragment thereof binds to a tumor antigen or a neoantigen;
      ii) an enzyme, such as a nuclease for example a TALEN;
      iii) a cytokine for example IL-10; or
      iv) a CRISPR associated protein, for example Cas9;
v) a bispecific protein, for example a bispecific antibody or optionally a T-cell engager (BiTE)
   b) a nucleic acid—in some embodiments the nucleic acid is:
      i) an RNA, for example selected from mRNA, a miRNA, shRNA, and a clustered regularly interspaced short palindromic repeats (CRISPR) sequence,
      optionally wherein the cargo is expressed from the Bcl-3 mRNA untranslated regions, optionally 5′UTR.

174. The chassis or engineered chassis according to any of the preceding paragraphs wherein the chassis or engineered chassis comprises one or more cargo, optionally wherein the engineered chassis has been:
a) loaded with one or more cargo; and/or
b) engineered so as to provide one or more cargo.

175. The chassis or engineered chassis according to any of the preceding paragraphs wherein the cargo is selected from any one or more of:
a) a protein or peptide—in some embodiments the protein or peptide is:
   i) an antibody or antigen binding fragment thereof, for example an antibody or antigen binding fragment thereof binds to a tumor antigen or a neoantigen;
   ii) an enzyme, such as a nuclease for example a TALEN;
   iii) a cytokine for example IL-10; or
   iv) a CRISPR associated protein, for example Cas9;
   v) a bispecific protein, for example a bispecific antibody or optionally a T-cell engager (BITE)
b) a nucleic acid—in some embodiments the nucleic acid is:
   i) an RNA, for example selected from mRNA, a miRNA, shRNA, and a clustered regularly interspaced short palindromic repeats (CRISPR) sequence; or
   ii) a DNA vector;
c) a toxin;
d) a small molecule drug, imaging agent, radionucleotide drug, radionucleotide tagged antibody, or any conjugate thereof;
e) a viral vector such as AAV;
f) a virus such as oncolytic virus;
g) agents for performing CRISPR mediated gene editing;
h) an exosome, for example an exosome pre-loaded with a second cargo; and/or
   i) or a nanoparticle or nanoparticles.
   or any combination thereof.

176. The chassis or engineered chassis according to any of the preceding paragraphs wherein the cargo is an endogenously expressed cargo, optionally wherein the endogenously expressed cargo is any one or more of:
a) a protein or peptide—in some embodiments the protein or peptide is:
   i) an antibody or antigen binding fragment thereof, for example an antibody or antigen binding fragment thereof binds to a tumor antigen or a neoantigen;
   ii) an enzyme, such as a nuclease for example a TALEN;
   iii) a cytokine for example IL-10; or
   iv) a CRISPR associated protein, for example Cas9;
   v) a bispecific protein, for example a bispecific antibody or optionally a T-cell engager (BiTE)
b) a nucleic acid—in some embodiments the nucleic acid is:
   i) an RNA, for example selected from mRNA, a miRNA, shRNA, and a clustered regularly interspaced short palindromic repeats (CRISPR) sequence.

177. The chassis or engineered chassis according to any of the preceding paragraphs wherein a cargo is exogenously loaded into the chassis or engineered chassis, optionally wherein exogenously loaded cargo is any one or more of:
a) a protein or peptide—in some embodiments the protein or peptide is:
   i) an antibody or antigen binding fragment thereof, for example an antibody or antigen binding fragment thereof binds to a tumor antigen or a neoantigen;
   ii) an enzyme, such as a nuclease for example a TALEN;
   iii) a cytokine for example IL-10; or
   iv) a CRISPR associated protein, for example Cas9;
   v) a bispecific protein, for example a bispecific antibody or optionally a T-cell engager (BITE)
b) a nucleic acid—in some embodiments the nucleic acid is:
   i) an RNA, for example selected from mRNA, a miRNA, shRNA, and a clustered regularly interspaced short palindromic repeats (CRISPR) sequence.

178. The chassis or engineered chassis according to any of the preceding paragraphs where the chassis or engineered chassis comprises a cargo and wherein the cargo has been exogenously loaded into or onto the chassis or engineered chassis, optionally into the cytoplasm, into the plasma membrane, or onto the extracellular surface.

179. The chassis or engineered chassis according to any of the preceding paragraphs wherein where the chassis or engineered chassis comprises a cargo, the cargo comprises an exosome targeting domain.

180. The chassis or engineered chassis according to paragraph 179 wherein the cargo is a protein or peptide that is a fusion protein comprising:
a) the cargo protein or peptide; and
b) an exosome targeting domain, optionally wherein the exosome targeting domain is selected from the group comprising or consisting of:
   i) an exosome specific membrane protein or exosome membrane targeting portion thereof, for example:
   a tetraspanin, for example CD63; or
   a non-tetraspanin such as PTGFRN or BASP1
   ii) an exosome targeting sequence from a soluble protein, optionally the WW domain of Nedd4 ubiquitin ligases;
   iii) a ubiquitin tag; and/or
   iv) a tag binding domain, optionally a nanobody directed against a tag, optionally a nanobody directed against GFP; and/or
   v) a protein selected from the proteins listed in Table A.

181. The chassis or engineered chassis according to paragraph 179 wherein the cargo is an RNA, and the exosome targeting domain is:
a) an exosome targeting hairpin or linear motif;
b) a viral exosome targeting RNA or exosome targeting fragment thereof;
c) an aptamer, optionally:
   i) a MS2 binding stem-loop;
   ii) a C/D box; and/or
   iii) an AU rich element, optionally wherein the RNA is an mRNA that encodes Cas9.

182. The chassis or engineered chassis according to any of the preceding paragraphs wherein the chassis or engineered chassis has been engineered to express a fusion protein, wherein the fusion protein comprises:
a) the bacteriophage coat protein MS2 fused to an exosome membrane protein, optionally wherein the exosome membrane protein is selected from the group comprising or consisting of Lamp2b, VSVG, CD63 or any of the proteins of Table A; and/or
b) a fusion protein comprising the archeal ribosomal protein L7Ae fused to an exosome membrane protein, optionally wherein the exosome membrane protein is selected from the group comprising or consisting of Lamp2b, VSVG, CD63 or any of the proteins of Table A; and/or
c) an aptamer binding protein fused to an exosome membrane protein, optionally wherein the exosome membrane protein is selected from the group comprising or consisting of Lamp2b, VSVG, CD63 or any of the proteins of Table A;

183. The chassis or engineered chassis according to paragraph 182 wherein the fusion protein further comprises a light activated dimerization protein.

184. The chassis or engineered chassis according to any of the preceding paragraphs wherein where the chassis or engineered chassis comprises a cargo that is an RNA that comprises an exosome targeting domain that is an MS2 binding stem-loop, the chassis or engineered chassis has been engineered to express a fusion protein, wherein the fusion protein comprises the bacteriophage coat protein MS2 fused to an exosome membrane protein, optionally wherein the exosome membrane protein is selected from the group comprising or consisting of Lamp2b, VSVG, CD63 or any of the proteins of Table A;
optionally wherein the fusion protein further comprises a light activated dimerization domain.

185. The chassis or engineered chassis according to any of the preceding paragraphs wherein where the chassis or engineered chassis comprises a cargo that is an RNA that comprises an exosome targeting domain that is a C/D box, the chassis or engineered chassis has been engineered to express a fusion protein, wherein the fusion protein comprises the archaeal ribosomal protein L7Ae fused to an exosome membrane protein, optionally wherein the exosome membrane protein is selected from the group comprising or consisting of Lamp2b, VSVG, CD63 or any of the proteins of Table A;
optionally wherein the fusion protein further comprises a light activated dimerization domain.

186. The chassis or engineered chassis according to any of the preceding paragraphs wherein where the chassis or engineered chassis comprises a cargo that is an RNA that comprises an aptamer, the chassis or engineered chassis has been engineered to express a fusion protein, wherein the fusion protein comprises a protein or fragment thereof capable of being bound by the aptamer fused to an exosome membrane protein, optionally wherein the exosome membrane protein is selected from the group comprising or consisting of Lamp2b, VSVG, CD63 or any of the proteins of Table A;
optionally wherein the fusion protein further comprises a light activated dimerization domain.

187. The chassis or engineered chassis according to any of the preceding paragraphs wherein where the chassis or engineered chassis comprises a cargo that is an RNA that comprises an exosome targeting domain that is an AU rich element, the producer or effector-chassis has been engineered to express a fusion protein, wherein the fusion protein is a CD9-HuR fusion protein.

188. The chassis or engineered chassis according to any of the preceding paragraphs wherein the cargo is an RNA that encodes a Cas protein, optionally a Cas9 protein.

189. The chassis or engineered chassis according to any of the preceding paragraphs wherein the progenitor, producer or effector-chassis has been engineered to express one or more sgRNAs.

190. The chassis or engineered chassis according to any of the preceding paragraphs wherein the chassis or engineered chassis comprises a nucleic acid encoding one or more cargo, optionally:
wherein the nucleic acid comprises a heterologous sequence;
wherein the cargo is a heterologous cargo; and/or
the cargo comprises one or more targeting sequences, optionally comprises an exosome targeting domain.

191. The chassis or engineered chassis according to any one of the preceding paragraphs wherein the chassis or engineered chasses comprises any one or more of a CPR, universal CPR, complex of universal CPR and tagged targeting peptide, SAPR or ePAR as defined by the preceding paragraphs, and wherein the chassis or engineered chassis endogenously expresses a cargo that is only expressed when:
a) the target binding domain of any one or more of the CPR, universal CPR, complex of universal CPR and tagged targeting peptide or SAPR binds to the target; and/or
b) the ePAR is cleaved by the protease;

optionally:
wherein the cargo is toxic to the chassis or to the subject; and/or
the cargo is expressed from the Bcl-3 mRNA untranslated regions, optionally 5′UTR.

192. The chassis or engineered chassis according to any of the preceding paragraphs where the chassis or engineered chassis comprises a cargo and wherein the cargo has been exogenously loaded into or onto the chassis or engineered chassis, optionally into the cytoplasm, into the plasma membrane, or onto the extracellular surface.

193. A nucleic acid encoding a cargo, optionally wherein the cargo is selected from:
a) a protein or peptide, optionally:
   i) an antibody or antigen binding fragment thereof, for example an antibody or antigen binding fragment thereof binds to a tumor antigen or a neoantigen;
   ii) an enzyme, such as a nuclease for example a TALEN;
   iii) a cytokine for example IL-10; or
   iv) a CRISPR associated protein, for example Cas9;
   v) a bispecific protein, for example a bispecific antibody or optionally a T-cell engager (BITE)
b) a nucleic acid, optionally an RNA, optionally an RNA selected from an mRNA, a miRNA, shRNA, and a clustered regularly interspaced short palindromic repeats (CRISPR) sequence;
optionally wherein the cargo comprises a targeting domain, optionally comprises an exosome targeting domain.

194. The nucleic acid according to paragraph 193 wherein the nucleic acid encodes the cargo in-frame with an alpha-granule localisation signal, optionally wherein the alpha-granule localisation signal is selected from PF4 of vWf.

195. The chassis or engineered chassis according to any of the preceding paragraphs where the chassis or engineered chassis comprises a nucleic acid according to paragraphs 93 or 194.

196. The chassis or engineered chassis according to any of the preceding paragraphs where the chassis or engineered chassis comprises a cargo and wherein the cargo is stored or located in the granules optionally in the alpha-granule, in the exosomes, optionally exosomes located with alpha-granules, in the cytoplasm, in the plasma membrane, and/or on the external surface of the plasma membrane.

197. The chassis or engineered chassis according to any of the preceding paragraphs where the chassis or engineered chassis comprises a cargo and the cargo is:
a therapeutic agent;
an imaging agent,
a non-therapeutic agent; and/or
a cosmetic-agent.

198. A targeted delivery system comprising a chassis or engineered chassis as defined in any of the preceding paragraphs wherein the chassis or engineered chassis is an effector-chassis that expresses one or more CPRs, universal CPRs, complexes of universal CPRs and tagged targeting peptides, SAPRs or ePARs according to any of the preceding paragraphs, optionally wherein the targeted delivery system is a therapeutic targeted delivery system or a non-therapeutic delivery system.

199. A non-thrombogenic targeted delivery system comprises an engineered chassis as defined in any of the preceding paragraphs wherein the engineered chassis is an engineered effector-chassis that expresses one or more CPRs, universal CPRs, complexes of universal CPRs and tagged targeting peptides, SAPRs or ePARs according to any of the preceding paragraphs and wherein the effector-chassis has been engineered to disrupt the thrombogenic pathway targeted delivery system is a non-thrombogenic therapeutic targeted delivery system or a non-thrombogenic non-therapeutic delivery system.

200. The targeted delivery system or the non-thrombogenic targeted delivery system of the preceding paragraphs wherein the system further comprises one or more cargo, optionally wherein the cargo comprises one or more targeting domains, optionally comprises an exosome targeting domain.

201. A chassis or engineered chassis according to any of the preceding paragraphs and/or CPR, universal CPR, complex of universal CPR and tagged targeting peptide, SAPR or ePAR according to any of the preceding paragraphs for use in medicine, optionally wherein the chassis is an effector-chassis.

201. A chassis or engineered chassis and/or CPR, universal CPR, complex of universal CPR and tagged targeting peptide, SAPR or ePAR according to any of the preceding paragraphs for use in delivering a therapeutic or imaging cargo; or treating or preventing cancer, an autoimmunity disease, genetic disease, cardiovascular disease and/or an infection, optionally wherein the chassis or engineered chassis is an effector-chassis or engineered effector-chassis.

202. A method of delivering a cargo comprising administering an effective amount of any one or more of a chassis or engineered chassis according to any of the preceding paragraphs, optionally wherein the chassis or engineered chassis comprises one or more CPRs, universal CPRs, complexes of universal CPRs and tagged targeting peptides, SAPRs or ePARs according to any of the preceding paragraphs, optionally wherein the chassis or engineered chassis is an effector-chassis or engineered effector-chassis.

203. A method of targeted cargo delivery to a target cell, tissue or site in the body wherein the method comprises administering an effective amount of any one or more of a chassis or engineered chassis according to any of the preceding paragraphs, optionally wherein the chassis or engineered chassis comprises one or more CPRs, universal CPRs, complexes of universal CPRs and tagged targeting peptides, SAPRs or ePARs according to any of the preceding paragraphs, wherein the targeting domain of the CPR, universal CPR, complex of universal CPR and tagged targeting peptide, SAPR or ePAR binds to the target cell, tissue or site in the body, optionally wherein the chassis or engineered chassis is an effector-chassis or engineered effector-chassis.

204. A non-therapeutic method of delivering cargo to a subject in need thereof.

205. A method of treatment comprising administering an effective amount of any one or more of a chassis or engineered chassis according to any of the preceding paragraphs, optionally wherein the chassis or engineered chassis comprises one or more CPRs, universal CPRs, complexes of universal CPRs and tagged targeting peptides, SAPRs or ePARs according to any of the preceding paragraphs, optionally wherein the method is for the treatment or prevention of any one or more of cancer, an autoimmunity disease, genetic disease, cardiovascular disease and/or an infection, optionally wherein the chassis or engineered chassis is an effector-chassis or engineered effector-chassis.

206. Use of any one or more of a chassis or an engineered chassis according to any of the preceding paragraphs, optionally wherein the chassis or engineered chassis comprises one or more CPRs, universal CPRs, complexes of universal CPRs and tagged targeting peptides, SAPRs or ePARs according to any of the preceding paragraphs, in the manufacture of a medicament for the treatment or prevention of disease or infection, optionally for the treatment or prevention of any one or more of cancer, an autoimmunity disease, genetic disease, cardiovascular disease and/or an infection, optionally wherein the chassis or engineered chassis is an effector-chassis or engineered effector-chassis.

207. A method of using the chassis or engineered chassis of any of the preceding paragraphs to deliver a cargo, optionally a therapeutic agent, by administering the chassis or engineered chassis to a patient in need thereof optionally wherein the chassis or engineered chassis comprises one or more CPRs, universal CPRs, complexes of universal CPRs and tagged targeting peptides, SAPRs or ePARs according to any of the preceding paragraphs, optionally wherein the chassis or engineered chassis is an effector-chassis or engineered effector-chassis.

208. A kit comprising any two or more of the following:
a) A chassis according to any one or more of the preceding paragraphs;
b) An engineered chassis according to any one or more of the preceding paragraphs; c) An engineered platelet or platelet-like membrane-bound cell fragment according to any one or more of the preceding paragraphs;
d) A therapeutic agent and/or an imaging agent;
e) A CPR, universal CPR, complex of universal CPR and tagged targeting peptide, SAPR or ePAR according to any one or more of the preceding paragraphs; and/or
f) A nucleic acid encoding a CPR, universal CPR, complex of universal CPR and tagged targeting peptide, SAPR or ePAR according to any one or more of the preceding paragraphs;
g) a nucleic acid encoding one or more cargo as defined in any one or more of the preceding paragraphs; and/or
h) one or more cargo as defined in any one or more of the preceding paragraphs.

209. A method for the targeted delivery of cargo-comprising exosomes wherein the method comprises administering a chassis or engineered chassis according to any of the preceding paragraphs to a subject in need thereof wherein:
a) the chassis or engineered chassis expresses one or more chimeric platelet receptors (CPRs), universal chimeric platelet receptors (universal CPRs), complexes of universal CPRs and tagged targeting peptides, synthetic antigen presenting receptors (SAPRs), or engineered protease activated receptors (ePARS), optionally comprises at least one CPR or universal CPR; and
b) the chassis or engineered chassis comprises a cargo that has been targeted to the exosomes by engineering of the cargo and/or chassis or engineered chassis.

210. The method according to paragraph 209 wherein the cargo is selected from any one or more of:
a) a protein or peptide—in some embodiments the protein or peptide is:
   i) an antibody or antigen binding fragment thereof, for example an antibody or antigen binding fragment thereof binds to a tumor antigen or a neoantigen;
   ii) an enzyme, such as a nuclease for example a TALEN;
   iii) a cytokine for example IL-10; or
   iv) a CRISPR associated protein, for example Cas9;
   v) a bispecific protein, for example a bispecific antibody or optionally a T-cell engager (BITE)
b) a nucleic acid—in some embodiments the nucleic acid is:
   i) an RNA, for example selected from mRNA, a miRNA, shRNA, and a clustered regularly interspaced short palindromic repeats (CRISPR) sequence; or
   ii) a DNA vector;
c) a toxin;
d) a small molecule drug, imaging agent, radionucleotide drug, radionucleotide tagged antibody, or any conjugate thereof;
e) a viral vector such as AAV;
f) a virus such as oncolytic virus;
g) agents for performing CRISPR mediated gene editing;
h) an exosome, for example an exosome pre-loaded with a second cargo; and/or
i) or a nanoparticle or nanoparticles;
   or any combination thereof.

211. The method according to paragraph 209 or 210 wherein the chassis or engineered chassis has been engineered to endogenously express a cargo that comprises an exosome targeting domain and:
A) where the cargo is a protein or peptide, the protein or peptide is a fusion protein comprising:
   i) the cargo protein or peptide; and
   ii) an exosome targeting domain, optionally wherein the exosome targeting domain is selected from the group comprising or consisting of:
      a) an exosome specific membrane protein or exosome membrane targeting portion thereof, optionally;
         a tetraspanin, for example CD63; or
         a non-tetraspanin such as PTGFRN or BASP1
      b) an exosome targeting sequence from a soluble protein, optionally the WW domain of Nedd4 ubiquitin ligases;
      c) a ubiquitin tag; and/or
      d) a tag binding domain, optionally a nanobody directed against a tag, optionally a nanobody directed against GFP; and/or
      e) a protein selected from the proteins listed in Table A;
B) where the cargo is an RNA, the exosome targeting domain is:
   a) an exosome targeting hairpin or linear motif;
   b) a viral exosome targeting RNA or exosome targeting fragment thereof;
   c) an aptamer, optionally:
      i) a MS2 binding stem-loop;
      ii) a C/D box; and/or
      iii) an AU rich element, optionally wherein the RNA is an mRNA that encodes Cas9,
optionally wherein:
   where the cargo is an RNA that comprises an MS2 binding stem-loop the chassis or engineered chassis is also engineered to express a fusion protein comprising the bacteriophage coat protein MS2 fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises a C/D box, the chassis or engineered chassis is also engineered to express a fusion protein comprising the archael L7 ribosomal L7Ae protein fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises an AU rich element the chassis or engineered chassis has been engineered to express a fusion protein that is a CD9-HuR fusion protein, optionally wherein the fusion protein further comprises a light activated dimerization protein;
   where the cargo is an RNA that comprises an aptamer the chassis or engineered chassis is engineered to express a fusion protein comprising an aptamer binding protein (that binds to the aptamer present in the RNA) fused to to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein.

210. The method according to paragraph 209 wherein chassis or engineered chassis has been exogenously loaded with a cargo that comprises an exosome targeting domain, optionally wherein:
A) where the cargo is a protein or peptide, the protein or peptide is a fusion protein comprising:
   i) the cargo protein or peptide; and
   ii) an exosome targeting domain, optionally wherein the exosome targeting domain is selected from the group comprising or consisting of:
      a) an exosome specific membrane protein or exosome membrane targeting portion thereof, optionally;
         a tetraspanin, for example CD63; or
         a non-tetraspanin such as PTGFRN or BASP1
      b) an exosome targeting sequence from a soluble protein, optionally the WW domain of Nedd4 ubiquitin ligases;
      c) a ubiquitin tag; and/or
      d) a tag binding domain, optionally a nanobody directed against a tag, optionally a nanobody directed against GFP; and/or
      e) a protein selected from the proteins listed in Table A;
B) where the cargo is an RNA, the exosome targeting domain is:
   a) an exosome targeting hairpin or linear motif;
   b) a viral exosome targeting RNA or exosome targeting fragment thereof;
   c) an aptamer, optionally:
      i) a MS2 binding stem-loop;
      ii) a C/D box; and/or
      iii) an AU rich element, optionally wherein the RNA is an mRNA that encodes Cas9,
optionally wherein:
   where the cargo is an RNA that comprises an MS2 binding stem-loop the chassis or engineered chassis is also engineered to express a fusion protein comprising the bacteriophage coat protein MS2 fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises a C/D box, the chassis or engineered chassis is also engineered to express a fusion protein comprising the archael L7 ribosomal L7Ae protein fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises an AU rich element the chassis or engineered chassis has been engineered to express a fusion protein that is a CD9-HuR fusion protein, optionally wherein the fusion protein further comprises a light activated dimerization protein;
   where the cargo is an RNA that comprises an aptamer the chassis or engineered chassis is engineered to express a fusion protein comprising an aptamer binding protein (that binds to the aptamer present in the RNA) fused to to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein.

211. An engineered chassis according to any of the preceding paragraphs for use in the targeted delivery of therapeutic cargo-comprising exosomes to a subject in need thereof for use in medicine, optionally for use in treating or preventing cancer, an autoimmunity disease, genetic disease, cardiovascular disease and/or an infection,
wherein the engineered chassis is an engineered effector-chassis that comprises a cargo that has been targeted to the exosomes by engineering of the cargo and/or chassis or engineered chassis.

212. The engineered chassis for use according to paragraph 211 wherein the cargo is selected from any one or more of:
a) a protein or peptide—in some embodiments the protein or peptide is:
   i) an antibody or antigen binding fragment thereof, for example an antibody or antigen binding fragment thereof binds to a tumor antigen or a neoantigen;
   ii) an enzyme, such as a nuclease for example a TALEN;
   iii) a cytokine for example IL-10; or
   iv) a CRISPR associated protein, for example Cas9;
   v) a bispecific protein, for example a bispecific antibody or optionally a T-cell engager (BITE)
b) a nucleic acid—in some embodiments the nucleic acid is:
   i) an RNA, for example selected from mRNA, a miRNA, shRNA, and a clustered regularly interspaced short palindromic repeats (CRISPR) sequence; or
   ii) a DNA vector;
c) a toxin;
d) a small molecule drug, imaging agent, radionucleotide drug, radionucleotide tagged antibody, or any conjugate thereof;
e) a viral vector such as AAV;
f) a virus such as oncolytic virus;
g) agents for performing CRISPR mediated gene editing;
h) an exosome, for example an exosome pre-loaded with a second cargo; and/or
i) or a nanoparticle or nanoparticles;
   or any combination thereof.

213. The engineered chassis for use according to paragraph 211 or 212 wherein the chassis or engineered chassis has been engineered to endogenously express a cargo that comprises an exosome targeting domain and:
A) where the cargo is a protein or peptide, the protein or peptide is a fusion protein comprising:
   i) the cargo protein or peptide; and
   ii) an exosome targeting domain, optionally wherein the exosome targeting domain is selected from the group comprising or consisting of:
      a) an exosome specific membrane protein or exosome membrane targeting portion thereof, optionally;
         a tetraspanin, for example CD63; or
         a non-tetraspanin such as PTGFRN or BASP1
      b) an exosome targeting sequence from a soluble protein, optionally the WW domain of Nedd4 ubiquitin ligases;
      c) a ubiquitin tag; and/or
      d) a tag binding domain, optionally a nanobody directed against a tag, optionally a nanobody directed against GFP; and/or
      e) a protein selected from the proteins listed in Table A;
B) where the cargo is an RNA, the exosome targeting domain is:
   a) an exosome targeting hairpin or linear motif;
   b) a viral exosome targeting RNA or exosome targeting fragment thereof;
   c) an aptamer, optionally:
      i) a MS2 binding stem-loop;
      ii) a C/D box; and/or
      iii) an AU rich element, optionally wherein the RNA is an mRNA that encodes Cas9,
optionally wherein:
   where the cargo is an RNA that comprises an MS2 binding stem-loop the chassis or engineered chassis is also engineered to express a fusion protein comprising the bacteriophage coat protein MS2 fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises a C/D box, the chassis or engineered chassis is also engineered to express a fusion protein comprising the archael L7 ribosomal L7Ae protein fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises an AU rich element the chassis or engineered chassis has been engineered to express a fusion protein that is a CD9-HuR fusion protein, optionally wherein the fusion protein further comprises a light activated dimerization protein;
   where the cargo is an RNA that comprises an aptamer the chassis or engineered chassis is engineered to express a fusion protein comprising an aptamer binding protein (that binds to the aptamer present in the RNA) fused to to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein.

214. The engineered chassis for use according to paragraph 211 or 212 wherein chassis or engineered chassis has been exogenously loaded with a cargo that comprises an exosome targeting domain, optionally wherein:
A) where the cargo is a protein or peptide, the protein or peptide is a fusion protein comprising:
   i) the cargo protein or peptide; and
   ii) an exosome targeting domain, optionally wherein the exosome targeting domain is selected from the group comprising or consisting of:
      a) an exosome specific membrane protein or exosome membrane targeting portion thereof, optionally;
         a tetraspanin, for example CD63; or
         a non-tetraspanin such as PTGFRN or BASP1
      b) an exosome targeting sequence from a soluble protein, optionally the WW domain of Nedd4 ubiquitin ligases;
      c) a ubiquitin tag; and/or
      d) a tag binding domain, optionally a nanobody directed against a tag, optionally a nanobody directed against GFP; and/or
      e) a protein selected from the proteins listed in Table A;
B) where the cargo is an RNA, the exosome targeting domain is:
   a) an exosome targeting hairpin or linear motif;
   b) a viral exosome targeting RNA or exosome targeting fragment thereof;
   c) an aptamer, optionally:
      i) a MS2 binding stem-loop;
      ii) a C/D box; and/or
      iii) an AU rich element, optionally wherein the RNA is an mRNA that encodes Cas9,
optionally wherein:
   where the cargo is an RNA that comprises an MS2 binding stem-loop the chassis or engineered chassis is also engineered to express a fusion protein comprising the bacteriophage coat protein MS2 fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises a C/D box, the chassis or engineered chassis is also engineered to express a fusion protein comprising the archael L7 ribosomal L7Ae protein fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises an AU rich element the chassis or engineered chassis has been engineered to express a fusion protein that is a CD9-HuR fusion protein, optionally wherein the fusion protein further comprises a light activated dimerization protein;
   where the cargo is an RNA that comprises an aptamer the chassis or engineered chassis is engineered to express a fusion protein comprising an aptamer binding protein (that binds to the aptamer present in the RNA) fused to to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein.

215. Use of an engineered chassis according to any of the preceding paragraphs in the manufacture of a medicament for use in the targeted delivery of therapeutic cargo-comprising exosomes to a subject in need thereof for use in medicine, optionally for use in treating or preventing cancer, an autoimmunity disease, genetic disease, cardiovascular disease and/or an infection,
wherein the engineered chassis is an engineered effector-chassis that comprises a cargo that has been targeted to the exosomes by engineering of the cargo and/or chassis or engineered chassis.

216. The use according to paragraph 215 wherein the cargo is selected from any one or more of:
a) a protein or peptide—in some embodiments the protein or peptide is:
   i) an antibody or antigen binding fragment thereof, for example an antibody or antigen binding fragment thereof binds to a tumor antigen or a neoantigen;
   ii) an enzyme, such as a nuclease for example a TALEN;
   iii) a cytokine for example IL-10; or
   iv) a CRISPR associated protein, for example Cas9;
v) a bispecific protein, for example a bispecific antibody or optionally a T-cell engager (BITE)
b) a nucleic acid—in some embodiments the nucleic acid is:
   i) an RNA, for example selected from mRNA, a miRNA, shRNA, and a clustered regularly interspaced short palindromic repeats (CRISPR) sequence; or
   ii) a DNA vector;
c) a toxin;
d) a small molecule drug, imaging agent, radionucleotide drug, radionucleotide tagged antibody, or any conjugate thereof;
e) a viral vector such as AAV;
f) a virus such as oncolytic virus;
g) agents for performing CRISPR mediated gene editing;
   or any combination thereof.

217. The use according to paragraph 215 or 216 wherein the chassis or engineered chassis has been engineered to endogenously express a cargo that comprises an exosome targeting domain and:
A) where the cargo is a protein or peptide, the protein or peptide is a fusion protein comprising:
   i) the cargo protein or peptide; and
   ii) an exosome targeting domain, optionally wherein the exosome targeting domain is selected from the group comprising or consisting of:
      a) an exosome specific membrane protein or exosome membrane targeting portion thereof, optionally;
         a tetraspanin, for example CD63; or
         a non-tetraspanin such as PTGFRN or BASP1
      b) an exosome targeting sequence from a soluble protein, optionally the WW domain of Nedd4 ubiquitin ligases;
      c) a ubiquitin tag; and/or
      d) a tag binding domain, optionally a nanobody directed against a tag, optionally a nanobody directed against GFP; and/or
      e) a protein selected from the proteins listed in Table A;
B) where the cargo is an RNA, the exosome targeting domain is:
   a) an exosome targeting hairpin or linear motif;
   b) a viral exosome targeting RNA or exosome targeting fragment thereof;
   c) an aptamer, optionally:
      i) a MS2 binding stem-loop;
      ii) a C/D box; and/or
      iii) an AU rich element, optionally wherein the RNA is an mRNA that encodes Cas9,
optionally wherein:
   where the cargo is an RNA that comprises an MS2 binding stem-loop the chassis or engineered chassis is also engineered to express a fusion protein comprising the bacteriophage coat protein MS2 fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises a C/D box, the chassis or engineered chassis is also engineered to express a fusion protein comprising the archael L7 ribosomal L7Ae protein fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises an AU rich element the chassis or engineered chassis has been engineered to express a fusion protein that is a CD9-HuR fusion protein, optionally wherein the fusion protein further comprises a light activated dimerization protein;
   where the cargo is an RNA that comprises an aptamer the chassis or engineered chassis is engineered to express a fusion protein comprising an aptamer binding protein (that binds to the aptamer present in the RNA) fused to to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein.

218. The use according to paragraph 215 or 216 wherein chassis or engineered chassis has been exogenously loaded with a cargo that comprises an exosome targeting domain, optionally wherein:
A) where the cargo is a protein or peptide, the protein or peptide is a fusion protein comprising:
   i) the cargo protein or peptide; and
   ii) an exosome targeting domain, optionally wherein the exosome targeting domain is selected from the group comprising or consisting of:
      a) an exosome specific membrane protein or exosome membrane targeting portion thereof, optionally;
         a tetraspanin, for example CD63; or
         a non-tetraspanin such as PTGFRN or BASP1
      b) an exosome targeting sequence from a soluble protein, optionally the WW domain of Nedd4 ubiquitin ligases;
      c) a ubiquitin tag; and/or
      d) a tag binding domain, optionally a nanobody directed against a tag, optionally a nanobody directed against GFP; and/or
      e) a protein selected from the proteins listed in Table A;
B) where the cargo is an RNA, the exosome targeting domain is:
   a) an exosome targeting hairpin or linear motif;
   b) a viral exosome targeting RNA or exosome targeting fragment thereof;
   c) an aptamer, optionally:
      i) a MS2 binding stem-loop;
      ii) a C/D box; and/or
      iii) an AU rich element, optionally wherein the RNA is an mRNA that encodes Cas9,
optionally wherein:
   where the cargo is an RNA that comprises an MS2 binding stem-loop the chassis or engineered chassis is also engineered to express a fusion protein comprising the bacteriophage coat protein MS2 fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises a C/D box, the chassis or engineered chassis is also engineered to express a fusion protein comprising the archael L7 ribosomal L7Ae protein fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
   where the cargo is an RNA that comprises an AU rich element the chassis or engineered chassis has been engineered to express a fusion protein that is a CD9-HuR fusion protein, optionally wherein the fusion protein further comprises a light activated dimerization protein;
   where the cargo is an RNA that comprises an aptamer the chassis or engineered chassis is engineered to express a fusion protein comprising an aptamer binding protein (that binds to the aptamer present in the RNA) fused to to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein.

1. An engineered chassis, wherein the chassis has been engineered:
A)
i) to disrupt a platelet inflammatory signaling pathway;
ii) to make the engineered chassis less immunogenic; and/or
iii) to enhance or disrupt one or more base functions of the chassis, wherein the one or more or base functions are involved in the innate and/or adaptive immune response, inflammation, angiogenesis, atherosclerosis, lymphatic development and/or tumour growth; and optionally
iv) engineered to disrupt a platelet thrombogenic pathway;
and wherein
B) the chassis has been further engineered to comprise any one or more of:
i) one or more chimeric platelet receptors (CPRs), universal chimeric platelet receptors (universal CPRs), complexes of universal CPRs and tagged targeting peptides, synthetic antigen presenting receptors (SAPRs), or engineered protease activated receptors (ePARS);
ii) one or more nucleic acids that encodes one or more CPR, universal CPR, SAPR, or ePAR; and/or
iii) one or more vectors that comprises one or more nucleic acids that encodes one or CPR, universal CPR, SAPR, or ePAR;
and wherein the engineered chassis is:
a) an engineered effector-chassis and is:
a platelet that comprises TUBB1;
a platelet-like membrane-bound cell fragment that comprises TUBB1; or
an anucleate cell fragment that comprises TUBB1;
b) an engineered producer-chassis and is:
a megakaryoblast that comprises TUBB1;
a megakaryocyte that comprises TUBB1;
a megakaryocyte-like cell that comprises TUBB1;
a cancer cell line that is capable of forming:
a platelet that comprises TUBB1;
a platelet-like membrane-bound cell fragment that comprises TUBB1; and/or
an anucleate cell fragment that comprises TUBB1,
optionally wherein the cancer cell line is a MEG01 or DAMI cancer cell line; or
other immortal cell that is capable of forming:
a platelet that comprises TUBB1;
a platelet-like membrane-bound cell fragment that comprises TUBB1; and/or
an anucleate cell fragment that comprises TUBB1;
or
c) an engineered progenitor-chassis and is a myeloid stem cell; an iPSC; a cancer cell-line that is capable of producing a producer-chassis; adipocyte; adipose-derived mesenchymal stromal/stem cell line (ASCL); or other immortal cell that is capable of producing a producer-chassis.

2. The engineered chassis according to any of the preceding claims wherein the engineered chassis has been:
a) loaded with one or more cargo; and/or
b) engineered so as to provide one or more cargo.

3. The engineered chassis according to any of the preceding claims wherein the engineered chassis is an engineered progenitor-chassis that has been driven to differentiate into a producer-chassis, optionally driven to differentiate into a megakaryocyte, a megakaryocyte-like cell, optionally wherein the engineered progenitor-chassis is an engineered myeloid stem cell; an iPSC; a cancer cell-line that is capable of producing a producer-chassis; adipocyte; adipose-derived mesenchymal stromal/stem cell line (ASCL); or other immortal cell that is capable of producing a producer-chassis.

4. The engineered chassis according to any of the preceding claims wherein the engineered chassis is a producer-chassis, wherein the producer chassis is a megakaryoblast; a megakaryocyte; a megakaryocyte-like cell; a cancer cell line that is capable of forming a platelet, a platelet-like membrane-bound cell fragment or an anucleate cell fragment optionally a MEG01 or DAMI cancer cell line; or other immortal cell that is capable of forming a platelet, a platelet-like membrane-bound cell fragment or an anucleate cell fragment,
and wherein the megakaryoblast; megakaryocyte; megakaryocyte-like cell; cancer cell line that is capable of forming a platelet, a platelet-like membrane-bound cell fragment or an anucleate cell fragment optionally MEG01 or DAMI; or other immortal cell that is capable of forming a platelet, a platelet-like membrane-bound cell fragment or an anucleate cell fragment can produce pseudopodal extensions.

5. The engineered chassis according to any of the preceding claims wherein the engineered chassis is an engineered effector-chassis and wherein the effector-chassis is a platelet, a platelet-like membrane-bound cell fragment, or anucleate cell fragment and wherein the platelet, a platelet-like membrane-bound cell fragment or anucleate cell fragment has been produced by fragmentation of an engineered producer-chassis, wherein the producer chassis is a megakaryoblast; a megakaryocyte; a megakaryocyte-like cell; a cancer cell line that is capable of forming a platelet, a platelet-like membrane-bound cell fragment or an anucleate cell fragment optionally a MEG01 or DAMI cancer cell line; or other immortal cell that is capable of forming a platelet, a platelet-like membrane-bound cell fragment or an anucleate cell fragment.

6. The engineered chassis according to any of the preceding claims wherein the engineered chassis is an engineered effector-chassis, wherein the engineered effector-chassis is a platelet, platelet-like membrane-bound cell fragment, or anucleate cell fragment and wherein the platelet, a platelet-like membrane-bound cell fragment or anucleate cell fragment does not aggregate in a platelet aggregation assay.

7. The engineered chassis according to any of the preceding claims wherein the engineered chassis is an engineered progenitor or producer-chassis and wherein the one or more nucleic acids are expressed from a position within the genomic nucleic acid of the engineered progenitor or producer-chassis, optionally wherein:
1) a nucleic acid encoding a first CPR, universal CPR, complex of universal CPR and tagged targeting peptide, SAPR, or ePAR has been introduced into a first allele of a first locus, and/or a nucleic acid encoding a first CPR, universal CPR, complex of universal CPR and tagged targeting peptide, SAPR, or ePAR has been be introduced to a second allele of a first locus; and/or
2) a nucleic acid encoding a first CPR, universal CPR, complex of universal CPR and tagged targeting peptide, SAPR, or ePAR has been introduced into a first allele of a first locus and a second nucleic acid encoding a second CPR, universal CPR, complex of universal CPR and tagged targeting peptide, SAPR, or ePAR has been introduced in to a first allele of a second locus; and/or
3) a nucleic acid encoding a first CPR, universal CPR, complex of universal CPR and tagged targeting peptide, SAPR, or ePAR has been introduced into a first allele of a first locus and a second nucleic acid encoding a second CPR, universal CPR, complex of universal CPR and tagged targeting peptide, SAPR, or ePAR has been introduced into a second allele of the first locus.

8. The engineered chassis according to any of claims 1-6 wherein the one or more nucleic acids are expressed episomally.

9. The engineered chassis according to any of the preceding claims wherein the engineered chassis has been engineered so as to have inhibited expression from the beta 2 microglobulin gene, optionally wherein the beta 2 microglobulin gene has been knocked out or deleted.

10. The engineered chassis according to any of the preceding claims wherein the chassis has been engineered to have disrupted expression from one or more HLA genes.

11. The engineered chassis according to claim 10 wherein the chassis has been engineered to have disrupted expression from any one or more of HLA-A, HLA-B and/or HLA-C, optionally wherein expression of HLA-A and HLA-B has been entirely disrupted but wherein expression of HLA-C has been partially disrupted, optionally wherein expression from both alleles of HLA-A and HLA-B have been disrupted but wherein expression from only one allele of HLA-C has been disrupted.

12. The engineered chassis according to any of the preceding claims wherein the chassis has been engineered to overexpress any one or more of the HLA class Ib genes, optionally any one or more of HLA-G, HLA-E, CD47 and PD-1.

13. The engineered chassis according to any of the preceding claims wherein the chassis has been engineered to overexpress any one or more of HLA-G, HLA-E, CD47 and PD-L1 and optionally engineered to disrupt expression from the Beta 2 microglobulin gene.

14. The engineered chassis according to any of the preceding claims wherein the chassis has been engineered to downregulate or inhibit expression of TGFb and/or GARP and/or CD40L.

15. The engineered chassis according to any of claims 2-14 wherein the cargo is selected from any one or more of:
a) a protein or peptide, optionally wherein the protein or peptide is:
i) an antibody or antigen binding fragment thereof, optionally an antibody or antigen binding fragment thereof binds to a tumor antigen or a neoantigen;
ii) an enzyme, such as a nuclease for example a TALEN;
iii) a cytokine for example IL-10; or
iv) a CRISPR associated protein, for example Cas9;
v) a bispecific protein, for example a bispecific antibody or optionally T-cell engager (BITE)
b) a nucleic acid—in some embodiments the nucleic acid is:
i) an RNA, for example selected from mRNA, a miRNA, shRNA, and a clustered regularly interspaced short palindromic repeats (CRISPR) sequence; or
ii) a DNA vector;
c) a toxin;
d) a small molecule drug, imaging agent, radionucleotide drugs, radionucleotide tagged antibodies, or conjugate any thereof;
e) a viral vector such as AAV;
f) a virus such as oncolytic virus;
g) agents for performing CRISPR mediated gene editing;
h) an exosome, for example an exosome pre-loaded with a second cargo; and/or
i) or a nanoparticle or nanoparticles;
or any combination thereof.

16. The engineered chassis according to any of claims 2-15 wherein the cargo is an endogenously expressed cargo,
optionally wherein the endogenously expressed cargo is any one or more of:
a) a protein or peptide—in some embodiments the protein or peptide is:
i) an antibody or antigen binding fragment thereof, for example an antibody or antigen binding fragment thereof binds to a tumor antigen or a neoantigen;
ii) an enzyme, such as a nuclease for example a TALEN;
iii) a cytokine for example IL-10; or
iv) a CRISPR associated protein, for example Cas9;
v) a bispecific protein, for example a bispecific antibody or optionally a T-cell engager (BiTE)
b) a nucleic acid—in some embodiments the nucleic acid is:
i) an RNA, for example selected from mRNA, a miRNA, shRNA, and a clustered regularly interspaced short palindromic repeats (CRISPR) sequence.

17. The engineered chassis according to any of claims 2 to 16 wherein the cargo is exogenously loaded into the chassis, optionally wherein exogenously loaded cargo is any one or more of:
a) a protein or peptide—in some embodiments the protein or peptide is:
i) an antibody or antigen binding fragment thereof, for example an antibody or antigen binding fragment thereof binds to a tumor antigen or a neoantigen;
ii) an enzyme, such as a nuclease for example a TALEN;
iii) a cytokine for example IL-10; or
iv) a CRISPR associated protein, for example Cas9;
v) a bispecific protein, for example a bispecific antibody or optionally a T-cell engager (BiTE)
b) a nucleic acid—in some embodiments the nucleic acid is:
i) an RNA, for example selected from mRNA, a miRNA, shRNA, and a clustered regularly interspaced short palindromic repeats (CRISPR) sequence;
c) a toxin;
d) a small molecule drug, imaging agent, radionucleotide drug, radionucleotide tagged antibody, or any conjugate thereof;
e) a viral vector such as AAV;
f) a virus such as oncolytic virus;
g) agents for performing CRISPR mediated gene editing;
h) an exosome, for example an exosome pre-loaded with a second cargo; and/or
i) or a nanoparticle or nanoparticles;
or any combination thereof.

18. The engineered chassis according to any of claims 2-17 wherein where the chassis comprises a cargo, the cargo comprises an exosome targeting domain, optionally wherein the cargo is a protein or peptide that is a fusion protein comprising:
a) the cargo protein or peptide; and
b) an exosome targeting domain, optionally wherein the exosome targeting domain is selected from the group comprising or consisting of:
i) an exosome specific membrane protein or exosome membrane targeting portion thereof, for example:
a tetraspanin, for example CD63; or
a non-tetraspanin such as PTGFRN or BASP1 ii) an exosome targeting sequence from a soluble protein, optionally the WW domain of Nedd4 ubiquitin ligases;
iii) a ubiquitin tag; and/or
iv) a tag binding domain, optionally a nanobody directed against a tag, optionally a nanobody directed against GFP; and/or
v) a protein selected from the proteins listed in Table A.
or wherein the cargo is an RNA, and the exosome targeting domain is:
a) an exosome targeting hairpin;
b) a viral exosome targeting RNA or exosome targeting fragment thereof;
c) an aptamer, optionally:
i) a MS2 binding stem-loop;
ii) a C/D box; and/or
iii) an AU rich element, optionally wherein the RNA is an mRNA that encodes Cas9.

19. The engineered chassis according to any of the preceding claims wherein the chassis has been engineered to:
a) have disrupted function of MHC Class 1 genes or proteins;
b) have disrupted expression from the β2 microglobulin gene, optionally to knock out the 02 microglobulin gene;
c) have disrupted expression from one or more HLA genes;
d) have disrupted expression from any one or more of HLA-A, HLA-B and/or HLA-C, optionally wherein expression of HLA-A and HLA-B has been entirely disrupted but wherein expression of HLA-C has been partially disrupted, optionally wherein expression from both alleles of HLA-A and HLA-B have been disrupted but wherein expression from only one allele of HLA-C has been disrupted;
e) overexpress any one or more of the HLA class Ib genes, optionally any one or more of HLA-G, HLA-E, CD47 and PD-1;
f) overexpress any one or more of HLA-G, HLA-E, CD47 and PD-L1 and optionally been engineered to have disrupted expression from the Beta 2 microglobulin gene; and/or
g) overexpress one or more immunomodulatory genes, optionally wherein the one or more immunomodulatory genes is selected from the group comprising CD47 and PD-1;
h) eliminate one or more genes or gene products for which the product(s) could negatively affect the potency of a cargo;
i) tune up or down the innate/adaptive response;
j) reduce inflammation, angiogenesis, atherosclerosis, lymphatic development and tumour growth;
k) have disrupted expression of one or more genes encoding adhesive proteins and/or cargo entities which are likely to indirectly counter the biological action of the engineered cargo, potentially leading to a greater net therapeutic effect;
l) downregulate or inhibit expression of TGFb and/or GARP and/or CD40L;
n) downregulate or inhibit expression of any one or more of CD36, NOD2, SRB1, TLR1, TLR2, TLR3, TLR4, TLR6, TLR9, CD40L, CD93 (C1qRp), C3aR, CD88 (C5aR), CD89 (FcαR1), CD23 (FcεR1), CD32 (FcγRIIa), MHC class1, CD191 (CCR1), CD193 (CCR3), CD194 (CCR4), CD184 (CXCR4), CX3CR1, CD102 (ICAM-2), JAM-C/JAM-3, CD62P (P-selectin), CD31 (PECAM-1), CD150 (SLAMF1), CCL2, CCL3, CCL5, CXCL1, CXCL12, CXCL4/PF4, CXCL5, CXCL8, NAP2 (CXCL7), IL-1β
o) disrupt or inhibit expression of TGFb and/or GARP;
q) disrupt or inhibit expression of any one or more of Siglec-7, Siglec-9, Siglec-11 or TGFβ
s) disrupt or inhibit expression of any one or more of GPIb/V/IX and GPVI (GP6), ITGA2B, CLEC2, integrins s aIIbb3, a2b1, a5b1 and a6b1, GPVI and ITGA2B;
t) disrupt or inhibit expression of any one or more of Par1, Par4, P2Y12, GPIb/V/IX, the Thromboxane receptor (TBXA2R), P2Y1, P2X1 and integrin aIIbb3 or from the group consisting of Par, Par4 and P2Y12;
u) disrupt or inhibit expression of any one or more of Cox1, Cox2, HPS, prothrombin, PDGF, EGF, von Willebrand Factor and thromboxane-A synthase (TBXAS1);
v) synthesise a protein or RNA of interest in response to activation of the platelet or platelet-like membrane-bound cell fragment, optionally wherein the protein or RNA of interest is expressed from the BCL-3 mRNA untranslated regions, optionally 5′UTR;
z) express one or more cargo proteins or cargo RNAs, optionally wherein the cargo protein or cargo RNA comprises an alpha-granule targeting signal, optionally comprises a platelet factor 4 (PF4) or von Willebrand factor (vWf);
aa) express at least two CPRs, universal CPRs, complexes of universal CPRs and tagged targeting peptides, SAPRs or ePARs, optionally express at least 3, 4, 5, 6, 7, 8, 9 or at least 10 different CPRs, universal CPRs, complexes of universal CPRs and tagged targeting peptides, SAPRs or ePARs;
bb) express at least two CPRs, universal CPRs, complexes of universal CPRs and tagged targeting peptides, SAPRs or ePARs, and wherein the target binding domain of the at least two CPRs, universal CPRs, complexes of universal CPRs and tagged targeting peptides, SAPRs or ePARs are directed towards different targets;
cc) express at least two CPRs, universal CPRs, complexes of universal CPRs and tagged targeting peptides, SAPRs or ePARs, and wherein the target binding domain of the at least two CPRs, universal CPRs, complexes of universal CPRs and tagged targeting peptides, SAPRs or ePARs are directed towards different targets, and wherein:
i) the platelet modulation domain of a first CPR, universal CPR, complex of universal CPR and tagged targeting peptide, SAPR or ePAR is a platelet activation domain optionally a degranulation triggering domain optionally an ITAM containing domain, and wherein the platelet modulation domain of a second CPR, universal CPR, complex of universal CPR and tagged targeting peptide, SAPR or ePAR is a platelet inhibition domain, optionally is a domain that prevents triggering of platelet degranulation, optionally is an ITAM containing domain;
ii) the platelet modulation domain of a first CPR, universal CPR, complex of universal CPR and tagged targeting peptide, SAPR or ePAR is a platelet activation domain optionally a degranulation triggering domain optionally an ITAM containing domain, and wherein the platelet modulation domain of a second CPR, universal CPR, complex of universal CPR and tagged targeting peptide, SAPR or ePAR is a platelet activation domain optionally a degranulation triggering domain optionally an ITAM containing domain;
dd) express at least two CPRs, universal CPRs, complexes of universal CPRs and tagged targeting peptides, SAPRs or ePARs that operate together to form a logic circuit;
ee) express one or more cargo, optionally wherein the cargo is selected from the group comprising:
a) a protein or peptide—optionally wherein the protein or peptide is:
i) an antibody or antigen binding fragment thereof, for example an antibody or antigen binding fragment thereof binds to a tumor antigen or a neoantigen;
ii) an enzyme, such as a nuclease for example a TALEN;
iii) a cytokine for example IL-10; or
iv) a CRISPR associated protein, for example Cas9;
v) a bispecific protein, for example a bispecific antibody or optionally a T-cell engager (BiTE);
vi) a fusion protein comprising an exosome targeting domain, optionally wherein the fusion protein comprises:
a) the cargo protein or peptide; and
b) an exosome targeting domain, optionally wherein the exosome targeting domain is selected from the group comprising or consisting of:
i) an exosome specific membrane protein or exosome membrane targeting portion thereof, for example:
a tetraspanin, for example CD63; or
a non-tetraspanin such as PTGFRN or BASP1
ii) an exosome targeting sequence from a soluble protein, optionally the WW domain of Nedd4 ubiquitin ligases;
iii) a ubiquitin tag; and/or
iv) a tag binding domain, optionally a nanobody directed against a tag, optionally a nanobody directed against GFP.
b) a nucleic acid, optionally wherein the nucleic acid is:
i) an RNA, for example selected from mRNA, a miRNA, shRNA, and a clustered regularly interspaced short palindromic repeats (CRISPR) sequence; and/or
ii) an RNA that comprises an exosome targeting domain, optionally wherein the exosome targeting domain is selected from the group comprising or consisting of:
a) an exosome targeting hairpin or linear motif;
b) a viral exosome targeting RNA or exosome targeting fragment thereof;
iii) an RNA that comprises an aptamer domain, optionally wherein the aptamer domain is selected from:
a) a MS2 binding stem-loop;
b) a C/D box; and/or
c) an AU rich element, optionally wherein the RNA is an mRNA that encodes Cas9;
ff) express a fusion protein wherein the fusion protein comprises:
i) the bacteriophage coat protein MS2 fused to an exosome membrane protein, optionally wherein the exosome membrane protein is selected from the group comprising or consisting of Lamp2b, VSVG, CD63; and/or
ii) the archaeal ribosomal protein L7Ae fused to an exosome membrane protein, optionally wherein the exosome membrane protein is selected from the group comprising or consisting of Lamp2b, VSVG, CD63; and/or
iii) a CD9-HuR fusion protein;
optionally wherein the fusion protein further comprises a light activated dimerization protein;
gg) translate one or more cargo from an mRNA only upon binding of one or more CPRs, universal CPRs, complexes of universal CPRs and tagged targeting peptides, SAPRs or ePARs to the target, optionally wherein the cargo is selected from the group comprising:
a) a protein or peptide, optionally
i) an antibody or antigen binding fragment thereof, for example an antibody or antigen binding fragment thereof binds to a tumor antigen or a neoantigen;
ii) an enzyme, such as a nuclease for example a TALEN;
iii) a cytokine for example IL-10; or
iv) a CRISPR associated protein, for example Cas9;
v) a bispecific protein, for example a bispecific antibody or optionally a T-cell engager (BiTE)
b) a nucleic acid—in some embodiments the nucleic acid is:
i) an RNA, for example selected from mRNA, a miRNA, shRNA, and a clustered regularly interspaced short palindromic repeats (CRISPR) sequence,
optionally wherein the cargo is expressed from the Bcl-3 mRNA untranslated regions, optionally 5′UTR.

20. The engineered chassis of any one of the preceding claims wherein the CPR comprises:
a) an intracellular domain that is a platelet modulation domain; and
b) a heterologous target binding domain that recognizes and binds a target, optionally wherein when the CPR is present in a platelet membrane, after binding of the target to the target binding domain the platelet modulation domain is activated.

21. The engineered chassis of any of the preceding claims, wherein the CPR platelet modulation domain is a platelet activation domain, optionally an ITAM comprising domain, optionally a platelet ITAM comprising domain, optionally is domain that has at least 75%, 80%, 85%, 90%, 92%, 94%, 96%, 98% or 100% sequence identity to an ITAM comprising domain or to a platelet ITAM comprising domain.

22. The engineered chassis of any of the preceding claims, wherein when the CPR is present in the membrane of a platelet, when activated, the platelet activation domain:
a) results in degranulation of the platelet;
b) results in the release of contents from the platelet;
c) results in the presence of intraplatelet contents on the plasma membrane of the platelet;
d) results in the release of extracellular vesicles via blebbing from the plasma membrane;
and/or a small molecule drug, imaging agent, radionucleotide drugs, radionucleotide tagged antibodies, or conjugate any thereof;
e) results in a change of shape of the platelet from a biconcave disk to fully spread cell fragments; and/or
f) results in an influx of calcium into the platelet.

23. The engineered chassis of any of the preceding claims wherein the universal CPR comprises:
a) an intracellular domain that is a platelet modulation domain; and
b) a heterologous tag binding domain optionally wherein the heterologous tag binding domain binds to a tag present on a tagged targeting peptide, wherein the tagged targeting peptide comprises the tag and a target binding domain, and
wherein when the Universal CPR is located in a platelet plasma membrane, binding of the targeting peptide to the universal CPR in the absence of simultaneous binding of the target binding domain to the target is not sufficient to activate the platelet modulation domain.

24. The engineered chassis of any of the preceding claims wherein the SAPR comprises a heterologous target binding domain wherein the target binding domain comprises:
a) an extracellular domain comprising:
i) the MHC-1 protein or fragment thereof, or a protein or fragment thereof that has at least 75%, 80%, 85%, 90%, 92%, 94%, 96%, 98% or 100% sequence identity to a human MHC-1 protein or fragment thereof; or
ii) the MHC-2 protein or fragment thereof or a protein or fragment thereof that has at least 75%, 80%, 85%, 90%, 92%, 94%, 96%, 98% or 100% sequence identity to a human MHC-2 protein or fragment thereof; and
b) an intracellular platelet modulation domain,
wherein said:
MHC-1 protein or fragment thereof or a protein or fragment thereof that has at least 75%, 80%, 85%, 90%, 92%, 94%, 96%, 98% or 100% sequence identity to a human MHC-1 protein or fragment thereof; or
MHC-2 protein or fragment thereof or a protein or fragment thereof that has at least 75%, 80%, 85%, 90%, 92%, 94%, 96%, 98% or 100% sequence identity to a human MHC-2 protein or fragment thereof;
is able to bind to a T Cell Receptor (TCR).

25. The engineered chassis of any of the preceding claims wherein the protease recognition site of the ePAR is engineered to be cleaved by a protease that is not the protease that cleaves the native recognition site, optionally wherein, when present in the plasma membrane of a platelet, cleavage of the protease recognition site results in:
a) degranulation of the platelet;
b) the release of contents from the platelet;
c) the presence of intracellular contents on the plasma membrane of the platelet;
d) the release of extracellular vesicles via blebbing from the plasma membrane; and/or
e) a change of shape of the platelet from a biconcave disk to fully spread cell fragments.

26. A targeted delivery system comprising an engineered chassis as defined in any of the preceding claims wherein the engineered chassis is an engineered effector-chassis, optionally wherein the targeted delivery system is a therapeutic targeted delivery system or a non-therapeutic delivery system, optionally wherein the system further comprises one or more cargo, optionally wherein the cargo comprises one or more targeting domains, optionally comprises an exosome targeting domain.

27. An engineered chassis according to any of the preceding claims for use in medicine.

28. An engineered chassis according to any of the preceding claims for use in delivering a therapeutic or imaging cargo; or treating or preventing cancer, an autoimmunity disease, genetic disease, cardiovascular disease and/or an infection, wherein the engineered chassis is an engineered effector-chassis.

29. A method of delivering a cargo comprising administering an effective amount of an engineered chassis or targeted delivery system according to any of the preceding claims wherein the engineered chassis is an engineered effector-chassis.

30. A method of targeted cargo delivery to a target cell, tissue or site in the body wherein the method comprises administering an effective amount of any one or more of an engineered chassis according to any of the preceding claims, wherein the targeting domain of the CPR, universal CPR, complex of universal CPR and tagged targeting peptide, SAPR or ePAR binds to the target cell, tissue or site in the body, wherein the engineered chassis is an engineered effector-chassis.

31. A non-therapeutic method of delivering cargo to a subject in need thereof herein the method comprises administering an effective amount of any one or more of an engineered chassis according to any of the preceding claims, wherein the targeting domain of the CPR, universal CPR, complex of universal CPR and tagged targeting peptide, SAPR or ePAR binds to the target cell, tissue or site in the body, wherein the engineered chassis is an engineered effector-chassis.

32. A method of treatment comprising administering an effective amount of an engineered chassis according to any of the preceding claims, optionally wherein the method is for the treatment or prevention of any one or more of cancer, an autoimmunity disease, genetic disease, cardiovascular disease and/or an infection, wherein the engineered chassis is an engineered effector-chassis.

33. Use of an engineered chassis according to any of the preceding claims in the manufacture of a medicament for the treatment or prevention of disease or infection, optionally for the treatment or prevention of any one or more of cancer, genetic disease, cardiovascular disease an autoimmunity disease, and/or an infection.

34. A method of using the chassis or engineered chassis of any of the preceding claims to deliver a cargo, optionally a therapeutic agent, by administering the engineered chassis to a patient in need thereof.

35. A kit comprising:
a) An engineered producer chassis according to any one or more of the preceding claims;
b) An engineered effector chassis according to any one or more of the preceding claims;
c) An engineered progenitor chassis according to any one or more of the preceding claims;
d) A therapeutic agent and/or an imaging agent and/or an exosome, optionally an exosome pre-loaded with a second cargo;
and/or
e) a nucleic acid encoding one or more cargo as defined in any one or more of the preceding claims; and/or
f) one or more cargo as defined in any one or more of the preceding claims.

36. An engineered chassis according to any of the preceding claims for use in the targeted delivery of therapeutic cargo-comprising exosomes to a subject in need thereof for use in medicine, optionally for use in treating or preventing cancer, an autoimmunity disease, genetic disease, cardiovascular disease and/or an infection,
wherein the engineered chassis is an engineered effector-chassis that comprises a cargo that has been targeted to the exosomes by engineering of the cargo and/or chassis or engineered chassis.

37. The engineered chassis for use according to claim 36 wherein the cargo is selected from any one or more of:
a) a protein or peptide—in some embodiments the protein or peptide is:
i) an antibody or antigen binding fragment thereof, for example an antibody or antigen binding fragment thereof binds to a tumor antigen or a neoantigen;
ii) an enzyme, such as a nuclease for example a TALEN;
iii) a cytokine for example IL-10; or
iv) a CRISPR associated protein, for example Cas9;
v) a bispecific protein, for example a bispecific antibody or optionally a T-cell engager (BITE)
b) a nucleic acid—in some embodiments the nucleic acid is:
i) an RNA, for example selected from mRNA, a miRNA, shRNA, and a clustered regularly interspaced short palindromic repeats (CRISPR) sequence; or
ii) a DNA vector;
c) a toxin;
d) a small molecule drug, imaging agent, radionucleotide drug, radionucleotide tagged antibody, or any conjugate thereof;
e) a viral vector such as AAV;
f) a virus such as oncolytic virus;
g) agents for performing CRISPR mediated gene editing;
h) an exosome, optionally an exosome pre-loaded with a second cargo; and/or
i) or a nanoparticle or nanoparticles;
or any combination thereof.

38. The engineered chassis for use according to claim 36 or 37 wherein the chassis or engineered chassis has been engineered to endogenously express a cargo that comprises an exosome targeting domain and:
A) where the cargo is a protein or peptide, the protein or peptide is a fusion protein comprising:
i) the cargo protein or peptide; and
ii) an exosome targeting domain, optionally wherein the exosome targeting domain is selected from the group comprising or consisting of:
a) an exosome specific membrane protein or exosome membrane targeting portion thereof, optionally;
a tetraspanin, for example CD63; or
a non-tetraspanin such as PTGFRN or BASP1
b) an exosome targeting sequence from a soluble protein, optionally the WW domain of Nedd4 ubiquitin ligases;
c) a ubiquitin tag; and/or
d) a tag binding domain, optionally a nanobody directed against a tag, optionally a nanobody directed against GFP; and/or
e) a protein selected from the proteins listed in Table A;
B) where the cargo is an RNA, the exosome targeting domain is:
a) an exosome targeting hairpin or linear motif;
b) a viral exosome targeting RNA or exosome targeting fragment thereof;
c) an aptamer, optionally:
i) a MS2 binding stem-loop;
ii) a C/D box; and/or
iii) an AU rich element, optionally wherein the RNA is an mRNA that encodes Cas9,
optionally wherein:
where the cargo is an RNA that comprises an MS2 binding stem-loop the chassis or engineered chassis is also engineered to express a fusion protein comprising the bacteriophage coat protein MS2 fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
where the cargo is an RNA that comprises a C/D box, the chassis or engineered chassis is also engineered to express a fusion protein comprising the archael L7 ribosomal L7Ae protein fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
where the cargo is an RNA that comprises an AU rich element the chassis or engineered chassis has been engineered to express a fusion protein that is a CD9-HuR fusion protein, optionally wherein the fusion protein further comprises a light activated dimerization protein;
where the cargo is an RNA that comprises an aptamer the chassis or engineered chassis is engineered to express a fusion protein comprising an aptamer binding protein (that binds to the aptamer present in the RNA) fused to to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein.

39. The engineered chassis for use according to claim 36 or 37 wherein chassis or engineered chassis has been exogenously loaded with a cargo that comprises an exosome targeting domain, optionally wherein:
A) where the cargo is a protein or peptide, the protein or peptide is a fusion protein comprising:
i) the cargo protein or peptide; and
ii) an exosome targeting domain, optionally wherein the exosome targeting domain is selected from the group comprising or consisting of:
a) an exosome specific membrane protein or exosome membrane targeting portion thereof, optionally;
a tetraspanin, for example CD63; or
a non-tetraspanin such as PTGFRN or BASP1
b) an exosome targeting sequence from a soluble protein, optionally the WW domain of Nedd4 ubiquitin ligases;
c) a ubiquitin tag; and/or
d) a tag binding domain, optionally a nanobody directed against a tag, optionally a nanobody directed against GFP; and/or
e) a protein selected from the proteins listed in Table A;
B) where the cargo is an RNA, the exosome targeting domain is:
a) an exosome targeting hairpin or linear motif;
b) a viral exosome targeting RNA or exosome targeting fragment thereof;
c) an aptamer, optionally:
i) a MS2 binding stem-loop;
ii) a C/D box; and/or
iii) an AU rich element, optionally wherein the RNA is an mRNA that encodes Cas9,
optionally wherein:
where the cargo is an RNA that comprises an MS2 binding stem-loop the chassis or engineered chassis is also engineered to express a fusion protein comprising the bacteriophage coat protein MS2 fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
where the cargo is an RNA that comprises a C/D box, the chassis or engineered chassis is also engineered to express a fusion protein comprising the archaeal L7 ribosomal L7Ae protein fused to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein;
where the cargo is an RNA that comprises an AU rich element the chassis or engineered chassis has been engineered to express a fusion protein that is a CD9-HuR fusion protein, optionally wherein the fusion protein further comprises a light activated dimerization protein;
where the cargo is an RNA that comprises an aptamer the chassis or engineered chassis is engineered to express a fusion protein comprising an aptamer binding protein (that binds to the aptamer present in the RNA) fused to to an exosome membrane protein, optionally wherein the exosome membrane protein is selected from the group comprising or consisting of Lamp2b, VSVG, CD63 or any of the proteins of Table A, optionally wherein the fusion protein further comprises a light activated dimerization protein.
